(19) United States
(12) Patent Application Publication
(10) Pub. No.: US 2026/0272925 A1
(43) Pub. Date: Sep. 17, 2026

(54) CDK2 MODULATORS, COMPOSITIONS, AND METHODS OF USE THEREOF

(71) Applicant: Type6 Therapeutics, Inc., Nicasio, CA (US)

(72) Inventors: Gert Kiss, Nicasio, CA (US); Alexander J. Bridges, Saline, MI (US)

(21) Appl. No.: 19/167,343

(22) PCT Filed: Mar. 19, 2024
(86) PCT No.: PCT/US2024/020597

§ 371 Date: Sep. 19, 2025

Related U.S. Application Data
(60) Provisional application No. 63/491,477, filed on Mar. 21, 2023.

(51) Int. Cl. A61K 31/506

(52) U.S. Cl. CPC A61K 31/506

(57) ABSTRACT

Provided herein, in some embodiments, are uses of compounds of Formula (I), or pharmaceutically acceptable salts thereof, or compositions comprising said compounds or pharmaceutically acceptable salts thereof and a pharmaceutically acceptable excipient, in methods of modulating cyclin-dependent kinase 2 (CDK2) activity, wherein the compound of Formula (I) is; wherein R¹, R^{2A}, R^{2B}, R³, R⁴, R⁵, R⁶, X¹, X² and n are as defined herein.

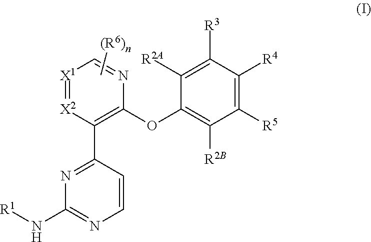

### CROSS REFERENCE

This application claims priority to and the benefit of U.S. Provisional Patent Application No. 63/491,477 filed Mar. 21, 2023, which is incorporated herein by reference in its entirety

### SUMMARY

Disclosed herein, in some embodiments, are uses of compounds of Formula (I), or pharmaceutically acceptable salts thereof, or compositions comprising said compounds or pharmaceutically acceptable salts thereof and a pharmaceutically acceptable excipient, in methods of modulating cyclin-dependent kinase 2 (CDK2) activity, wherein the compound of Formula (I) is:

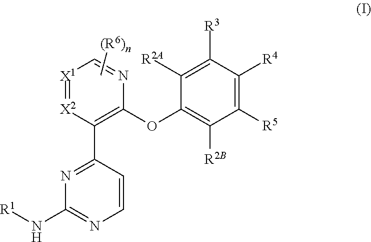

wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1c}, or —(C₁₋₆ alkylene)-NR^{1a}R^{1b}; wherein the C₃₋₁₂ cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the C₁₋₆ alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}—, C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen. C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{e}, —S(O)₂R^{e}, —S(O)(═NH)R^{e}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, —N(R^{f})S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{e} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(R^{f1})C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(RU)S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³; each R is independently hydrogen or C₁₋₆ alkyl;
each R^{f2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
X¹ and X² are both C, optionally substituted with R⁶.
or X¹ is C, optionally substituted with R⁶, and X² is N;
or X¹ and X² taken together are a sulfur atom;
or a pharmaceutical composition comprising: (a) a compound of Formula (I) or a pharmaceutically acceptable salt thereof, and (b) a pharmaceutically acceptable excipient.

Also provided herein, in some embodiments, is a method of inhibiting CDK2 activity in a subject in need thereof, comprising administering to the subject an effective amount of a compound of Formula (I), or a pharmaceutically acceptable salt thereof, or a pharmaceutical composition comprising: (a) a compound of Formula (I) or a pharmaceutically acceptable salt thereof, and (b) a pharmaceutically acceptable excipient.

Also provided herein, in some embodiments, is a method of treating, preventing, and/or reducing the risk or severity of a CDK2-related disease or disorder (e.g., diseases or disorders mediated by unwanted CDK2 activity, diseases or disorders involving over-expression or amplification of cyclin E, diseases or disorders involving over-expression or amplification of cyclin A2, or diseases or disorders that are otherwise treatable with a CDK2 inhibitor) in a subject in need thereof, comprising administering to the subject an effective amount of a compound of Formula (I), or a pharmaceutically acceptable salt thereof, or a pharmaceutical composition comprising: (a) a compound of Formula (I) or a pharmaceutically acceptable salt thereof, and (b) a pharmaceutically acceptable excipient, or a pharmaceutically acceptable salt thereof, or a pharmaceutical composition comprising: (a) a compound of Formula (I) or a pharmaceutically acceptable salt thereof, and (b) a pharmaceutically acceptable excipient.

Also provided herein, in some embodiments, is a method of killing a cell over-expressing or amplifying cyclin E, comprising contacting the cell with an effective amount of a compound of Formula (I), or a pharmaceutically acceptable salt thereof, or a pharmaceutical composition comprising: (a) a compound of Formula (I) or a pharmaceutically acceptable salt thereof, and (b) a pharmaceutically acceptable excipient.

Also provided herein, in some embodiments, is a method of killing a cell over-expressing or amplifying cyclin A, comprising contacting the cell with an effective amount of a compound of Formula (I), or a pharmaceutically acceptable salt thereof, or a pharmaceutical composition comprising: (a) a compound of Formula (I) or a pharmaceutically acceptable salt thereof, and (b) a pharmaceutically acceptable excipient.

Other objects and advantages will become apparent to those skilled in the art from a consideration of the ensuing Detailed Description, Examples. and Claims.

### DETAILED DESCRIPTION

Cyclin-dependent kinases (CDKs) are important cellular enzymes that perform essential functions in regulating cell division and proliferation. The CDK catalytic units are activated by binding to regulatory subunits, known as cyclins, followed by phosphorylation. The cyclins are divided into four general classes, G₁, G₂/S, S and M cyclins, whose expression levels vary at different points in the cell cycle.

In particular, CDK2 becomes active when cyclin E or cyclin A binds at the active site of the kinase. The cyclin E/CDK2 complex plays an important role in regulation of the G₁/S transition, histone biosynthesis, and centrosome duplication. Progressive phosphorylation of retinoblastoma (Rb) by cyclin D/Cdk4/6 and cyclin E/CDK2 releases the G₁ transcription factor, E2F, and promotes S-phase entry. Activation of cyclin A/CDK2 during early S-phase promotes phosphorylation of endogenous substrates that permit DNA replication and inactivation of E2F, for S-phase completion.

CDK2 is critical to the abnormal growth processes of certain disease or disorders such as cancer (e.g., cancer mediated by CDK2 activity). The CCNE1 gene which produces cyclin E is frequently overexpressed in various cancers, causing the cells to become dependent on Cdk2 and cyclin E. In addition, abnormal expression of cyclin A2 is associated with chromosomal instability and tumor proliferation. However, Cyclin A also binds CDK1 during the late S/G2 phase. CDK1 is an essential cyclin dependent kinase in the cell cycle, and its inhibition could lead to undesired toxicity. Thus, there is a need for methods of treatment, prevention, and reduction of risk or severity of diseases or disorders mediated by unwanted CDK2 activity, comprising use of CDK2 inhibitors that are selective for CDK2 over CDK1.

In addition, CDK2 has been implicated in regulatory events in autoimmune diseases such as pemphigus vulgaris, inflammation, and neurodegenerative diseases or disorders. Thus, inhibitors of CDK2 are viable candidates for the methods of treating, preventing, or reducing the risk or severity of certain autoimmune diseases or disorders, inflammatory diseases or disorders, and neurodegenerative diseases or disorders.

The present disclosure provides, in some embodiments, uses of compounds of Formula (I), or pharmaceutically acceptable salts thereof, or compositions comprising said compounds or pharmaceutically acceptable salts thereof and a pharmaceutically acceptable excipient in methods of modulating cyclin-dependent kinase 2 (CDK2) activity, wherein the compound of Formula (I) is:

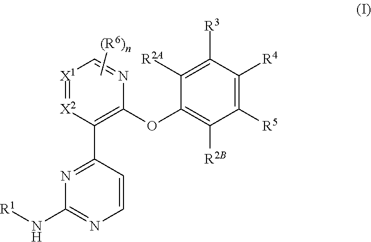

wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1e}, or —(C₁₋₆ alkylene)-NR^{1a}R^{1b}; wherein the C₃₋₁₂ cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the C₁₋₆ alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}—, C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen, C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{e}, —S(O)₂R^{e}, —S(O)(═NH)R^{e}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, —N(Rt)S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{o} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(R^{f1})C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkenyl, C₃₋₆ cycloalkyl, C₆₋₁₀ aryl, 5- to 10-membered heteroaryl and 3- to 8-membered heterocyclyl of R^{a} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen. C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(R^{f2})S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl,
X¹ and X² are both C, optionally substituted with R⁶;
or X¹ is C, optionally substituted with R⁶, and X2 is N;
or X¹ and X² taken together are a sulfur atom.

In some embodiments, the compound of Formula (I) or a pharmaceutically acceptable salt thereof is other than Compound Nos. 1x-12x of Table 1. In some embodiments, R^{8B} is other than hydrogen or optionally substituted phenyl. In some embodiments, R⁴ is other than —NHC(O)NH—R^{8B} wherein R^{8B} is optionally substituted phenyl or optionally substituted alkyl.

TABLE 1

| No. | Name |
|---|---|
| 1x | Benzamide, 3-[[3-[2-[[2-(diethylamino)ethyl]amino]-4-pyrimidinyl]-2- |
| | pyridinyl]oxy]-4-methyl-N-[2-methyl-3-(1-methylethyl)phenyl]- |
| 2x | 2-Pyrrolidinone, 1-[3-[[4-[2-(4-amino-2-methylphenoxy)-3-pyridinyl]-2- |
| | pyrimidinyl]amino]propyl] - |
| 3x | 4-Morpholinepropanamine, N-[4-[2-(4-amino-2-methylphenoxy)-3-pyridinyl]-2- |
| | pyrimidinyl]- |
| 4x | Urea, N-(3-fluoro-4-methylphenyl)-N′-[4-[[3-[2-[[3-(4-morpholinyl)propyl]amino]- |
| | 4-pyrimidinyl]-2-pyridinyl]oxy]phenyl]- |
| 5x | Urea, N-(2,3-dihydro-1H-inden-5-yl)-N′-[4-[[3-[2-[[3-(4- |
| | morpholinyl)propyl]amino]-4-pyrimidinyl]-2-pyridinyl]oxy]phenyl]- |
| 6x | Urea, N-(4-chlorophenyl)-N′-[4-[[3-[2-[[3-(4-morpholinyl)propyl]amino]-4- |
| | pyrimidinyl]-2-pyridinyl]oxy]phenyl]- |
| 7x | Urea, N-[3-methyl-4-[[3-[2-[[3-(2-oxo-1-pyrrolidinyl)propyl]amino]-4-pyrimidinyl]- |
| | 2-pyridinyl]oxy]phenyl]-N′-[3-(trifluoromethyl)phenyl]- |
| 8x | Urea, N-(5-chloro-2-methoxyphenyl)-N′-[3-methyl-4-[[3-[2-[[3-(2-oxo-1- |
| | pyrrolidinyl)propyl]amino]-4-pyrimidinyl]-2-pyridinyl]oxy]phenyl]- |
| 9x | Urea, N-(5-chloro-2-methoxyphenyl)-N′-[3-methyl-4-[[3-[2-[[3-(4- |
| | morpholinyl)propyl] amino]-4-pyrimidinyl]-2-pyridinyl]oxy] phenyl]- |
| 10x | Benzamide, 3-[[3-[2-[[2-(diethylamino)ethyl]amino]-4-pyrimidinyl]-2- |
| | pyridinyl]oxy]-4-methyl-N-[2-(4-morpholinyl)-5-(trifluoromethyl)phenyl]- |
| 11x | Benzamide, N-(5-cyclohexyl-2-methoxyphenyl)-3-[[3-[2-[[2- |
| | (diethylamino)ethyl]amino]-4-pyrimidinyl]-2-pyridinyl]oxy]-4-methyl- |
| 12x | Benzamide, 3-[[3-[2-[[2-(diethylamino)ethyl]amino]-4-pyrimidinyl]-2- |
| | pyridinyl]oxy]-4-methyl-N-[3-(1-methylethyl)phenyl]- |

In some embodiments, the compound is of the Formula (I), or a pharmaceutically acceptable salt thereof, wherein R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl). In some embodiments, R^{2A} and R^{2B} are independently hydrogen, halogen, cyano, C₁₋₆ alkyl, or —O(C₁₋₆ alkyl). In some embodiments. R^{2A} and R^{2B} are independently H, F, Cl, or C₁-C₆ alkyl (e.g., methyl). In some embodiments, one of R^{2A} and R^{2B} is hydrogen and the other one of R^{2A} and R^{2B} is hydrogen, fluoro or methyl. In some embodiments. R^{2A} and R^{2B} are each hydrogen. In some embodiments, R^{2A} and R^{2B} are each fluoro. In some embodiments, R^{2A} and R^{2B} are each methyl. In some embodiments, R^{2A} is H, F or methyl, and R^{2B} is H, F, Cl or —CH₃.

In some embodiments, the compound is of the Formula (I) or a pharmaceutically acceptable salt thereof, wherein R³ is hydrogen, halogen, cyano, C₁₋₆ alkyl. C₁₋₆ haloalkyl, C₃₋₆ cycloalkyl, —O(C₁₋₆ alkyl), or —O(C₁₋₆ haloalkyl). In some embodiments, R³ is hydrogen, halogen, cyano, C₁₋₆ alkyl, C₁₋₆ haloalkyl, or —O(C₁₋₆ alkyl). In some embodiments, R³ is H, F, Cl, —CN, C₁₋₆ alkyl (e.g., methyl), or C₁₋₆ haloalkyl (e.g., trifluoromethyl). In some embodiments, R³ is H, F, Cl, —CN, —CH₃, or —CF₃.

In some embodiments, the compound is of the Formula (I) or a pharmaceutically acceptable salt thereof, wherein 0, 1, 2, or 3; and each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl). In some embodiments, n is 0. (i.e., R⁶ is absent). In some embodiments, n is 1 and R⁶ is F, Cl, —CN, —NO₂, —O—(C₁₋₆ alkyl) or C₁₋₆ alkyl. In some embodiments, n is 1 and R⁶ is F, Cl, —CN, —O—(C₁₋₆ alkyl) (e.g., —OCH₃), or C₁₋₆ alkyl (e.g., methyl and ethyl).

It is intended and understood that each and every variation of R^{2A}, R^{2B}, R³, R⁶ and n described for the Formula (I) can be combined, the same as if each and every combination is specifically and individually described. For example, in some embodiments, R^{2A} and R^{2B} are independently H, F, Cl or C₁-C₆ alkyl (e.g., methyl); R³ is H, F, Cl, —CN, C₁₋₆ alkyl (e.g., methyl), or C₁₋₆ haloalkyl (e.g., trifluoromethyl); n is 0 and R⁶ is absent. In some embodiments, R^{2A} and R^{2B} are independently H, F, Cl or methyl; R³ is H, F, Cl, —CN, methyl, or trifluoromethyl; and n is 0.

In some embodiments, the compound is of the Formula (I) or a pharmaceutically acceptable salt thereof, wherein R¹ is hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NRSC(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}, —C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁵ are optionally substituted with 1, 2, 3, 4 or 5 substituents independently selected from R¹⁰. In some embodiments, R⁵ is H, F, Cl, —CN, or C₁₋₆ alkyl optionally substituted with 1, 2, 3, 4 or 5 substituents independently selected from R¹⁰. In some embodiments, R⁵ is H, F, Cl, —CN, C₁₋₆ alkyl optionally substituted with 1, 2, 3, 4 or 5 substituents independently selected from R¹⁰, —NR⁸SO₂R⁹, —NR^{8A}R^{8B}, or —C(O)N(R⁸)SO₂R⁹. In some embodiments, R⁵ is H, F, Cl, —CN, C₁₋₆ alkyl (e.g., —CH₃) or C₁₋₆ haloalkyl (e.g., —CF₃). In some embodiments, R⁵ is H, F, Cl, —CN, —CH₃, or —CF₃. In some embodiments, R⁵ is NHC(O)NHR⁹, where R⁹ is as described herein.

In some embodiments, R⁵ is C₁₋₆ alkyl substituted with 1, 2, 3, 4 or 5 substituents independently selected from R¹⁰, —NR⁸SO₂R⁹, —NR^{8A}R^{8B}, or —C(O)N(R⁸)SO₂R⁹;

In some of these embodiments, R⁴ is H, F, Cl, —CN, C₁₋₆ alkyl (e.g., —CH₃) or C₁₋₆ haloalkyl (e.g., —CF₃). In some of these embodiments, R⁴ is H, F, Cl, —CN, —CH₃, or —CF₃. In some embodiments, R⁴ is H, and R⁵ is —(C₁₋₆ alkylene)-N(R^{f})C(O)R^{a}; C₁₋₆ alkyl substituted with 1, 2, 3, 4 or 5 substituents independently selected from R¹⁰, —NH—SO₂R⁹, —NH—R^{8B}, or —C(O)NH—SO₂R⁹. In a specific variation. R⁵ is —CH₂NHC(O)-(cyclopropyl), —NHCH₂CH(OH)CF₃ or —C(O)NHSO₂-(2-chlorophenyl). In another variation, R⁵ is —NHSO₂R⁹, and R⁹ is C₁₋₆ alkyl optionally substituted with 1, 2, 3, 4 or 5 substituents independently selected from R¹⁰, or C₆₋₁₀ aryl optionally substituted with 1, 2, 3, 4 or 5 substituents independently selected from R¹⁰. In another specific variation. R⁵ is —NHSO₂-(2-chlorophenyl), —NHSO₂—CH₂CH₂CH₃ or —NHSO₂—CH₂-(phenyl).

In some embodiments, the compound is of the Formula (I) or a pharmaceutically acceptable salt thereof, wherein R⁴ is hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}, —C(O)R⁷, —C(O)OR^{7A}, —SO₂R⁹, —NR⁸S(O)(═NR^{8C})R⁹, or —SO₂NR^{8A}R^{8B}, wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰. In some embodiments, R⁴ is hydrogen, halogen, cyano, nitro. C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}, —C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰; wherein R⁸ and R^{8A} are independently hydrogen or C₁-C₆ alkyl, and R^{8B} is independently C₁₋₆ alkyl, C₂₋₆ alkenyl, C₃₋₈ cycloalkyl, or 3- to 12-membered heterocyclyl; wherein the C₁₋₆ alkyl, C₂₋₆ alkenyl, C₃₋₈ cycloalkyl, and 3- to 12-membered heterocyclyl of R^{8B} are optionally substituted with 1, 2, 3, 4 or 5 substituents independently selected from R¹⁰. In some embodiments, R⁴ is —NR⁸C(O)R⁷ or —NR⁸SO₂R⁹. In some of these embodiments, R⁵ is H, F, Cl, —CN, —CH₃, or —CF₃.

In some embodiments, R⁴ is —NHC(O)R⁷, wherein R⁷ is C₁₋₆ alkyl optionally substituted with 1, 2, 3, 4 or 5 substituents independently selected from R¹⁰, or C₃₋₈ cycloalkyl optionally substituted with 1, 2, 3, 4 or 5 substituents independently selected from R¹⁰. In some particular embodiments, R⁷ is cyclopropyl, spiro[2.2]pentyl, cyclohexylmethyl or 4-chlorobenzyl. In some embodiments, R⁷ is cyclopentyl, cyclohexyl, phenyl, 2-chlorophenyl, 3-cyanophenyl, 4-cyanophenyl, 2-methoxyphenyl, 3-methoxyphenyl, 4-methoxyphenyl, 3-(difluoromethoxy)phenyl, 3-(trifluoromethoxy)phenyl, 4-(trifluoromethoxy)phenyl, 3,5-difluorophenyl, 3-pyridyl, 1-methyl-1H-imidazol-4-yl, 1-methyl-TH-pyrazol-4-yl, benzyl, 2-fluorobenzyl, 3-fluorobenzyl, 4-fluorobenzyl, 2-cyanobenzyl, 4-cyanobenzyl, 3-chlorobenzyl, 4-chlorobenzyl, (1-methyl-1H-pyrazol-3-yl)methyl, (5-methylisoxazol-3-yl)methyl, (pyridin-2-yl)methyl, (pyridin-3-yl)methyl, (pyridin-4-yl)methyl, (6-(trifluoromethyl)pyridin-3-yl)methyl, (1-fluorocyclopropyl)methyl, cyclobutylmethyl, (2,2-difluorocyclobutyl)methyl, (3,3-difluorocyclobutyl)methyl, cyclopentylmethyl, cyclohexylmethyl, (spiro[3.3]heptan-2-yl)methyl, 2-(cyclohexyl)ethyl, 2-(2,2-difluorocyclopropyl)ethyl, n-propyl, 3-cyano-2,2-dimethylpropyl, 3,3,3-trifluoropropyl, n-butyl, 2,2-difluorobutyl, 3,3-difluorobutyl, 3,3-dimethylbutyl, 3-cyano-3-methylbutyl, or 4,4-dimethylpentyl.

In some embodiments, R⁴ is —NH—SO₂R⁹, wherein R⁹ is C₁₋₆ alkyl, C₃₋₈ cycloalkyl, C₆₋₁₀ aryl, or 5- to 14-membered heteroaryl, wherein the C₁₋₆ alkyl, C₃₋₈ cycloalkyl, C₆₋₁₀ aryl and 5- to 14-membered heteroaryl of R⁹ are independently optionally substituted with 1, 2, 3, 4 or 5 substituents independently selected from R¹⁰. In some of these embodiments, R⁹ is C₁₋₆ alkyl optionally substituted with 1, 2, 3, 4 or 5 substituents independently selected from R¹⁰. In some of these embodiments, R⁹ is C₁₋₆ haloalkyl (e.g., C₁₋₆ fluoroalkyl). In some of these embodiments, R⁹ is C₃₋₈ cycloalkyl optionally substituted with 1, 2, 3, 4 or 5 substituents independently selected from R¹⁰. In some of these embodiments, R⁹ is C₆₋₁₀ aryl optionally substituted with 1, 2, 3, 4 or 5 substituents independently selected from R¹⁰. In some of these embodiments, R⁹ is phenyl substituted with 1, 2, 3, 4 or 5 substituents independently selected from R¹⁰. In some of these embodiments, R⁹ is 5- to 14-membered heteroaryl optionally substituted with 1, 2, 3, 4 or 5 substituents independently selected from R¹⁰.

In some embodiments, R⁹ is C₁₋₆ haloalkyl (e.g., C₁₋₆ fluoroalkyl). In some embodiments, R⁹ is benzyl optionally substituted with 1, 2, 3, 4 or 5 substituents independently selected from R¹⁰. In some embodiments, R⁹ is benzyl where the phenyl ring is optionally substituted with 1, 2 or 3 substituents independently selected from R¹⁰. In some embodiments, R⁹ is benzyl optionally substituted with 1, 2, 3, 4 or 5 substituents independently selected from the group consisting of fluoro, chloro, bromo, —CH₃, —CH₂CH₃, —CH₂OH, and —CN.

In some particular embodiments, R⁹ is cyclopentyl, cyclohexyl, phenyl, 2-chlorophenyl, 3-cyanophenyl, 4-cyanophenyl, 2-methoxyphenyl, 3-methoxyphenyl, 4-methoxyphenyl, 3-(difluoromethoxy)phenyl, 3-(trifluoromethoxy)phenyl, 4-(trifluoromethoxy)phenyl, 3,5-difluorophenyl. 3-pyridyl, 1-methyl-1H-imidazol-4-yl, 1-methyl-1H-pyrazol-4-yl, benzyl, 2-fluorobenzyl, 3-fluorobenzyl, 4-fluorobenzyl, 2-cyanobenzyl, 4-cyanobenzyl, 3-chlorobenzyl, 4-chlorobenzyl, (1-methyl-1H-pyrazol-3-yl)methyl, (5-methylisoxazol-3-yl)methyl. (pyridin-2-yl)methyl, (pyridin-3-yl)methyl, (pyridin-4-yl)methyl, (6-(trifluoromethyl)pyridin-3-yl)methyl, (1-fluorocyclopropyl)methyl, cyclobutylmethyl, (2,2-difluorocyclobutyl)methyl, (3,3-difluorocyclobutyl)methyl, cyclopentylmethyl, cyclohexylmethyl, (spiro[3.3]heptan-2-yl)methyl, 2-(cyclohexyl)ethyl, 2-(2,2-difluorocyclopropyl)ethyl, n-propyl, 3-cyano-2,2-dimethylpropyl, 3,3,3-trifluoropropyl, n-butyl, 2,2-difluorobutyl, 3,3-difluorobutyl, 3,3-dimethylbutyl, 3-cyano-3-methylbutyl, or 4,4-dimethylpentyl. In some particular embodiments. R⁹ is cyclopropyl, spiro[2.2]pentyl, cyclohexylmethyl or 4-chlorobenzyl. In some embodiments, R⁹ is selected from the group consisting of benzyl, cyclopropyl, cyclopropylmethyl, cyclobutyl, cyclobutylmethyl, cyclopentanyl, cyclopentanylmethyl, cyclohexyl, cyclohexylmethyl, pyrrolidin-1-yl, piperidin-1-yl, pyridyl, pyridylmethyl, tetrahydrofuranyl, tetrahydrofuranylmethyl, and tetrahydropyranyl, tetrahydropyranylmethyl, thiazolyl, and thiazolylmethyl; each of which is optionally and independently substituted with one or more substituents selected from fluoro, chloro, bromo, —CH₃, —CH₂CH₃, —CH₂OH, and —CN.

In some embodiments, R⁴ is —NHC(O)R⁷, wherein R⁷ is cyclopropyl, spiro[2.2]pentyl, cyclohexylmethyl or 4-chlorobenzyl. In some embodiments, R⁴ is NHC(O)NHR⁹, where R⁹ is as described herein.

In some embodiments, R⁴ is —NH—SO₂R⁹, wherein R⁹ is cyclopentyl, cyclohexyl, phenyl, 2-chlorophenyl, 3-cyanophenyl, 4-cyanophenyl, 2-methoxyphenyl, 3-methoxyphenyl, 4-methoxyphenyl, 3-(difluoromethoxy)phenyl, 3-(trifluoromethoxy)phenyl, 4-(trifluoromethoxy)phenyl, 3,5-difluorophenyl, 3-pyridyl, 1-methyl-1H-imidazol-4-yl, 1-methyl-1H-pyrazol-4-yl, benzyl, 2-fluorobenzyl, 3-fluorobenzyl, 4-fluorobenzyl, 2-cyanobenzyl. 4-cyanobenzyl, 3-chlorobenzyl, 4-chlorobenzyl, (1-methyl-1H-pyrazol-3-yl)methyl, (5-methylisoxazol-3-yl)methyl. (pyridin-2-yl)methyl, (pyridin-3-yl)methyl, (pyridin-4-yl)methyl, (6-(trifluoromethyl)pyridin-3-yl)methyl, (1-fluorocyclopropyl)methyl, cyclobutylmethyl, (2,2-difluorocyclobutyl)methyl, (3,3-difluorocyclobutyl)methyl, cyclopentylmethyl, cyclohexylmethyl, (spiro[3.3]heptan-2-yl)methyl, 2-(cyclohexyl)ethyl, 2-(2,2-difluorocyclopropyl)ethyl, n-propyl, 3-cyano-2,2-dimethylpropyl, 3.3,3-trifluoropropyl, n-butyl, 2,2-difluorobutyl, 3,3-difluorobutyl, 3,3-dimethylbutyl, 3-cyano-3-methylbutyl, or 4,4-dimethylpentyl.

In some embodiments, R⁵ is cyclopropanecarboxamidomethyl, (3,3,3-trifluoro-2-hydroxy-1-propyl)amino, or 2-chlorobenzenesulfonamidocarbonyl. In some embodiments, R⁵ is —NH—SO₂R⁹, wherein R⁹ is 2-chlorophenyl, benzyl or n-propyl. In some of these embodiments, R⁴ is H.

It is intended and understood that each and every variation of R^{2A}, R^{2B}, R³, R⁶ and n, or a combination thereof, described for the Formula (I) can be combined with each and every variation of R⁴ and R⁵, or combinations thereof, described for the Formula (I), the same as if each and every combination is specifically and individually described. For example, in some embodiments, R^{2A} and R^{2B} are independently H, F, Cl or C₁-C₆ alkyl (e.g., methyl); R³ is H, F, Cl, —CN, C₁₋₆ alkyl (e.g., methyl), or C₁₋₆ haloalkyl (e.g., trifluoromethyl); n is 0; R⁴ is —NR⁸C(O)R⁷ or —NR⁸SO₂R⁹; and R⁵ is H, F, Cl, —CN, C₁₋₆ alkyl (e.g., —CH₃) or C₁₋₆ haloalkyl (e.g., —CF₃). In some embodiments, R^{2A} and R^{2B} are independently H, F, Cl or methyl; R³ is H, F, Cl, —CN, methyl, or trifluoromethyl; n is 0; R⁴ is —NR⁸C(O)R⁷ or —NR⁸SO₂R⁹; R⁵ is H, F, Cl, —CN, —CH₃, or —CF₃; R⁷ is C₁₋₆ alkyl optionally substituted with 1, 2, 3, 4 or 5 substituents independently selected from R¹⁰, or C₃₋₈ cycloalkyl optionally substituted with 1, 2, 3, 4 or 5 substituents independently selected from R¹⁰; and R⁹ is C₁₋₆ alkyl, C₃₋₈ cycloalkyl, C₆₋₁₀ aryl, or 5- to 14-membered heteroaryl, wherein the C₁₋₆ alkyl, C₃₋₈ cycloalkyl, C₆₋₁₀ aryl and 5- to 14-membered heteroaryl of R⁹ are independently optionally substituted with 1, 2, 3, 4 or 5 substituents independently selected from R¹⁰. In some embodiments, R^{2A} and R^{2B} are independently H, F, Cl or C₁-C₆ alkyl (e.g., methyl); R³ is H, F, Cl, —CN, C₁₋₆ alkyl (e.g., methyl), or C₁₋₆ haloalkyl (e.g., trifluoromethyl); n is 0; R⁴ is H, F, Cl, —CN, C₁₋₆ alkyl (e.g., —CH₃) or C₁₋₆ haloalkyl (e.g., —CF₃); and R⁵ is C₁₋₆ alkyl substituted with 1, 2, 3, 4 or 5 substituents independently selected from R¹⁰, —NR⁸SO₂R⁹, —NR^{8A}R^{8B}, or —C(O)N(R⁸)SO₂R⁹. In some embodiments, R^{2A} and R^{2B} are independently H, F, Cl or methyl; R³ is H, F, Cl, —CN, methyl, or trifluoromethyl; n is 0; R⁴ is H, and R⁵ is —(C₁₋₆ alkylene)-N(R^{f})C(O)R^{a}; C₁₋₆ alkyl substituted with 1, 2, 3, 4 or 5 substituents independently selected from R¹⁰, —NH—SO₂R⁹, —NH—R^{8B}, or —C(O)NH—SO₂R⁹.

In some embodiments, the compound is of the Formula (I) or a pharmaceutically acceptable salt thereof, wherein R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1c}, or —(C₁₋₆ alkylene)-NR^{1a}R^{1b}; wherein the C₃₋₁₂ cycloalkyl, 3-to 14-membered heterocyclyl, 5- to 14-membered heteroaryl, and C₁₋₆ alkylene of R¹ are independently optionally substituted with 1, 2, 3, 4 or 5 substituents independently selected from R¹⁰. In some embodiments, R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), or —(C₁₋₆ alkylene)-NR^{1a}R^{1b}; wherein the C₃₋₁₂ cycloalkyl, 3-to 14-membered heterocyclyl, 5- to 14-membered heteroaryl, and C₁₋₆ alkylene of R¹ are independently optionally substituted with 1, 2, 3, 4 or 5 substituents independently selected from R¹⁰. In some embodiments, R¹ is C₃₋₁₂ cycloalkyl; 3- to 14-membered heterocyclyl; —(C₁₋₆ alkylene)-(3- to 14-membered heterocyclyl), or —(C₁₋₆ alkylene)-NR^{1a}R^{1b}; wherein the C₃₋₁₂ cycloalkyl, 3- to 14-membered heterocyclyl, and C₁₋₆ alkylene of R¹ are independently optionally substituted with 1, 2, 3, 4 or 5 substituents independently selected from R¹⁰. In some embodiments, R¹ is C₃₋₁₂ cycloalkyl optionally substituted with 1, 2, 3, 4 or 5 substituents independently selected from R¹⁰. In some embodiments, R¹ is 3- to 14-membered heterocyclyl optionally substituted with 1, 2, 3, 4 or 5 substituents independently selected from R¹⁰. In some embodiments. R¹ is cyclohexyl or piperidinyl, each is independently optionally substituted with 1, 2, 3, 4 or 5 substituents independently selected from the group consisting of F, —CH₃, —OH, oxo, and —NH₂. In some of these embodiments, R¹ is selected from the group consisting of piperidin-3-yl, 5-fluoropiperidin-3-yl, 5-methylpiperidin-3-yl and 5-fluoro-5-methylpiperidin-3-yl. In some of these embodiments. R¹ is —(C₁₋₆ alkylene)-(3- to 14-membered heterocyclyl) optionally substituted with 1, 2, 3, 4 or 5 substituents independently selected from R¹⁰. In some embodiments, R¹ is —(C₁₋₆ alkylene)-NR^{1a}R^{1b}. In some of these embodiments, R^{1a} and R^{1b} are independently hydrogen or C₁₋₆ alkyl.

It is intended and understood that each and every variation of R^{2A}, R^{2B}, R³, R⁴, R⁵, R⁶ and n, or a combination thereof, described for the Formula (I) can be combined with each and every variation of R¹ described for the Formula (I), the same as if each and every combination is specifically and individually described. For example, in some embodiments, R¹ is C₃₋₁₂ cycloalkyl; 3- to 14-membered heterocyclyl; —(C₁₋₆ alkylene)-(3- to 14-membered heterocyclyl), or —(C₁₋₆ alkylene)-NR^{1a}R^{1b}; wherein the C₃₋₁₂ cycloalkyl, 3- to 14-membered heterocyclyl, and C₁₋₆ alkylene of R¹ are independently optionally substituted with 1, 2, 3, 4 or 5 substituents independently selected from R¹⁰; R^{2A} and R^{2B} are independently H, F, Cl or C₁-C₆ alkyl (e.g., methyl); R³ is H, F, Cl, —CN, C₁₋₆ alkyl (e.g., methyl), or C₁₋₆ haloalkyl (e.g., trifluoromethyl); n is 0; R⁴ is —NR⁸C(O)R⁷ or —NR⁸SO₂R⁹; and R⁵ is H, F, Cl, —CN, C₁₋₆ alkyl (e.g., —CH₃) or C₁₋₆ haloalkyl (e.g., —CF₃). In some embodiments. R¹ is piperidin-3-yl, 5-fluoropiperidin-3-yl, 5-methylpiperidin-3-yl or 5-fluoro-5-methylpiperidin-3-yl; R^{2A} and R^{2B} are independently H, F, Cl or methyl; R³ is H, F, Cl, —CN, methyl, or trifluoromethyl; n is 0; R⁴ is —NR⁸C(O)R⁷ or —NR⁸SO₂R⁹; R⁵ is H, F, Cl, —CN, —CH₃, or —CF₃; R⁷ is C₁₋₆ alkyl optionally substituted with 1, 2, 3, 4 or 5 substituents independently selected from R¹⁰, or C₃₋₈ cycloalkyl optionally substituted with 1, 2, 3, 4 or 5 substituents independently selected from R¹⁰; and R⁹ is C₁₋₆ alkyl, C₃₋₈ cycloalkyl, C₆₋₁₀ aryl, or 5- to 14-membered heteroaryl, wherein the C₁₋₆ alkyl, C₃₋₈ cycloalkyl, C₆₋₁₀ aryl and 5- to 14-membered heteroaryl of R⁹ are independently optionally substituted with 1, 2, 3, 4 or 5 substituents independently selected from R¹⁰.

In some embodiments, R¹ is C₃₋₁₂ cycloalkyl; 3- to 14-membered heterocyclyl; —(C₁₋₆ alkylene)-(3- to 14-membered heterocyclyl), or —(C₁₋₆ alkylene)-NR^{1a}R^{1b}; wherein the C₃₋₁₂ cycloalkyl, 3- to 14-membered heterocyclyl, and C₁₋₆ alkylene of R¹ are independently optionally substituted with 1, 2, 3, 4 or 5 substituents independently selected from R¹⁰; R^{2A} and R^{2B} are independently H, F, Cl or C₁-C₆ alkyl (e.g., methyl); R³ is H, F, Cl, —CN, C₁₋₆ alkyl (e.g., methyl), or C₁₋₆ haloalkyl (e.g., trifluoromethyl); n is 0; R⁴ is H, F, Cl, —CN, C₁₋₆ alkyl (e.g., —CH₃) or C₁₋₆ haloalkyl (e.g., —CF₃); and R⁵ is C₁₋₆ alkyl substituted with 1, 2, 3, 4 or 5 substituents independently selected from R¹⁰, —NR⁸SO₂R⁹, —NR^{8A}R^{8B}, or —C(O)N(R′)SO₂R⁹. In some embodiments. R¹ is piperidin-3-yl, 5-fluoropiperidin-3-yl, 5-methylpiperidin-3-yl or 5-fluoro-5-methylpiperidin-3-yl; R^{2A} and R^{2B} are independently H, F, Cl or methyl; R³ is H, F, Cl, —CN, methyl, or trifluoromethyl; n is 0; R⁴ is H, and R⁵ is —(C₁₋₆ alkylene)-N(R)C(O)R^{a}; C₁₋₆ alkyl substituted with 1, 2, 3, 4 or 5 substituents independently selected from R¹⁰, —NH—SO₂R⁹, —NH—R^{8B}, or —C(O)NH—SO₂R⁹.

In some embodiments, the compound is of the Formula (I) or a pharmaceutically acceptable salt thereof, wherein the compound is of the Formula (Ia):

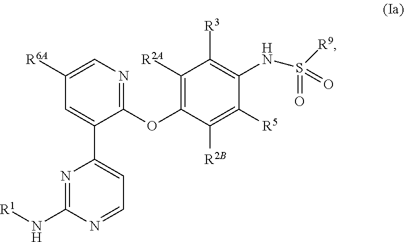

wherein R^{6A} is hydrogen or R⁶; and R¹, R^{2A}, R^{2B}, R³, R⁵, R⁶ and R⁹ are as detailed herein for Formula (I) or variations thereof. In some embodiments, each R^{6A} and R^{2A} is H, each R^{2B}, R³ and R⁵ is F, and R¹ and R⁹ are as detailed herein for Formula (I).

In some embodiments, the compound is of the Formula (I) or a pharmaceutically acceptable salt thereof, wherein the compound is of the Formula (Ib):

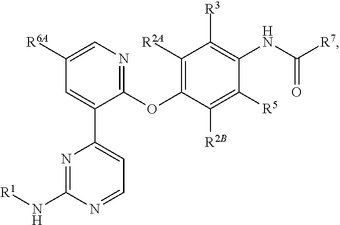

wherein R^{6A} is hydrogen or R⁶; and R¹, R^{2A}, R^{2B}, R³, R⁵, R⁶ and R⁷ are as detailed herein for Formula (I) or variations thereof. In some embodiments, each R^{6A} and R^{2A} is H, each R^{2B}, R³ and R⁵ is F, and R¹ and R⁷ are as detailed herein for Formula (I). In some embodiments, R⁷ is NHR⁹, where R⁹ is as described herein.

In some embodiments, the compound is of the Formula (I) or a pharmaceutically acceptable salt thereof, wherein the compound is of the Formula (Ic):

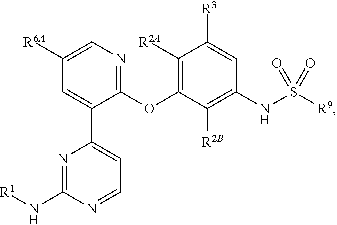

wherein R^{6A} is hydrogen or R⁶; and R¹, R^{2A}, R^{2B}, R³, R⁶ and R⁹ are as detailed herein for Formula (I).

In some embodiments, the compound is of the Formula (I) or a pharmaceutically acceptable salt thereof, wherein the compound is of the Formula (Id):

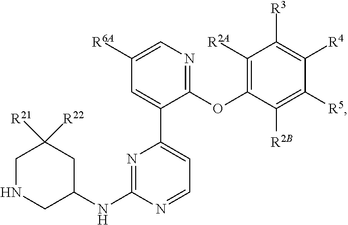

wherein R^{6A} is hydrogen or R⁶; R²¹ and R²² are independently H, F, —CH₃ or —NH₂; and R^{2A}, R^{2B}, R³. R⁴, R⁵ and R⁶ are as detailed herein for Formula (I) or variations thereof. In some embodiments, each R^{6A} and R^{2A} is H, each R^{2B}, R³ and R⁵ is F, and R⁴ is as detailed herein for Formula (I).

In some embodiments, the compound is of the Formula (Id-1), (Id-2), (Id-3), (Id-4), (Id-5), (Id-6) or (Id-7):

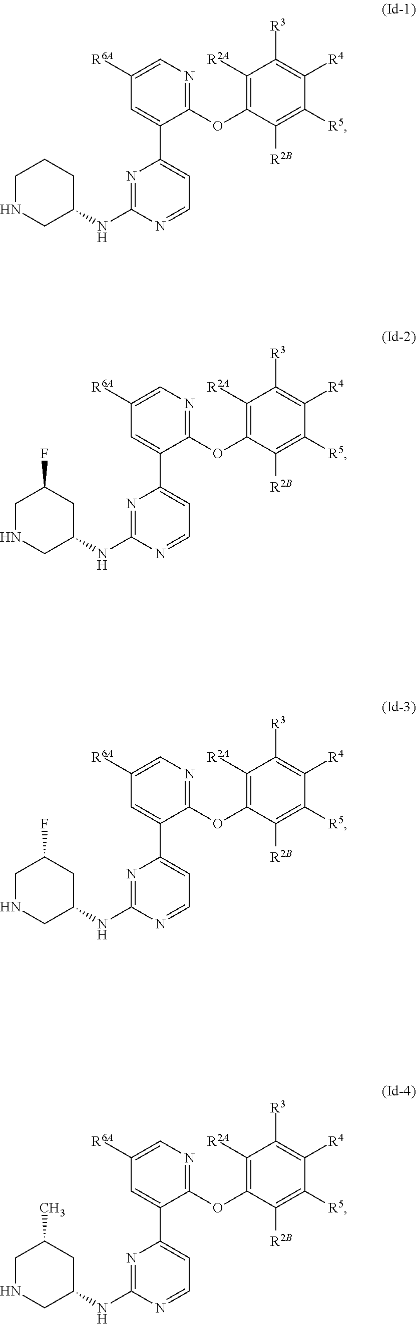

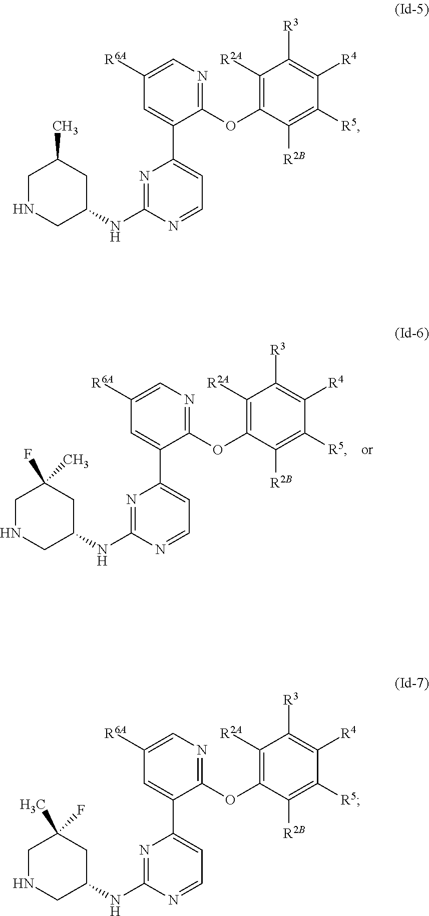

or a pharmaceutically acceptable salt thereof, wherein R^{6A} is hydrogen or R⁶; and R^{2A}, R^{2B}, R³, R⁴, R⁵ and R⁶ are as detailed herein for Formula (I) or variations thereof. In some embodiments, each R^{6A} and R^{2A} is H, each R^{2B}, R³ and R⁵ is F, and R⁴ is as detailed herein for Formula (I).

In some embodiments, the compound is of the Formula (I) or a pharmaceutically acceptable salt thereof, wherein the compound is of the Formula (Ie):

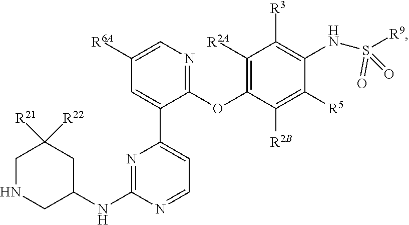

wherein R^{6A} is hydrogen or R⁶; R²¹ and R²² are independently H, F, —CH₃ or —NH₂; and R^{2A}, R^{2B}, R², R⁵, R⁶ and R⁹ are as detailed herein for Formula (I) or variations thereof. In some embodiments, each R^{6A} and R^{2A} is H, each R^{2B}, R³ and R⁵ is F, and R⁹ is as detailed herein for Formula (I).

In some embodiments, the compound is of the Formula (Ie-1). (Ie-2), (Ie-3), (Ie-4), (Ie-5). (Ie-6) or (Ie-7):

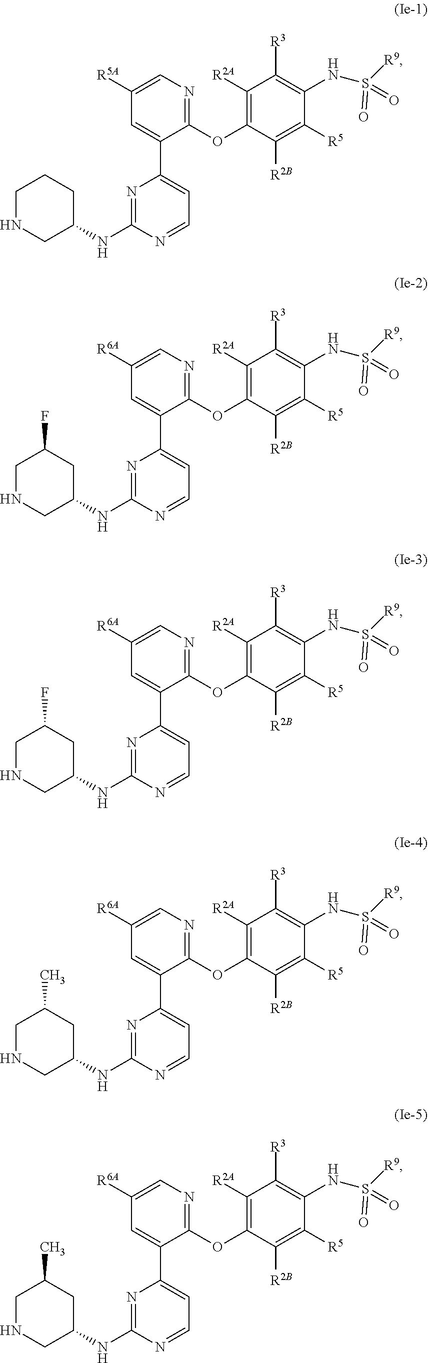

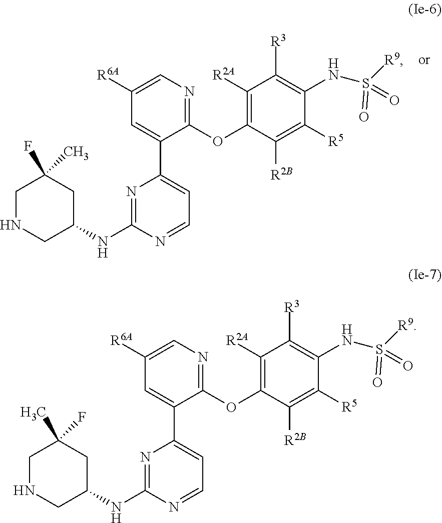

or a pharmaceutically acceptable salt thereof, wherein R^{6A} is hydrogen or R⁶; and R^{2A}, R^{2B}, R³, R⁵, R⁶ and R⁹ are as detailed herein for Formula (I). In some embodiments, each R^{6A} and R^{2A} is H, each R^{2B}, R³ and R⁵ is F, and R⁹ is as detailed herein for Formula (I).

In some embodiments of the compound or pharmaceutically acceptable salt thereof of the Formula (Ia), (Ib), (Ic), (Id), (Id-1), (Id-2), (Id-3), (Id-4), (Id-5), (Id-6), (Id-7), (Ie), (Ie-1), (Ie-2), (Ie-3), (Ie-4), (Ie-5), (Ie-6) or (Ie-7), or variations thereof, R^{6A} is H.

In some embodiments of the compound or pharmaceutically acceptable salt thereof of the Formula (I), or variations thereof where applicable, or a salt (e.g., a pharmaceutically acceptable salt) thereof, each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{e}, —S(O)₂R^{e}, —S(O)(═NH)R^{e}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, or —N(R^{f})S(O)₂NR^{c}R^{d}; wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹.

In some embodiments, R¹⁰ is independently oxo; C₁₋₆ alkyl optionally substituted with 1, 2, 3 or 4 substituents independently selected from R¹¹; 5- to 10-membered heteroaryl optionally substituted with 1, 2, 3 or 4 substituents independently selected from R¹¹; halogen, cyano, —OR^{b}, —NR^{c}R^{d}, —N(R)C(O)R^{a}, or —N(R^{f})S(O)₂R^{e}.

In some embodiments, R¹⁰ is independently oxo, halogen, cyano, C₁₋₆ alkyl optionally substituted with 1, 2, 3 or 4 substituents independently selected from R¹¹, or —OR^{b}.

In some embodiments, R¹⁰ is independently —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, or —N(R^{f})S(O)₂NR^{c}R^{d}.

In some embodiments, R¹⁰ is independently oxo. —OR^{b}, —OC(O)R^{a}, —OC(O)NR^{c}R^{d}, —SR^{b}, —S(O)R^{e}, —S(O)₂R^{e}, or —S(O)₂NR^{c}R^{d}.

In some embodiments, each R¹⁰ is independently C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}; wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹.

In some embodiments, each R¹⁰ is independently C₁₋₆ alkyl, C₂₋₆ alkenyl, C₂₋₆ alkynyl, each optionally substituted with 1, 2, 3 or 4 substituents independently selected from R¹¹.

In some embodiments, R¹⁰ is C₁₋₆ alkyl optionally substituted with 1, 2, 3 or 4 substituents independently selected from R¹¹. In some embodiments, R¹⁰ is 3- to 12-membered heterocyclyl optionally substituted with 1, 2, 3 or 4 substituents independently selected from R¹¹.

In some embodiments, R¹⁰ is halogen, cyano, —NR^{c}R^{d}, —C(O)NR^{c}R^{d}, —OR^{b}, —S(O)₂R^{e}, C₁₋₆ haloalkyl, —(C₁₋₆ alkylene)-OH, or —(C₁₋₆ alkylene)-OH.

In some embodiments, R¹⁰ is hydroxyl, cyano, halogen, —CHF₂, —CF₃, —NH₂, —NH(C₁₋₆ alkyl), —N(C₁₋₆ alkyl)₂, —O(C₁₋₆ alkyl), —SO₂(C₁₋₆ alkyl), —S(O)₂NR^{c}R^{d}, —C(O)NR^{c}R^{d}, or —N(R^{f})C(O)R^{a}.

In some embodiments, each R^{a} is independently hydrogen, C₁₋₆ alkyl. C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹. In some embodiments, R^{a} is independently hydrogen or C₁₋₆ alkyl. In some embodiments, R^{a} is independently C₁₋₆ alkyl (e.g., methyl).

In some embodiments, each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹. In some embodiments, R^{b} is independently hydrogen or C₁₋₆ alkyl. In some embodiments, R^{b} is independently C₁₋₆ haloalkyl (e.g., —CF₃).

In some embodiments, each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹; or R^{c} and R^{d} are taken together with the nitrogen atom to which they are attached to form a 4- to 12-membered heterocyclyl optionally substituted with 1, 2, 3 or 4 substituents independently selected from R¹¹. In some embodiments, each R^{c} and R^{d} is independently hydrogen or C₁₋₆ alkyl.

In some embodiments, each R^{e} is independently C₁₋₆ alkyl, C₃₋₈ cycloalkyl, C₆-10 aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{e} are each optionally substituted with 1, 2, 3 or 4 substituents independently selected from R¹¹. In some embodiments, R^{e} is independently C₁₋₆ alkyl. In some embodiments, R^{e} is phenyl optionally substituted with 1, 2, 3 or 4 substituents independently selected from R¹¹.

In some embodiments, each R^{f} is independently hydrogen or C₁₋₆ alkyl. In some embodiments, R^{f} is hydrogen.

In some embodiments, each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(Rⁿ)C(O)R^{a1}, —N(R^{f1})C(O)OR^{b1}, —N(R^{f1})C(O)NR^{c}R^{d1}, —N(R^{f1})S(O)₂R^{e1}, or —N(R^{f1})S(O)₂NR^{c1}R^{d1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹².

In some embodiments, each R¹¹ is independently oxo, C₁₋₆ alkyl, C₃₋₆ cycloalkyl, 3- to 8-membered heterocyclyl, halogen, cyano, or —OR^{b1}; wherein the C₁₋₆ alkyl, C₃₋₆ cycloalkyl, and 3- to 14-membered heterocyclyl of R¹¹ are each optionally substituted with 1, 2, 3 or 4 substituents independently selected from R¹².

In some embodiments, R¹¹ is C₁₋₆ alkyl optionally substituted with 1, 2, 3 or 4 substituents independently selected from R¹². In some embodiments. R¹¹ is 3- to 8-membered heterocyclyl optionally substituted with 1, 2, 3 or 4 substituents independently selected from R¹².

In some embodiments, R¹¹ is halogen, cyano, —NR^{c1}R^{d1}, —C(O)NR^{c1}R^{d1}, —OR^{b1}, —S(O)₂R^{e1}, C₁₋₆ haloalkyl, —(C₁₋₆ alkylene)-OH, or —(C₁₋₆ alkylene)-OH.

In some embodiments, R¹¹ is hydroxyl, cyano, halogen, —CHF₂, —CF₃, —NH₂, —NH(C₁₋₆ alkyl), —N(C₁₋₆ alkyl)₂, —O(C₁₋₆ alkyl), —SO₂(C₁₋₆ alkyl), —S(O)₂NR^{c1}R^{d1}, —C(O)NR^{c1}R^{d1}, or —N(R^{f1})C(O)R^{a1}.

In some embodiments, R¹¹ is halogen, cyano, —O(C₁₋₆ alkyl), —O(C₁₋₆ alkylene)-NH₂, or —(C₁₋₆ alkylene)-OH.

In some embodiments, each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹².

In some embodiments, each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹². In some embodiments, R^{b1} is independently hydrogen or C₁₋₆ alkyl.

In some embodiments, each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²; or R^{c1} and R^{d1} are taken together with the nitrogen atom to which they are attached to form a 4- to 8-membered heterocyclyl optionally substituted with 1, 2, 3 or 4 substituents independently selected from R¹². In some embodiments, each R^{c1} and R^{d1} is independently hydrogen or C₁₋₆ alkyl.

In some embodiments, each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹². In some embodiments. R^{e1} is independently C₁₋₆ alkyl.

In some embodiments, each R^{f1} is independently hydrogen or C₁₋₆ alkyl. In some embodiments, Rⁿ is hydrogen.

In some embodiments, each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f1})C(O)NR^{c2}R^{d2}, —N(R^{f2})S(O)₂R^{e2}, or —N(R^{f2})S(O)₂NR^{c2}R^{d2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³.

In some embodiments, each R¹² is independently oxo, halogen, cyano, —OR^{b2}, or C₁₋₆ alkyl optionally substituted with 1, 2, 3 or 4 substituents independently selected from R¹³. In some embodiments, each R¹² is independently oxo, halogen, cyano, or hydroxyl.

In some embodiments, R¹² is C₁₋₆ alkyl optionally substituted with 1, 2, 3 or 4 substituents independently selected from R¹³.

In some embodiments, R¹² is oxo, hydroxyl, C₁₋₆ alkyl, or —O(C₁₋₆ alkyl).

In some embodiments, each R^{a2} is independently hydrogen, C₁₋₆ alkyl, C₃₋₆ cycloalkyl, Ce aryl, 5- to 6-membered heteroaryl or 3- to 6-membered heterocyclyl; wherein the C₁₋₆ alkyl, C₃₋₆ cycloalkyl, Ce aryl, 5- to 6-membered heteroaryl and 3- to 6-membered heterocyclyl of R^{a2} are each optionally substituted with 1, 2, 3 or 4 substituents independently selected from R¹³. In some embodiments, R^{a2} is independently hydrogen or C₁₋₆ alkyl.

In some embodiments, each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³. In some embodiments, R^{b2} is hydrogen.

In some embodiments, each R^{c2} and R^{d2} is independently hydrogen. C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³; or R^{c2} and R^{d2} are taken together with the nitrogen atom to which they are attached to form a 4- to 6-membered heterocyclyl optionally substituted with 1, 2, 3 or 4 substituents independently selected from R¹³. In some embodiments, each R^{c2} and R^{d2} is independently hydrogen or C₁₋₆ alkyl.

In some embodiments, each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³. In some embodiments, R^{e2} is independently C₁₋₆ alkyl.

In some embodiments, each R^{o} is independently hydrogen or C₁₋₆ alkyl. In some embodiments, R^{c} is hydrogen.

In some embodiments, each R¹³ is independently oxo, halogen, hydroxyl, —O(C₁₋₆ alkyl), cyano, C₁₋₆ alkyl or C₁₋₆ haloalkyl.

In some embodiments, each R¹³ is independently halogen, hydroxyl, —O(C₁₋₆ alkyl), cyano, or C₁₋₆ alkyl.

In some embodiments, R¹³ is oxo, hydroxyl, C₁₋₆ alkyl, or —O(C₁₋₆ alkyl).

In some embodiments, the compound is selected from compounds listed in Table 2. It is understood that individual enantiomers and diastereomers are included in the table below by Compound No. and Compound Name, and their corresponding structures can be readily determined therefrom. In some instances, the enantiomers or diastereomers are identified by their respective properties, for example, retention times on a chiral HPLC or its biological activities, and the absolute stereo configurations of the chiral centers are arbitrarily assigned.

TABLE 2

| No. | Structure | Name |
|---|---|---|
| 101 | 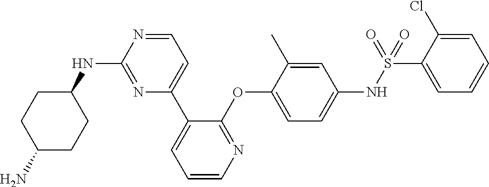 [SMILES: Cc1cc(NS(=O)(=O)c2ccccc2Cl)ccc1Oc1ncccc1-c1ccnc(N[C@H]2CC[C@H](N)CC2)n1] | N-[4-[[3-[2-[(4- aminocyclohexyl)amino\|pyrimidin-4-yl]-2- pyridyl]oxy]-3-methyl-phenyl]-2-chloro- benzenesulfonamide |
| 102 | 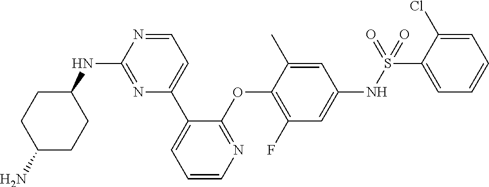 [SMILES: Cc1cc(NS(=O)(=O)c2ccccc2Cl)cc(F)c1Oc1ncccc1-c1ccnc(N[C@H]2CC[C@H](N)CC2)n1] | N-[4-[[3-[2-[(4-aminocy- clohexyl)amino]pyrimidin-4-yl]-2-pyridyl]oxy]- 3-fluoro-5-methyl-phenyl]-2-chloro- benzenesulfonamide |
| 103 | 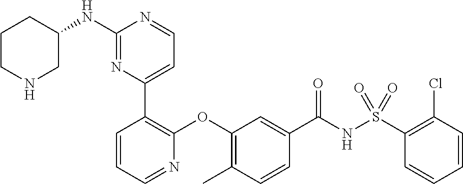 [SMILES: Cc1ccc(C(=O)NS(=O)(=O)c2ccccc2Cl)cc1Oc1ncccc1-c1ccnc(N[C@H]2CCCNC2)n1] | (S)-N-((2-chlorophenyl)sulfonyl)-4- methyl-3-((3-(2-(piperidin-3-ylamino)pyrimidin- 4-yl)pyridin-2-yl)oxy)benzamide |
| 104 | 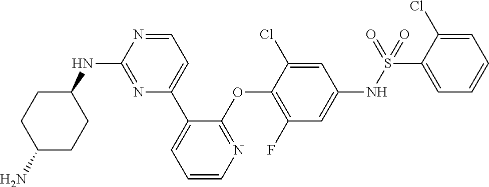 [SMILES: N[C@H]1CC[C@H](Nc2nccc(-c3cccnc3Oc3c(F)cc(NS(=O)(=O)c4ccccc4Cl)cc3Cl)n2)CC1] | N-[4-[[3-[2-[(4-aminocy- clohexyl)amino]pyrimidin-4-yl]-2-pyridyl]oxy]- 3-chloro-5-fluoro-phenyl]-2-chloro- benzenesulfonamide |
| 105 | 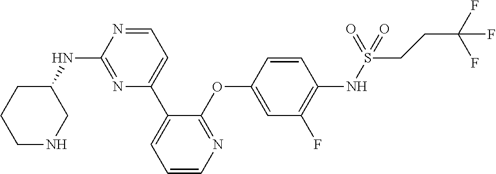 [SMILES: O=S(=O)(CCC(F)(F)F)Nc1ccc(Oc2ncccc2-c2ccnc(N[C@H]3CCCNC3)n2)cc1F] | (S)-3,3,3-trifluoro-N-(2-fluoro-4-((3-(2- (piperidin-3-ylamino)pyrimidin-4- yl)pyridin-2-yl)oxy)phenyl)propane-1- sulfonamide |
| 106 | 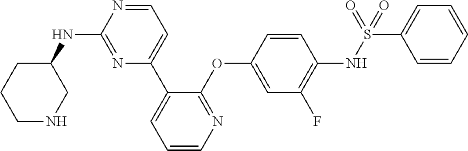 [SMILES: O=S(=O)(Nc1ccc(Oc2ncccc2-c2ccnc(N[C@@H]3CCCNC3)n2)cc1F)c1ccccc1] | (S)-N-(2-fluoro-4-((3-(2-(piperidin-3- ylamino)pyrimidin- 4-yl)pyridin-2-yl)oxy)phenyl)-1- phenylmethanesulfonamide |
| 107 | 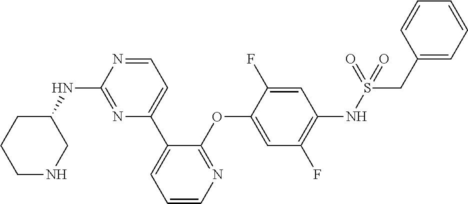 [SMILES: O=S(=O)(Cc1ccccc1)Nc1cc(F)c(Oc2ncccc2-c2ccnc(N[C@H]3CCCNC3)n2)cc1F] | (S)-N-(2,5-difluoro-4-((3-(2-(piperidin-3- ylamino)pyrimidin- 4-yl)pyridin-2-yl)oxy)phenyl)-1- phenylmethanesulfonamide |
| 108 | 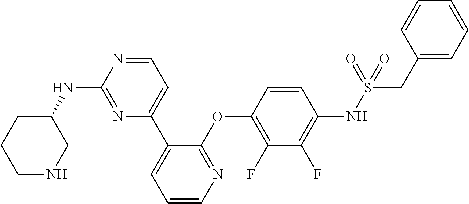 [SMILES: O=S(=O)(Cc1ccccc1)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin- 4-yl)pyridin-2-yl)oxy)phenyl)-1- phenylmethanesulfonamide |
| 109 | 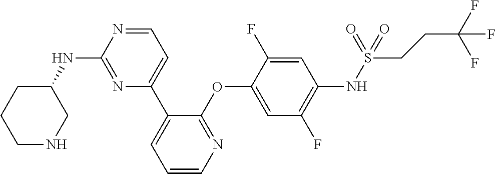 [SMILES: O=S(=O)(CCC(F)(F)F)Nc1cc(F)c(Oc2ncccc2-c2ccnc(N[C@H]3CCCNC3)n2)cc1F] | (S)-N-(2,5-difluoro-4-((3-(2-(piperidin-3- ylamino)pyrimidin-4-yl)pyridin-2- yl)oxy)phenyl)-3,3,3-trifluoropropane-1- sulfonamide |
| 110 | 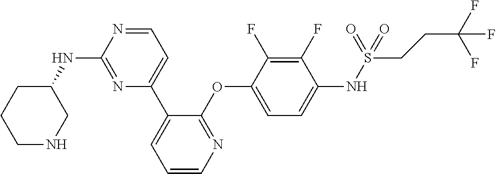 [SMILES: O=S(=O)(CCC(F)(F)F)Nc1ccc(Oc2ncccc2-c2ccnc(N[C@H]3CCCNC3)n2)c(F)c1F] | (S)-N-(2,5-difluoro-4-((3-(2-(piperidin-3- ylamino)pyrimidin- 4-yl)pyridin-2-yl)oxy)phenyl)-3,3,3- trifluoropropane-1-sulfonamide |
| 111 | 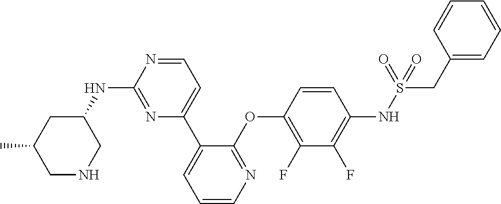 [SMILES: C[C@H]1CNC[C@@H](Nc2nccc(-c3cccnc3Oc3ccc(NS(=O)(=O)Cc4ccccc4)c(F)c3F)n2)C1] | N-(23-difluoro-4-((3-(2-(((3S,5R)-5- methylpiperidin-3-yl)amino)pyrimidin-4- yl)pyridin-2-yl)oxy)phenyl)-1- phenylmethanesulfonamide |
| 112 | 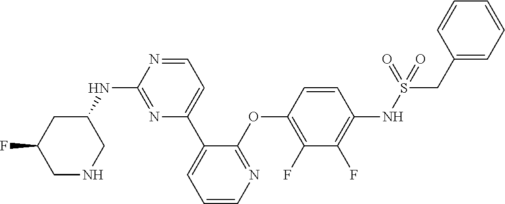 [SMILES: O=S(=O)(Cc1ccccc1)Nc1ccc(Oc2ncccc2-c2ccnc(N[C@@H]3CNC[C@@H](F)C3)n2)c(F)c1F] | N-(2,3-difluoro-4-((3-(2-(((3S5S)-5- fluoropiperidin-3-yl)amino)pyrimidin-4- yl)pyridin-2-yl)oxy)phenyl)-1- phenylmethanesulfonamide |
| 113 | 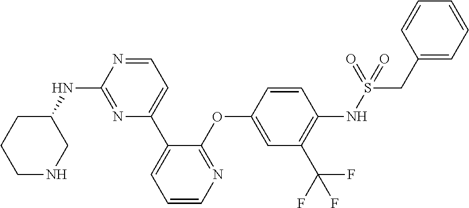 [SMILES: O=S(=O)(Cc1ccccc1)Nc1ccc(Oc2ncccc2-c2ccnc(N[C@H]3CCCNC3)n2)cc1C(F)(F)F] | (S)-1-phenyl-N-(4-((3-(2-(piperidin-3- ylamino)pyrimidin-4-yl)pyridin-2- yl)oxy)-2- (trifluoromethy1)phenyl)methanesulfonamide |
| 114 | 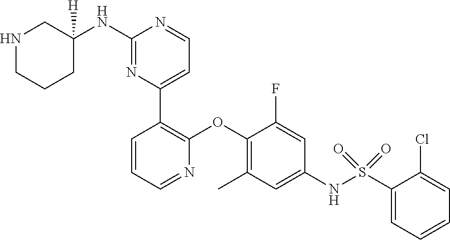 [SMILES: Cc1cc(NS(=O)(=O)c2ccccc2Cl)cc(F)c1Oc1ncccc1-c1ccnc(N[C@H]2CCCNC2)n1] | (S)-2-chloro-N-(3-fluoro-5-methyl-4-((3-(2- (piperidin-3-ylamino)pyrimidin-4- yl)pyridin-2-yl)oxy)phenyl)benzene- sulfonamide |
| 115 | 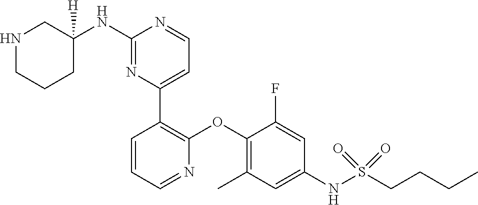 [SMILES: CCCCS(=O)(=O)Nc1cc(C)c(Oc2ncccc2-c2ccnc(N[C@H]3CCCNC3)n2)c(F)c1] | (S)-N-(3-fluoro-5-methyl-4-((3-(2-(piperidin-3- ylamino)pyrimidin-4- yl)pyridin-2-yl)oxy)phenyl)butane-1- sulfonamide |
| 116 | 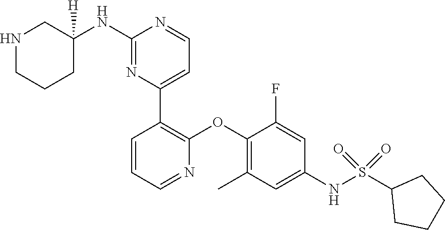 [SMILES: Cc1cc(NS(=O)(=O)C2CCCC2)cc(F)c1Oc1ncccc1-c1ccnc(N[C@H]2CCCNC2)n1] | (S)-N-(3-fluoro-5-methyl-4-((3-(2- (piperidin-3-ylamino)pyrimidin-4- yl)pyridin-2- yl)oxy)phenyl)cyclopentanesulfonamide |
| 117 | 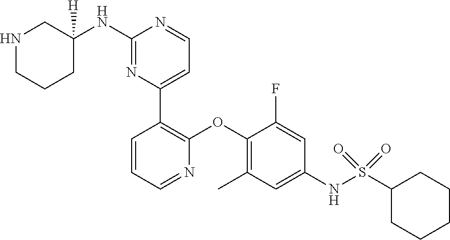 [SMILES: Cc1cc(NS(=O)(=O)C2CCCCC2)cc(F)c1Oc1ncccc1-c1ccnc(N[C@H]2CCCNC2)n1] | (S)-N-(3-fluoro-5-methyl-4-((3-(2- (piperidin-3-ylamino)pyrimidin-4- yl)pyridin-2- yl)oxy)phenyl)cyclohexanesulfonamide |
| 118 | 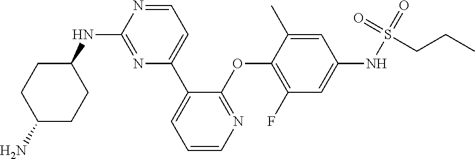 [SMILES: CCCS(=O)(=O)Nc1cc(C)c(Oc2ncccc2-c2ccnc(N[C@H]3CC[C@H](N)CC3)n2)c(F)c1] | N-(4-((3-(2-(((1r,4r)-4-amino- cyclohexyl)amino)pyrimidin-4-yl)pyridin- 2-yl)oxy)-3-fluoro-5-methylphenyl)propane-1- sulfonamide |
| 119 | 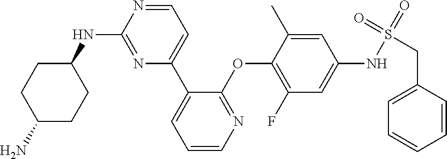 [SMILES: Cc1cc(NS(=O)(=O)Cc2ccccc2)cc(F)c1Oc1ncccc1-c1ccnc(N[C@H]2CC[C@H](N)CC2)n1] | N-(4-((3-(2-(((1r,4r)-4- aminocyclohexyl)amino)pyrimidin-4-yl)pyridin- 2-yl)oxy)-3-fluoro-5-methylphenyl)-1- phenylmethanesulfonamide |
| 120 | 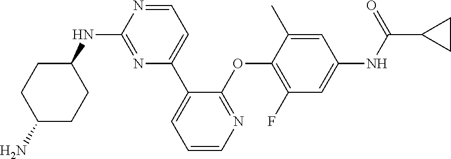 [SMILES: Cc1cc(NC(=O)C2CC2)cc(F)c1Oc1ncccc1-c1ccnc(N[C@H]2CC[C@H](N)CC2)n1] | N-(4-((3-(2-(((1r,4r)-4- aminocyclohexyl)amino)pyrimidin-4-yl)pyridin- 2-yl)oxy)-3-fluoro-5- methylphenyl)cyclopropanecarboxamide |
| 121 | 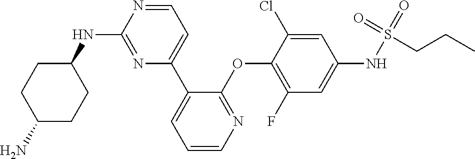 [SMILES: CCCS(=O)(=O)Nc1cc(F)c(Oc2ncccc2-c2ccnc(N[C@H]3CC[C@H](N)CC3)n2)c(Cl)c1] | N-(4-((3-(2-(((1r,4r)-4- aminocyclohexyl)amino)pyrimidin-4-yl)pyridin- 2-yl)oxy)-3-chloro-5-fluorophenyl)propane-1- sulfonamide |
| 122 | 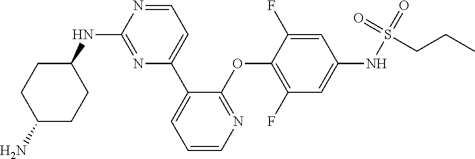 [SMILES: CCCS(=O)(=O)Nc1cc(F)c(Oc2ncccc2-c2ccnc(N[C@H]3CC[C@H](N)CC3)n2)c(F)c1] | N-(4-((3-(2-(((1r,4r)-4- aminocyclohexyl)amino)pyrimidin-4-yl)pyridin- 2-yl)oxy)-3,5-difluorophenyl)propane-1- sulfonamide |
| 123 | 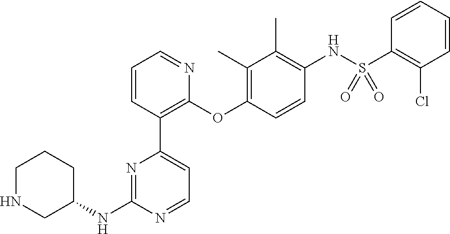 [SMILES: Cc1c(NS(=O)(=O)c2ccccc2Cl)ccc(Oc2ncccc2-c2ccnc(N[C@H]3CCCNC3)n2)c1C] | (S)-2-Chloro-N-(2,3-dimethy1-4-((3-(2- (piperidin-3-ylamino)pyrimidin-4- yl)pyridin-2- yl)oxy)phenyl)benzenesulfonamide |
| 124 | 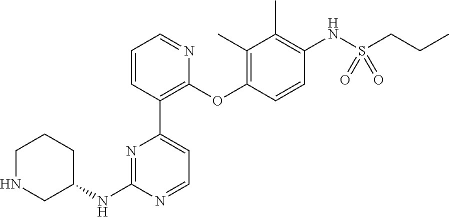 [SMILES: CCCS(=O)(=O)Nc1ccc(Oc2ncccc2-c2ccnc(N[C@H]3CCCNC3)n2)c(C)c1C] | (S)-N-(2,5-Dimethyl-4-((3-(2-(piperidin-3- ylamino)pyrimidin-4- yl)pyridin-2-yl)oxy)phenyl)propane-1- sulfonamide |
| 125 | 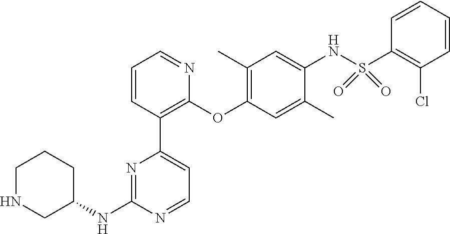 [SMILES: Cc1cc(Oc2ncccc2-c2ccnc(N[C@H]3CCCNC3)n2)c(C)cc1NS(=O)(=O)c1ccccc1Cl] | (S)-2-chloro-N-(2,5-dimethyl-4-((3-(2- (piperidin-3-ylamino)pyrimidin-4- yl)pyridin-2-yl)oxy)phenyl)benzene- sulfonamide |
| 126 | 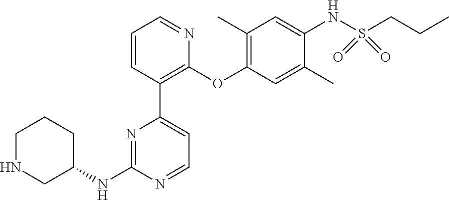 [SMILES: CCCS(=O)(=O)Nc1cc(C)c(Oc2ncccc2-c2ccnc(N[C@H]3CCCNC3)n2)cc1C] | (S)-N-(2,5-Dimethyl-4-((3-(2-(piperidin-3- ylamino)pyrimidin-4-yl)pyridin-2- yl)oxy)phenyl)propane-1-sulfonamide |
| 127 | 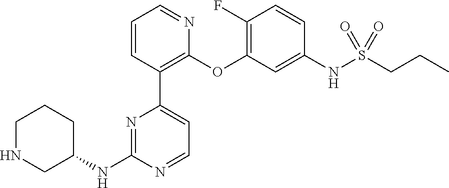 [SMILES: CCCS(=O)(=O)Nc1ccc(F)c(Oc2ncccc2-c2ccnc(N[C@H]3CCCNC3)n2)c1] | (S)-N-(4-fluoro-3-((3-(2-(piperidin-3- ylamino)pyrimidin-4-yl)pyridin-2- yl)oxy)phenyl)propane-1-sulfonamide |
| 128 | 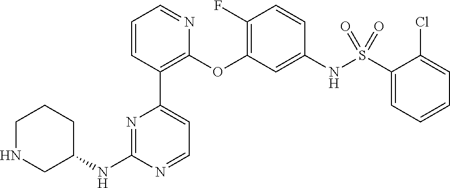 [SMILES: O=S(=O)(Nc1ccc(F)c(Oc2ncccc2-c2ccnc(N[C@H]3CCCNC3)n2)c1)c1ccccc1Cl] | (S)-2-Chloro-N-(4-fluoro-3-((3- (2-(piperidin-3-ylamino)pyrimidin-4- yl)pyridin-2-yl)oxy)phenyl)benzene- sulfonamide |
| 129 | 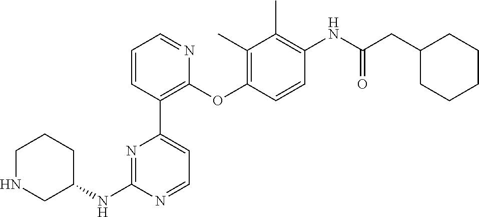 [SMILES: Cc1c(NC(=O)CC2CCCCC2)ccc(Oc2ncccc2-c2ccnc(N[C@H]3CCCNC3)n2)c1C] | (S)-2-Cyclohexy1-N- (2,3-dimethyl-4-((3-(2-(piperidin-3- ylamino)pyrimidin-4- yl)pyridin-2-yl)oxy)phenyl)acetamide |
| 130 | 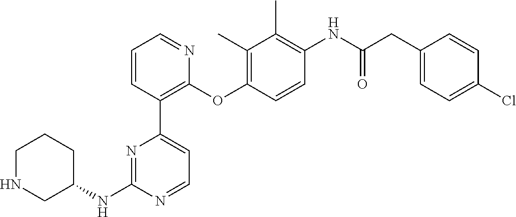 [SMILES: Cc1c(NC(=O)Cc2ccc(Cl)cc2)ccc(Oc2ncccc2-c2ccnc(N[C@H]3CCCNC3)n2)c1C] | (S)-2-(4-Chlorophenyl)-N-(2,3-dimethyl- 4-((3-(2-(piperidin-3- ylamino)pyrimidin-4-yl)pyridin-2- yl)oxy)phenyl)acetamide |
| 131 | 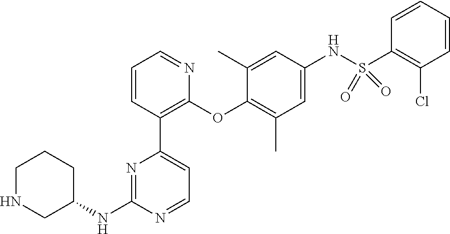 [SMILES: Cc1cc(NS(=O)(=O)c2ccccc2Cl)cc(C)c1Oc1ncccc1-c1ccnc(N[C@H]2CCCNC2)n1] | (S)-2-chloro-N-(3,5-dimethyl-4-((3-(2- (piperidin-3-ylamino)pyrimidin-4- yl)pyridin-2-yl)oxy)phenyl)benzenesulfonamide |
| 132 | 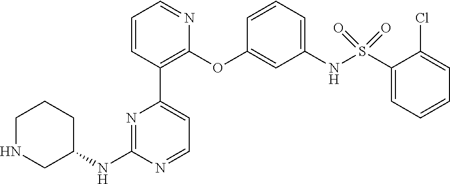 [SMILES: O=S(=O)(Nc1cccc(Oc2ncccc2-c2ccnc(N[C@H]3CCCNC3)n2)c1)c1ccccc1Cl] | (S)-2-chloro-N-(3-((3-(2-(piperidin-3- ylamino)pyrimidin-4- yl)pyridin-2-yl)oxy)phe- nyl)benzenesulfonamide |
| 133 | 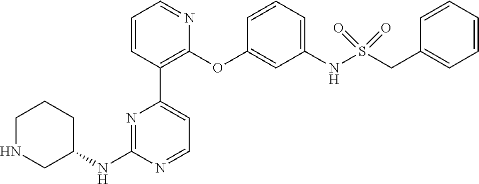 [SMILES: O=S(=O)(Cc1ccccc1)Nc1cccc(Oc2ncccc2-c2ccnc(N[C@H]3CCCNC3)n2)c1] | (S)-1-phenyl-N-(3-((3-(2-(piperidin-3- ylamino)pyrimidin-4- yl)pyridin-2-yl)oxy)phe- nyl)methanesulfonamide |
| 134 | 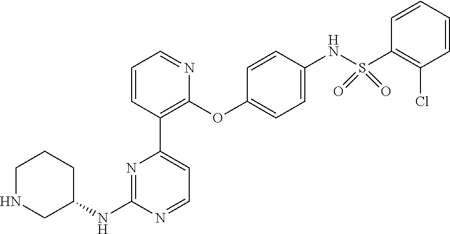 [SMILES: O=S(=O)(Nc1ccc(Oc2ncccc2-c2ccnc(N[C@H]3CCCNC3)n2)cc1)c1ccccc1Cl] | (S)-2-chloro-N-(4-((3-(2-(piperidin-3- ylamino)pyrimidin-4- yl)pyridin-2-yl)oxy)phe- nyl)benzenesulfonamide |
| 135 | 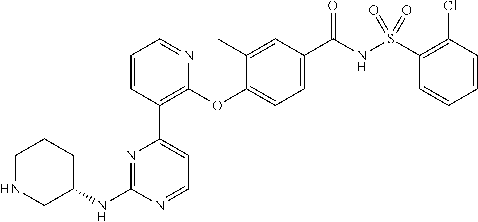 [SMILES: Cc1cc(C(=O)NS(=O)(=O)c2ccccc2Cl)ccc1Oc1ncccc1-c1ccnc(N[C@H]2CCCNC2)n1] | (S)-chloro-N-(4-methyl-3-((3-(2- (piperidin-3-ylamino)pyrimidin-4- yl)pyridin-2-yl)oxy)phe- nyl)benzenesulfonamide |
| 136 | 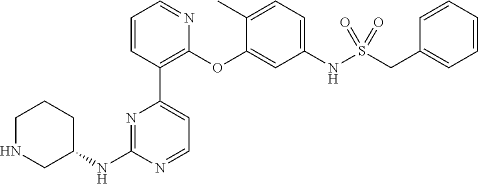 [SMILES: Cc1ccc(NS(=O)(=O)Cc2ccccc2)cc1Oc1ncccc1-c1ccnc(N[C@H]2CCCNC2)n1] | (S)-N-(4-methyl-3-((3-(2-(piperidin-3- ylamino)pyrimidin-4-yl)pyridin-2- yl)oxy)phenyl)-1- phenylmethanesulfonamide |
| 137 | 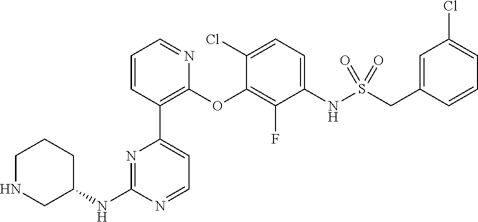 [SMILES: O=S(=O)(Cc1cccc(Cl)c1)Nc1ccc(Cl)c(Oc2ncccc2-c2ccnc(N[C@H]3CCCNC3)n2)c1F] | (S)-2-chloro-N-(4-chloro-2-fluoro-3-((3- (2-(piperidin-3-ylamino)pyrimi- din-4-yl)pyridin-2-yl)oxy)phe- nyl)benzenesulfonamide |
| 138 | 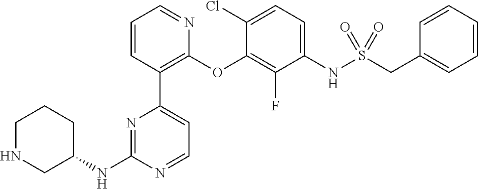 [SMILES: O=S(=O)(Cc1ccccc1)Nc1ccc(Cl)c(Oc2ncccc2-c2ccnc(N[C@H]3CCCNC3)n2)c1F] | (S)-N-(4-chloro-2-fluoro-3-((3-(2- (piperidin-3-ylamino)pyrimidin-4- yl)pyridin-2-yl)oxy)phenyl)-1- phenylmethanesulfonamide |
| 139 | 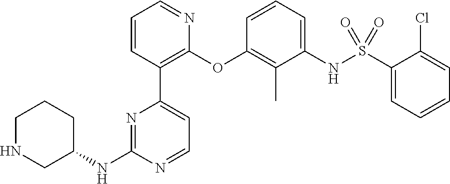 [SMILES: Cc1c(NS(=O)(=O)c2ccccc2Cl)cccc1Oc1ncccc1-c1ccnc(N[C@H]2CCCNC2)n1] | (S)-2-chloro-N-(2-methyl-3-((3-(2- (piperidin-3-ylamino)pyrimidin-4- yl)pyridin-2-yl)oxy)phe- nyl)benzenesulfonamide |
| 140 | 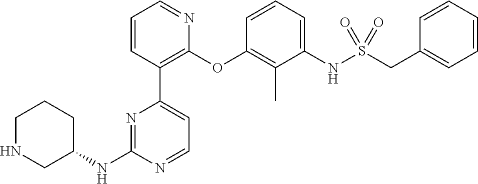 [SMILES: Cc1c(NS(=O)(=O)Cc2ccccc2)cccc1Oc1ncccc1-c1ccnc(N[C@H]2CCCNC2)n1] | (S)-N-(2-methy1-3-((3-(2-(piperidin-3- ylamino)pyrimidin-4- yl)pyridin-2-yl)oxy)phenyl)-1- phenylmethanesulfonamide |
| 141 | 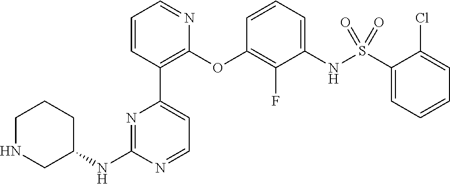 [SMILES: O=S(=O)(Nc1cccc(Oc2ncccc2-c2ccnc(N[C@H]3CCCNC3)n2)c1F)c1ccccc1Cl] | (S)-2-chloro-N-(2-fluoro-3-((3-(2- (piperidin-3-ylamino)pyrimidin-4- yl)pyridin-2-yl)oxy)phe- nyl)benzenesulfonamide |
| 142 | 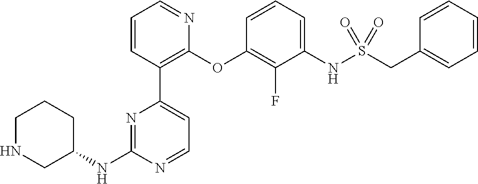 [SMILES: O=S(=O)(Cc1ccccc1)Nc1cccc(Oc2ncccc2-c2ccnc(N[C@H]3CCCNC3)n2)c1F] | (S)-N-(2-fluoro-3-((3-(2-(piperidin-3- ylamino)pyrimidin-4-yl)pyridin-2- yl)oxy)phenyl)-1- phenylmethanesulfonamide |
| 143 | 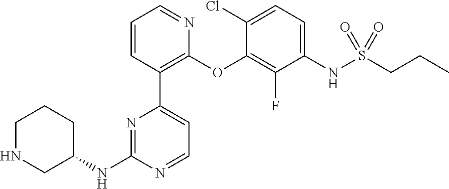 [SMILES: CCCS(=O)(=O)Nc1ccc(Cl)c(Oc2ncccc2-c2ccnc(N[C@H]3CCCNC3)n2)c1F] | (S)-N-(4-chloro-2-fluoro-3-((3-(2- (piperidin-3-ylamino)pyrimidin-4- yl)pyridin-2-yl)oxy)phenyl)propane-1- sulfonamide |
| 144 | 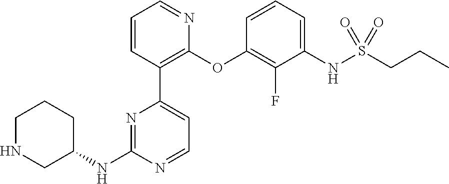 [SMILES: CCCS(=O)(=O)Nc1cccc(Oc2ncccc2-c2ccnc(N[C@H]3CCCNC3)n2)c1F] | (S)-N-(2-fluoro-3-((3-(2-(piperidin-3- ylamino)pyrimidin-4- yl)pyridin-2-yl)oxy)phenyl)propane-1- sulfonamide |
| 145 | 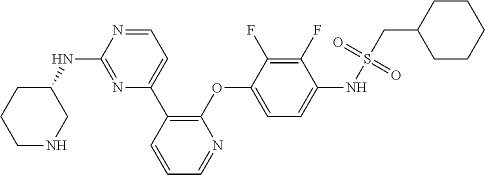 [SMILES: O=S(=O)(CC1CCCCC1)Nc1ccc(Oc2ncccc2-c2ccnc(N[C@H]3CCCNC3)n2)c(F)c1F] | (S)-1-cyclohexyl-N-(2,3-difluoro-4-((3- (2-(piperidin-3-ylamino)pyrimidin-4- yl)pyridin-2-yl)oxy)phenyl)methane- sulfonamide |
| 146 | 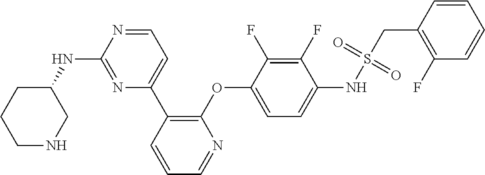 [SMILES: O=S(=O)(Cc1ccccc1F)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-1-(2- fluorophenyl)methanesulfonamide |
| 147 | 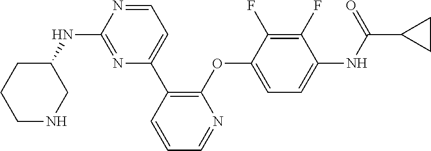 [SMILES: O=C(Nc1ccc(Oc2ncccc2-c2ccnc(N[C@H]3CCCNC3)n2)c(F)c1F)C1CC1] | (S)-N-(2,3-difluoro-4-(3-(2-(piperidin-3- ylamino)pyrimidin-4-yl)pyridin-2- yloxy )phenyl)cyclopropanecarboxamide |
| 148 | 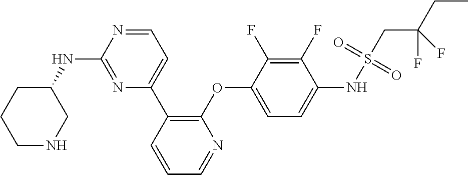 [SMILES: CCC(F)(F)CS(=O)(=O)Nc1ccc(Oc2ncccc2-c2ccnc(N[C@H]3CCCNC3)n2)c(F)c1F] | (S)-N-(2,3-Difluoro-4-((3-(2-(piperidin-3- ylamino)pyrimidin-4-yl)pyridin-2- yl)oxy)phenyl)-2,2-difluorobutane-1- sulfonamide |
| 149 | 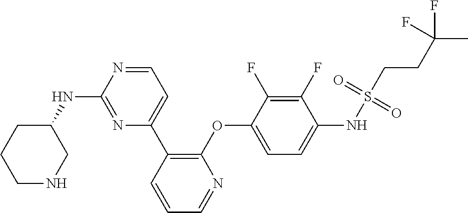 [SMILES: CC(F)(F)CCS(=O)(=O)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-3,3-difluorobutane-1- sulfonamide |
| 150 | 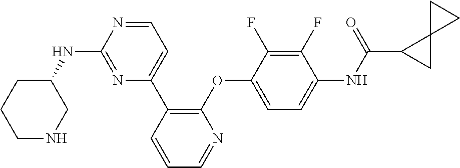 [SMILES: O=C(Nc1ccc(Oc2ncccc2-c2ccnc(N[C@H]3CCCNC3)n2)c(F)c1F)C1CC12CC2] | N-(2,3-difluoro-4-((3-(2-(((S)-piperidin-3- yl)amino)pyrimidin-4-yl)pyridin-2- yl)oxy)phenyl)spiro[2.2]pentane-1- carboxamide |
| 151 | 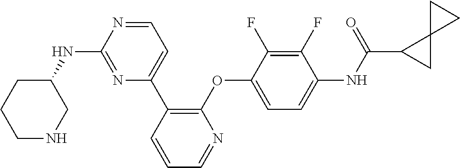 [SMILES: O=C(Nc1ccc(Oc2ncccc2-c2ccnc(N[C@H]3CCCNC3)n2)c(F)c1F)C1CC12CC2] | N-(2,3-difluoro-4-((3-(2-(((S)-piperidin-3- yl)amino)pyrimidin-4-yl)pyridin-2- yl)oxy)phenyl)spiro[2.2]pentane-1- carboxamide |
| 152 | 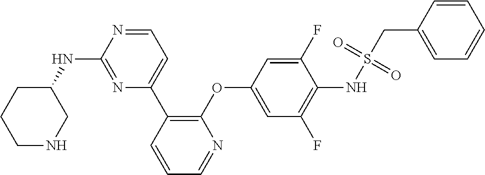 [SMILES: O=S(=O)(Cc1ccccc1)Nc1c(F)cc(Oc2ncccc2-c2ccnc(N[C@H]3CCCNC3)n2)cc1F] | (S)-N-(2,6-difluoro-4-((3-(2-(piperidin-3- ylamino)pyrimidin-4- yl)pyridin-2-yl)oxy)phenyl)-1- phenylmethanesulfonamide |
| 153 | 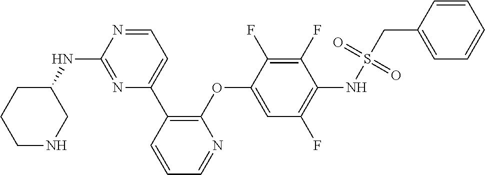 [SMILES: O=S(=O)(Cc1ccccc1)Nc1c(F)cc(Oc2ncccc2-c2ccnc(N[C@H]3CCCNC3)n2)c(F)c1F] | (S)-1-phenyl-N-(2,3,6-trifluoro-4-((3-(2- (piperidin-3-ylamino)pyrimidin-4- yl)pyridin-2-yl)oxy)phenyl)methane- sulfonamide |
| 154 | 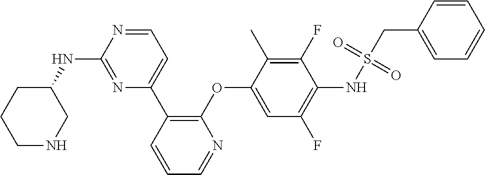 [SMILES: Cc1c(Oc2ncccc2-c2ccnc(N[C@H]3CCCNC3)n2)cc(F)c(NS(=O)(=O)Cc2ccccc2)c1F] | (S)-N-(2,6-difluoro-3-methyl-4-(3-(2- (piperidin-3-ylamino)pyrimidin-4- yl)pyridin-2- yloxy )phenyl)(phenyl)methanesulfonamide |
| 155 | 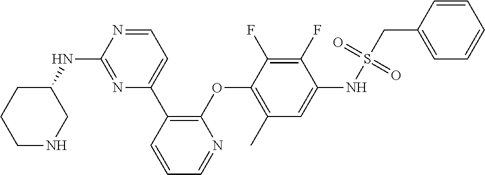 [SMILES: Cc1cc(NS(=O)(=O)Cc2ccccc2)c(F)c(F)c1Oc1ncccc1-c1ccnc(N[C@H]2CCCNC2)n1] | (S)-N-(2,3-difluoro-5-methy1-4-((3-(2- (piperidin-3-ylamino)pyrimidin-4- yl)pyridin-2-yl)oxy)phenyl)-1- phenylmethanesulfonamide |
| 156 | 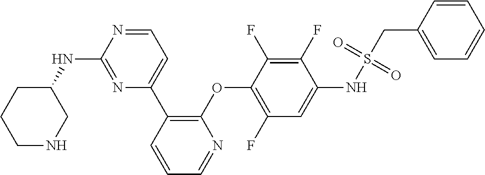 [SMILES: O=S(=O)(Cc1ccccc1)Nc1cc(F)c(Oc2ncccc2-c2ccnc(N[C@H]3CCCNC3)n2)c(F)c1F] | (S)-1-phenyl-N-(2.3,5 -trifluoro-4-((3-(2- (piperidin-3-ylamino)pyrimidin-4- yl)pyridin-2-yl)oxy)phenyl)methane- sulfonamide |
| 157 | 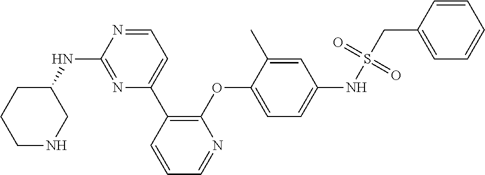 [SMILES: Cc1cc(NS(=O)(=O)Cc2ccccc2)ccc1Oc1ncccc1-c1ccnc(N[C@H]2CCCNC2)n1] | (S)-N-(3-methy1-4-((3-(2-(piperidin-3- ylamino)pyrimidin-4- yl)pyridin-2-yl)oxy)phenyl)-1- phenylmethanesulfonamide |
| 158 | 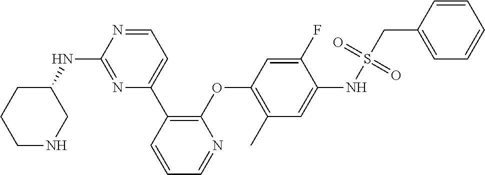 [SMILES: Cc1cc(NS(=O)(=O)Cc2ccccc2)c(F)cc1Oc1ncccc1-c1ccnc(N[C@H]2CCCNC2)n1] | (S)-N-(2-fluoro-5-methyl-4-(3-(2-(piperidin- 3-ylamino)pyrimidin-4- yl)pyridin-2- yloxy)phenyl)(phenyl)methanesulfonamide |
| 159 | 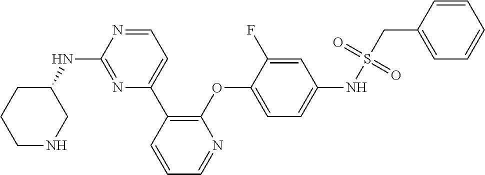 [SMILES: O=S(=O)(Cc1ccccc1)Nc1ccc(Oc2ncccc2-c2ccnc(N[C@H]3CCCNC3)n2)c(F)c1] | (S)-N-(3-fluoro-4-((3-(2-(piperidin-3- ylamino)pyrimidin-4- yl)pyridin-2-yl)oxy)phenyl)-1- phenylmethanesulfonamide |
| 160 | 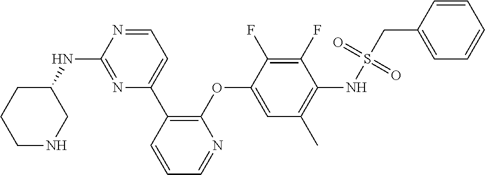 [SMILES: Cc1cc(Oc2ncccc2-c2ccnc(N[C@H]3CCCNC3)n2)c(F)c(F)c1NS(=O)(=O)Cc1ccccc1] | (S)-N-(2,3-difluoro-6-methyl-4-((3- (2-(piperidin-3-ylamino)pyrimidin-4- yl)pyridin-2-yl)oxy)phenyl)-1- phenylmethanesulfonamide |
| 161 | 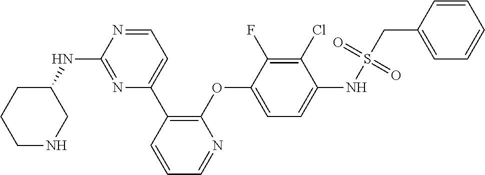 [SMILES: O=S(=O)(Cc1ccccc1)Nc1ccc(Oc2ncccc2-c2ccnc(N[C@H]3CCCNC3)n2)c(F)c1Cl] | (S)-N-(2-chloro-3-fluoro-4-((3-(2- (piperidin-3-ylamino)pyrimidin-4- yl)pyridin-2-yl)oxy)phenyl)-1- phenylmethanesulfonamide |
| 162 | 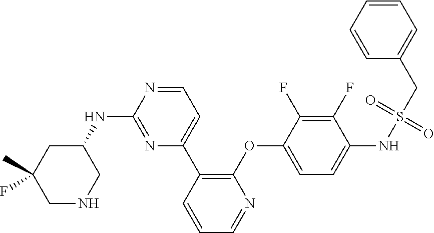 [SMILES: C[C@]1(F)CNC[C@@H](Nc2nccc(-c3cccnc3Oc3ccc(NS(=O)(=O)Cc4ccccc4)c(F)c3F)n2)C1] | N-(2,3-difluoro-4-((3-(2-(((3S,5R)-5- fluoro-5-methylpiperidin-3-yl)amino)py- rimidin-4-yl)pyridin-2-yl)oxy)phenyl)-1- phenylmethanesulfonamide |
| 163 | 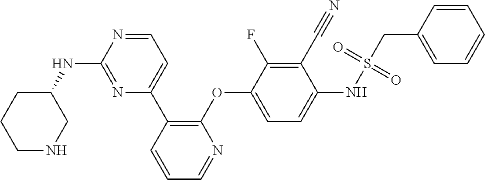 [SMILES: N#Cc1c(NS(=O)(=O)Cc2ccccc2)ccc(Oc2ncccc2-c2ccnc(N[C@H]3CCCNC3)n2)c1F] | (S)-N-(2-cyano-3-fluoro-4-((3-(2- (piperidin-3-ylamino)pyrimidin-4- yl)pyridin-2-yl)oxy)phenyl)-1- phenylmethanesulfonamide |
| 164 | 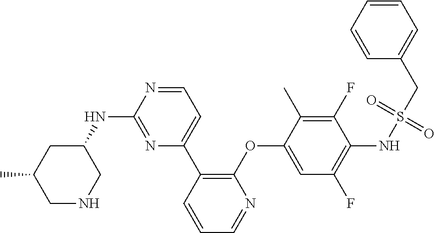 [SMILES: Cc1c(Oc2ncccc2-c2ccnc(N[C@@H]3CNC[C@H](C)C3)n2)cc(F)c(NS(=O)(=O)Cc2ccccc2)c1F] | N-(2,6-difluoro-3-methyl-4-((3- (2-(((3S,5R)-5-methyl-3-piperidyl)ami- no)pyrimidin-4-yl)-2- pyridyl)oxy)phenyl)-1-phenyl- methanesulfonamide |
| 165 | 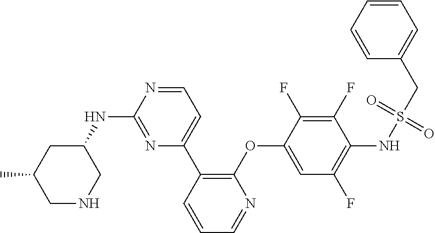 [SMILES: C[C@H]1CNC[C@@H](Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2- (((3S,5R)-5-methyl-3- piperidyl)amino)pyrimidin-4-yl)-2- pyridyl)oxy)phenyl)methanesulfonamide |
| 166 | 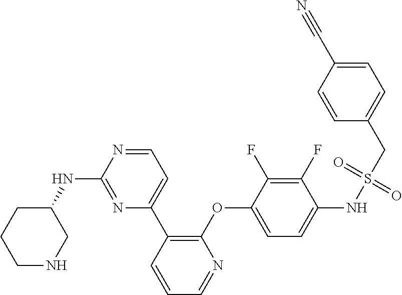 [SMILES: N#Cc1ccc(CS(=O)(=O)Nc2ccc(Oc3ncccc3-c3ccnc(N[C@H]4CCCNC4)n3)c(F)c2F)cc1] | (S)-1-(4-cyanophenyl)-N-(2,3-difluoro- 4-((3-(2-(piperidin-3-ylamino)py- rimidin-4-yl)pyridin-2- yl)oxy)phenyl)methanesulfonamide |
| 167 | 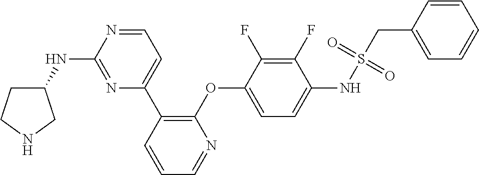 [SMILES: O=S(=O)(Cc1ccccc1)Nc1ccc(Oc2ncccc2-c2ccnc(N[C@H]3CCNC3)n2)c(F)c1F] | (S)-N-(2,3-difluoro-4-((3-(2-(pyrrolidin-3- ylamino)pyrimidin-4- yl)pyridin-2-yl)oxy)phenyl)-1- phenylmethanesulfonamide |
| 168 | 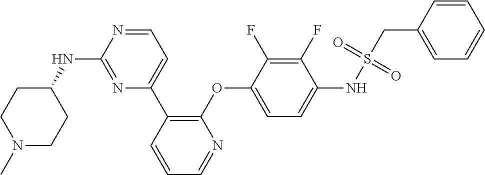 | N-(2,3-difluoro-4-((3-(2-((1- methylpiperidin-4-yl)amino)pyrimidin-4- yl)pyridin-2-yl)oxy)phenyl)-1- phenylmethanesulfonamide |
| 169 | 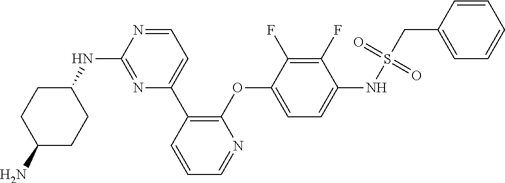 [SMILES: N[C@H]1CC[C@H](Nc2nccc(-c3cccnc3Oc3ccc(NS(=O)(=O)Cc4ccccc4)c(F)c3F)n2)CC1] | N-(4-((3-(2-(((1r,4r)-4-amino- cyclohexyl)amino)pyrimidin-4-yl)pyridin- 2-yl)oxy)-2,3-difluorophenyl)-1- phenylmethanesulfonamide |
| 170 | 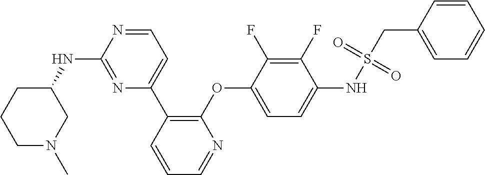 [SMILES: CN1CCC[C@H](Nc2nccc(-c3cccnc3Oc3ccc(NS(=O)(=O)Cc4ccccc4)c(F)c3F)n2)C1] | (S)-N-(2,3-difluoro-4-((3-(2- ((1-methylpiperidin-3-yl)ami- no)pyrimidin-4-yl)pyridin-2- yl)oxy)phenyl)-1-phenylmethane- sulfonamide |
| 171 | 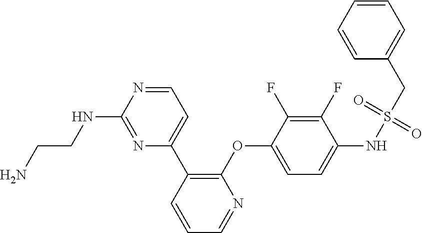 [SMILES: NCCNc1nccc(-c2cccnc2Oc2ccc(NS(=O)(=O)Cc3ccccc3)c(F)c2F)n1] | N-(4-((3-(2-((2- aminoethyl)amino)pyrimidin-4-yl)pyridin- 1-yl)oxy)-2,3-difluorophenyl)-1- phenylmethanesulfonamide |
| 172 | 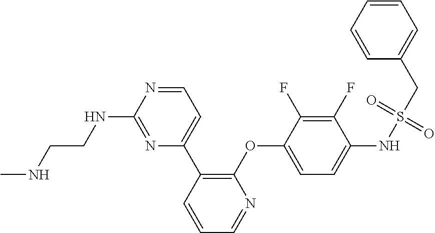 [SMILES: CNCCNc1nccc(-c2cccnc2Oc2ccc(NS(=O)(=O)Cc3ccccc3)c(F)c2F)n1] | N-(2,3-difluoro-4-((3-(2-(2- (methylamino)ethylarnino)pyrimidin-4-yl)-2- pyridyl)oxy)phenyl)-1-phenyl- methanesulfonamide |
| 173 | 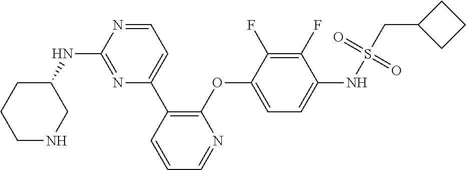 [SMILES: O=S(=O)(CC1CCC1)Nc1ccc(Oc2ncccc2-c2ccnc(N[C@H]3CCCNC3)n2)c(F)c1F] | (S)-1-cyclobutyl-N-(2,3-difluoro-4-((3-(2- (piperidin-3-ylamino)pyrimidin-4- yl)pyridin-2-yl)oxy)phenyl)methane- sulfonamide |
| 174 | 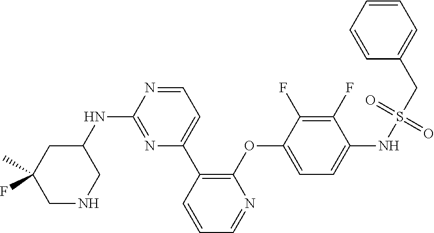 [SMILES: C[C@@]1(F)CNCC(Nc2nccc(-c3cccnc3Oc3ccc(NS(=O)(=O)Cc4ccccc4)c(F)c3F)n2)C1] | N-(2,3-difluoro-4-((3-(2-(((3S,5S)-5-fluoro-5- methylpiperidin-3-yl)amino)pyrimidin-4- yl)pyridin-2-yl)oxy)phenyl)-1- phenylmethanesulfonamide |
| 175 | 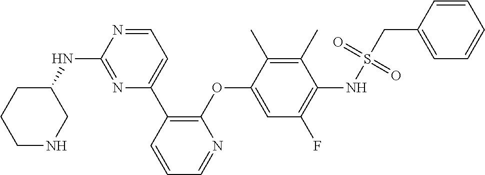 [SMILES: Cc1c(Oc2ncccc2-c2ccnc(N[C@H]3CCCNC3)n2)cc(F)c(NS(=O)(=O)Cc2ccccc2)c1C] | (S)-N-(6-fluoro-2,3-dimethy1-4-((3-(2-(piperidin- 3-ylamino)pyrimidin-4-yl)pyridin-2- yl)oxy)phenyl)-1-phenylmethanesulfonamide |
| 176 | 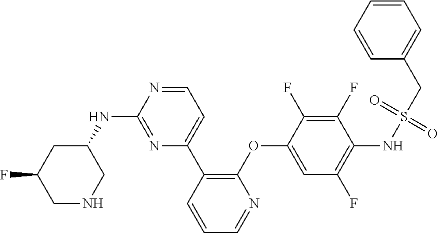 [SMILES: O=S(=O)(Cc1ccccc1)Nc1c(F)cc(Oc2ncccc2-c2ccnc(N[C@@H]3CNC[C@@H](F)C3)n2)c(F)c1F] | 1-phenyl-N-(2,3,6-trifluoro-4-((3-(2-(((3,5S)-5- fluoropiperidin-3-yl)amino)pyrimidin-4- yl)pyridin-2-yl)oxy)phenyl)methanesulfonamide |
| 177 | 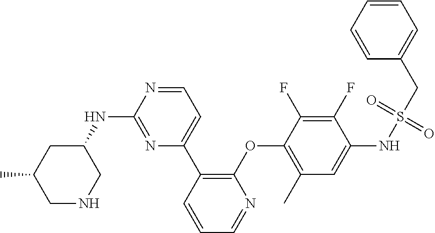 [SMILES: Cc1cc(NS(=O)(=O)Cc2ccccc2)c(F)c(F)c1Oc1ncccc1-c1ccnc(N[C@@H]2CNC[C@H](C)C2)n1] | N-(2,3-difluoro-5-methyl-4-((3-(2-(((3S,5R)-5- methyl-3-piperidyl)amino)pyrimidin-4-yl)-2- pyridyl)oxy)phenyl)-1-phenyl- methanesulfonamide |
| 178 | 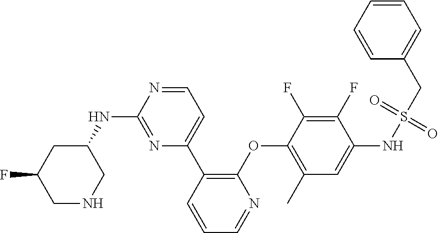 [SMILES: Cc1cc(NS(=O)(=O)Cc2ccccc2)c(F)c(F)c1Oc1ncccc1-c1ccnc(N[C@@H]2CNC[C@@H](F)C2)n1] | N-(2,3-difluoro-4-((3-(2-(((3S,5S)-5-fluoro-3- piperidyl)amino)pyrimidin-4-yl)-2-pyridyl)oxy)- 5-methyl-phenyl)-1-phenyl-methanesulfonamide |
| 179 | 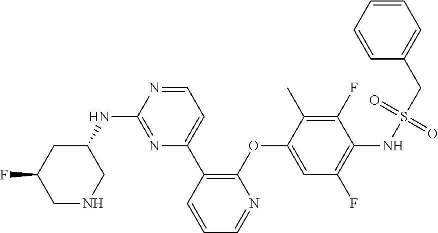 [SMILES: Cc1c(Oc2ncccc2-c2ccnc(N[C@@H]3CNC[C@@H](F)C3)n2)cc(F)c(NS(=O)(=O)Cc2ccccc2)c1F] | N-(2,6-difluoro-4-((3-(2-(((35,5S)-5-fluoro-3- piperidyl)amino)pyrimidin-4-yl)-2-pyridyl)oxy)- 3-methyl-phenyl)-1-phenyl-methanesulfonamide |
| 180 | 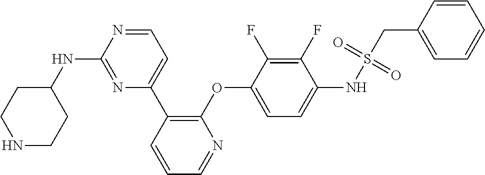 [SMILES: O=S(=O)(Cc1ccccc1)Nc1ccc(Oc2ncccc2-c2ccnc(NC3CCNCC3)n2)c(F)c1F] | N-(2,3-difluoro-4-((3-(2-(4- piperidylamino)pyrimidin-4- yl)-2-pyridyl)oxy)phenyl)-1-phenyl- methanesulfonamide |
| 181 | 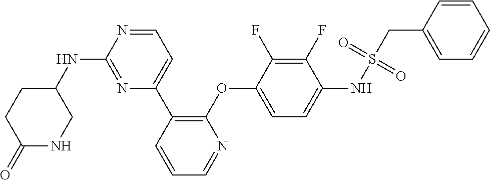 [SMILES: O=C1CCC(Nc2nccc(-c3cccnc3Oc3ccc(NS(=O)(=O)Cc4ccccc4)c(F)c3F)n2)CN1] | (S)-N-(2,3-difluoro-4-((3-(2-((6- oxopiperidin-3-yl)amino)pyrimidin-4-yl)pyridin- 2-yl)oxy)phenyl)-1-phenylmethanesulfonamide |
| 182 | 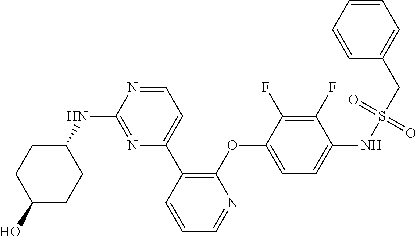 [SMILES: O=S(=O)(Cc1ccccc1)Nc1ccc(Oc2ncccc2-c2ccnc(N[C@H]3CC[C@H](O)CC3)n2)c(F)c1F] | N-(2,3-difluoro-4-((3-(2-(((1r,4r)-4- hydroxycyclohexyl)amino)pyrimidin-4- yl)pyridin-2-yl)oxy)phenyl)-1- phenylmethanesulfonamide |
| 183 | 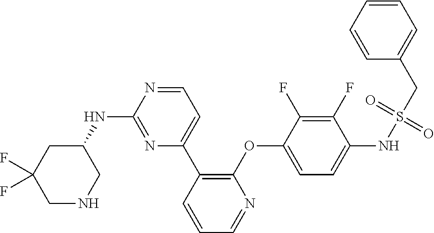 [SMILES: O=S(=O)(Cc1ccccc1)Nc1ccc(Oc2ncccc2-c2ccnc(N[C@@H]3CNCC(F)(F)C3)n2)c(F)c1F] | (S)-N-(4-((3-(2-((5,5-difluoropiperidin-3- yl)amino)pyrimidin-4-yl)pyridin-2-yl)oxy)-2,3- difluorophenyl)-1-phenylmethanesulfonamide |
| 184 | 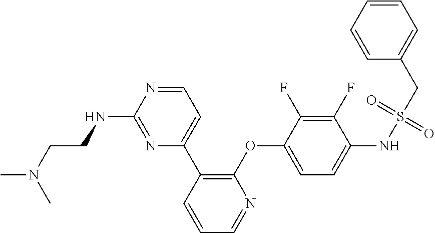 | N-(4-((3-(2-((2- (dimethylamino)ethyl)amino)pyrimidin-4- yl)pyridin-2-yl)oxy)-2,3-difluorophenyl)-1- phenylmethanesulfonamide |
| 185 | 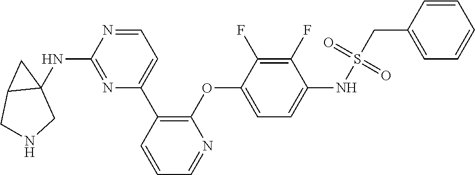 [SMILES: O=S(=O)(Cc1ccccc1)Nc1ccc(Oc2ncccc2-c2ccnc(NC34CNCC3C4)n2)c(F)c1F] | N-(4-((3-(2-(3-azabicyclo[3.1.0]hexan-5- ylamino)pyrimidin-4-yl)-2-pyridyl)oxy)-2,3- difluoro-phenyl)-1-phenyl-methanesulfonamide |
| 187 | 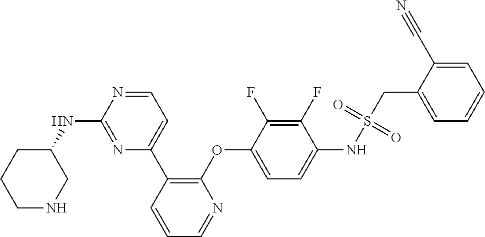 [SMILES: N#Cc1ccccc1CS(=O)(=O)Nc1ccc(Oc2ncccc2-c2ccnc(N[C@H]3CCCNC3)n2)c(F)c1F] | (S)-1-(2-cyanophenyl)-N-(2,3-difluoro-4- ((3-(2-(piperidin-3-ylamino)pyrimidin-4- yl)pyridin-2-yl)oxy)phenyl)methanesulfonamide |
| 188 | 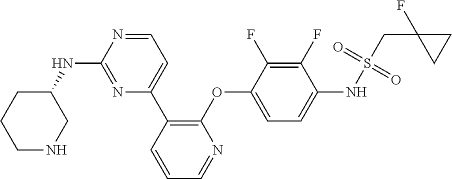 [SMILES: O=S(=O)(CC1(F)CC1)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-1-(1- fluorocyclopropyl)methanesulfonamide |
| 189 | 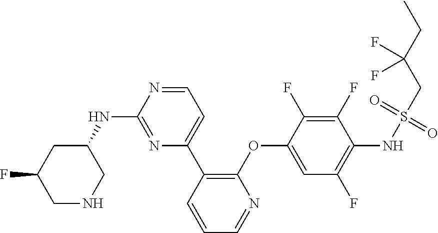 [SMILES: CCC(F)(F)CS(=O)(=O)Nc1c(F)cc(Oc2ncccc2-c2ccnc(N[C@@H]3CNC[C@@H](F)C3)n2)c(F)c1F] | 2,2-difluoro-N-(2,3,6-trifluoro-4-((3-(2-(((3S,5S)- 5-fluoro-3-piperidyl)amino)pyrimidin-4-yl)-2- pyridyl)oxy)phenyl)butane-1-Sulfonamide |
| 190 | 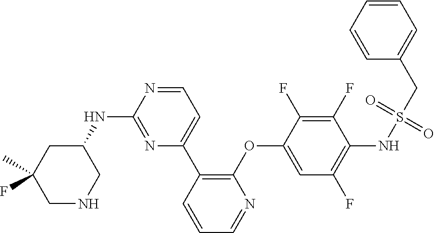 [SMILES: C[C@@]1(F)CNC[C@@H](Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-(((3S,5S)-5- fluoro-5-methyl-3-piperidyl)amino)pyrimidin-4- yl)-2-pyridyl)oxy)phenyl)methanesulfonamide |
| 191 | 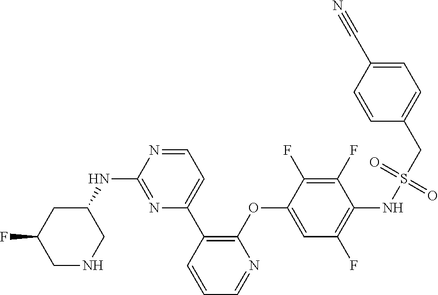 [SMILES: N#Cc1ccc(CS(=O)(=O)Nc2c(F)cc(Oc3ncccc3-c3ccnc(N[C@@H]4CNC[C@@H](F)C4)n3)c(F)c2F)cc1] | 1-(4-cyanophenyl)-N-(2,3,6-trifluoro-4-((3-(2- (((3S,5S)-5-fluoropiperidin-3- yl)amino)pyrimidin-4-yl)pyridin-2- yl)oxy)phenyl)methanesulfonamide |
| 192 | 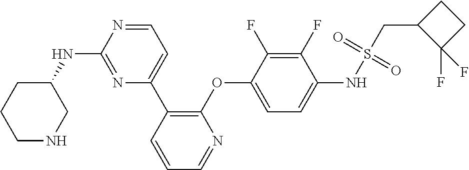 [SMILES: O=S(=O)(CC1CCC1(F)F)Nc1ccc(Oc2ncccc2-c2ccnc(N[C@H]3CCCNC3)n2)c(F)c1F] | N-(2,3-difluoro-4-((3-(2-(((S)-piperidin-3- yl)amino)pyrimidin-4-yl)pyridin-2- yl)oxy)phenyl)-1-(2,2- difluorocyclobutyl)methanesulfonamide |
| 193 | 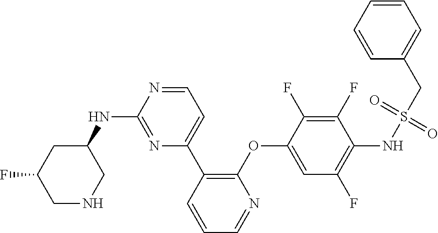 [SMILES: O=S(=O)(Cc1ccccc1)Nc1c(F)cc(Oc2ncccc2-c2ccnc(N[C@H]3CNC[C@H](F)C3)n2)c(F)c1F] | 1-phenyl-N-(2,3,6-trifluoro-4-((3-(2-(((3R,5R)-5- fluoro-3-piperidyl)amino)pyrimidin-4-yl)-2- pyridyl)oxy)phenyl)methanesulfonamide |
| 194 | 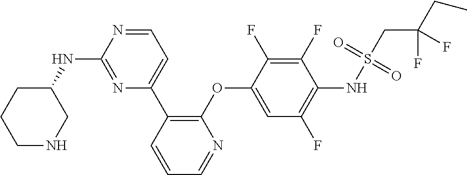 [SMILES: CCC(F)(F)CS(=O)(=O)Nc1c(F)cc(Oc2ncccc2-c2ccnc(N[C@H]3CCCNC3)n2)c(F)c1F] | (S)-2,2-difluoro-N-(2,3,6-trifluoro-4-((3- (2-(piperidin-3-ylamino)pyrimidin-4- yl)pyridin-2-yl)oxy)phenyl)butane-1-sulfonamide |
| 195 | 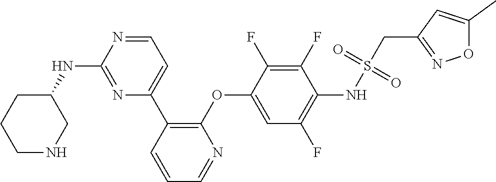 [SMILES: Cc1cc(CS(=O)(=O)Nc2c(F)cc(Oc3ncccc3-c3ccnc(N[C@H]4CCCNC4)n3)c(F)c2F)no1] | (S)-1-(5-methylisoxazol-3-y1)-N-(2,3,6-trifluoro- 4-((3-(2-(piperidin-3-ylamino)pyrimidin-4- yl)pyridin-2-yl)oxy)phenyl)methanesulfonamide |
| 196 | 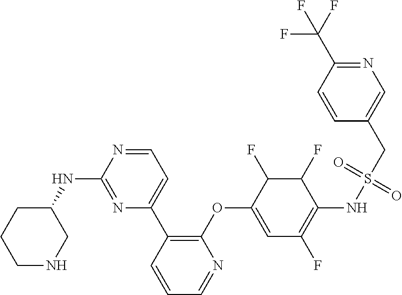 [SMILES: O=S(=O)(Cc1ccc(C(F)(F)F)nc1)NC1=C(F)C=C(Oc2ncccc2-c2ccnc(N[C@H]3CCCNC3)n2)C(F)C1F] | (S)-N-(2,3,6-trifluoro-4-((3-(2-(piperidin-3- ylamino)pyrimidin-4-yl)pyridin-2- yl)oxy)phenyl)-1-(6-(trifluoromethyl)pyridin-3- yl)methanesulfonamide |
| 197 | 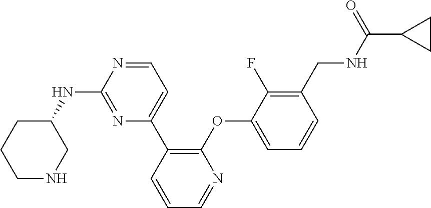 [SMILES: O=C(NCc1cccc(Oc2ncccc2-c2ccnc(N[C@H]3CCCNC3)n2)c1F)C1CC1] | (S)-N-(2-fluoro-3-((3-(2- (piperidin-3-ylamino)pyrimidin-4-yl)pyridin-2- yl)oxy)benzyl)cyclopropanecarboxamide |
| 198 | 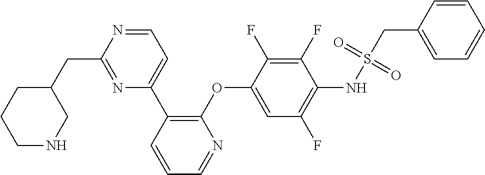 [SMILES: O=S(=O)(Cc1ccccc1)Nc1c(F)cc(Oc2ncccc2-c2ccnc(CC3CCCNC3)n2)c(F)c1F] | 1-phenyl-N-(2,3,6-trifluoro-4-((3-(2-(piperidin-3- ylmethyl)pyrimidin-4- yl)pyridin-2-yl)oxy)phenyl)methanesulfonamide |
| 200 | 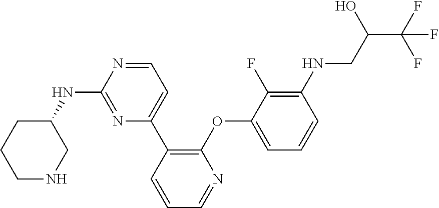 [SMILES: OC(CNc1cccc(Oc2ncccc2-c2ccnc(N[C@H]3CCCNC3)n2)c1F)C(F)(F)F] | 1,1,1-trifluoro-3-((2-fluoro-3-((3-(2- (((S)-piperidin-3-yl)amino) pyrimidin-4-yl)pyridin-2- yl)oxy)phenyl)amino)propan-2-ol |
| 202 | 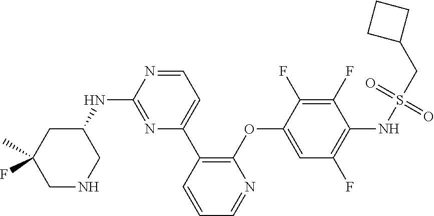 [SMILES: C[C@@]1(F)CNC[C@@H](Nc2nccc(-c3cccnc3Oc3cc(F)c(NS(=O)(=O)CC4CCC4)c(F)c3F)n2)C1] | 1-cyclobutyl-N-(2,3,6-trifluoro-4-((3-(2- (((3S,5S)-5-fluoro-5-methylpiperidin-3- yl)amino)pyrimidin-4-yl)pyridin-2- yl)oxy)phenyl)methanesulfonamide |
| 203 | 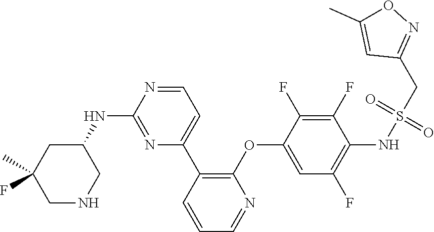 [SMILES: Cc1cc(CS(=O)(=O)Nc2c(F)cc(Oc3ncccc3-c3ccnc(N[C@@H]4CNC[C@@](C)(F)C4)n3)c(F)c2F)no1] | 1-(5-methylisoxazol-3-yl)-N-(2,3,6- trifluoro-4- ((3-(2-(((3S,5S)-5-fluoro-5-methylpiperidin-3- yl)amino)pyrimidin-4-yl)pyridin-2- yl)oxy)phenyl)methanesulfonamide |
| 204 | 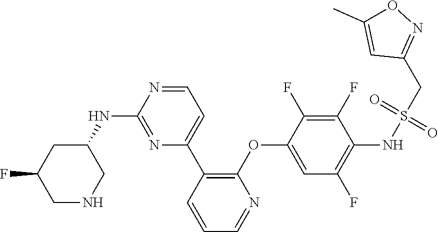 [SMILES: Cc1cc(CS(=O)(=O)Nc2c(F)cc(Oc3ncccc3-c3ccnc(N[C@@H]4CNC[C@@H](F)C4)n3)c(F)c2F)no1] | 1-(5-methylisoxazol-3-yl)-N-(2,3,6- trifluoro-4- ((3-(2-(((3S,5S)-5-fluoropiperidin-3- yl)amino)pyrimidin-4-yl)pyridin-2- yl)oxy)phenyl)methanesulfonamide |
| 205 | 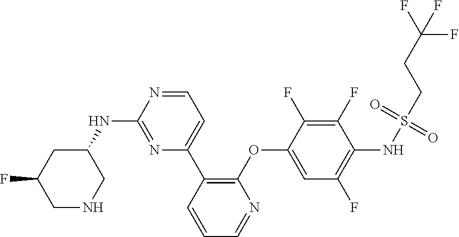 [SMILES: O=S(=O)(CCC(F)(F)F)Nc1c(F)cc(Oc2ncccc2-c2ccnc(N[C@@H]3CNC[C@@H](F)C3)n2)c(F)c1F] | 3,3,3-trifluoro-N-(2,3,6-trifluoro-4-((3-(2- (((3S,5S)-5-fluoro-3-piperidyl)amino)pyrimidin- 4-yl)-2-pyridyl)oxy)phenyl)propane-1- sulfonamide |
| 206 | 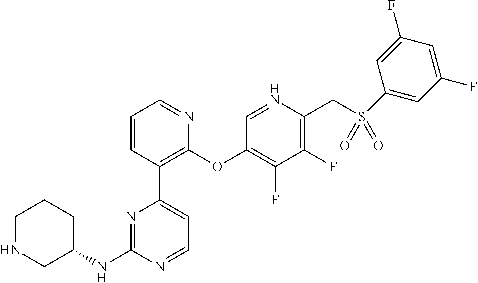 | (S)-N-(2,3-difluoro-4-((3-(2-(piperidin-3- ylamino)pyrimidin-4-yl)pyridin-2- yl)oxy)phenyl)-3,5-difluorobenzenesulfonamide |
| 207 | 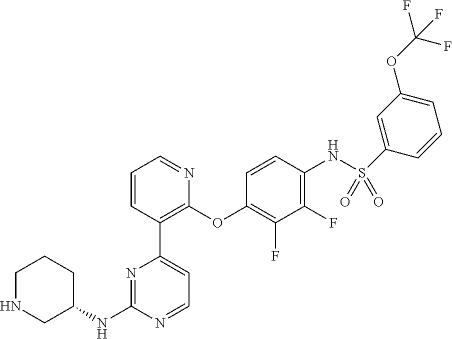 [SMILES: O=S(=O)(Nc1ccc(Oc2ncccc2-c2ccnc(N[C@H]3CCCNC3)n2)c(F)c1F)c1cccc(OC(F)(F)F)c1] | (S)-N-(2,3-difluoro-4-((3-(2-(piperidin-3- ylamino)pyrimidin-4-yl)pyridin-2- yl)oxy)phenyl)-3- (trifluoromethoxy)benzenesulfonamide |
| 208 | 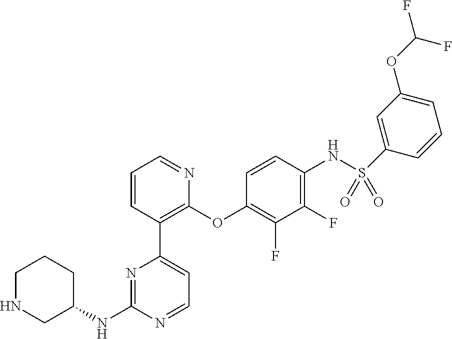 [SMILES: O=S(=O)(Nc1ccc(Oc2ncccc2-c2ccnc(N[C@H]3CCCNC3)n2)c(F)c1F)c1cccc(OC(F)F)c1] | (S)-N-(2,3-difluoro-4-((3-(2-(piperidin-3- ylamino)pyrimidin-4-yl)pyridin-2- yl)oxy)phenyl)-3- (difluoromethoxy)benzenesulfonamide |
| 209 | 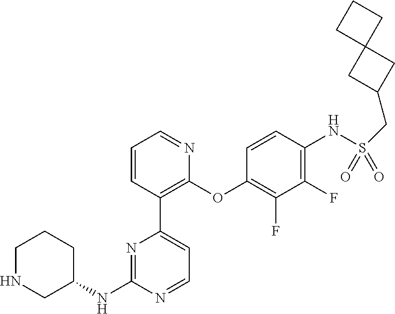 [SMILES: O=S(=O)(CC1CC2(CCC2)C1)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-1-(spiro[3.3]heptan-2- yl)methanesulfonamide |
| 210 | 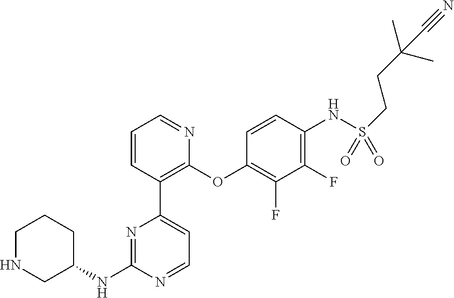 [SMILES: CC(C)(C#N)CCS(=O)(=O)Nc1ccc(Oc2ncccc2-c2ccnc(N[C@H]3CCCNC3)n2)c(F)c1F] | (S)-3-cyano-N-(2,3-difluoro-4-((3-(2-(piperidin- 3-ylamino)pyrimidin-4-yl)pyridin-2- yl)oxy)phenyl)-3-methylbutane-1-sulfonamide |
| 211 | 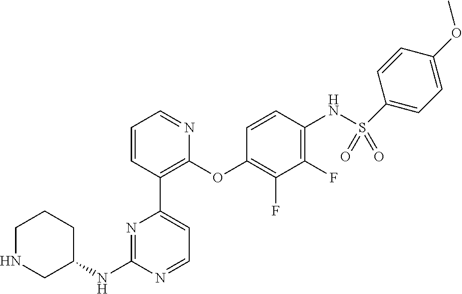 [SMILES: COc1ccc(S(=O)(=O)Nc2ccc(Oc3ncccc3-c3ccnc(N[C@H]4CCCNC4)n3)c(F)c2F)cc1] | (S)-N-(2,3-difluoro-4-((3-(2-(piperidin-3- ylamino)pyrimidin-4-yl)pyridin-2- yl)oxy)phenyl)-4-methoxybenzenesulfonamide |
| 212 | 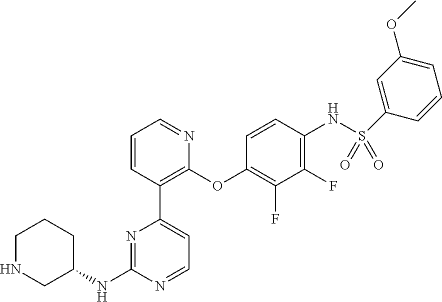 [SMILES: COc1cccc(S(=O)(=O)Nc2ccc(Oc3ncccc3-c3ccnc(N[C@H]4CCCNC4)n3)c(F)c2F)c1] | (S)-N-(2,3-difluoro-4-((3-(2-(piperidin-3- ylamino)pyrimidin-4-yl)pyridin-2- yl)oxy)phenyl)-3-methoxybenzenesulfonamide |
| 213 | 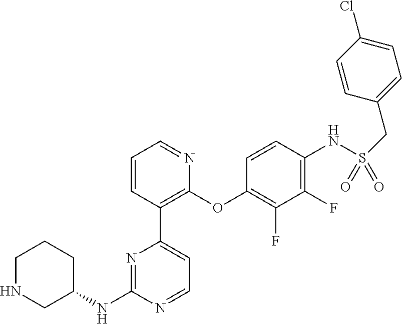 [SMILES: O=S(=O)(Cc1ccc(Cl)cc1)Nc1ccc(Oc2ncccc2-c2ccnc(N[C@H]3CCCNC3)n2)c(F)c1F] | (S)-1-(4-chlorophenyl)-N-(2,3-difluoro-4-((3-(2- (piperidin-3-ylamino)pyrimidin-4-yl)pyridin-2- yl)oxy)phenyl)methanesulfonamide |
| 214 | 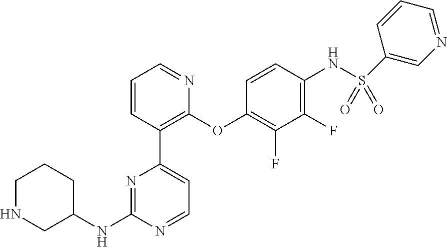 [SMILES: O=S(=O)(Nc1ccc(Oc2ncccc2-c2ccnc(NC3CCCNC3)n2)c(F)c1F)c1cccnc1] | (S)-N-(2,3-difluoro-4-((3-(2-(piperidin-3- ylamino)pyrimidin- 4-yl)pyridin-2-yl)oxy)phenyl)pyridine-3- sulfonamide |
| 215 | 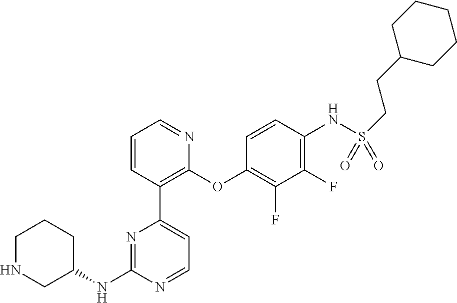 | (S)-2-cyclohexyl-N-(2,3-difluoro-4-((3-(2- (piperidin-3-ylamino)pyrimidin-4- yl)pyridin-2-yl)oxy)phenyl)ethane-1-sulfonamide |
| 216 | 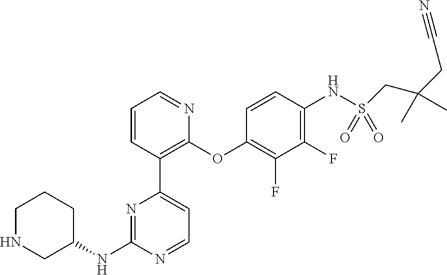 | (S)-3-cyano-N-(2,3-difluoro-4-((3-(2-(piperidin- 3-ylamino)pyrimidin-4-yl)pyridin-2- yl)oxy)phenyl)-2,2-dimethylpropane-1- sulfonamide |
| 217 | 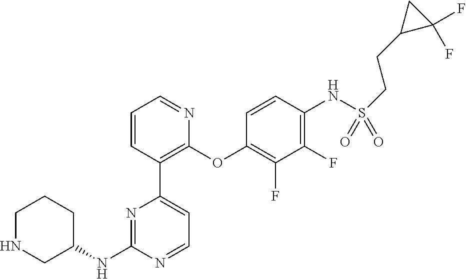 | N-(2,3-difluoro-4-((3-(2-(((S)-piperidin-3- yl)amino)pyrimidin-4-yl)pyridin-2- yl)oxy)phenyl)-2-(2,2- difluorocyclopropyl)ethane-1-sulfonamide |
| 218 | 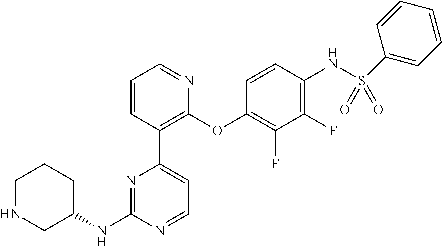 | (S)-N-(2,3-difluoro-4-((3-(2-(piperidin-3- ylamino)pyrimidin-4- yl)pyridin-2-yl)oxy)phenyl)benzenesulfonamide |
| 219 | 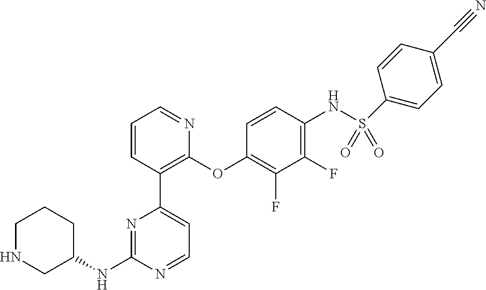 | (S)-4-cyano-N-(2,3-difluoro-4-((3-(2- (piperidin- 3-ylamino)pyrimidin-4-yl)pyridin-2- yl)oxy)phenyl)benzenesulfonamide |
| 220 | 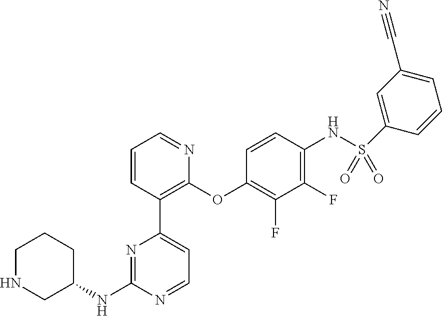 [SMILES: N#Cc1cccc(S(=O)(=O)Nc2ccc(Oc3ncccc3-c3ccnc(N[C@H]4CCCNC4)n3)c(F)c2F)c1] | (S)-3-cyano-N-(2,3-difluoro-4-((3-(2-(piperidin- 3-ylamino)pyrimidin-4- yl)pyridin-2-yl)oxy)phenyl)benzenesulfonamide |
| 221 | 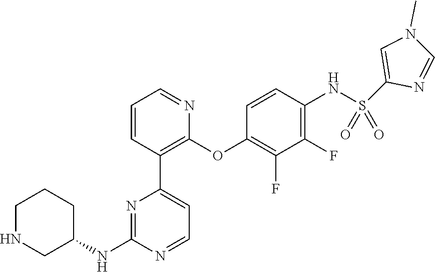 [SMILES: Cn1cnc(S(=O)(=O)Nc2ccc(Oc3ncccc3-c3ccnc(N[C@H]4CCCNC4)n3)c(F)c2F)c1] | (S)-N-(2,3-difluoro-4-((3-(2-(piperidin-3- ylamino)pyrimidin-4-yl)pyridin-2- yl)oxy)phenyl)-1-methyl-1H-imidazole-4- sulfonamide |
| 222 | 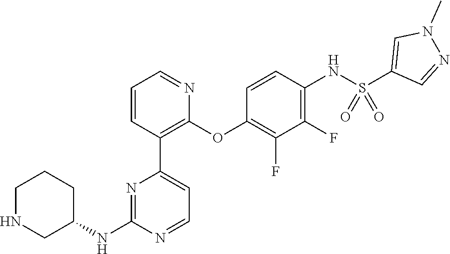 [SMILES: Cn1cc(S(=O)(=O)Nc2ccc(Oc3ncccc3-c3ccnc(N[C@H]4CCCNC4)n3)c(F)c2F)cn1] | (S)-N-(2,3-difluoro-4-((3-(2-(piperidin-3- ylamino)pyrimidin-4-yl)pyridin-2- yl)oxy)phenyl)-1-methyl-1H-pyrazole-4- sulfonamide |
| 223 | 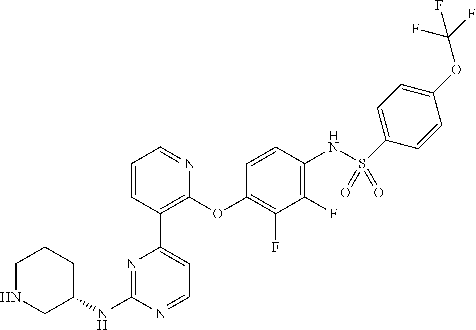 [SMILES: O=S(=O)(Nc1ccc(Oc2ncccc2-c2ccnc(N[C@H]3CCCNC3)n2)c(F)c1F)c1ccc(OC(F)(F)F)cc1] | (S)-N-(2,3-difluoro-4-((3-(2-(piperidin-3- ylamino)pyrimidin-4-yl)pyridin-2- yl)oxy)phenyl)-4- (trifluoromethoxy)benzenesulfonamide |
| 224 | 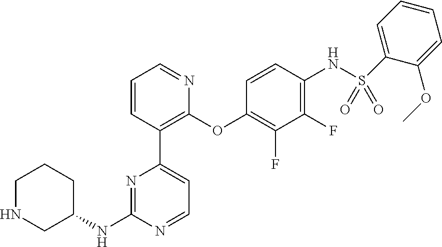 [SMILES: COc1ccccc1S(=O)(=O)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-2-methoxybenzenesulfonamide |
| 225 | 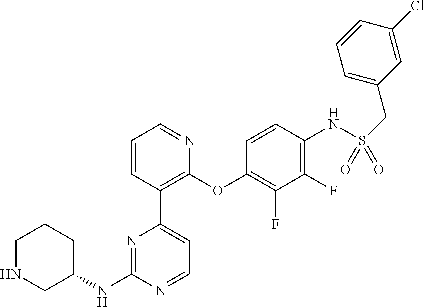 [SMILES: O=S(=O)(Cc1cccc(Cl)c1)Nc1ccc(Oc2ncccc2-c2ccnc(N[C@H]3CCCNC3)n2)c(F)c1F] | (S)-1-(3-chlorophenyl)-N-(2,3-difluoro-4-((3-(2- (piperidin-3-ylamino)pyrimidin-4-yl)pyridin-2- yl)oxy)phenyl)methanesulfonamide |
| 226 | 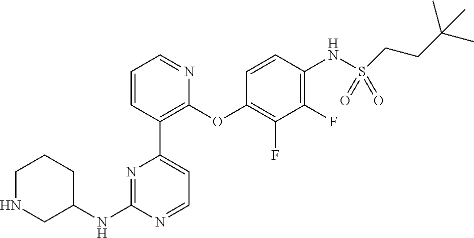 [SMILES: CC(C)(C)CCS(=O)(=O)Nc1ccc(Oc2ncccc2-c2ccnc(NC3CCCNC3)n2)c(F)c1F] | (S)-N-(2,3-difluoro-4-((3-(2-(piperidin-3- ylamino)pyrimidin-4-yl)pyridin-2- yl)oxy)phenyl)-3,3-dimethylbutane-1- sulfonamide |
| 227 | 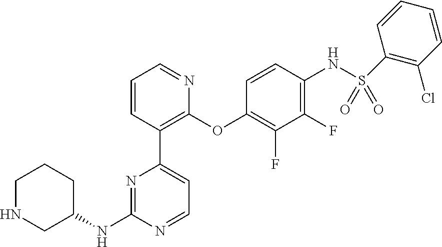 | (S)-2-chloro-N-(2,3-difluoro-4-((3-(2- (piperidin-3-ylamino)pyrimidin-4- yl)pyridin-2-yl)oxy)phenyl)benzenesulfonamide |
| 228 | 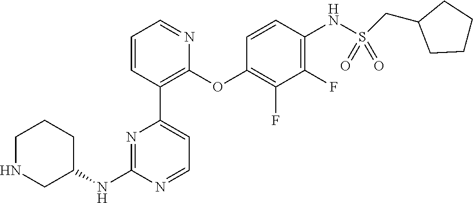 | (S)-1-cyclopentyl-N-(2,3-difluoro-4-((3- (2-(piperidin-3-ylamino)pyrimidin-4-yl)pyridin- 2-yl)oxy)phenyl)methanesulfonamide |
| 229 | 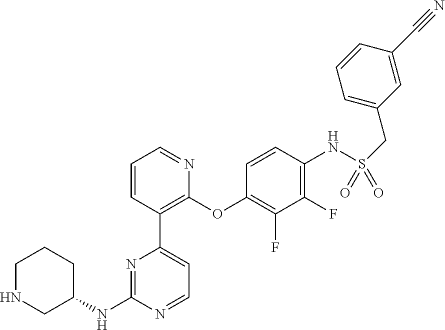 [SMILES: N#Cc1cccc(CS(=O)(=O)Nc2ccc(Oc3ncccc3-c3ccnc(N[C@H]4CCCNC4)n3)c(F)c2F)c1] | (S)-1-(3-cyanophenyl)-N-(2,3-difluoro-4- ((3-(2-(piperidin-3-ylamino)pyrimidin-4- yl)pyridin-2-yl)oxy)phenyl)methanesulfonamide |
| 230 | 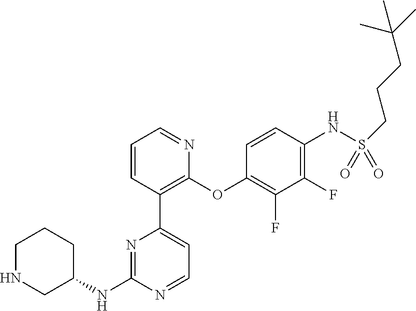 [SMILES: CC(C)(C)CCCS(=O)(=O)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-4,4-dimethylpentane-1- sulfonamide |
| 231 | 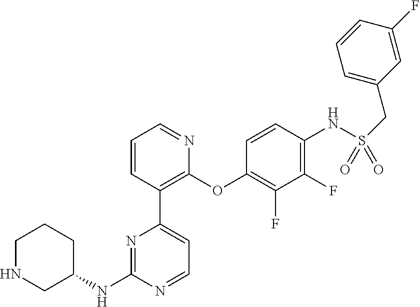 [SMILES: O=S(=O)(Cc1cccc(F)c1)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-1-(3- fluorophenyl)methanesulfonamide |
| 232 | 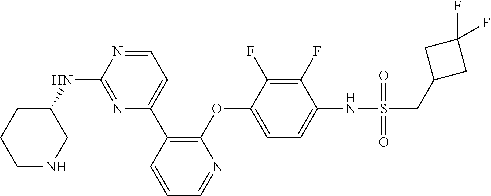 [SMILES: O=S(=O)(CC1CC(F)(F)C1)Nc1ccc(Oc2ncccc2-c2ccnc(N[C@H]3CCCNC3)n2)c(F)c1F] | (S)-N-(2,3-difluoro-4-(3-(2-(piperidin-3- ylamino)pyrimidin-4-yl)pyridin-2-yloxy)phenyl)- 1-(3,3-difluorocyclobutyl)methanesulfonamide |
| 233 | 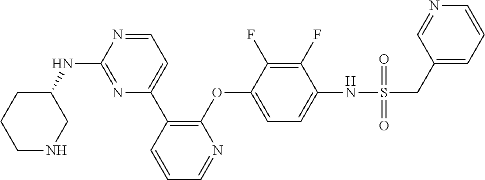 [SMILES: O=S(=O)(Cc1cccnc1)Nc1ccc(Oc2ncccc2-c2ccnc(N[C@H]3CCCNC3)n2)c(F)c1F] | (S)-N-(2,3-difluoro-4-(3-(2-(piperidin-3- ylamino)pyrimidin-4-yl)pyridin-2-yloxy)phenyl)- 1-(pyridin-3-yl)methanesulfonamide |
| 234 | 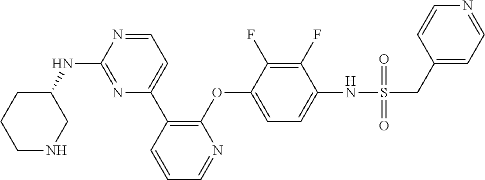 [SMILES: O=S(=O)(Cc1ccncc1)Nc1ccc(Oc2ncccc2-c2ccnc(N[C@H]3CCCNC3)n2)c(F)c1F] | (S)-N-(2,3-difluoro-4-(3-(2-(piperidin-3- ylamino)pyrimidin-4-yl)pyridin-2-yloxy)phenyl)- 1-(pyridin-4-yl)methanesulfonamide |
| 235 | 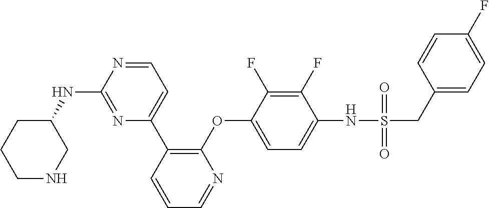 [SMILES: O=S(=O)(Cc1ccc(F)cc1)Nc1ccc(Oc2ncccc2-c2ccnc(N[C@H]3CCCNC3)n2)c(F)c1F] | (S)-N-(2,3-difluoro-4-(3-(2-(piperidin-3- ylamino)pyrimidin-4-yl)pyridin-2-yloxy)phenyl)- 1-(4-fluorophenyl)methanesulfonamide |
| 236 | 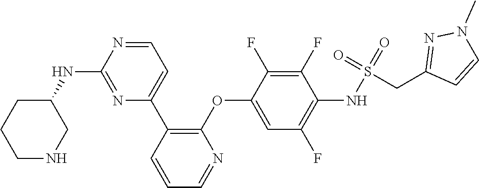 [SMILES: Cn1ccc(CS(=O)(=O)Nc2c(F)cc(Oc3ncccc3-c3ccnc(N[C@H]4CCCNC4)n3)c(F)c2F)n1] | (S)-1-(1-methyl-1H-pyrazol-3-yl)-N-(2,3,6- trifluoro-4-(3-(2-(piperidin-3-ylamino)pyrimidin- 4-yl)pyridin-2-yloxy)phenyl)methanesulfonamide |
| 237 | 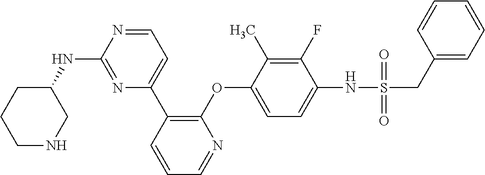 [SMILES: Cc1c(Oc2ncccc2-c2ccnc(N[C@H]3CCCNC3)n2)ccc(NS(=O)(=O)Cc2ccccc2)c1F] | N-[2-fluoro-3-methyl-4-[3-[2-[[(3S)-3- piperidyl]amino]pyrimidin-4-yl]-2- pyridyl]oxy]phenyl]-1- phenylmethanesulfonamide |
| 238 | 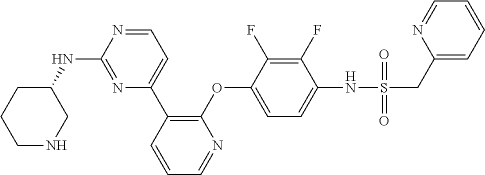 [SMILES: O=S(=O)(Cc1ccccn1)Nc1ccc(Oc2ncccc2-c2ccnc(N[C@H]3CCCNC3)n2)c(F)c1F] | (S)-N-(2,3-difluoro-4-(3-(2-(piperidin-3- ylamino)pyrimidin-4-yl)pyridin-2-yloxy)phenyl)- 1-(pyridin-2-yl)methanesulfonamide |
| 239 | 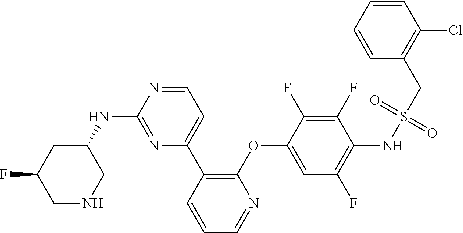 [SMILES: O=S(=O)(Cc1ccccc1Cl)Nc1c(F)cc(Oc2ncccc2-c2ccnc(N[C@@H]3CNC[C@@H](F)C3)n2)c(F)c1F] | 1-(2-chlorophenyl)-N-(2,3,6-trifluoro-4- ((3-(2-(((3S,5S)-5-fluoropiperidin-3- yl)amino)pyrimidin-4-y1)pyridin-2- yl)oxy)phenyl)methanesulfonamide |
| 240 | 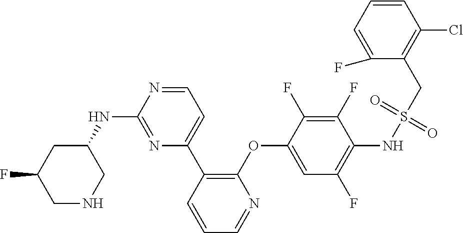 [SMILES: O=S(=O)(Cc1c(F)cccc1Cl)Nc1c(F)cc(Oc2ncccc2-c2ccnc(N[C@@H]3CNC[C@@H](F)C3)n2)c(F)c1F] | 1-(2-chloro-6-fluorophenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 241 | 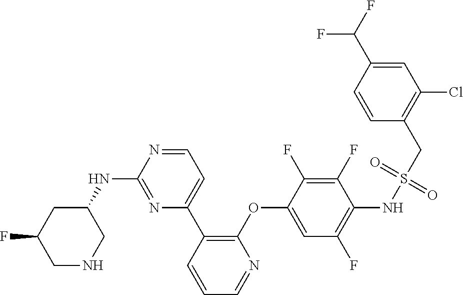 [SMILES: O=S(=O)(Cc1ccc(C(F)F)cc1Cl)Nc1c(F)cc(Oc2ncccc2-c2ccnc(N[C@@H]3CNC[C@@H](F)C3)n2)c(F)c1F] | 1-(4-(difluoromethyl)phenyl)-N-(2,3,6-trifluoro- 4-((3-(2-(((3S,5S)-5-fluoropiperidin-3- yl)amino)pyrimidin-4-yl)pyridin-2- yl)oxy)phenyl)methanesulfonamide |
| 242 | 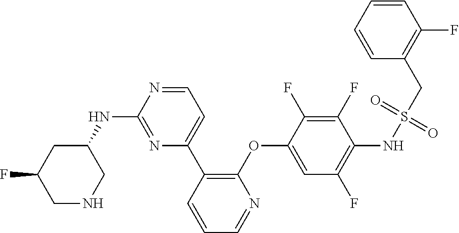 [SMILES: O=S(=O)(Cc1ccccc1F)Nc1c(F)cc(Oc2ncccc2-c2ccnc(N[C@@H]3CNC[C@@H](F)C3)n2)c(F)c1F] | 1-(2-fluorophenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 243 | 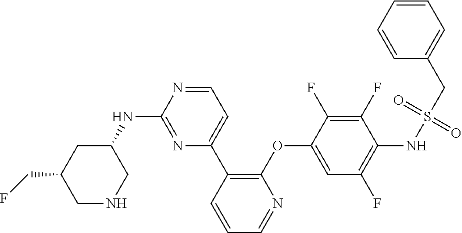 [SMILES: O=S(=O)(Cc1ccccc1)Nc1c(F)cc(Oc2ncccc2-c2ccnc(N[C@@H]3CNC[C@H](CF)C3)n2)c(F)c1F] | 1-phenyl-N-(2,3,6-trifluoro-4-((3-(2-(((3S,5R)-5- (fluoromethyl)piperidin-3-yl)amino)pyrimidin-4- yl)pyridin-2-yl)oxy)phenyl)methanesulfonamide |
| 244 | 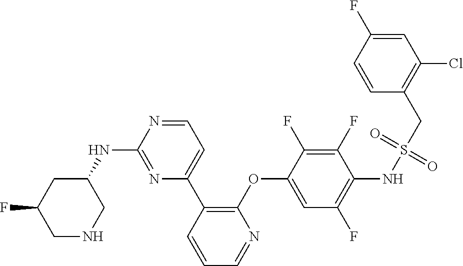 [SMILES: O=S(=O)(Cc1ccc(F)cc1Cl)Nc1c(F)cc(Oc2ncccc2-c2ccnc(N[C@@H]3CNC[C@@H](F)C3)n2)c(F)c1F] | 1-(2-chloro-4-fluorophenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 245 | 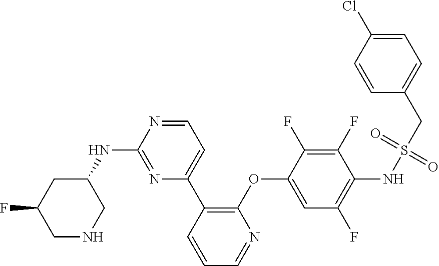 [SMILES: O=S(=O)(Cc1ccc(Cl)cc1)Nc1c(F)cc(Oc2ncccc2-c2ccnc(N[C@@H]3CNC[C@@H](F)C3)n2)c(F)c1F] | 1-(4-chlorophenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 246 | 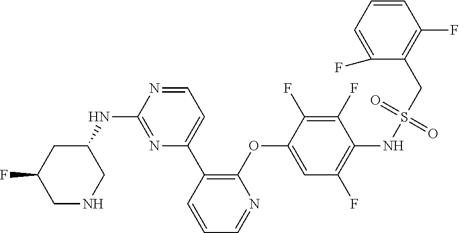 [SMILES: O=S(=O)(Cc1c(F)cccc1F)Nc1c(F)cc(Oc2ncccc2-c2ccnc(N[C@@H]3CNC[C@@H](F)C3)n2)c(F)c1F] | 1-(2,6-difluorophenyl)-N-(2,3,6-trifluoro-4-((3- (2-(((3S,5S)-5-fluoropiperidin-3- yl)amino)pyrimidin-4-yl)pyridin-2- yl)oxy)phenyl)methanesulfonamide |
| 247 | 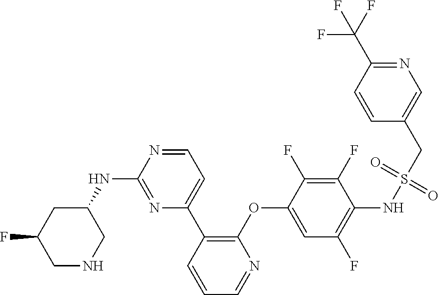 [SMILES: O=S(=O)(Cc1ccc(C(F)(F)F)nc1)Nc1c(F)cc(Oc2ncccc2-c2ccnc(N[C@@H]3CNC[C@@H](F)C3)n2)c(F)c1F] | N-(2,3,6-trifluoro-4-((3-(2-(((35,5S)-5- fluoropiperidin-3-yl)amino)pyrimidin-4- yl)pyridin-2-yl)oxy)phenyl)-1-(6- (trifluoromethyl)pyridin-3- yl)methanesulfonamide |
| 248 | 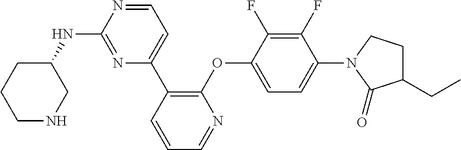 [SMILES: CCC1CCN(c2ccc(Oc3ncccc3-c3ccnc(N[C@H]4CCCNC4)n3)c(F)c2F)C1=O] | 1-(2,3-difluoro-4-((3-(2-(((S)-piperidin- 3-yl)amino)pyrimidin-4- yl)pyridin-2-yl)oxy)phenyl)-3-ethylpyrrolidin-2- one |
| 250 | 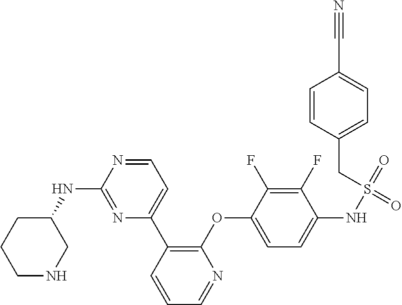 [SMILES: N#Cc1ccc(CS(=O)(=O)Nc2ccc(Oc3ncccc3-c3ccnc(N[C@H]4CCCNC4)n3)c(F)c2F)cc1] | (S)-1-(4-cyanophenyl)-N-(2,3-difluoro-4- ((3-(2-(piperidin-3-ylamino)pyrimidin-4- yl)pyridin-2-yl)oxy)phenyl)methanesulfonamide |
| 251 | 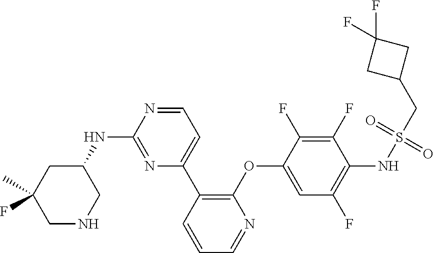 [SMILES: C[C@@]1(F)CNC[C@@H](Nc2nccc(-c3cccnc3Oc3cc(F)c(NS(=O)(=O)CC4CC(F)(F)C4)c(F)c3F)n2)C1] | 1-(3,3-difluorocyclobutyl)-N-(2,3,6-trifluoro-4- ((3-(2-(((3S,5S)-5-fluoro-5-methylpiperidin-3- yl)amino)pyrimidin-4-yl)pyridin-2- yl)oxy)phenyl)methanesulfonamide |
| 252 | 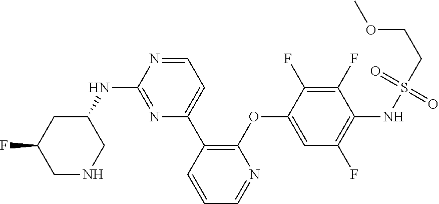 [SMILES: COCCS(=O)(=O)Nc1c(F)cc(Oc2ncccc2-c2ccnc(N[C@@H]3CNC[C@@H](F)C3)n2)c(F)c1F] | 2-methoxy-N-(2,3,6-trifluoro-4-((3-(2-(((35,5S)- 5-fluoropiperidin-3-yl)amino)pyrimidin-4- yl)pyridin-2-yl)oxy)phenyl)ethane-1-sulfonamide |
| 253 | 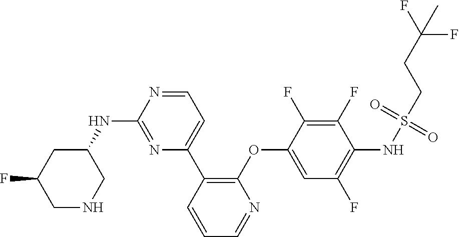 [SMILES: CC(F)(F)CCS(=O)(=O)Nc1c(F)cc(Oc2ncccc2-c2ccnc(N[C@@H]3CNC[C@@H](F)C3)n2)c(F)c1F] | 3,3-difluoro-N-(2,3,6-trifluoro-4-((3-(2-(((3S,5S)- 5-fluoropiperidin-3-yl)amino)pyrimidin-4- yl)pyridin-2-yl)oxy)phenyl)butane-1-sulfonamide |
| 254 | 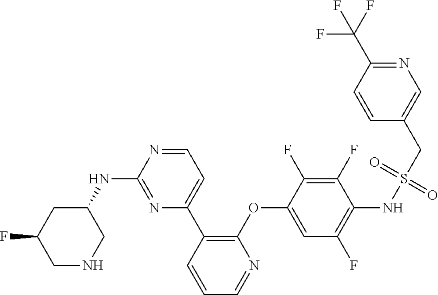 [SMILES: O=S(=O)(Cc1ccc(C(F)(F)F)nc1)Nc1c(F)cc(Oc2ncccc2-c2ccnc(N[C@@H]3CNC[C@@H](F)C3)n2)c(F)c1F] | N-(2,3,6-trifluoro-4-((3-(2-(((3S,5S)-5- fluoropiperidin-3-yl)amino)pyrimidin-4- yl)pyridin-2-yl)oxy)phenyl)-1-(6- (trifluoromethyl)pyridin-3- yl)methanesulfonamide |
| 255 | 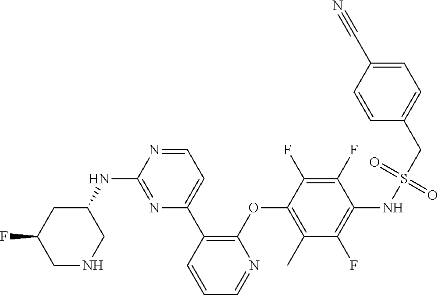 [SMILES: Cc1c(F)c(NS(=O)(=O)Cc2ccc(C#N)cc2)c(F)c(F)c1Oc1ncccc1-c1ccnc(N[C@@H]2CNC[C@@H](F)C2)n1] | 1-(4-cyanophenyl)-N-(2,3,6-trifluoro-4- ((3-(2-(((35,5S)-5-fluoropiperidin-3- yl)amino)pyrimidin-4-yl)pyridin-2-yl)oxy)-5- methylphenyl)methanesulfonamide |
| 256 | 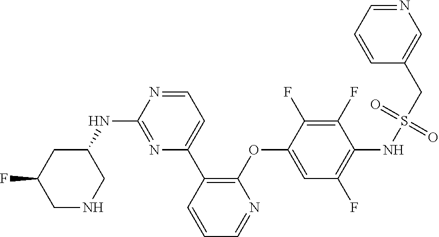 [SMILES: O=S(=O)(Cc1cccnc1)Nc1c(F)cc(Oc2ncccc2-c2ccnc(N[C@@H]3CNC[C@@H](F)C3)n2)c(F)c1F] | 1-(pyridin-3-yl)-N-(2,3,6-trifluoro-4-((3- (2-(((3S,5S)-5-fluoropiperidin-3- yl)amino)pyrimidin-4-yl)pyridin-2- yl)oxy)phenyl)methanesulfonamide |
| 257 | 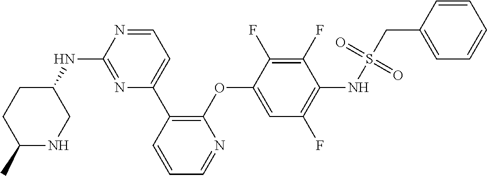 [SMILES: C[C@H]1CC[C@H](Nc2nccc(-c3cccnc3Oc3cc(F)c(NS(=O)(=O)Cc4ccccc4)c(F)c3F)n2)CN1] | 1-phenyl-N-(2,3,6-trifluoro-4-((3-(2- (((3S6S)-6-methylpiperidm-3- yl)amino)pyrimidin-4-yl)pyridin-2- yl)oxy)phenyl)methanesulfonamide |
| 258 | 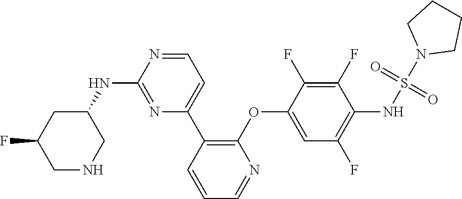 [SMILES: O=S(=O)(Nc1c(F)cc(Oc2ncccc2-c2ccnc(N[C@@H]3CNC[C@@H](F)C3)n2)c(F)c1F)N1CCCC1] | N-(2,3,6-trifluoro-4-((3-(2-(((3S,5S)-5- fluoropiperidin-3-yl)amino)pyrimidin-4- yl)pyridin-2-yl)oxy)phenyl)pyrrolidine-1- sulfonamide |
| 259 | 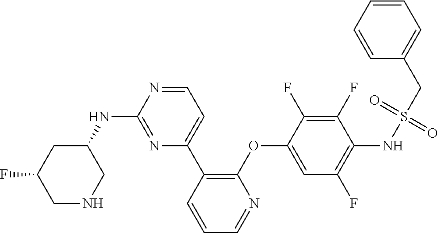 [SMILES: O=S(=O)(Cc1ccccc1)Nc1c(F)cc(Oc2ncccc2-c2ccnc(N[C@@H]3CNC[C@H](F)C3)n2)c(F)c1F] | 1-phenyl-N-(2,3,6-trifluoro-4-((3-(2-(((3S,5R)-5- fluoropiperidin-3-yl)amino)pyrimidin-4- yl)pyridin-2-yl)oxy)phenyl)methanesulfonamide |
| 260 | 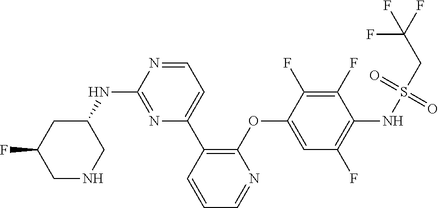 [SMILES: O=S(=O)(CC(F)(F)F)Nc1c(F)cc(Oc2ncccc2-c2ccnc(N[C@@H]3CNC[C@@H](F)C3)n2)c(F)c1F] | 2,2,2-trifluoro-N-(2,3,6-trifluoro-4-((3- (2-(((35,5S)-5-fluoropiperidin-3- yl)amino)pyrimidin-4-yl)pyridin-2- yl)oxy)phenyl)ethane-1- sulfonamidehydrochloride |
| 261 | 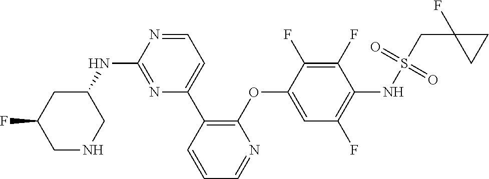 [SMILES: O=S(=O)(CC1(F)CC1)Nc1c(F)cc(Oc2ncccc2-c2ccnc(N[C@@H]3CNC[C@@H](F)C3)n2)c(F)c1F] | 1-(1-fluorocyclopropyl)-N-(2,3,6- trifluoro-4-((3-(2-(((3S,5S)-5-fluoropiperidin-3- yl)amino)pyrimidin-4-yl)pyridin-2- yl)oxy)phenyl)methanesulfonamide |
| 262 | 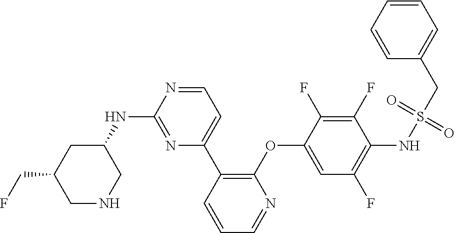 [SMILES: O=S(=O)(Cc1ccccc1)Nc1c(F)cc(Oc2ncccc2-c2ccnc(N[C@@H]3CNC[C@H](CF)C3)n2)c(F)c1F] | 1-phenyl-N-(2,3,6-trifluoro-4-((3-(2-(((3S,5R)-5- (fluoromethyl)piperidin-3-yl)amino)pyrimidin-4- yl)pyridin-2-yl)oxy)phenyl)methanesulfonamide |
| 263 | 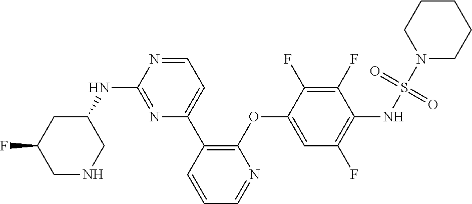 [SMILES: O=S(=O)(Nc1c(F)cc(Oc2ncccc2-c2ccnc(N[C@@H]3CNC[C@@H](F)C3)n2)c(F)c1F)N1CCCCC1] | N-(2,3,6-trifluoro-4-((3-(2-(((3S,5S)-5- fluoropiperidin-3-yl)amino)pyrimidin-4- yl)pyridin-2-yl)oxy)phenyl)piperidine-1- sulfonamide |
| 264 | 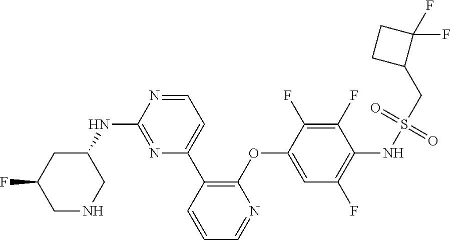 [SMILES: O=S(=O)(CC1CCC1(F)F)Nc1c(F)cc(Oc2ncccc2-c2ccnc(N[C@@H]3CNC[C@@H](F)C3)n2)c(F)c1F] | 1-(2,2-difluorocyclobutyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 266 | 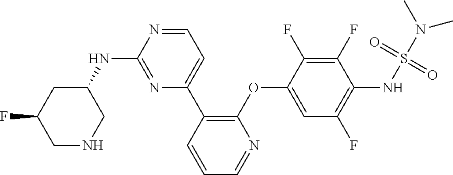 [SMILES: CN(C)S(=O)(=O)Nc1c(F)cc(Oc2ncccc2-c2ccnc(N[C@@H]3CNC[C@@H](F)C3)n2)c(F)c1F] | 4-(2-(4-(dimethylsulfamoylamino)-2,3,5- trifluoro-phenoxy)-3-pyridyl)-2- (((3S,5S)-5-fluoro-3-piperidyl)amino)pyrimidine |
| 267 | 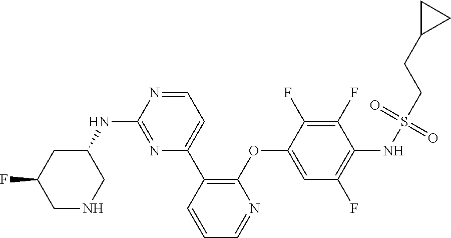 [SMILES: O=S(=O)(CCC1CC1)Nc1c(F)cc(Oc2ncccc2-c2ccnc(N[C@@H]3CNC[C@@H](F)C3)n2)c(F)c1F] | 2-cyclopropyl-N-(2,3,6-trifluoro-4-((3- (2-(((3S,5S)-5-fluoropiperidin-3- yl)amino)pyrimidin-4-yl)pyridin-2- yl)oxy)phenyl)ethane-1-sulfonamide |
| 268 | 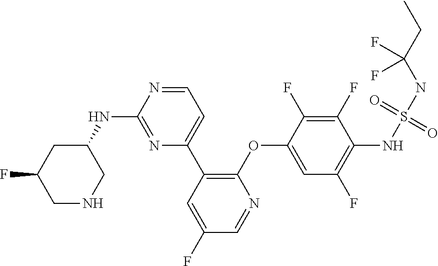 [SMILES: CCC(F)(F)NS(=O)(=O)Nc1c(F)cc(Oc2ncc(F)cc2-c2ccnc(N[C@@H]3CNC[C@@H](F)C3)n2)c(F)c1F] | 2,2-difluoro-N-(2,3,6-trifluoro-4-((5-fluoro-3-(2- ([(3S,5S)-5-fluoropiperidin-3- yl)amino)pyrimidin-4-yl)pyridin-2- yl)oxy)phenyl)butane-1-sulfonamide |
| 269 | 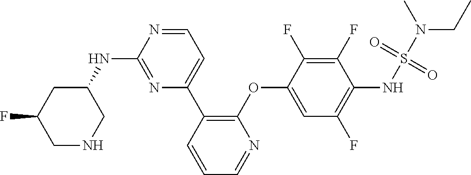 [SMILES: CCN(C)S(=O)(=O)Nc1c(F)cc(Oc2ncccc2-c2ccnc(N[C@@H]3CNC[C@@H](F)C3)n2)c(F)c1F] | 4-(2-(4-((ethyl(methyl)sulfamoyl)amino)-2,3,5- trifluoro-phenoxy)-3-pyridyl)-2-(((3S,5S)-5- fluoro-3-piperidyl)amino)pyrimidine |
| 270 | 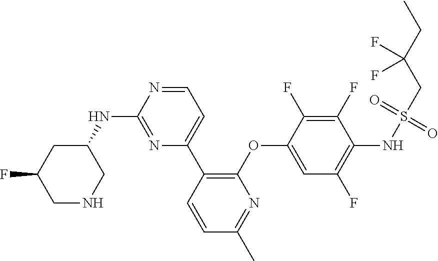 [SMILES: CCC(F)(F)CS(=O)(=O)Nc1c(F)cc(Oc2nc(C)ccc2-c2ccnc(N[C@@H]3CNC[C@@H](F)C3)n2)c(F)c1F] | 2,2-difluoro-N-(2,3,6-trifluoro-4-((3-(2-(((3S,5S)- 5-fluoropiperidin-3-yl)amino)pyrimidin-4-yl)-6- methylpyridin-2-yl)oxy)phenyl)butane-1- sulfonamide |
| 271 | 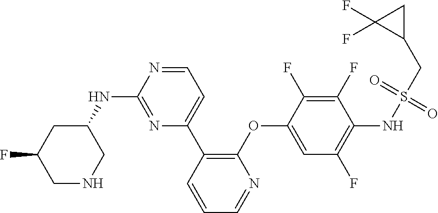 [SMILES: O=S(=O)(CC1CC1(F)F)Nc1c(F)cc(Oc2ncccc2-c2ccnc(N[C@@H]3CNC[C@@H](F)C3)n2)c(F)c1F] | 1-(2,2-difluorocyclopropyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 272 | 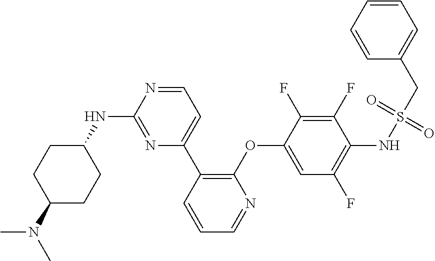 [SMILES: CN(C)[C@H]1CC[C@H](Nc2nccc(-c3cccnc3Oc3cc(F)c(NS(=O)(=O)Cc4ccccc4)c(F)c3F)n2)CC1] | N-(4-((3-(2-(((1r,4r)-4- (dimethylamino)cyclohexyl)amino)pyrimidin-4- yl)pyridin-2-yl)oxy)-2,3,6-trifluorophenyl)-1- phenylmethanesulfonamide hydrochloride |
| 273 | 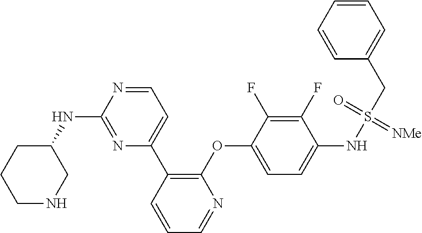 | N-(2,3-difluoro-4-((3-(2-(((S)-piperidin-3- yl)amino)pyrimidin-4-yl)pyridin-2- yl)oxy)phenyl)-N-methyl-1- phenylmethanesulfonimidamide |
| 275 | 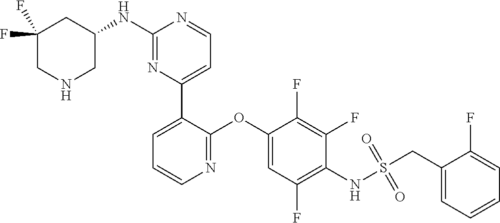 | 1-(2-fluorophenyl)-N-(2,3,6-trifluoro-4- ((3-(2-(((3S,5S)-5-fluoro-5-methylpiperidin-3- yl)amino)pyrimidin-4-yl)pyridin-2- yl)oxy)phenyl)methanesulfonamide |
| 276 | 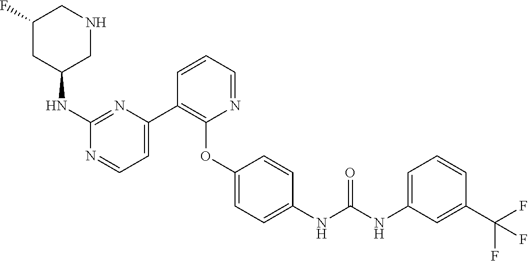 [SMILES: O=C(Nc1ccc(Oc2ncccc2-c2ccnc(N[C@@H]3CNC[C@@H](F)C3)n2)cc1)Nc1cccc(C(F)(F)F)c1] | 1-(4-((3-(2-(((3S,5S)-5-Fluoropiperidin-3- yl)amino)pyrimidin-4-yl)pyridin-2- yl)oxy)phenyl)-3-(3-(trifluoromethyl)phenyl)urea |
| 277 | 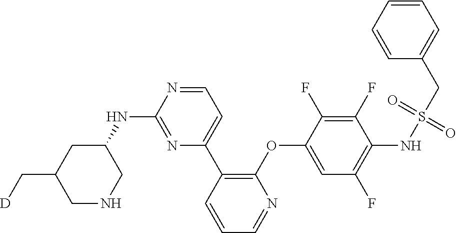 [SMILES: [2H]CC1CNC[C@@H](Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5- (fluoromethyl)piperidin-3-yl)amino)pyrimidin-4- yl)pyridin-2-yl)oxy)phenyl)methanesulfonamide |
| 279 | 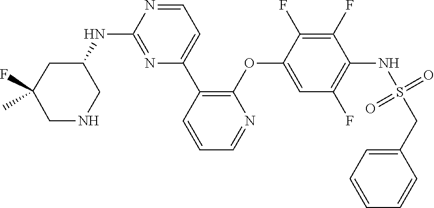 [SMILES: C[C@@]1(F)CNC[C@@H](Nc2nccc(-c3cccnc3Oc3cc(F)c(NS(=O)(=O)Cc4ccccc4)c(F)c3F)n2)C1] | N-(2,3-difluoro-4-((3-(2-(((3S,5S)-5-fluoro-5- methylpiperidin-3-yl)amino)pyrimidin-4- yl)pyridin-2-yl)oxy)-6-methylphenyl)-1- phenylmethanesulfonamide |
| 280 | 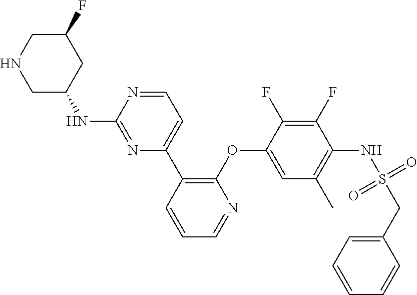 [SMILES: Cc1cc(Oc2ncccc2-c2ccnc(N[C@@H]3CNC[C@@H](F)C3)n2)c(F)c(F)c1NS(=O)(=O)Cc1ccccc1] | N-(2,3-difluoro-4-((3-(2-(((3S,5S)-5- fluoropiperidin-3-y1)amino)pyrimidin-4- yl)pyridin-2-yl)oxy)-6-methylphenyl)-1- phenylmethanesulfonamide |
| 281 | 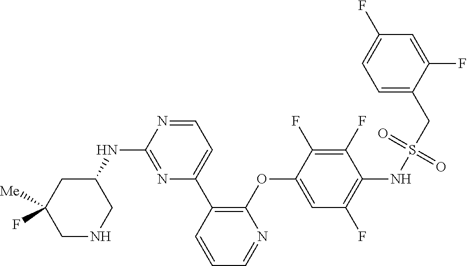 [SMILES: C[C@@]1(F)CNC[C@@H](Nc2nccc(-c3cccnc3Oc3cc(F)c(NS(=O)(=O)Cc4ccc(F)cc4F)c(F)c3F)n2)C1] | 1-(2,4-difluorophenyl)-N-(2,3,6-trifluoro-4-((3- (2-(((3S5S)-5-fluoro-5-methylpiperidin-3- yl)amino)pyrimidin-4-yl)pyridin-2- yl)oxy)phenyl)methanesulfonamide |
| 282 | 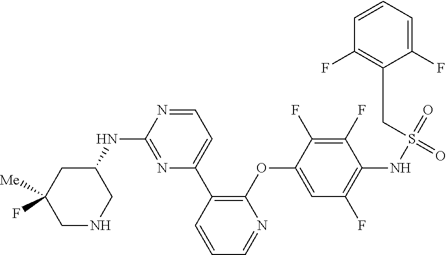 [SMILES: C[C@@]1(F)CNC[C@@H](Nc2nccc(-c3cccnc3Oc3cc(F)c(NS(=O)(=O)Cc4c(F)cccc4F)c(F)c3F)n2)C1] | 1-(2,6-difluorophenyl)-N-(2,3,6-trifluoro- 4-((3-(2-(((3S5S)-5-fluoro-5-methylpiperidin-3- yl)amino)pyrimidin-4-yl)pyridin-2- yl)oxy)phenyl)methanesulfonamide |
| 283 | 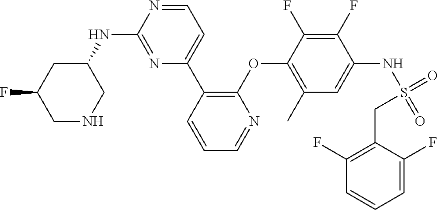 [SMILES: Cc1cc(NS(=O)(=O)Cc2c(F)cccc2F)c(F)c(F)c1Oc1ncccc1-c1ccnc(N[C@@H]2CNC[C@@H](F)C2)n1] | N-(2,3-difluoro-4-((3-(2-(((3S,5S)-5- fluoropiperidin-3-yl)amino)pyrimidin-4- yl)pyridin-2-yl)oxy)-5-methylphenyl)-1-(2,6- difluorophenyl)methanesulfonamide |
| 284 | 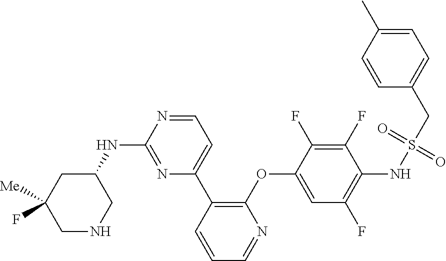 [SMILES: Cc1ccc(CS(=O)(=O)Nc2c(F)cc(Oc3ncccc3-c3ccnc(N[C@@H]4CNC[C@@](C)(F)C4)n3)c(F)c2F)cc1] | 1-p-toly1-N-(2,3,6-trifluoro-4-((3-(2-(((3S,5S)-5- fluoro-5-methylpiperidin-3-yl)amino)pyrimidin- 4-yl)pyridin-2- yl)oxy)phenyl)methanesulfonamide |
| 285 | 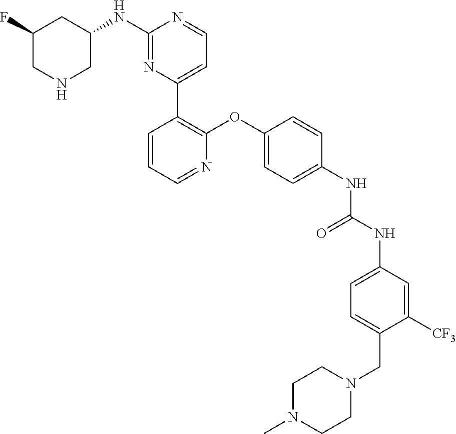 [SMILES: CN1CCN(Cc2ccc(NC(=O)Nc3ccc(Oc4ncccc4-c4ccnc(N[C@@H]5CNC[C@@H](F)C5)n4)cc3)cc2C(F)(F)F)CC1] | 1-(4-((3-(2-(((3S,5S)-5-fluoropiperidin-3- yl)amino)pyrimidin-4-yl)pyridin-2- yl)oxy)phenyl)-3-(4-((4-methylpiperazin-1- yl)methyl)-3- (trifluoromethyl)phenyl)ureahydrochloride |
| 286 | 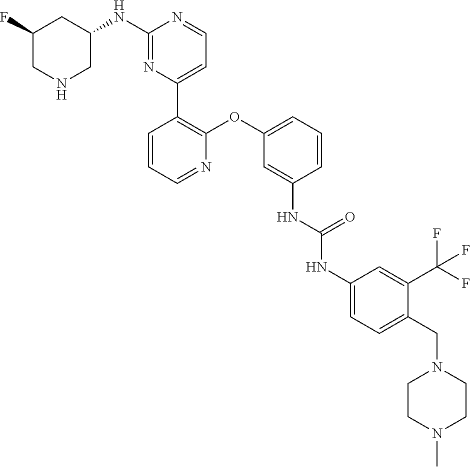 [SMILES: CN1CCN(Cc2ccc(NC(=O)Nc3cccc(Oc4ncccc4-c4ccnc(N[C@@H]5CNC[C@@H](F)C5)n4)c3)cc2C(F)(F)F)CC1] | 1-(3-((3-(2-(((3S,5S)-5-fluoropiperidin-3- yl)amino)pyrimidin-4-yl)pyridin-2- yl)oxy)phenyl)-3-(4-((4-methylpiperazin-1- yl)methyl)-3- (trifluoromethyl)phenyl)ureahydrochloride |
| 287 | 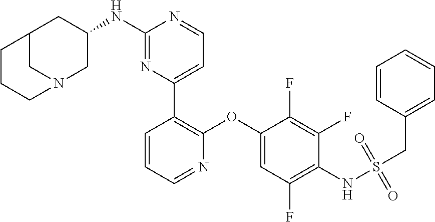 [SMILES: O=S(=O)(Cc1ccccc1)Nc1c(F)cc(Oc2ncccc2-c2ccnc(N[C@H]3CC4CCCN(C4)C3)n2)c(F)c1F] | N-(4-((3-(2-((1-azabicyclo[3.3.1]nonan-3- yl)amino)pyrimidin-4-yl)pyridin-2-yl)oxy)-2,3,6- trifluorophenyl)-1-phenylmethanesulfonamide hydrochloride |
| 290 | 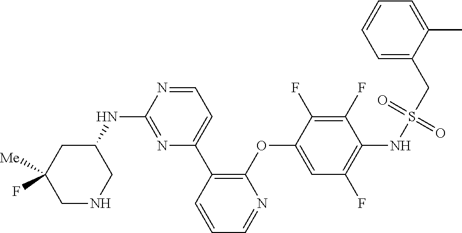 [SMILES: Cc1ccccc1CS(=O)(=O)Nc1c(F)cc(Oc2ncccc2-c2ccnc(N[C@@H]3CNC[C@@](C)(F)C3)n2)c(F)c1F] | 1-o-tolyl-N-(2,3,6-trifluoro-4-((3-(2-(((3S,5S)-5- fluoro-5-methylpiperidin-3-yl)amino)pyrimidin- 4-yl)pyridin-2- yl)oxy)phenyl)methanesulfonamide |
| 291 | 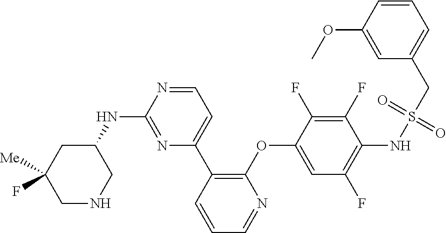 [SMILES: COc1cccc(CS(=O)(=O)Nc2c(F)cc(Oc3ncccc3-c3ccnc(N[C@@H]4CNC[C@@](C)(F)C4)n3)c(F)c2F)c1] | 1-(3-methoxyphenyl)-N-(2,3,6-trifluoro-4-((3-(2- (((3S5S)-5-fluoro-5-methylpiperidin-3- yl)amino)pyrimidin-4-yl)pyridin-2- yl)oxy)phenyl)methanesulfonamide |
| 292 | 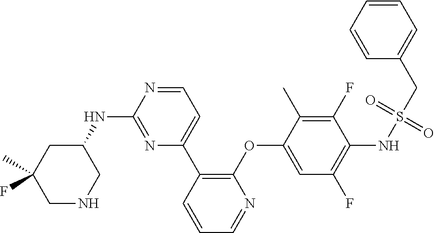 [SMILES: Cc1c(Oc2ncccc2-c2ccnc(N[C@@H]3CNC[C@@](C)(F)C3)n2)cc(F)c(NS(=O)(=O)Cc2ccccc2)c1F] | N-(2,6-difluoro-4-((3-(2-(((3S,5S)-5-fluoro-5- methylpiperidin-3-yl)amino)pyrimidin-4- yl)pyridin-2-yl)oxy)-3-methylphenyl)-1- phenylmethanesulfonamide |
| 293 | 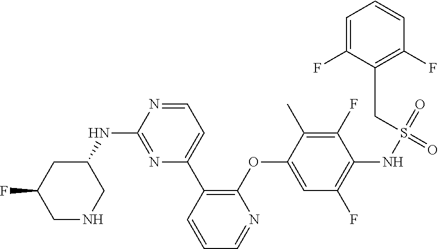 [SMILES: Cc1c(Oc2ncccc2-c2ccnc(N[C@@H]3CNC[C@@H](F)C3)n2)cc(F)c(NS(=O)(=O)Cc2c(F)cccc2F)c1F] | N-(2,6-difluoro-4-((3-(2-(((3S,5S)-5- fluoropiperidin-3-yl)amino)pyrimidin-4- yl)pyridin-2-yl)oxy)-3-methylphenyl)-1-(2,6- difluorophenyl)methanesulfonamide |
| 294 | 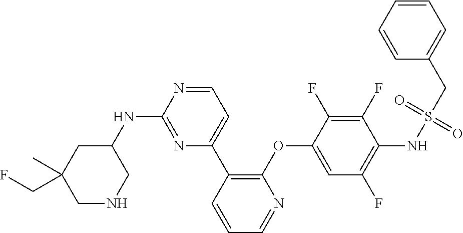 [SMILES: CC1(CF)CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5- (fluoromethyl)-5-methylpiperidin-3- yl)amino)pyrimidin-4-yl)pyridin-2- yl)oxy)phenyl)methanesulfonamide |
| 296 | 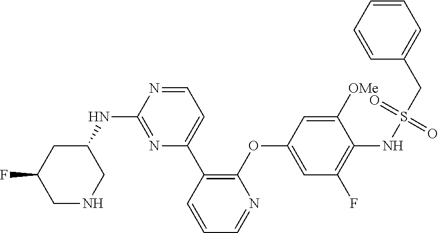 [SMILES: COc1cc(Oc2ncccc2-c2ccnc(N[C@@H]3CNC[C@@H](F)C3)n2)cc(F)c1NS(=O)(=O)Cc1ccccc1] | N-(2-fluoro-4-((3-(2-(((3S,5S)-5-fluoropiperidin- 3-yl)amino)pyrimidin-4-yl)pyridin-2-yl)oxy)-6- methoxyphenyl)-1-phenylmethanesulfonamide |
| 297 | 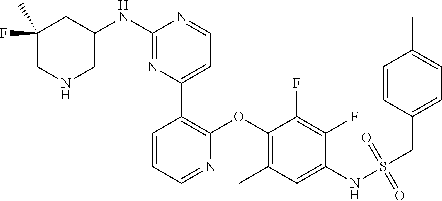 [SMILES: Cc1ccc(CS(=O)(=O)Nc2cc(C)c(Oc3ncccc3-c3ccnc(NC4CNC[C@@](C)(F)C4)n3)c(F)c2F)cc1] | N-(2,3-difluoro-4-((3-(2-(((3S,5S)-5-fluoro-5- methylpiperidin-3-yl)amino)pyrimidin-4- yl)pyridin-2-yl)oxy)-5-methylphenyl)-1-(p- tolyl)methanesulfonamide |
| 298 | 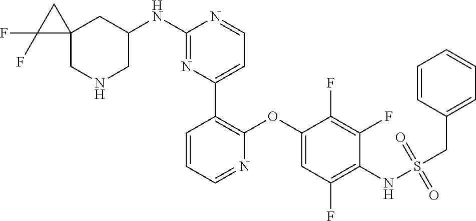 [SMILES: O=S(=O)(Cc1ccccc1)Nc1c(F)cc(Oc2ncccc2-c2ccnc(NC3CNCC4(C3)CC4(F)F)n2)c(F)c1F] | N-(4-((3-(2-((1,1-difluoro-5-azaspiro[2.5]octan-7- yl)amino)pyrimidin-4-yl)pyridin-2-yl)oxy)-2,3,6- trifluorophenyl)-1-phenylmethanesulfonamide |
| 302 | 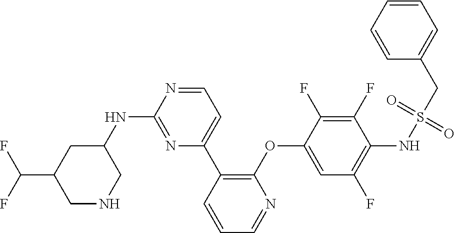 [SMILES: O=S(=O)(Cc1ccccc1)Nc1c(F)cc(Oc2ncccc2-c2ccnc(NC3CNCC(C(F)F)C3)n2)c(F)c1F] | N-(4-((3-(2-((5-(difluoromethyl)piperidin-3- yl)amino)pyrimidin-4-yl)pyridin-2-yl)oxy)-2,3,6- trifluorophenyl)-1-phenylmethanesulfonamide |
| 304 | 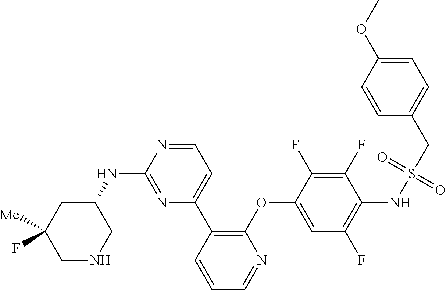 [SMILES: COc1ccc(CS(=O)(=O)Nc2c(F)cc(Oc3ncccc3-c3ccnc(N[C@@H]4CNC[C@@](C)(F)C4)n3)c(F)c2F)cc1] | 1-(4-methoxyphenyl)-N-(2,3,6-trifluoro- 4-((3-(2-(((3S5S)-5-fluoro-5-methylpiperidin-3- yl)amino)pyrimidin-4-yl)pyridin-2- yl)oxy)phenyl)methanesulfonamide |
| 305 | 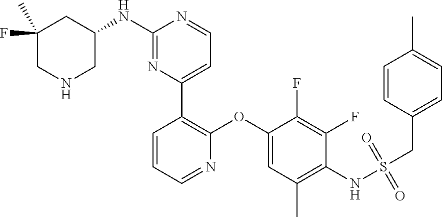 [SMILES: Cc1ccc(CS(=O)(=O)Nc2c(C)cc(Oc3ncccc3-c3ccnc(N[C@@H]4CNC[C@@](C)(F)C4)n3)c(F)c2F)cc1] | N-(2,3-difluoro-4-((3-(2-(((3S,5S)-5-fluoro-5- methylpiperidin-3-yl)amino)pyrimidin-4- yl)pyridin-2-yl)oxy)-6-methylphenyl)-1-(p- tolyl)methanesulfonamide |
| 306 | 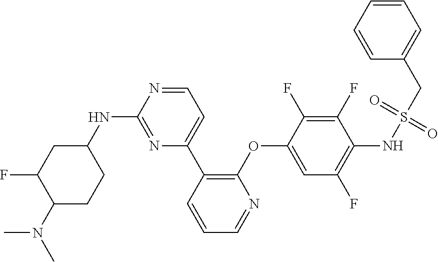 [SMILES: CN(C)C1CCC(Nc2nccc(-c3cccnc3Oc3cc(F)c(NS(=O)(=O)Cc4ccccc4)c(F)c3F)n2)CC1F] | N-(4-((3-(2-((4-(dimethylamino)-3- fluorocyclohexyl)amino)pyrimidin-4-yl)pyridin- 2-yl)oxy)-2,3,6-trifluorophenyl)-1- phenylmethanesulfonamide |
| 309 | 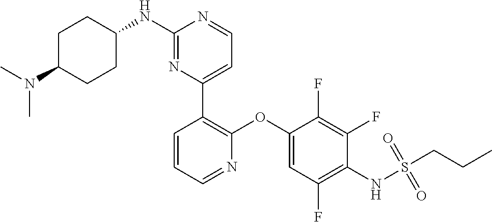 [SMILES: CCCS(=O)(=O)Nc1c(F)cc(Oc2ncccc2-c2ccnc(N[C@H]3CC[C@H](N(C)C)CC3)n2)c(F)c1F] | N-(4-((3-(2-(((1r,4r)-4- (dimethylamino)cyclohexyl)amino)pyrimidin-4- yl)pyridin-2-yl)oxy)-2,3,6- trifluorophenyl)propane-1-sulfonamide |
| 310 | 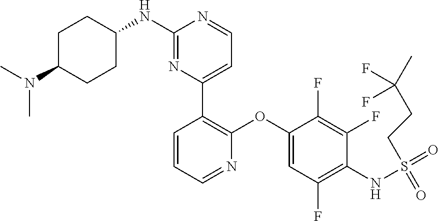 [SMILES: CN(C)[C@H]1CC[C@H](Nc2nccc(-c3cccnc3Oc3cc(F)c(NS(=O)(=O)CCC(C)(F)F)c(F)c3F)n2)CC1] | N-(4-((3-(2-(((1r,4r)-4- (dimethylamino)cyclohexyl)amino)pyrimidin-4- yl)pyridin-2-yl)oxy)-2,3,6-trifluorophenyl)-3,3- difluorobutane-1-sulfonamide |
| 311 | 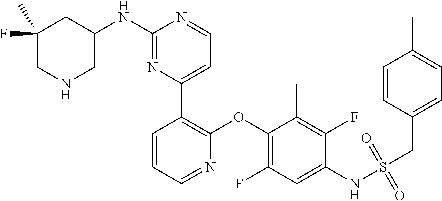 [SMILES: Cc1ccc(CS(=O)(=O)Nc2cc(F)c(Oc3ncccc3-c3ccnc(NC4CNC[C@@](C)(F)C4)n3)c(C)c2F)cc1] | N-(2,5-difluoro-4-((3-(2-(((3S,5S)-5-fluoro-5- methylpiperidin-3-yl)amino)pyrimidin-4- yl)pyridin-2-yl)oxy)-3-methylphenyl)-1 -(p- tolyl)methanesulfonamide |
| 312 | 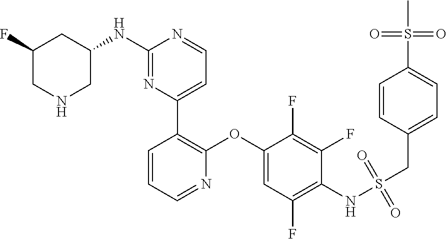 [SMILES: CS(=O)(=O)c1ccc(CS(=O)(=O)Nc2c(F)cc(Oc3ncccc3-c3ccnc(N[C@@H]4CNC[C@@H](F)C4)n3)c(F)c2F)cc1] | 1-(4-(methylsulfonyl)phenyl)-N-(2,3,6-trifluoro- 4-((3-(2-(((3S,5S)-5-fluoropiperidin-3- yl)amino)pyrimidin-4-yl)pyridin-2- yl)oxy)phenyl)methanesulfonamide |
| 313 | 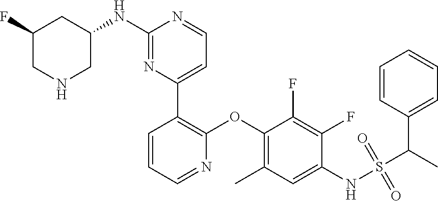 [SMILES: Cc1cc(NS(=O)(=O)C(C)c2ccccc2)c(F)c(F)c1Oc1ncccc1-c1ccnc(N[C@@H]2CNC[C@@H](F)C2)n1] | N-(2,3-difluoro-4-((3-(2-(((3S,5S)-5- fluoropiperidin-3-yl)amino)pyrimidin-4- yl)pyridin-2-yl)oxy)-5-methylphenyl)-1- phenylethane-1-sulfonamidehydrochloride |
| 314 | 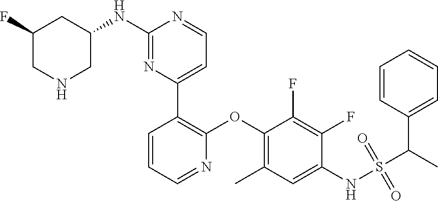 [SMILES: Cc1cc(NS(=O)(=O)C(C)c2ccccc2)c(F)c(F)c1Oc1ncccc1-c1ccnc(N[C@@H]2CNC[C@@H](F)C2)n1] | N-(2,3-difluoro-4-((3-(2-(((3S,5S)-5- fluoropiperidin-3-yl)amino)pyrimidin-4- yl)pyridin-2-yl)oxy)-5-methylphenyl)-1- phenylethane-1-sulfonamide |
| 315 | 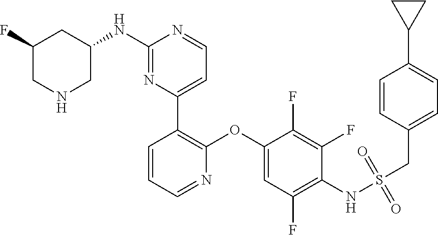 [SMILES: O=S(=O)(Cc1ccc(C2CC2)cc1)Nc1c(F)cc(Oc2ncccc2-c2ccnc(N[C@@H]3CNC[C@@H](F)C3)n2)c(F)c1F] | 1-(4-cyclopropylphenyl)-N-(2,3,6- trifluoro-4-((3- (2-(((35,5S)-5-fluoropiperidin-3- yl)amino)pyrimidin-4-yl)pyridin-2- yl)oxy)phenyl)methanesulfonamide |
| 316 | 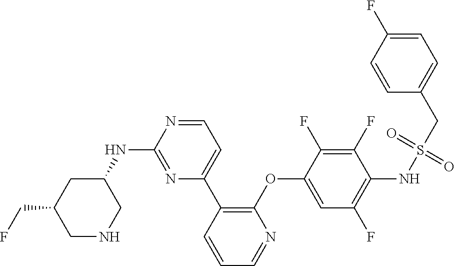 [SMILES: O=S(=O)(Cc1ccc(F)cc1)Nc1c(F)cc(Oc2ncccc2-c2ccnc(N[C@@H]3CNC[C@H](CF)C3)n2)c(F)c1F] | 1-(4-fluorophenyl)-N-(2.3.6-trifluoro-4-((3-(2- (((3S,5R)-5-(fluoromethyl)piperidin-3- yl)amino)pyrimidin-4-yl)pyridin-2- yl)oxy)phenyl)methanesulfonamide |
| 317 | 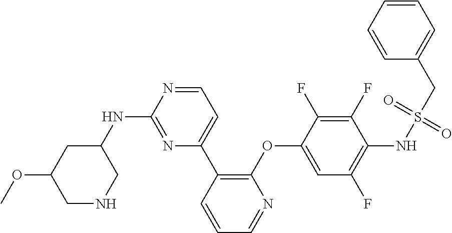 [SMILES: COC1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5- methoxypiperidin-3-yl)amino)pyrimidin-4- yl)pyridin-2-yl)oxy)phenyl)methanesulfonamide |
| 320 | 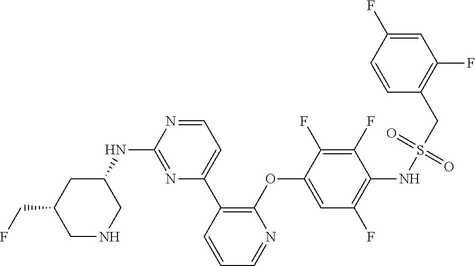 [SMILES: O=S(=O)(Cc1ccc(F)cc1F)Nc1c(F)cc(Oc2ncccc2-c2ccnc(N[C@@H]3CNC[C@H](CF)C3)n2)c(F)c1F] | 1-(2,4-difluorophenyl)-N-(2,3,6-trifluoro-4-((3- (2-(((3S,5R)-5-(fluoromethyl)piperidin-3- yl)amino)pyrimidin-4-yl)pyridin-2- yl)oxy)phenyl)methanesulfonamide |
| 321 | 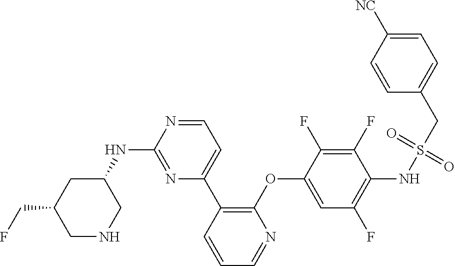 [SMILES: N#Cc1ccc(CS(=O)(=O)Nc2c(F)cc(Oc3ncccc3-c3ccnc(N[C@@H]4CNC[C@H](CF)C4)n3)c(F)c2F)cc1] | 1-(4-cyanophenyl)-N-(2,3,6-trifluoro-4-((3-(2- (((3S,5R)-5-(fluoromethyl)piperidin-3- yl)amino)pyrimidin-4-yl)pyridin-2- yl)oxy)phenyl)methanesulfonamide |
| 322 | 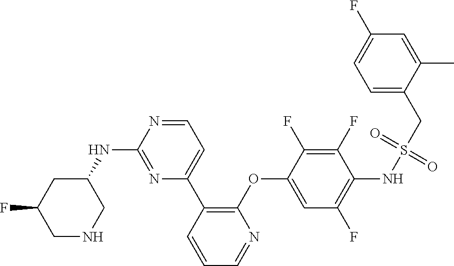 [SMILES: Cc1cc(F)ccc1CS(=O)(=O)Nc1c(F)cc(Oc2ncccc2-c2ccnc(N[C@@H]3CNC[C@@H](F)C3)n2)c(F)c1F] | 1-(4-fluoro-2-methylphenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 323 | 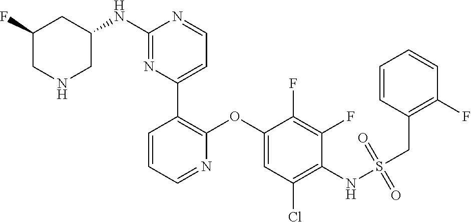 [SMILES: O=S(=O)(Cc1ccccc1F)Nc1c(Cl)cc(Oc2ncccc2-c2ccnc(N[C@@H]3CNC[C@@H](F)C3)n2)c(F)c1F] | N-(6-chloro-2,3-difluoro-4-((3-(2- (((3S,5S)-5-fluoropiperidin-3- yl)amino)pyrimidin-4-yl)pyridin-2- yl)oxy)phenyl)-1-(2- fluorophenyl)methanesulfonamide |
| 324 | 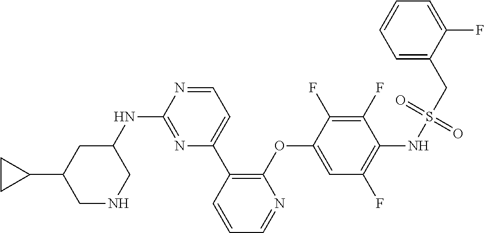 [SMILES: O=S(=O)(Cc1ccccc1F)Nc1c(F)cc(Oc2ncccc2-c2ccnc(NC3CNCC(C4CC4)C3)n2)c(F)c1F] | N-(4-((3-(2-((5-cyclopropylpiperidin-3- yl)amino)pyrimidin-4-yl)pyridin-2-yl)oxy)-2,3,6- trifluorophenyl)-1-(2- fluorophenyl)methanesulfonamide |
| 328 | 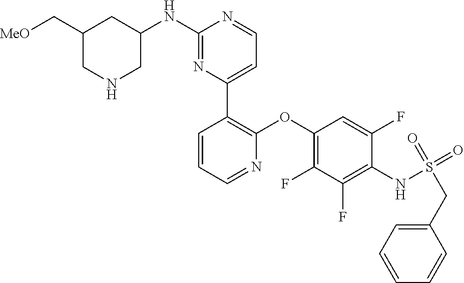 [SMILES: COCC1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5- (methoxymethyl)piperidin-3-yl)amino)pyrimidin- 4-yl)pyridin-2- yl)oxy)phenyl)methanesulfonamide |
| 332 | 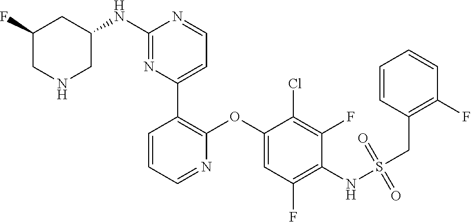 [SMILES: O=S(=O)(Cc1ccccc1F)Nc1c(F)cc(Oc2ncccc2-c2ccnc(N[C@@H]3CNC[C@@H](F)C3)n2)c(Cl)c1F] | N-(3-chloro-2,6-difluoro-4-((3-(2-(((3S,5S)-5- fluoropiperidin-3-yl)amino)pyrimidin-4- yl)pyridin-2-yl)oxy)phenyl)-1-(2- fluorophenyl)methanesulfonamide |
| 333 | 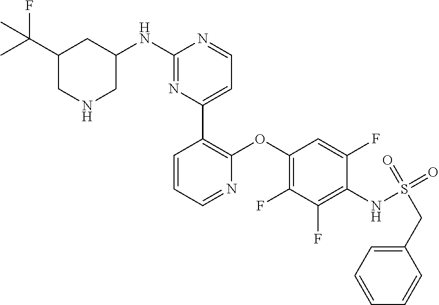 [SMILES: CC(C)(F)C1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5-(2- fluoropropan-2-yl)piperidin-3- yl)amino)pyrimidin-4-yl)pyridin-2- yl)oxy)phenyl)methanesulfonamidehydrochloride |
| 334 | 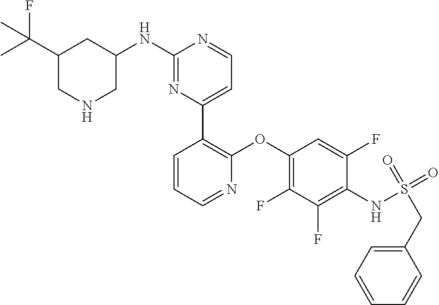 [SMILES: CC(C)(F)C1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5-(2- fluoropropan-2-yl)piperidin-3- yl)amino)pyrimidin-4-yl)pyridin-2- yl)oxy)phenyl)methanesulfonamide |
| 337 | 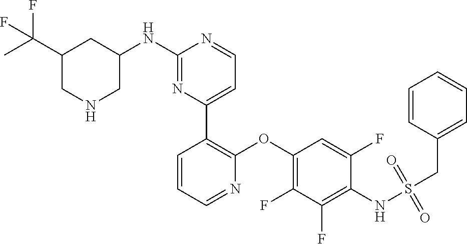 [SMILES: CC(F)(F)C1CNCC(Nc2nccc(-c3cccnc3Oc3cc(F)c(NS(=O)(=O)Cc4ccccc4)c(F)c3F)n2)C1] | N-(4-((3-(2-((5-(1,1-difluoroethyl)piperidin-3- yl)amino)pyrimidin-4-yl)pyridin-2-yl)oxy)-2,3,6- trifluorophenyl)-1- phenylmethanesulfonamidehydrochloride |
| 338 | 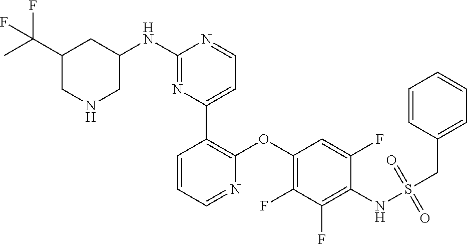 [SMILES: CC(F)(F)C1CNCC(Nc2nccc(-c3cccnc3Oc3cc(F)c(NS(=O)(=O)Cc4ccccc4)c(F)c3F)n2)C1] | N-(4-((3-(2-((5-(1,1-difluoroethyl)piperidin-3- yl)amino)pyrimidin-4- yl)pyridin-2-yl)oxy)-2,3,6-trifluorophenyl)-1- phenylmethanesulfonamide |
| 340 | 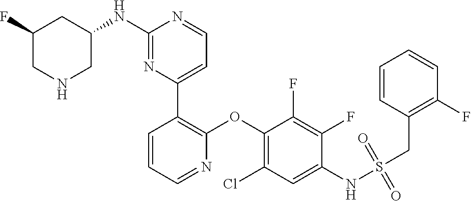 [SMILES: O=S(=O)(Cc1ccccc1F)Nc1cc(Cl)c(Oc2ncccc2-c2ccnc(N[C@@H]3CNC[C@@H](F)C3)n2)c(F)c1F] | N-(5-chloro-2,3-difluoro-4-((3-(2-(((3S,5S)-5- fluoropiperidin-3-yl)amino)pyrimidin-4- yl)pyridin-2-yl)oxy)phenyl)-1-(2- fluorophenyl)methanesulfonamide |
| 341 | 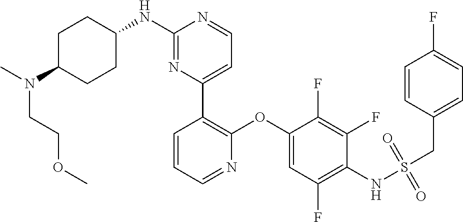 [SMILES: COCCN(C)[C@H]1CC[C@H](Nc2nccc(-c3cccnc3Oc3cc(F)c(NS(=O)(=O)Cc4ccc(F)cc4)c(F)c3F)n2)CC1] | 1-(4-fluorophenyl)-N-(2,3,6-trifluoro-4- ((3-(2-(((1r,4r)-4-((2- methoxyethyl)(methyl)amino)cyclohexyl) amino)pyrimidin-4-yl)pyridin-2- yl)oxy)phenyl)methanesulfonamidehydrochloride |
| 342 | 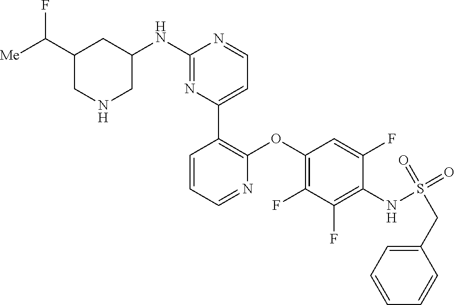 [SMILES: CC(F)C1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5-(1- fluoroethyl)piperidin-3-yl)amino)pyrimidin-4- yl)pyridin-2-yl)oxy)phenyl)methanesulfonamide hydrochloride |
| 346 | 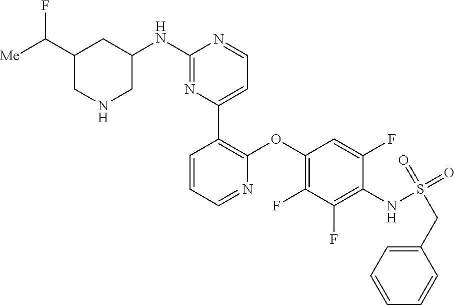 [SMILES: CC(F)C1CNCC(Nc2nccc(-c3cccnc3Oc3cc(F)c(NS(=O)(=O)Cc4ccccc4)c(F)c3F)n2)C1] | 1-phenyl-N-(2,3,6-trifluoro-4-((3-(2-((5-(1- fluoroethyl)piperidin-3- yl)amino)pyrimidin-4-yl)pyridin-2- yl)oxy)phenyl)methanesulfonamide |
| 350 | 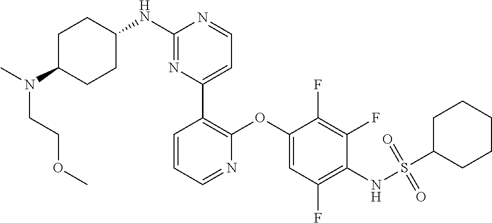 [SMILES: COCCN(C)[C@H]1CC[C@H](Nc2nccc(-c3cccnc3Oc3cc(F)c(NS(=O)(=O)C4CCCCC4)c(F)c3F)n2)CC1] | N-(2,3,6-trifluoro-4-((3-(2-(((1r,4r)-4-((2- methoxyethyl)(methyl)amino) cyclohexyl)amino)pyrimidin-4-yl)pyridin-2- yl)oxy)phenyl)cyclohexanesulfonamide |
| 351 | 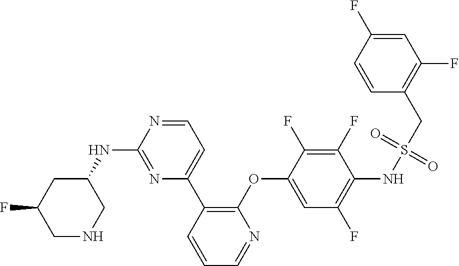 [SMILES: O=S(=O)(Cc1ccc(F)cc1F)Nc1c(F)cc(Oc2ncccc2-c2ccnc(N[C@@H]3CNC[C@@H](F)C3)n2)c(F)c1F] | 1-(2,4-difluorophenyl)-N-[2,3,6-trifluoro-4-[[3- [2-[[(35,5S)-5-fluoro-3- piperidyl]amino]pyrimidin-4-yl]-2- pyridyl]oxy]phenyl\|methanesulfonamide |
| 352 | 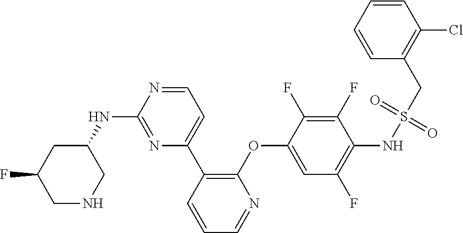 [SMILES: O=S(=O)(Cc1ccccc1Cl)Nc1c(F)cc(Oc2ncccc2-c2ccnc(N[C@@H]3CNC[C@@H](F)C3)n2)c(F)c1F] | 1-(2-chlorophenyl)-N-[2,3,6-trifluoro-4-[[3-[2- [[(3S,5S)-5-fluoro-3- piperidyl]amino]pyrimidin-4-yl]-2- pyridyl]oxy]phenyl]methanesulfonamide |
| 353 | 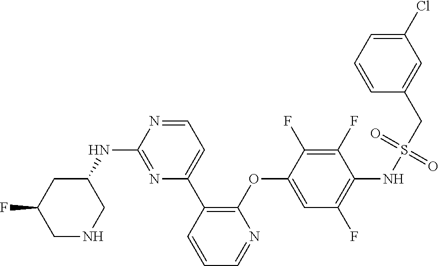 [SMILES: O=S(=O)(Cc1cccc(Cl)c1)Nc1c(F)cc(Oc2ncccc2-c2ccnc(N[C@@H]3CNC[C@@H](F)C3)n2)c(F)c1F] | 1-(3-chlorophenyl)-N-[2,3,6-trifluoro-4-[[3-[2- [[(3S,5S)-5-fluoro-3- piperidyl]amino]pyrimidin-4-yl]-2- pyridyl]oxy]phenyl]methanesulfonamide |
| 354 | 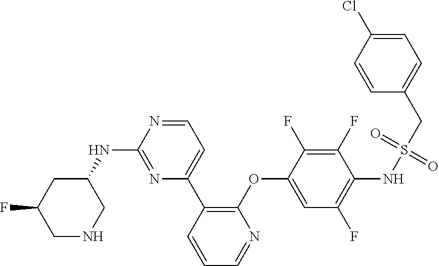 [SMILES: O=S(=O)(Cc1ccc(Cl)cc1)Nc1c(F)cc(Oc2ncccc2-c2ccnc(N[C@@H]3CNC[C@@H](F)C3)n2)c(F)c1F] | 1-(4-chlorophenyl)-N-[2,3,6-trifluoro-4-[[3-[2- [[(3S,5S)-5-fluoro-3- piperidyl]amino]pyrimidin-4-yl]-2- pyridyl]oxy]phenyl]methanesulfonamide |
| 355 | 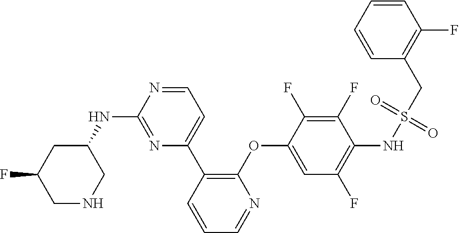 [SMILES: O=S(=O)(Cc1ccccc1F)Nc1c(F)cc(Oc2ncccc2-c2ccnc(N[C@@H]3CNC[C@@H](F)C3)n2)c(F)c1F] | 1-(2-fluorophenyl)-N-[2,3,6-trifluoro-4-[3-[2- [[(3S,5S)-5-fluoro-3- piperidyl]amino]pyrimidin-4-yl]-2- pyridyl]oxy]phenyl]methanesulfonamide |
| 356 | 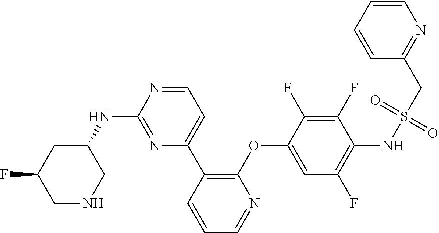 [SMILES: O=S(=O)(Cc1ccccn1)Nc1c(F)cc(Oc2ncccc2-c2ccnc(N[C@@H]3CNC[C@@H](F)C3)n2)c(F)c1F] | 1-(2-pyridyl)-N-[2,3,6-trifluoro-4- [[3-[2-[[(3S,5S-5-fluoro-3- piperidyl]amino]pyrimidin-4-yl]-2- pyridyl]oxy]phenyl]methanesulfonamide |
| 357 | 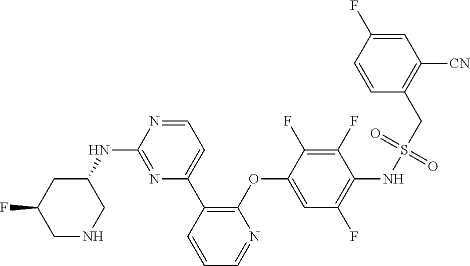 [SMILES: N#Cc1cc(F)ccc1CS(=O)(=O)Nc1c(F)cc(Oc2ncccc2-c2ccnc(N[C@@H]3CNC[C@@H](F)C3)n2)c(F)c1F] | 1-(2-cyano-4-fluoro-phenyl)-N-[2,3,6-trifluoro-4- [[3-[2-[[(3S,5S)-5-fluoro-3- piperidyl]amino]pyrimidin-4-yl]-2- pyridyl]oxy]phenyl]methanesulfonamide |
| 358 | 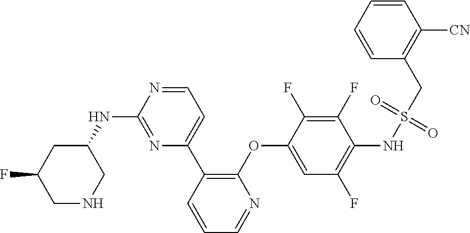 [SMILES: N#Cc1ccccc1CS(=O)(=O)Nc1c(F)cc(Oc2ncccc2-c2ccnc(N[C@@H]3CNC[C@@H](F)C3)n2)c(F)c1F] | 1-(2-cyanophenyl)-N-[2,3,6-trifluoro-4- [[3-[2-[[(3S,5S)-5-fluoro-3- piperidyl]amino]pyrimidin-4-yl]-2- pyridyl]oxy]phenyl]methanesulfonamide |
| 359 | 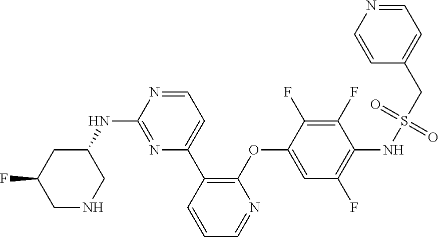 [SMILES: O=S(=O)(Cc1ccncc1)Nc1c(F)cc(Oc2ncccc2-c2ccnc(N[C@@H]3CNC[C@@H](F)C3)n2)c(F)c1F] | 1-(4-pyridyl)-N-[2,3,6-trifluoro-4-[[3-[2- [[(3S,5S)-5-fluoro-3- piperidyl]amino]pyrimidin-4-yl]-2- pyridyl]oxy]phenyl]methanesulfonamide |
| 360 | 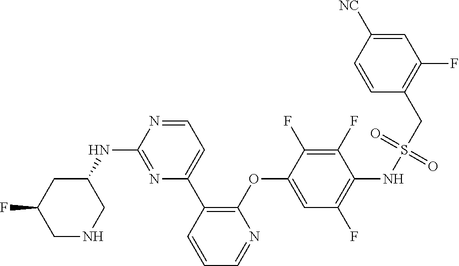 [SMILES: N#Cc1ccc(CS(=O)(=O)Nc2c(F)cc(Oc3ncccc3-c3ccnc(N[C@@H]4CNC[C@@H](F)C4)n3)c(F)c2F)c(F)c1] | 1-(4-cyano-2-fluoro-phenyl)-N-[2,3,6-trifluoro-4- [[3-[2-[[(3S,5S)-5-fluoro-3- piperidyl]amino]pyrimidin-4-yl]-2- pyridyl]oxy]phenyl]methanesulfonamide |
| 361 | 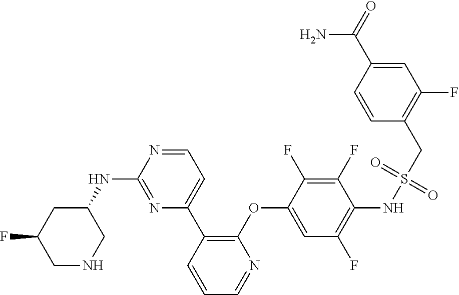 [SMILES: NC(=O)c1ccc(CS(=O)(=O)Nc2c(F)cc(Oc3ncccc3-c3ccnc(N[C@@H]4CNC[C@@H](F)C4)n3)c(F)c2F)c(F)c1] | 3-fluoro-4-[[2,3,6-trifluoro-4- [[3-[2-[[(3S,5S)-5-fluoro-3- piperidyl]amino]pyrimidin-4-yl]-2- pyridyl]oxy]phenyl]sulfamoylmethyl]benzamide |
| 362 | 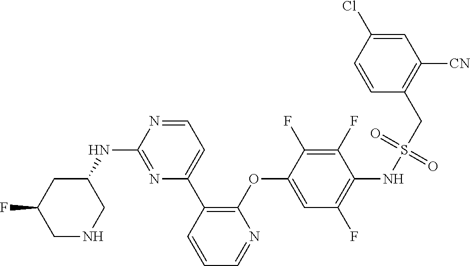 [SMILES: N#Cc1cc(Cl)ccc1CS(=O)(=O)Nc1c(F)cc(Oc2ncccc2-c2ccnc(N[C@@H]3CNC[C@@H](F)C3)n2)c(F)c1F] | 1-(4-chloro-2-cyano-phenyl)-N-[2,3,6-trifluoro-4- [3-[2-[(3S′,5S)-5-fluoro-3- piperidyl]amino]pyrimidin-4-yl]-2- pyridyl]oxy]phenyl]methanesulfonamide |
| 363 | 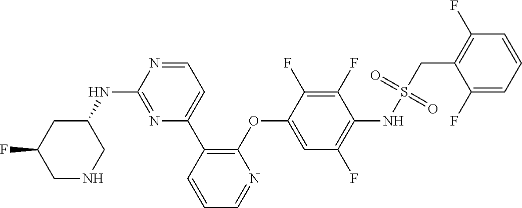 [SMILES: O=S(=O)(Cc1c(F)cccc1F)Nc1c(F)cc(Oc2ncccc2-c2ccnc(N[C@@H]3CNC[C@@H](F)C3)n2)c(F)c1F] | 1-(2,6-difluorophenyl)-N-[2,3,6-trifluoro-4-[[3- [2-[[(3S,5S)-5-fluoro-3- piperidyl]amino]pyrimidin-4-yl]-2- pyridyl]oxy]phenyl]methanesulfonamide |
| 364 | 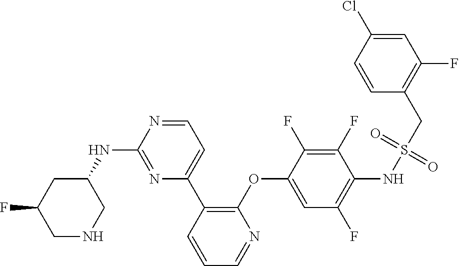 [SMILES: O=S(=O)(Cc1ccc(Cl)cc1F)Nc1c(F)cc(Oc2ncccc2-c2ccnc(N[C@@H]3CNC[C@@H](F)C3)n2)c(F)c1F] | 1-(4-chloro-2-fluoro-phenyl)-N-[2,3,6-trifluoro-4- [[3-[2-[[(3S,5S)-5-fluoro-3- piperidyl]amino]pyrimidin-4-yl]-2- pyridyl]oxy]phenyl]methanesulfonamide |
| 365 | 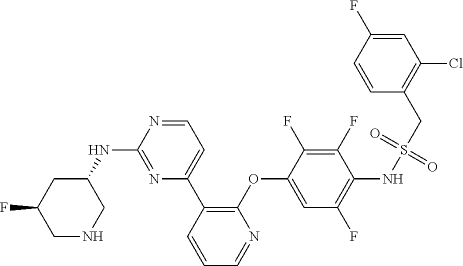 [SMILES: O=S(=O)(Cc1ccc(F)cc1Cl)Nc1c(F)cc(Oc2ncccc2-c2ccnc(N[C@@H]3CNC[C@@H](F)C3)n2)c(F)c1F] | 1-(2-chloro-4-fluoro-phenyl)-N-[2,3,6-trifluoro-4- [[3-[2-[[(3S,5S)-5-fluoro-3- piperidyl]amino]pyrimidin-4-yl]-2- pyridyl]oxy]phenyl]methanesulfonamide |
| 366 | 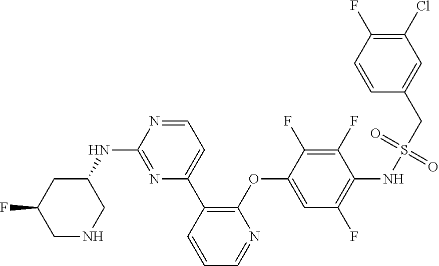 [SMILES: O=S(=O)(Cc1ccc(F)c(Cl)c1)Nc1c(F)cc(Oc2ncccc2-c2ccnc(N[C@@H]3CNC[C@@H](F)C3)n2)c(F)c1F] | 1-(3-chloro-4-fluoro-phenyl)-N-[2,3,6-trifluoro-4- [[3-[2-[(3S,5S)-5-fluoro-3- piperidyl]amino]pyrimidin-4-yl]-2- pyridyl]oxy]phenyl]methanesulfonamide |
| 367 | 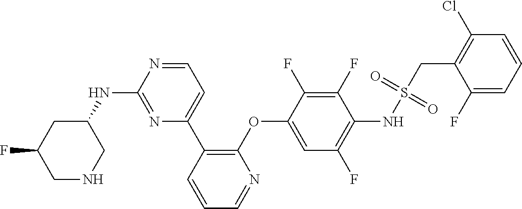 [SMILES: O=S(=O)(Cc1c(F)cccc1Cl)Nc1c(F)cc(Oc2ncccc2-c2ccnc(N[C@@H]3CNC[C@@H](F)C3)n2)c(F)c1F] | 1-(2-chloro-6-fluorophenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 368 | 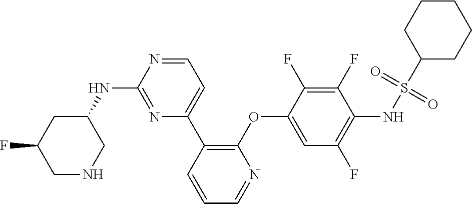 [SMILES: O=S(=O)(Nc1c(F)cc(Oc2ncccc2-c2ccnc(N[C@@H]3CNC[C@@H](F)C3)n2)c(F)c1F)C1CCCCC1] | N-(2,3,6-trifluoro-4-((3-(2-(((3S5S)-5- fluoropiperidin-3-yl)amino)pyrimidin-4- yl)pyridin-2- yl)oxy)phenyl)cyclohexanesulfonamide |
| 369 | 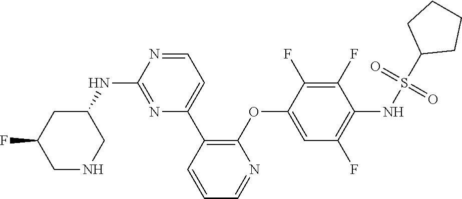 [SMILES: O=S(=O)(Nc1c(F)cc(Oc2ncccc2-c2ccnc(N[C@@H]3CNC[C@@H](F)C3)n2)c(F)c1F)C1CCCC1] | N-(2,3,6-trifluoro-4-((3-(2-(((3S,5S)-5- fluoropiperidin-3-yl)amino)pyrimidin-4- yl)pyridin-2- yl)oxy)phenyl)cyclopentanesulfonamide |
| 370 | 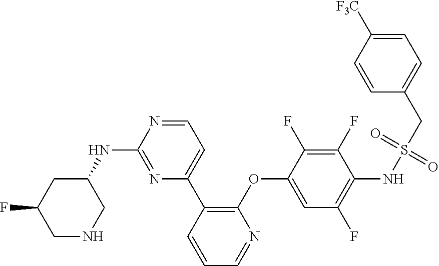 [SMILES: O=S(=O)(Cc1ccc(C(F)(F)F)cc1)Nc1c(F)cc(Oc2ncccc2-c2ccnc(N[C@@H]3CNC[C@@H](F)C3)n2)c(F)c1F] | N-(2,3,6-trifluoro-4-((3-(2-(((3S,5S)-5- fluoropiperidin-3-yl)amino)pyrimidin-4- yl)pyridin-2-yl)oxy)phenyl)-1-(4- (trifluoromethyl)phenyl)methanesulfonamide |
| 371 | 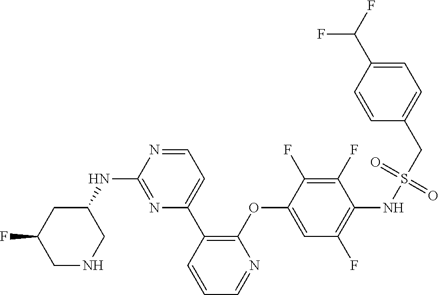 [SMILES: O=S(=O)(Cc1ccc(C(F)F)cc1)Nc1c(F)cc(Oc2ncccc2-c2ccnc(N[C@@H]3CNC[C@@H](F)C3)n2)c(F)c1F] | 1-(4-(difluoromethyl)phenyl)-N-(2,3,6-trifluoro- 4-((3-(2-(((3S,5S)-5-fluoropiperidin-3- yl)amino)pyrimidin-4-yl)pyridin-2- yl)oxy)phenyl)methanesulfonamide |
| 372 | 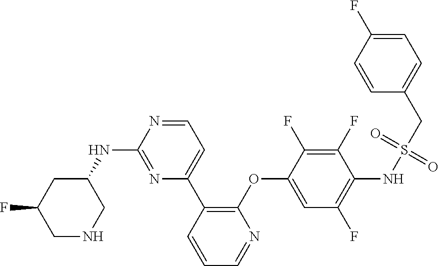 [SMILES: O=S(=O)(Cc1ccc(F)cc1)Nc1c(F)cc(Oc2ncccc2-c2ccnc(N[C@@H]3CNC[C@@H](F)C3)n2)c(F)c1F] | 1-(4-fluorophenyl)-N-(2,3,6-trifluoro-4-((3-(2- (((3S,5S)-5-fluoropiperidin-3- yl)amino)pyrimidin-4-yl)pyridin-2- yl)oxy)phenyl)methanesulfonamide |
| 373 | 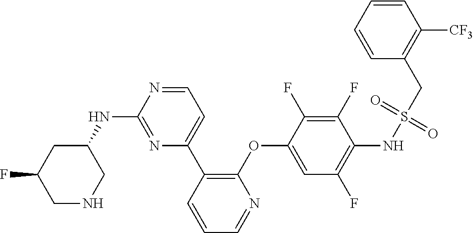 [SMILES: O=S(=O)(Cc1ccccc1C(F)(F)F)Nc1c(F)cc(Oc2ncccc2-c2ccnc(N[C@@H]3CNC[C@@H](F)C3)n2)c(F)c1F] | N-(2,3,6-trifluoro-4-((3-(2-(((3S5S)-5- fluoropiperidin-3-yl)amino)pyrimidin-4- yl)pyridin-2-yl)oxy)phenyl)-1-(2- (trifluoromethyl)phenyl)methanesulfonamide |
| 374 | 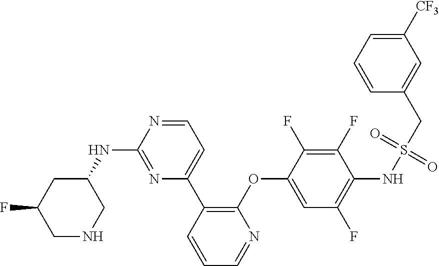 [SMILES: O=S(=O)(Cc1cccc(C(F)(F)F)c1)Nc1c(F)cc(Oc2ncccc2-c2ccnc(N[C@@H]3CNC[C@@H](F)C3)n2)c(F)c1F] | N-(2,3,6-trifluoro-4-((3-(2-(((3S5S)-5- fluoropiperidin-3-yl)amino)pyrimidin-4- yl)pyridin-2-yl)oxy)phenyl)-1-(3- (trifluoromethyl)phenyl)methanesulfonamide |
| 375 | 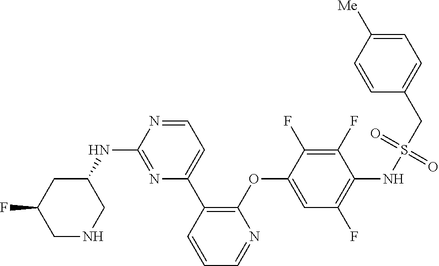 [SMILES: Cc1ccc(CS(=O)(=O)Nc2c(F)cc(Oc3ncccc3-c3ccnc(N[C@@H]4CNC[C@@H](F)C4)n3)c(F)c2F)cc1] | 1-p-tolyl-N-(2,3,6-trifluoro-4-((3-(2-(((3S,5S)-5- fluoropiperidin-3-yl)amino)pyrimidin-4- yl)pyridin-2-yl)oxy)phenyl)methanesulfonamide |
| 376 | 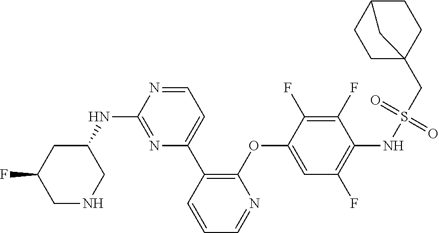 [SMILES: O=S(=O)(CC12CCC(CC1)C2)Nc1c(F)cc(Oc2ncccc2-c2ccnc(N[C@@H]3CNC[C@@H](F)C3)n2)c(F)c1F] | 1-(bicyclo[2.2.1]heptan-1-yl)-N-(2,3,6-trifluoro- 4-((3-(2-(((3S,5S)-5-fluoropiperidin-3- yl)amino)pyrimidin-4-yl)pyridin-2- yl)oxy)phenyl)methanesulfonamide |
| 377 | 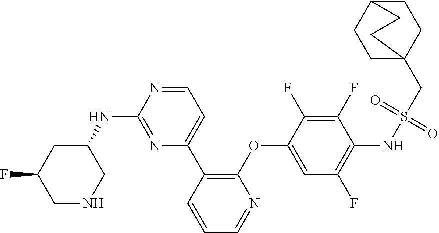 [SMILES: O=S(=O)(CC12CCC(CC1)CC2)Nc1c(F)cc(Oc2ncccc2-c2ccnc(N[C@@H]3CNC[C@@H](F)C3)n2)c(F)c1F] | 1-(bicyclo[2.2.2]octan-1-yl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 378 | 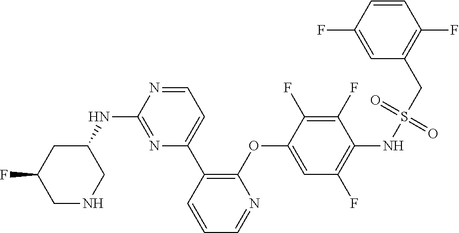 [SMILES: O=S(=O)(Cc1cc(F)ccc1F)Nc1c(F)cc(Oc2ncccc2-c2ccnc(N[C@@H]3CNC[C@@H](F)C3)n2)c(F)c1F] | 1-(2,5-difluorophenyl)-N-(2,3,6-trifluoro-4-((3- (2-(((3S,5S)-5-fluoropiperidin-3- yl)amino)pyrimidin-4-yl)pyridin-2- yl)oxy)phenyl)methanesulfonamide |
| 379 | 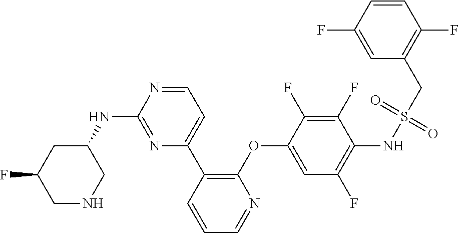 [SMILES: O=S(=O)(Cc1cc(F)ccc1F)Nc1c(F)cc(Oc2ncccc2-c2ccnc(N[C@@H]3CNC[C@@H](F)C3)n2)c(F)c1F] | 1-(4-cyanophenyl)-N-(2,3-difluoro-4- ((3-(2-(((S)-piperidin-3- yl)amino)pyrimidin-4-yl)pyridin-2- yl)oxy)phenyl)ethane-1-sulfonamide |
| 380 | 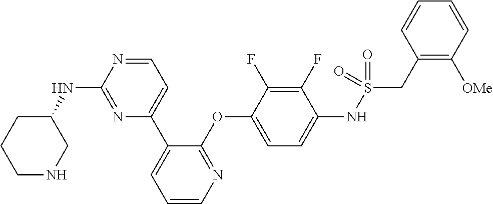 [SMILES: COc1ccccc1CS(=O)(=O)Nc1ccc(Oc2ncccc2-c2ccnc(N[C@H]3CCCNC3)n2)c(F)c1F] | (S)-N-(2,3-difluoro-4-((3-(2-(piperidin-3- ylamino)pyrimidin-4-yl)pyridin-2- yl)oxy)phenyl)-1-(2- methoxyphenyl)methanesulfonamide |
| 381 | 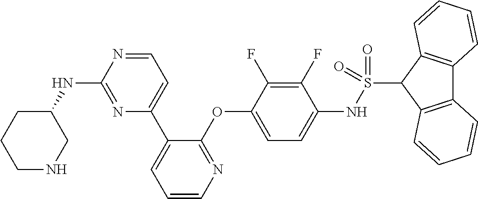 [SMILES: O=S(=O)(Nc1ccc(Oc2ncccc2-c2ccnc(N[C@H]3CCCNC3)n2)c(F)c1F)C1c2ccccc2-c2ccccc21] | (S)-N-(2,3-difluoro-4-((3-(2-(piperidin-3- ylamino)pyrimidin-4- yl)pyridin-2-yl)oxy)phenyl)-9H-fluorene-9- sulfonamide |
| 382 | 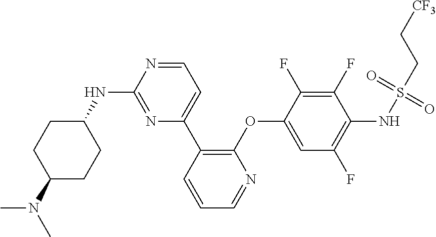 [SMILES: CN(C)[C@H]1CC[C@H](Nc2nccc(-c3cccnc3Oc3cc(F)c(NS(=O)(=O)CCC(F)(F)F)c(F)c3F)n2)CC1] | N-(4-((3-(2-(((1r,4r)-4- (dimethylamino)cyclohexyl)amino)pyrimidin-4- yl)pyridin-2-yl)oxy)-2,3,6-trifluorophenyl)-3,3,3- trifluoropropane-1-sulfonamide |
| 383 | 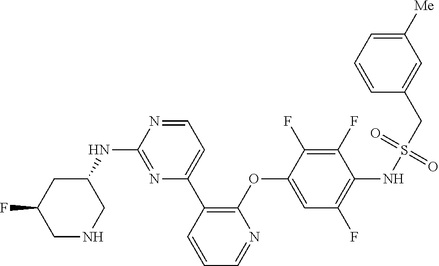 [SMILES: Cc1cccc(CS(=O)(=O)Nc2c(F)cc(Oc3ncccc3-c3ccnc(N[C@@H]4CNC[C@@H](F)C4)n3)c(F)c2F)c1] | 1-m-tolyl-N-(2,3,6-trifluoro-4-((3-(2-(((3S,5S)-5- fluoropiperidin-3- yl)amino)pyrimidin-4-yl)pyridin-2- yl)oxy)phenyl)methanesulfonamide |
| 384 | 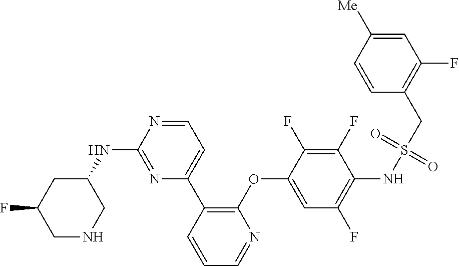 [SMILES: Cc1ccc(CS(=O)(=O)Nc2c(F)cc(Oc3ncccc3-c3ccnc(N[C@@H]4CNC[C@@H](F)C4)n3)c(F)c2F)c(F)c1] | 1-(2-fluoro-4-methylphenyl)-N-(2,3,6-trifluoro-4- ((3-(2-(((3S,5S)-5-fluoropiperidin-3- yl)amino)pyrimidin-4-yl)pyridin-2- yl)oxy)phenyl)methanesulfonamide |
| 385 | 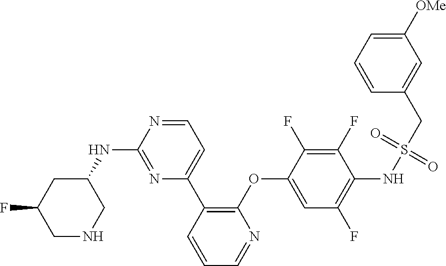 [SMILES: COc1cccc(CS(=O)(=O)Nc2c(F)cc(Oc3ncccc3-c3ccnc(N[C@@H]4CNC[C@@H](F)C4)n3)c(F)c2F)c1] | 1-(3-methoxyphenyl)-N-(2,3,6-trifluoro-4-((3-(2- (((3S,5S)-5-fluoropiperidin-3- yl)amino)pyrimidin-4-yl)pyridin-2- yl)oxy)phenyl)methanesulfonamide |
| 386 | 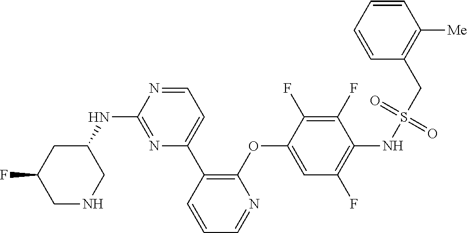 [SMILES: Cc1ccccc1CS(=O)(=O)Nc1c(F)cc(Oc2ncccc2-c2ccnc(N[C@@H]3CNC[C@@H](F)C3)n2)c(F)c1F] | 1-o-tolyl-N-(2,3,6-trifluoro-4-((3-(2-(((3S,5S)-5- fluoropiperidin-3- yl)amino)pyrimidin-4-yl)pyridin-2- yl)oxy)phenyl)methanesulfonamide |
| 387 | 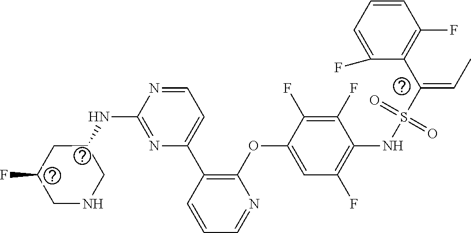 [SMILES: C/C=C(\c1c(F)cccc1F)S(=O)(=O)Nc1c(F)cc(Oc2ncccc2-c2ccnc(N[C@@H]3CNC[C@@H](F)C3)n2)c(F)c1F] | (*S)-1 -pheny 1-N-(2,3,6-trifluoro-4-((3-(2- (((3S,5S)-5-flucropiperidin-3- yl)amino)pyrimidin-4-yl)pyridin-2- yl)oxy)phenyl)ethane-1-sulfonamide |
| 388 | 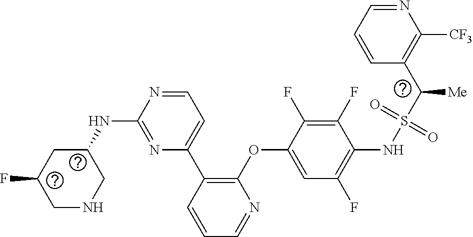 [SMILES: C[C@H](c1cccnc1C(F)(F)F)S(=O)(=O)Nc1c(F)cc(Oc2ncccc2-c2ccnc(N[C@@H]3CNC[C@@H](F)C3)n2)c(F)c1F] | (*R)-1-phenyl-N-(2,3,6-trifluoro-4-((3-(2- ((3S,5S)-5-flucropiperidin-3-yl)amino)pyrimidin- 4-yl)pyridin-2-yl)oxy)phenyl)ethane-1- sulfonamide |
| 389 | 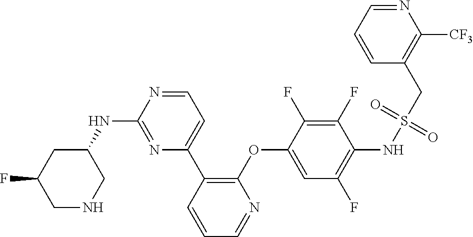 [SMILES: O=S(=O)(Cc1cccnc1C(F)(F)F)Nc1c(F)cc(Oc2ncccc2-c2ccnc(N[C@@H]3CNC[C@@H](F)C3)n2)c(F)c1F] | N-(2,3,6-trifluoro-4-((3-(2-((3S,5S)-5- fluoropiperidin-3-yl)amino)pyrimidin-4- yl)pyridin-2-yl)oxy)phenyl)-1-(2- (trifluoromethyl)pyridin-3- yl)methanesulfonamide |
| 390 | 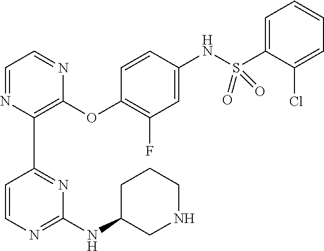 [SMILES: O=S(=O)(Nc1ccc(Oc2nccnc2-c2ccnc(N[C@H]3CCCNC3)n2)c(F)c1)c1ccccc1Cl] | (S)-N-(3-fluoro-4-((3-(2- (piperidin-3-ylamino)pyrimidin-4- yl)pyrazin-2-yl)oxy)phenyl)2- chlorobenzenesulfonamide |
| 391 | 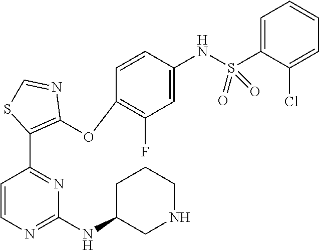 [SMILES: O=S(=O)(Nc1ccc(Oc2ncsc2-c2ccnc(N[C@H]3CCCNC3)n2)c(F)c1)c1ccccc1Cl] | (S)-N-(3-fluoro-4-((3-(2- (piperidin-3-ylamino)pyrimidin-4- yl)pyrazin-2-yl)oxy)phenyl)2- chlorobenzenesulfonamide |
| 392 | 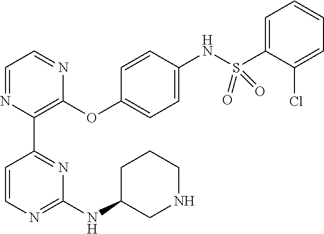 [SMILES: O=S(=O)(Nc1ccc(Oc2nccnc2-c2ccnc(N[C@H]3CCCNC3)n2)cc1)c1ccccc1Cl] | (S)-N-(4-((3-(2- (piperidin-3-ylamino)pyrimidin-4- yl)pyrazin-2-yl)oxy)phenyl)2- chlorobenzenesulfonamide |
| 393 | 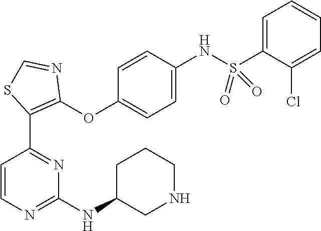 [SMILES: O=S(=O)(Nc1ccc(Oc2ncsc2-c2ccnc(N[C@H]3CCCNC3)n2)cc1)c1ccccc1Cl] | (S)-N-(4-((5-(2- (piperidin-3-ylamino)pyrimidin-4- yl)thiazol-4-yl)oxy)phenyl)2- chlorobenzenesulfonamide |
| 394 | 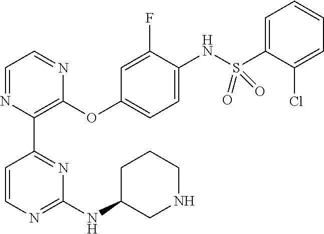 [SMILES: O=S(=O)(Nc1ccc(Oc2nccnc2-c2ccnc(N[C@H]3CCCNC3)n2)cc1F)c1ccccc1Cl] | N-[2-Fluoro-4-[[3-[2-[[(3S)-3- piperidinyl]amino]pyrimidin-4-yl]-2- pyrazinyl]oxy]phenyl]2- chlorobenzenesulfonamide |
| 395 | 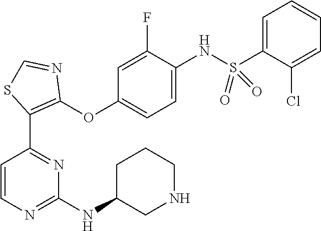 [SMILES: O=S(=O)(Nc1ccc(Oc2ncsc2-c2ccnc(N[C@H]3CCCNC3)n2)cc1F)c1ccccc1Cl] | N-[2-Fluoro-4-[[5-[2-[(3S)-3- piperidinyl\|amino]pyrimidin-4-yl]-4- thiazolyl]oxy]phenyl]2- chlorobenzenesulfonamide |
| 396 | 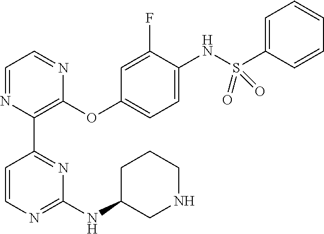 [SMILES: O=S(=O)(Nc1ccc(Oc2nccnc2-c2ccnc(N[C@H]3CCCNC3)n2)cc1F)c1ccccc1] | (S)-N-(3-fluoro-4-((3-(2- (piperidin-3-ylamino)pyrimidin-4- yl)pyrazin-2-yl)oxy)phenyl)benzenesulfonamide |
| 397 | 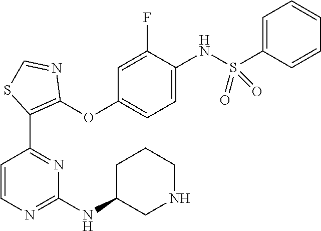 [SMILES: O=S(=O)(Nc1ccc(Oc2ncsc2-c2ccnc(N[C@H]3CCCNC3)n2)cc1F)c1ccccc1] | (S)-N-(2-fluoro-4-((5-(2- (piperidin-3-ylamino)pyrimidin-4- yl)thiazol-4-yl)oxy)phenyl)benzenesulfonamide |
| 398 | 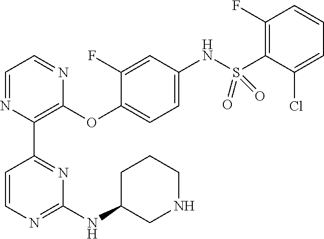 [SMILES: O=S(=O)(Nc1ccc(Oc2nccnc2-c2ccnc(N[C@H]3CCCNC3)n2)c(F)c1)c1c(F)cccc1Cl] | (S)-N-(3-fluoro-4-((3-(2- (piperidin-3-ylamino)pyrimidin-4- yl)pyrazin-2-yl)oxy)phenyl)2-chloro-6- fluorobenzenesulfonamide |
| 399 | 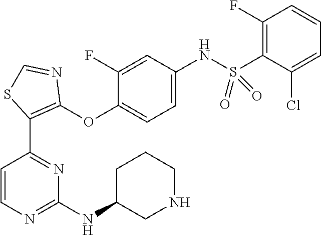 [SMILES: O=S(=O)(Nc1ccc(Oc2ncsc2-c2ccnc(N[C@H]3CCCNC3)n2)c(F)c1)c1c(F)cccc1Cl] | (S)-N-(3-fluoro-4-((5-(2- (piperidin-3-ylamino)pyrimidin-4- yl)thiazol-4-yl)oxy)phenyl)2-chloro-6- fluorobenzenesulfonamide |
| 400 | 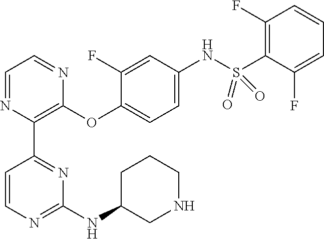 [SMILES: O=S(=O)(Nc1ccc(Oc2nccnc2-c2ccnc(N[C@H]3CCCNC3)n2)c(F)c1)c1c(F)cccc1F] | (S)-N-(3-fluoro-4-((3-(2- (piperidin-3-ylamino)pyrimidin-4- yl)pyrazin-2-yl)oxy)phenyl) 2,6- difluorobenzenesulfonamide |
| 401 | 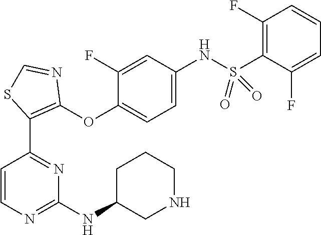 [SMILES: O=S(=O)(Nc1ccc(Oc2ncsc2-c2ccnc(N[C@H]3CCCNC3)n2)c(F)c1)c1c(F)cccc1F] | (S)-N-(3-fluoro-4-((5-(2- (piperidin-3-ylamino)pyrimidin-4- yl)thiazol-4-yl)oxy)phenyl)2,6- difluorobenzenesulfonamide |
| 402 | 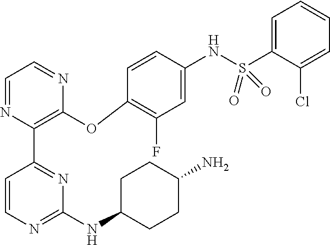 [SMILES: N[C@H]1CC[C@H](Nc2nccc(-c3nccnc3Oc3ccc(NS(=O)(=O)c4ccccc4Cl)cc3F)n2)CC1] | N-[4-[(1r,4r)[3-[2-[(4- Aminocyclohexyl)amino]pyrimidin-4-yl]pyrazin- 2-yl]oxy]-3-fluorophenyl]2- chlorobenzenesulfonamide |
| 403 | 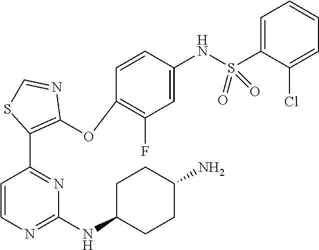 [SMILES: N[C@H]1CC[C@H](Nc2nccc(-c3scnc3Oc3ccc(NS(=O)(=O)c4ccccc4Cl)cc3F)n2)CC1] | N-[4-[(1r,4r)[5-[2-[(4- Aminocyclohexyl)amino]pyrimidin-4-yl]thiazol- 4-yl]oxy]-3-fluorophenyl]2- chlorobenzenesulfonamide |
| 404 | 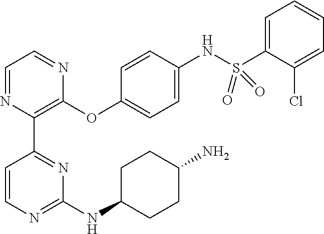 [SMILES: N[C@H]1CC[C@H](Nc2nccc(-c3nccnc3Oc3ccc(NS(=O)(=O)c4ccccc4Cl)cc3)n2)CC1] | N-[4-[(1r,4r)[3-[2-[(4- Aminocyclohexyl)amino]pyrimidin-4-yl]pyrazin- 2-yl]oxy]phenyl] 2-chlorobenzenesulfonamide |
| 405 | 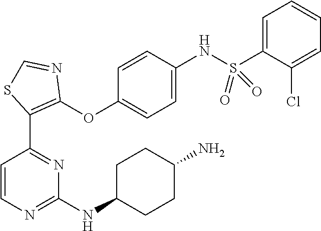 [SMILES: N[C@H]1CC[C@H](Nc2nccc(-c3scnc3Oc3ccc(NS(=O)(=O)c4ccccc4Cl)cc3)n2)CC1] | N-[4-[(1r,4r)[5-[2-[(4- Aminocyclohexyl)amino]pyrimidin-4-yl]thiazol- 4-yl]oxy]phenyl]2-chlorobenzenesulfonamide |
| 406 | 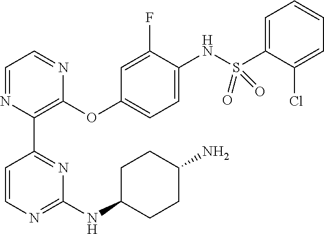 [SMILES: N[C@H]1CC[C@H](Nc2nccc(-c3nccnc3Oc3ccc(NS(=O)(=O)c4ccccc4Cl)c(F)c3)n2)CC1] | N-[4-[(1r,4r)[3-[2-[(4- Aminocyclohexyl)amino]pyrimidin-4-yl]pyrazin- 2-yl]oxy]-2-fluorophenyl]2- chlorobenzenesulfonamide |
| 407 | 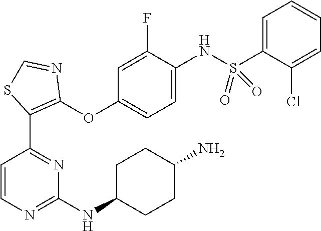 [SMILES: N[C@H]1CC[C@H](Nc2nccc(-c3scnc3Oc3ccc(NS(=O)(=O)c4ccccc4Cl)c(F)c3)n2)CC1] | N-[4-[(1r,4r)[5-[2-[(4- Aminocyclohexyl)amino]pyrimidin-4-yl]thiazol- 4-yl]oxy]-2-fluorophenyl]2- chlorobenzenesulfonamide |
| 408 | 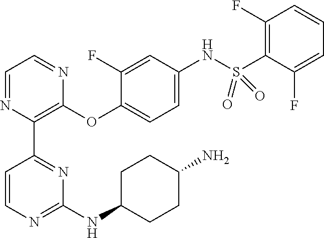 [SMILES: N[C@H]1CC[C@H](Nc2nccc(-c3nccnc3Oc3ccc(NS(=O)(=O)c4c(F)cccc4F)cc3F)n2)CC1] | N-[4-[(1r,4r)[3-[2-[(4- Aminocyclohexyl)amino]\|pyrimidin-4-yl]pyrazin- 2-yl]oxy]-3-fluorophenyl]2,6- difluorobenzenesulfonamide |
| 409 | 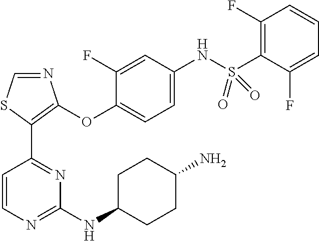 [SMILES: N[C@H]1CC[C@H](Nc2nccc(-c3scnc3Oc3ccc(NS(=O)(=O)c4c(F)cccc4F)cc3F)n2)CC1] | N-[4-[(1r,4r)[5-[2-[(4- Aminocyclohexyl)amino]pyrimidin-4-yl]thiazol- 4-yl]oxy]-3-fluorophenyl]2,6- difluorobenzenesulfonamide |
| 410 | 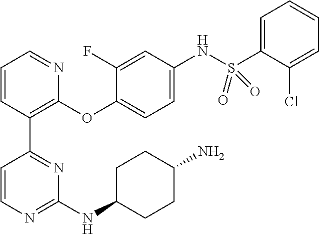 [SMILES: N[C@H]1CC[C@H](Nc2nccc(-c3cccnc3Oc3ccc(NS(=O)(=O)c4ccccc4Cl)cc3F)n2)CC1] | N-[4-[(1r,4r)[3-[2-[(4- Aminocyclohexyl)amino]pyrimidin-4-yl]pyrid-2- yl]oxy]-3-fluorophenyl] 2- chlorobenzenesulfonamide |
| 411 | 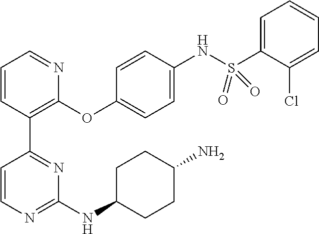 [SMILES: N[C@H]1CC[C@H](Nc2nccc(-c3cccnc3Oc3ccc(NS(=O)(=O)c4ccccc4Cl)cc3)n2)CC1] | N-[4-[(1r,4r)[3-[2-[(4- Aminocyclohexyl)amino]pyrimidin-4-yl]-2- pyridyl]oxy]phenyl]2-chlorobenzenesulfonamide |
| 412 | 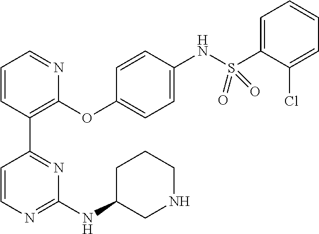 [SMILES: O=S(=O)(Nc1ccc(Oc2ncccc2-c2ccnc(N[C@H]3CCCNC3)n2)cc1)c1ccccc1Cl] | N-((S)-4-((3-(2-(Piperidin-3-ylamino)pyrimidin- 4-yl)pyridin-2-yl)oxy)phenyl)2- chlorobenzenesulfonamide |
| 413 | 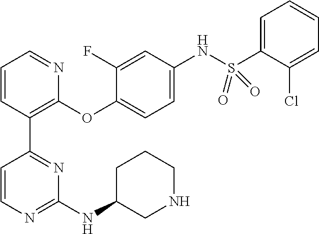 [SMILES: O=S(=O)(Nc1ccc(Oc2ncccc2-c2ccnc(N[C@H]3CCCNC3)n2)c(F)c1)c1ccccc1Cl] | N-((S)-4-((3-(2-(piperidin-3-ylamino)pyrimidin- 4-yl)pyridin-2-yl)oxy)-3-fluorophenyl)2- chlorobenzenesulfonamide |
| 414 | 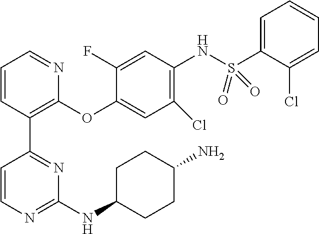 [SMILES: N[C@H]1CC[C@H](Nc2nccc(-c3cccnc3Oc3cc(Cl)c(NS(=O)(=O)c4ccccc4Cl)cc3F)n2)CC1] | N-[4-[(1r,4r)[3-[2-[(4- Aminocyclohexyl)amino]pyrimidin-4-yl]pyrid-2- yl]oxy]-6-chloro-3-fluorophenyl]2- chlorobenzenesulfonamide |
| 415 | 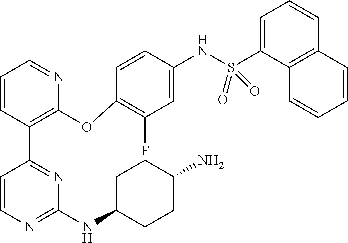 [SMILES: N[C@H]1CC[C@H](Nc2nccc(-c3cccnc3Oc3ccc(NS(=O)(=O)c4cccc5ccccc45)cc3F)n2)CC1] | N-[4-[(1r,4r)[3-[2-[(4- Aminocyclohexyl)amino]pyrimidin-4-yl]pyrid-2- yl]oxy]-3-fluorophenyl]naphthalene-1- sulfonamide |
| 416 | 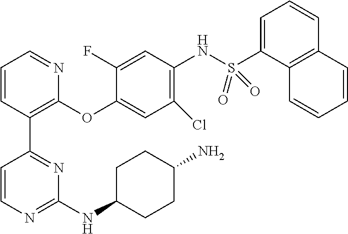 [SMILES: N[C@H]1CC[C@H](Nc2nccc(-c3cccnc3Oc3cc(Cl)c(NS(=O)(=O)c4cccc5ccccc45)cc3F)n2)CC1] | N-[4-[(1r,4r)[3-[2-[(4- Aminocyclohexyl)amino]pyrimidin-4-yl]pyrid-2- yl]oxy]-6-chloro-3-fluorophenyl]naphthalene-1- sulfonamide |
| 417 | 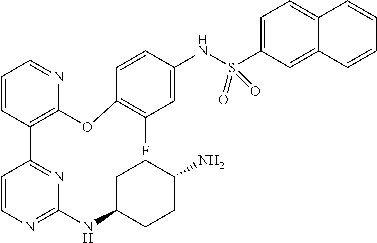 [SMILES: N[C@H]1CC[C@H](Nc2nccc(-c3cccnc3Oc3ccc(NS(=O)(=O)c4ccc5ccccc5c4)cc3F)n2)CC1] | N-[4-[(1r,4r)[3-[2-[(4- Aminocyclohexyl)amino]pyrimidin-4-yl]pyrid-2- yl]oxy ]-3-fluorophenyl]naphthalene-2- sulfonamide |

*arbitrarily assigned

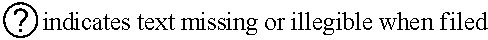

In some embodiments, a compound of the present disclosure is:

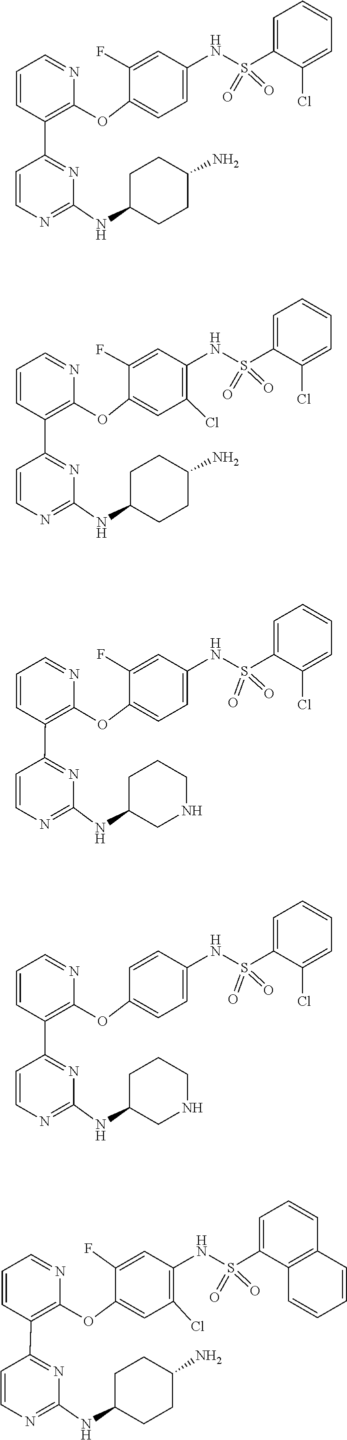 [SMILES: N[C@H]1CC[C@H](Nc2nccc(-c3cccnc3Oc3ccc(NS(=O)(=O)c4ccccc4Cl)cc3F)n2)CC1 ; N[C@H]1CC[C@H](Nc2nccc(-c3cccnc3Oc3cc(Cl)c(NS(=O)(=O)c4ccccc4Cl)cc3F)n2)CC1 ; O=S(=O)(Nc1ccc(Oc2ncccc2-c2ccnc(N[C@H]3CCCNC3)n2)c(F)c1)c1ccccc1Cl ; O=S(=O)(Nc1ccc(Oc2ncccc2-c2ccnc(N[C@H]3CCCNC3)n2)cc1)c1ccccc1Cl ; N[C@H]1CC[C@H](Nc2nccc(-c3cccnc3Oc3cc(Cl)c(NS(=O)(=O)c4cccc5ccccc45)cc3F)n2)CC1]

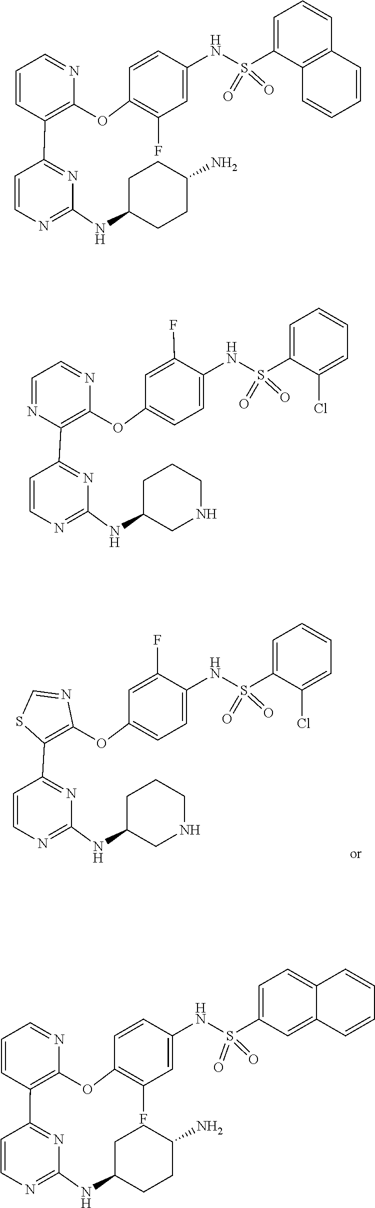 [SMILES: N[C@H]1CC[C@H](Nc2nccc(-c3cccnc3Oc3ccc(NS(=O)(=O)c4cccc5ccccc45)cc3F)n2)CC1 ; O=S(=O)(Nc1ccc(Oc2nccnc2-c2ccnc(N[C@H]3CCCNC3)n2)cc1F)c1ccccc1Cl ; O=S(=O)(Nc1ccc(Oc2ncsc2-c2ccnc(N[C@H]3CCCNC3)n2)cc1F)c1ccccc1Cl ; N[C@H]1CC[C@H](Nc2nccc(-c3cccnc3Oc3ccc(NS(=O)(=O)c4ccc5ccccc5c4)cc3F)n2)CC1]

or a pharmaceutically acceptable salt thereof.

In some embodiments, the methods provided herein comprise use of a compound selected from Compound Nos. 101-417 in Table 2, or a pharmaceutically acceptable salt thereof. In some embodiments, the methods provided herein comprise use of a compound selected from Compound Nos. 101-238 or 248-417 in Table 2, or a pharmaceutically acceptable salt thereof. In some embodiments, the methods provided herein comprise use of a compound selected from Compound Nos. 101-238, 248-380, 382-417 in Table 2, or a pharmaceutically acceptable salt thereof. In some embodiments, the methods provided herein comprise use of a compound selected from Compound Nos. 248-380 and 382-389 or 390-417 in Table 2, or a pharmaceutically acceptable salt thereof.

In some embodiments, the methods provided herein comprise use of the compound of the Formula (I) excluding compounds of Table 1. In some other embodiments, the methods provided herein comprise use of the Formula (I) including compounds of Table 1. In some other embodiments, the methods provided herein comprise use of a compound selected from Table 2, or a pharmaceutically acceptable salt thereof.

In some embodiments, the compounds or pharmaceutically acceptable salts thereof and compositions disclosed herein are useful in methods of inhibiting CDK2 activity. Thus, in some embodiments, provided herein is a method of inhibiting CDK2 activity in a subject in need thereof, comprising administering to the subject an effective amount of a compound of Formula (I), or a pharmaceutically acceptable salt thereof, or a pharmaceutical composition thereof.

In some embodiments, the compounds or pharmaceutically acceptable salts thereof and compositions disclosed herein are useful in methods of reducing the activity of CDK2. Thus, in some embodiments, provided herein is a method of reducing the activity of CDK2 in a subject in need thereof, comprising administering to the subject an effective amount of a compound of Formula (I), or a pharmaceutically acceptable salt thereof, or a pharmaceutical composition thereof.

In some embodiments, the compounds and compositions disclosed herein are selective against CDK2 (e.g., have a lower inhibitory constant, Kᵢ or IC₅₀ for CDK2) relative to other enzymatic targets of CDK such as CDK1. Compounds that are selective against CDK2 provide improved safety profile, improved dosing schedule, and/or enhanced overall efficacy. Similarly selective inhibitors of CDK2 have reduced risk of certain toxicities linked to inhibition of other CDKs. In some embodiments, the compounds and compositions disclosed herein are selective against CDK2 relative to CDK1. Thus, in some embodiments, the methods disclosed herein selectively inhibit CDK2 over other cyclin-dependent kinases (CDKs), such as CDK1.

Also provided herein, in some embodiments, are uses of compounds or pharmaceutically acceptable salts thereof and compositions disclosed herein in methods of treating, preventing, and/or reducing the risk or severity of certain diseases or disorders mediated by unwanted CDK2 activity, diseases or disorders involving over-expression or amplification of cyclin E, diseases or disorders involving over-expression or amplification of cyclin A2, or diseases or disorders that are otherwise treatable with a CDK2 inhibitor.

In some embodiments, provided herein is a method of treatment of a CDK2-related disease or disorder (e.g., diseases or disorders mediated by unwanted CDK2 activity, diseases or disorders involving over-expression or amplification of cyclin E, diseases or disorders involving over-expression or amplification of cyclin A2, or diseases or disorders that are otherwise treatable with a CDK2 inhibitor) in a subject in need thereof, comprising administering to the subject an effective amount of a compound of Formula (I), or a pharmaceutically acceptable salt thereof, or a pharmaceutical composition thereof.

Also provided herein, in some embodiments, is a method of prevention of a CDK2-related disease or disorder (e.g., diseases or disorders mediated by unwanted CDK2 activity, diseases or disorders involving over-expression or amplification of cyclin E, diseases or disorders involving over-expression or amplification of cyclin A2, or diseases or disorders that are otherwise treatable with a CDK2 inhibitor) in a subject in need thereof, comprising administering to the subject an effective amount of a compound of Formula (I), or a pharmaceutically acceptable salt thereof, or a pharmaceutical composition thereof.

Also provided herein, in some embodiments, is a method of reducing the risk or severity of a CDK2-related disease or disorder (e.g., diseases or disorders mediated by unwanted CDK2 activity, diseases or disorders involving over-expression or amplification of cyclin E, diseases or disorders involving over-expression or amplification of cyclin A2, or diseases or disorders that are otherwise treatable with a CDK2 inhibitor) in a subject in need thereof, comprising administering to the subject an effective amount of a compound of Formula (I), or a pharmaceutically acceptable salt thereof, or a pharmaceutical composition thereof.

In some embodiments, the CDK2-related disease or disorder is identified as having amplification or overexpression of cyclin E.

In some embodiments, the CDK2-related disease or disorder is identified as having amplification or overexpression of cyclin A.

In some embodiments, the CDK2-related disease or disorder is identified as having amplification or overexpression of cyclin A and cyclin E.

In some embodiments of the methods provided herein, the CDK2-related disease or disorder is cancer. In some embodiments, the cancer is mediated by unwanted activity of CDK2. In some embodiments, the cancer is mediated by over-expression or amplification of cyclin E. In some embodiments, the cancer is mediated by over-expression or amplification of cyclin A. In some embodiments, the cancer is identified as having amplification or overexpression of cyclin E. In some embodiments, the cancer is identified as having amplification or overexpression of cyclin A.

In some embodiments of the methods provided herein, the CDK2-related disease or disorder is a hematological malignancy. In some embodiments, the hematological malignancy is mediated by unwanted activity of CDK2. In some embodiments, the hematological malignancy is mediated by over-expression or amplification of cyclin E. In some embodiments, the hematological malignancy is mediated by over-expression or amplification of cyclin A. In some embodiments, the methods further comprise administering an anti-cancer agent to the subject.

In some embodiments, the cancer is breast cancer, ovary cancer, cervix cancer, prostate cancer, testis cancer, genitourinary tract cancer, esophagus cancer, larynx cancer, glioblastoma, neuroblastoma, stomach cancer, skin cancer, keratoacanthoma, lung cancer, epidermoid carcinoma, large cell cancer, non-small cell lung cancer (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, buccal cavity cancer, naso-pharyngeal cancer, pharynx cancer, lip cancer, tongue cancer, mouth cancer, small intestine cancer, colon-rectum cancer, large intestine cancer, rectum cancer, bronchial cancer, hepatocellular cancer, gastric cancer, endometrial cancer, melanoma, renal cancer, urinary bladder cancer, uterine corpus cancer, or uterine cervix cancer.

In some embodiments, the cancer is squamous cell carcinoma, small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

In some embodiments, the cancer is breast cancer. The breast cancer can be Stage I, II, III, or IV as understood in the art. In some embodiments, the breast cancer is triple negative breast cancer (TNBC). In another embodiment, the breast cancer is Her2 negative breast cancer.

In some embodiments, the CDK2-related disease or disorder is a hematological malignancy such as lymphoma, lymphocytic leukemia (acute (ALL) and chronic (CLL), multiple myeloma (MM), acute myelogenous leukemia (AML), chronic myelogenous leukemia (CML), myelodysplastic syndrome (MDS), myeloproliferative disease (MPD), or non-Hodgkin lymphoma. In some embodiments, the methods herein include treatment of lymphoma, lymphocytic leukemia, multiple myeloma (MM), acute myelogenous leukemia (AML), chronic myelogenous leukemia (CML), myelodysplastic syndrome (MDS), or myeloproliferative disease (MPD).

In some embodiments, the hematological malignancy is identified as having amplification or overexpression of cyclin E. In some embodiments, the hematological malignancy is identified as having amplification or overexpression of cyclin A.

In some embodiments, the methods provided herein further comprises administering to the subject an additional therapeutic agent(s) selected from the group consisting of an anti-inflammatory agent, a corticosteroid, an immunomodulatory agent, anti-cancer agent as described herein, an apoptosis-enhancer, a neurotropic factor, an agent for treating cardiovascular disease, an agent for treating liver disease, an anti-viral agent, an agent for treating blood disorders, an agent for treating diabetes, an agent for treating metabolic disorders, an agent for treating autoimmune disorders, an agent for treating immunodeficiency disorders, and combinations thereof. In some embodiments, the additional therapeutic agent is a corticosteroid, a proteasome inhibitor, an IMiD, an antibody, or a combination thereof. In some embodiments, the additional therapeutic agent is a proteasome inhibitor (e.g. carfilzomib, bortezomib, or ixazomib). In some embodiments, the additional therapeutic agent is an IMiD (e.g. lenalidomide or pomalidomide). In some embodiments, the additional therapeutic agent is an antibody (e.g., an anti-CD38 antibody, an anti-VEGF-A antibody, an anti-PD-1 antibody, an anti-PD-L1 antibody or an anti-interleukin-6 antibody). In some embodiments, the additional therapeutic agent is a corticosteroid (e.g., dexamethasone). In some embodiments, the method further comprises radiotherapy.

In some embodiments, the CDK2-related disease or disorder is an autoimmune disorder or disease. In some embodiments, the autoimmune disease or disorder is identified as having amplification or overexpression of cyclin E. In some embodiments, the autoimmune disease or disorder is identified as having amplification or overexpression of cyclin A. Exemplary autoimmune disorder or disease contemplated in the methods provided herein includes, but not limited to, rheumatoid arthritis, Lupus, Crohn's Disease, Addison disease, celiac disease, dermatomyositis, Graves' disease, thyroiditis, multiple sclerosis, pernicious anemia, reactive arthritis, pemphigus vulgaris, diabetes mellitus type 1 (IDDM), systemic lupus erythematosus (SLE), Sjogren's syndrome, Churg-Strauss Syndrome, Hashimoto's thyroiditis, idiopathic thrombocytopenic purpura, or rheumatoid arthritis.

In some embodiments, the CDK2-related disease or disorder is an inflammatory disease or disorder. In some embodiments, the inflammatory disease or disorder is identified as having amplification or overexpression of cyclin E. In some embodiments, the inflammatory disease or disorder is identified as having amplification or overexpression of cyclin A. Exemplary inflammatory disease or disorder contemplated in the methods provided herein includes, but not limited to, asthma, chronic peptic ulcers, psoriasis, inflammatory bowel disease, tuberculosis, rheumatoid arthritis, periodontitis, ulcerative colitis, hepatitis chronic prostatitis, glomerulonephritis, hypersensitivities, pelvic inflammatory disease, reperfusion injury, transplant rejection, or vasculitis.

In some embodiments, the CDK2-related disease or disorder a neurodegenerative disease or disorder. In some embodiments, the neurodegenerative disease or disorder is identified as having amplification or overexpression of cyclin E. In some embodiments, the neurodegenerative disease or disorder is identified as having amplification or overexpression of cyclin A. Exemplary neurodegenerative disease or disorder contemplated in the methods provided herein includes, but not limited to, Alzheimer's disease, AIDS-related dementia, Parkinson's disease, amyotrophic lateral sclerosis, retinitis pigmentosa, spinal muscular atrophy, cerebellar degeneration, Alexander's disease, Alper's disease, Ataxia telangiectasia, Batten disease (also known as Spielmeyer-Vogt-Sjogren-Batten disease), Bovine spongiform encephalopathy (BSF), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Pelizaeus-Merzbacher Disease, Pick's disease. Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

Also provided herein, in some embodiments, is a method of killing a cell over-expressing cyclin E in a subject in need thereof, comprising administering to the subject an effective amount of a compound of Formula (I), or a pharmaceutically acceptable salt thereof, or a pharmaceutical composition thereof. In some embodiments, also disclosed herein is a method of killing a cell over-expressing or amplifying cyclin E, comprising contacting the cell with an effective amount of a compound of Formula (I), or a pharmaceutically acceptable salt thereof, or a pharmaceutical composition thereof.

In some embodiments, the cyclin E is CCNE1 or CCNE2.

Also provided herein, in some embodiments, is a method of killing a cell over-expressing cyclin A in a subject in need thereof, comprising administering to the subject an effective amount of a compound of Formula (I), or a pharmaceutically acceptable salt thereof, or a pharmaceutical composition thereof. In some embodiments, also disclosed herein is a method of killing a cell over-expressing or amplifying cyclin A, comprising contacting the cell with an effective amount of a compound of Formula (I), or a pharmaceutically acceptable salt thereof, or a pharmaceutical composition thereof.

Also provided herein, in some embodiments, is a method of inhibiting CDK2 in a cell over-expressing cyclin E in a subject in need thereof, comprising administering to the subject an effective amount of a compound of Formula (I), or a pharmaceutically acceptable salt thereof, or a pharmaceutical composition thereof. In some embodiments, also disclosed herein is a method of inhibiting CDK2 in cell over-expressing or amplifying cyclin E, comprising contacting the cell with an effective amount of a compound of Formula (I), or a pharmaceutically acceptable salt thereof, or a pharmaceutical composition thereof.

In some embodiments, the cyclin E is CCNE1 or CCNE2.

Also provided herein, in some embodiments, is a method of inhibiting CDK2 in a cell over-expressing cyclin A in a subject in need thereof, comprising administering to the subject an effective amount of a compound of Formula (I), or a pharmaceutically acceptable salt thereof, or a pharmaceutical composition thereof. In some embodiments, also disclosed herein is a method of inhibiting CDK2 in a cell over-expressing or amplifying cyclin A, comprising contacting the cell with an effective amount of a compound of Formula (I), or a pharmaceutically acceptable salt thereof, or a pharmaceutical composition thereof.

Also provided herein, in some embodiments, is a method of treating a disease or disorder characterized by overexpression or amplification of cyclin E in a subject in need thereof, the method comprising administering to the subject an effective amount of a compound of Formula (I), or a pharmaceutically acceptable salt thereof, or a pharmaceutical composition thereof. In some embodiments, the cyclin E is CCNE1 or CCNE2.

In some embodiments, provided herein is a method of treating a disease or disorder characterized by overexpression or amplification of cyclin A in a subject in need thereof, the method comprising administering to the subject an effective amount of a compound of Formula (I), or a pharmaceutically acceptable salt thereof, or a pharmaceutical composition thereof

### Pharmaceutical Formulations

Compounds provided in accordance with the present disclosure, in some embodiments, are administered in the form of pharmaceutical compositions. This disclosure therefore provides pharmaceutical compositions that comprise, as the active ingredient, one or more of the compounds described, or a pharmaceutically acceptable salt thereof, and one or more pharmaceutically acceptable excipients, including inert solid diluents and fillers, diluents, including sterile aqueous solution and various organic solvents, permeation enhancers, solubilizers and adjuvants. In some embodiments, the pharmaceutical compositions are administered in combination with other therapeutic agents.

In some embodiments, the pharmaceutical compositions are administered in a single dose by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. In some embodiments, the pharmaceutical compositions are administered in multiple doses.

One mode for administration is parenteral, for example by injection. Administration by injection comprises, in some embodiments, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. In some embodiments, compositions suitable for injection comprise ethanol, glycerol, propylene glycol, liquid polyethylene glycol, or the like (and suitable mixtures thereof), cyclodextrin derivatives, or vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, or the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the disclosure. Oral administration includes, for example, capsule or enteric coated tablets, or the like. In making the pharmaceutical compositions that include at least one compound described herein, the active ingredient is, in some embodiments, diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments comprising the active compound, soft and hard gelatin capsules, sterile injectable solutions, or sterile packaged powders.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Another formulation for use in the methods of the present disclosure employs transdermal delivery devices (“patches”). Such transdermal patches may be used to provide continuous or discontinuous infusion of the compounds of the present disclosure in controlled amounts. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

In some embodiments, the tablets or pills of the present disclosure are coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. In some embodiments, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Combination Therapy

In some embodiments, the methods provided herein further comprises administering additional therapeutic agents for the treatment, prevention, or reduction of risk or severity of a disease or disorder described herein, such as cancer. In some embodiments, the additional therapeutic agent has anti-inflammatory or anti-cancer properties or that is useful for treating an inflammation, immune-response disorder, or cancer.

In some embodiments, the additional therapeutic agent is selected from a Bcl-2 inhibitor, a JAK inhibitor, a PI3K inhibitor, an mTOR inhibitor, an anti-inflammatory agent, an immunomodulatory agent, anti-cancer agent as described herein, an apoptosis-enhancer, a neurotropic factor, an agent for treating cardiovascular disease, an agent for treating liver disease, an anti-viral agent, an agent for treating blood disorders, an agent for treating diabetes, and an agent for treating immunodeficiency disorders. The additional therapeutic agent can be an NSAID anti-inflammatory agent. The additional therapeutic agent can be an anti-cancer agent as described herein. The second compound of the pharmaceutical combination formulation or dosing regimen preferably has complementary activities to the compound of Formula (I) such that they do not adversely affect each other. Such compounds are suitably present in combination in amounts that are effective for the purpose intended. In some embodiments, a composition provided herein comprises a compound of Formula (I), or a pharmaceutically acceptable salt thereof, in combination with an additional therapeutic agent.

The combination therapy can be administered as a simultaneous or sequential regimen. When administered sequentially, the combination can be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and can be lowered due to the combined action (synergy) of the newly identified agent and other therapeutic agents or treatments.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alteration or in parallel as separate formulations; or (3) by some other regimen. When delivered in alternation therapy, a synergistic effect can be attained when the compounds are administered or delivered sequentially, e.g., by different injections in separate syringes, separate pills or capsules, or separate infusions. In general, during alternation therapy, an effective dosage of each active ingredient is administered sequentially, i.e., serially, whereas in combination therapy, effective dosages of two or more active ingredients are administered together.

In a particular embodiment of therapy, a compound of Formula (I) or a pharmaceutically acceptable salt thereof, can be combined with other therapeutic, hormonal or antibody agents such as those described herein, as well as combined with surgical therapy and radiotherapy. Combination therapies provided herein thus comprise the administration of at least one compound of Formula (I) or pharmaceutically acceptable salt thereof, and the use of at least one other cancer treatment method. The amounts of the compound(s) of Formula (I) or pharmaceutically acceptable salts thereof described herein, and the other pharmaceutically active therapeutic agent(s) and the relative timings of administration will be selected in order to achieve the desired combined therapeutic effect.

In some embodiments, a compound of Formula (I), or a pharmaceutically acceptable salt thereof, is used in combination with an aromatase inhibitor, a phosphoinositide 3-kinase (PI3K)/mTOR pathway inhibitor, a CDK 4/6 inhibitor, a HER-2 inhibitor, a SERM, a SERD, an EGFR inhibitor, a PD-1 inhibitor, poly ADP-ribose polymerase (PARP) inhibitor, a histone deacetylase (HDAC) inhibitor, an HSP90 inhibitor, a VEGFR inhibitor, an AKT inhibitor, chemotherapy, or any combination thereof.

In some embodiments, a pharmaceutical composition comprising a compound of Formula (I), or a pharmaceutically acceptable salt thereof, is administered in combination with a therapeutic agent selected from paclitaxel, anastrozole, exemestane, cyclophosphamide, epirubicin, fulvestrant, letrozole, palbociclib, gemcitabine, trastuzumab (HERCEPTIN®, Genentech), trastuzumab emtansine (KADCYLA®, Genentech), pegfilgrastim, filgrastim, tamoxifen, docetaxel, toremifene, vinorelbine, capecitabine, and ixabepilone.

In some embodiments, a compound of Formula (I), or a pharmaceutically acceptable salt thereof, is used in combination with hormone blocking therapy, chemotherapy, radiation therapy, monoclonal antibodies, or combinations thereof.

### Metabolites of Compounds of Formula I

In some embodiments, the methods provided herein comprise in vivo metabolic products of compounds or pharmaceutically acceptable salts thereof as described herein. Such products can result for example from the oxidation, reduction, hydrolysis, amidation, deamidation, esterification, deesterification, enzymatic cleavage, and the like, of the administered compound. Accordingly, provided herein are compounds produced by a process comprising contacting a compound or pharmaceutically acceptable salt thereof described herein with a mammal for a period of time sufficient to yield a metabolic product thereof.

Metabolite products typically are identified by preparing a radiolabelled (e.g., 14C or 3H) isotope of a compound or pharmaceutically acceptable salt thereof as described herein, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS. LC/MS or NMR analysis. In general, analysis of metabolites is done in the same way as conventional drug metabolism studies. The metabolite products, so long as they are not otherwise found in vivo, are useful in diagnostic assays for therapeutic dosing of the compounds or pharmaceutically acceptable salts thereof described herein.

### Dosage Regimens

Dosage regimens may be adjusted to provide the optimum desired response. The skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of the compound of the disclosure administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day. In some instances, dosage levels below the lower limit of the aforesaid range may be more than adequate, while in other cases still larger doses may be employed without causing any harmful side effect, provided that such larger doses are first divided into several small doses for administration throughout the day.

### Kits

In another aspect provided herein is a kit, comprising materials useful for the treatment or prevention of the diseases and disorders described above. In some embodiments, the kit comprises a container comprising a compound of Formula (I) or a pharmaceutically acceptable salt thereof. In some embodiments, the kit further comprises a label or package insert on or associated with the container. Suitable containers include, for example, bottles, vials, syringes, blister pack, etc. The container can be formed from a variety of materials such as glass or plastic. The container can hold a compound of Formula (I) or a composition thereof which is effective for treating or preventing the condition and can have a sterile access port (for example, the container can be an intravenous solution bag or a vial having a stopper pierceable by a hypodermic injection needle). At least one active agent in the composition is a compound of Formula (I) or a pharmaceutically acceptable salt thereof. The label or package insert indicates that the composition is used for treating the condition of choice. The label or package insert can also indicate that the composition can be used to treat other disorders. Alternatively, or additionally, the article of manufacture can further comprise a second container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

The kit can further comprise directions for the administration of the compound of Formula (I) or a pharmaceutically acceptable salt thereof and, if present, the second pharmaceutical formulation. For example, if the kit comprises a first composition comprising a compound of Formula (I) or a pharmaceutically acceptable salt thereof and a second pharmaceutical formulation, the kit can further comprise directions for the simultaneous, sequential or separate administration of the first and second pharmaceutical compositions to a patient in need thereof.

In another embodiment, the kits are suitable for the delivery of solid oral forms of a compound of Formula (I) or a pharmaceutically acceptable salt thereof, such as tablets or capsules. Such a kit preferably includes a number of unit dosages. Such kits can include a card having the dosages oriented in the order of their intended use. An example of such a kit is a blister pack. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

In some embodiments, a kit comprises (a) a first container with a compound of Formula (I) or a pharmaceutically acceptable salt thereof contained therein; and optionally (b) a second container with a second pharmaceutical formulation contained therein. Alternatively, or additionally, the kit can further comprise a third container comprising a pharmaceutically-acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

In some embodiments wherein the kit comprises a composition of Formula (I) or a pharmaceutically acceptable salt thereof and a second therapeutic agent, the kit can comprise a container for containing the separate compositions such as a divided bottle or a divided foil packet, however, the separate compositions can also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein the specification. “a” or “an” may mean one or more. As used herein, when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method, compound, or composition described herein can be implemented with respect to any other method, compound, or composition described herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999: Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge. 1987.

The following terms are intended to have the meanings presented therewith below and are useful in understanding the description and intended scope of the present disclosure. When describing the disclosure, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below.

Unless otherwise indicated, any heteroatom with unsatisfied valences is assumed to have hydrogen atoms sufficient to satisfy the valences.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should be further understood that the terms “groups” and “radicals” can be considered interchangeable when used herein.

“Alkyl” as used herein refers to a saturated linear (i.e. unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having a 1 to 8 carbon atoms (a “C₁₋₈ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl group include, but are not limited to, groups such as methyl, ethyl, n-propyl, isopropyl, n-butyl, t-butyl, isobutyl, secbutyl, homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂₋₁₀ means two to ten carbon atoms). The alkenyl group can be in “cis” or “trans” configurations, or alternatively in “E” or “Z” configurations. Particular alkenyl groups are those having 2 to 20 carbon atoms (a “C₂₋₂₀ alkenyl”), having a 2 to 8 carbon atoms (a “C₂₋₈ alkenyl”), having 2 to 6 carbon atoms (a “C₂₋₆ alkenyl”), or having 2 to 4 carbon atoms (a “C₂₋₄ alkenyl”). Example of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e. unbranched) or branched univalent hydrocarbon chain or combination thereof, having at least one site of acetylenic unsaturation (i.e., having at least one moiety of the formula C═C) having the number of carbon atoms designated (i.e., C₂₋₁₀ means two to ten carbon atoms). Particular alkynyl groups are those having 2 to 20 carbon atoms (a “C₂₋₂₀ alkynyl”), having a 2 to 8 carbon atoms (a “C₂₋₆ alkynyl”), having 2 to 6 carbon atoms (a “C₂₋₆ alkynyl”), having 2 to 4 carbon atoms (a “C₂₋₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylene” as used herein refers to the same residues as alkyl, but having bivalency. Particular alkylene groups are those having 1 to 6 carbon atoms (a “C₁₋₆ alkylene”), 1 to 5 carbon atoms (a “C₁₋₅ alkylene”), having 1 to 4 carbon atoms (a “C₁₋₄ alkylene”), or 1 to 3 carbon atoms (a “C₁₋₃ alkylene”). Examples of alkylene include, but are not limited to, groups such as methylene (—CH₂—), ethylene (—CH₂—CH₂—), propylene (—CH₂—CH₂—CH₂—), butylene (—CH₂—CH₂—CH₂—CH₂—), and the like.

“Cycloalkyl” as used herein refers to non-aromatic, saturated or unsaturated cyclic univalent hydrocarbon structures having the number of carbon atoms designated (i.e., (C₃₋₁₀ means three to ten carbon atoms). Cycloalkyl can consist of one ring, such as cyclohexyl, or multiple rings, such as adamantly, but excludes aryl groups. A cycloalkyl comprising more than one ring can be fused, spiro, or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃₋₈ cycloalkyl”), or having 3 to 6 carbon atoms (a “C₃₋₆ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Aryl” as used herein refers to an unsaturated aromatic carbocyclic group having a single ring (e.g., phenyl) or multiple condensed rings (e.g., naphthyl or anthryl) which condensed rings can or can not be aromatic. Particular aryl groups are those having from 6 to 14 annular (i.e., ring) carbon atoms (a “C₆₋₁₄ aryl”). An aryl group having more than one ring where at least one ring is non-aromatic can be connected to the parent structure at either an aromatic ring position or at a non-aromatic ring position. In some embodiments, an aryl group having more than one ring where at least one ring is non-aromatic is connected to the parent structure at an aromatic ring position.

“Heteroaryl” as used herein refers to an unsaturated aromatic cyclic group having from 1 to 14 annular (i.e., ring) carbon atoms and at least one annular heteroatom, including but not limited to heteroatoms such as nitrogen, phosphorus, oxygen and sulfur. A heteroaryl group can have a single ring (e.g., pyridyl, furyl) or multiple condensed rings (e.g., indolizinyl, benzothienyl) which condensed rings can or can not be aromatic. Particular heteroaryl groups are 5- to 14-membered rings having 1 to 12 annular (i.e., ring) carbon atoms and 1 to 6 annular (i.e., ring) heteroatoms independently selected from nitrogen, phosphorus, oxygen and sulfur; 5- to 10-membered rings having 1 to 8 annular carbon atoms and 1 to 4 annular heteroatoms independently selected from nitrogen, phosphorus, oxygen and sulfur; and 5-, 6- or 7-membered rings having 1 to 5 annular carbon atoms and 1 to 4 annular heteroatoms independently selected from nitrogen, oxygen and sulfur In some embodiments, heteroaryl include monocyclic aromatic 5-, 6- or 7-membered rings having from 1 to 6 annular carbon atoms and 1 to 4 annular heteroatoms independently selected from nitrogen, oxygen and sulfur. In another variation, heteroaryl includes polycyclic aromatic rings having from 1 to 12 annular carbon atoms and 1 to 6 annular heteroatoms independently selected from nitrogen, phosphorus, oxygen and sulfur. A heteroaryl group having more than one ring where at least one ring is non-aromatic can be connected to the parent structure at either an aromatic ring position or at a non-aromatic ring position. In some embodiments, a heteroaryl group having more than one ring where at least one ring is non-aromatic is connected to the parent structure at an aromatic ring position.

“Heterocycle”, “heterocyclic”, or “heterocyclyl” as used herein refers to a saturated or an unsaturated non-aromatic cyclic group having a single ring or multiple condensed rings, and having from 1 to 14 annular (i.e., ring) carbon atoms and from 1 to 6 annular (i.e., ring) heteroatoms, such as nitrogen, phosphorus, sulfur or oxygen, and the like. A heterocycle comprising more than one ring can be fused, spiro or bridged, or any combination thereof. In fused ring systems, one or more can be fused rings can be cycloalkyl, Particular heterocyclyl groups are 3- to 14-membered rings having 1 to 13 annular carbon atoms and 1 to 6 annular heteroatoms independently selected from nitrogen, phosphorus, oxygen and sulfur; 3- to 12-membered rings having 1 to 11 annular carbon atoms and 1 to 6 annular heteroatoms independently selected from nitrogen, phosphorus, oxygen and sulfur; 3- to 10-membered rings having 1 to 9 annular carbon atoms and 1 to 4 annular heteroatoms independently selected from nitrogen, phosphorus, oxygen and sulfur; 3- to 8-membered rings having 1 to 7 annular carbon atoms and 1 to 4 annular heteroatoms independently selected from nitrogen, phosphorus, oxygen and sulfur; and 3- to 6-membered rings having 1 to 5 annular carbon atoms and 1 to 4 annular heteroatoms independently selected from nitrogen, phosphorus, oxygen and sulfur. In some embodiments, heterocyclyl include monocyclic 3-, 4-, 5-, 6- or 7-membered rings having from 1 to 2, 1 to 3, 1 to 4, 1 to 5 or 1 to 6 annular carbon atoms and 1 to 2, 1 to 3 or 1 to 4 annular heteroatoms independently selected from nitrogen, phosphorus, oxygen and sulfur. In another variation, heterocyclyl includes polycyclic non-aromatic rings having from 1 to 12 annular carbon atoms and 1 to 6 annular heteroatoms independently selected from nitrogen, phosphorus, oxygen and sulfur.

“Halo” or Halogen” refers to fluoro, chloro, bromo and/or iodo. Where a residue is substituted with more than one halogen, it can be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which can be but are not necessarily the same halo; thus 4-chloro-3-fluorophenyl is within the scope of dihaloaryl. An alkyl group in which one or more hydrogen is replaced with a halo group is referred to as a “haloalkyl”, for example. “C₁₋₆ haloalkyl.” An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O.

Certain compounds of the present disclosure may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

All stereoisomers of the present compounds (for example, those which may exist due to asymmetric carbons on various substituents), including enantiomeric forms and diastereomeric forms, are contemplated within the scope of this disclosure. Individual stereoisomers of the compounds of the disclosure may, for example, be substantially free of other isomers (e.g., as a pure or substantially pure optical isomer having a specified activity), or may be admixed, for example, as racemates or with all other, or other selected, stereoisomers. The chiral centers of the present disclosure may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present disclosure are contemplated, either in admixture or in pure or substantially pure form. The definition of compounds of the present disclosure embraces both cis (Z) and trans (E) alkene isomers, as well as cis and trans isomers of cyclic hydrocarbon or heterocyclic rings.

Isomeric mixtures containing any of a variety of isomer ratios may be utilized in accordance with the present disclosure. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present disclosure.

The present disclosure also includes isotopically-labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorous, sulfur, fluorine, and chlorine, such as 2H, 3H, 13C, 11C, 14C, 15N, 18O, 17O, 31P, 32P, 35S, 18F, and 36Cl, respectively. Compounds of the present disclosure, or a stereoisomer, tautomer, or pharmaceutically acceptable salt or solvate thereof, which contain the aforementioned isotopes and/or other isotopes of other atoms are within the scope of this disclosure.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” “subject,” and “individual” are used interchangeably herein. None of these terms require the active supervision of medical personnel.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or reverses or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. A “therapeutically effective amount” of a compound is an amount sufficient to provide a therapeutic benefit (e.g., treating, preventing, and/or ameliorating cancer in a subject, or inhibiting protein-protein interactions mediated by CDK2 in a subject, at a reasonable benefit/risk ratio applicable to any medical treatment) in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. A “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent. A “prophylactic treatment” contemplates an action that occurs before a subject begins to suffer from the specified disease, disorder or condition.

The term “cancer” refers to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), small-cell lung cancer, non-small cell lung cancer (NSCLC), lung adenocarcinoma, squamous cell lung cancer, peritoneum cancer, hepatocellular cancer, stomach cancer, gastrointestinal cancer, esophageal cancer, pancreatic cancer, glioblastoma, cervical cancer, ovarian cancer, liver cancer, bladder cancer, breast cancer, colon cancer, rectal cancer, colorectal cancer, endometrial cancer, uterine cancer, salivary gland carcinoma, renal cancer, prostate cancer, vulval cancer, thyroid cancer, hepatocellular carcinoma (HCC), anal carcinoma, penile carcinoma, or head and neck cancer.

“Hematological malignancy” is a type of cancer that affect blood, bone marrow, and lymph nodes. As the three are intimately connected through the immune system, a disease affecting one of the three will often affect the others as well: although lymphoma is a disease of the lymph nodes, it often spreads to the bone marrow, affecting the blood. Hematological malignancies are malignant neoplasms (i.e. cancer), and they are generally treated by specialists in hematology and/or oncology. Hematological malignancies can derive from either of the two major blood cell lineages: myeloid and lymphoid cell lines. Lymphomas, lymphocytic leukemias, and myeloma are from the lymphoid line, while acute and chronic myelogenous leukemia, myelodysplastic syndromes and myeloproliferative diseases are myeloid in origin. Exemplary leukemias include acute lymphoblastic leukemia (ALL), acute myelogenous leukemia (AML), chronic lymphocytic leukemia (CLL), chronic myelogenous leukemia (CML), acute monocytic leukemia (AMOL) and small lymphocytic lymphoma (SLL). Exemplary lymphomas include Hodgkin's lymphomas (all four subtypes) and Non-Hodgkin's lymphomas (NHL, all subtypes).

An “anti-cancer agent” is a chemical compound useful in the treatment of cancer, regardless of mechanism of action. Classes of anti-cancer agents include, but are not limited to: alkylating agents, antimetabolites, anti-hormone therapies, endocrine therapies, immunomodulatory agents, spindle poison plant alkaloids, cytotoxic/antitumor antibiotics, topoisomerase inhibitors, antibodies, photosensitizers, and kinase inhibitors. Anti-cancer agents include compounds used in targeted therapy and conventional chemotherapy. Exemplary anti-cancer agents include proteasome inhibitors such as bortezomib (VELCADE), carfilzomib (KYPROLIS) and ixazomib (NINLARO). Other examples include immunomodulatory agents such as lenalidomide (REVLIMID) and pomalidomide (POMALYST). Other exemplary anti-cancer agents include inhibitors of B-cell receptor targets such as BTK. Bcl-2 and JAK inhibitors and include, for example, venetoclax (VENCLEXTA) and ibrutinib (IMBRUVICA). Additional anti-cancer agents include, for example, Abemaciclib (VERZENIO); abiraterone (ZYTIGA, YONS A); aclarubicin; aci vicin; acodazole; acronine; actinomycin; acylfulvene; adecypenol; adozelesin; adriamycin; aldesleukin; altretamine; ambamustine; ambomycin; ametantrone; amidox; amifostine; aminoglutethimide; aminolevulinic acid; amrubicin; amsacrine: anagrelide: anastrozole; andrographolide; antarelix: anthramycin; aphidicolin glycinate; apurinic acid; ARRY-300; arabinoside; asperlin; asulacrine; atamestane; atrimustine; azasetron; azatoxin; azatyrosine; azacitidine; AZD6244; AZD8330; azetepa; azotomycin; balanol; batimastat; bendamustine; benzochlorins; benzodopa: benzoylstaurosporine; beta-alethine; betaclamycin B: betulinic acid; bicalutamide; binimetinib; bisantrene; bisaziridinylspermine; bisnafide; bistratene; bleomycin; busulfan: bizelesin; breflate; bortezomib; brequinar: bropirimine; budotitane; buthionine: bryostatin; cactinomycin; calusterone: calcipotriol; calphostin C; camptothecin; capecitabine (XELODA); caracemide: carbetimer; carboplatin; carboquone; carmustine; carubicin: carzelesin: castanospermine: celecoxib; cetrorelix; cetuximab (ERBITUX); chloroquinoxaline; cicaprost; chlorambucil; chlorofusin; cisplatin; cladribine; clomifene; clotrimazole; crisnatol; crisnatol; cypemycin; cyclophosphamide; cytarabine; cytostatin; dacarbazine; dactinomycin; daratumamab; daunorubicin; decarbazine; dacliximab; dasatinib; decitabine; deslorelin; dexamethasone; dexifosfamide; dexrazoxane; dexverapamil; dexormaplatin; dezaguanine: diaziquone: dihydrotaxol; docosanol; dolasetron; docetaxel; doxorubicin; doxifluridine; droloxifene; dromostanolone; dronabinol; duazomycin; ebselen; ecomustine; edelfosine; edrecolomab; edatrexate; eflomithine; elemene; emitefur; elsamitrucin; enloplatin; enpromate; epipropidine; epirubicin: epristeride; erbulozole; erlotinib (TARCEVA); esorubicin; estramustine; etanidazole: etoposide; etoprine; exemestane; fadrozole: fazarabine; fenretinide; filgrastim: finasteride: flavopiridol; flezelastine; fluasterone; floxuridine; fludarabine; fludarabine; fluorodaunorubicin; forfenimex; formestane; fluorouracil; floxouridine; flurocitabine; fosquidone; fostriecin; fotemustine; fulvestrant (FASLODEX); gadolinium; gallium: galocitabine: ganirelix; gemcitabine; geldanamvcin; gefitinib; gossyphol; hydroxyurea; hepsulfam; heregulin; ibandronate; ibrutinib; idarubicin; idelalisib (ZYDELIG), ifosfamide; canfosfamide; ilmofosine; iproplatin; idoxifene; idramantone; ilmofosine; ilomastat; imatinib mesylate (GLEEVEC); imiquimod; iobenguane; iododoxorubicin; ipomeanol; irinotecan; itasetron; iimofosine: lanreotide: lapatinib (TYKERB); leinamycin: lenograstim; lentinan; leptolstatin; letrozole; leuprorelin; levamisole; liarozole; lobaplatin; lombricine; lometrexol: lonidamine; lonafamib (SARASAR); losoxantrone; lovastatin; loxoribine; lurtotecan; lapatinib; leucovorin: lometrexol; lomustine; maitansine; marimastat; masoprocol; maspin; menogaril; merbarone: meterelin; methioninase; metoclopramide; mifepristone; miltefosine: mirimostim; mitoguazone: mitolactol: mitonafide: mitoxantrone; mofarotene; molgramostim; mopidamol; maytansine; megestrol acetate; melengestrol acetate; melphalan; mercaptopurine; methotrexate; methotrexate sodium; metoprine; meturedepa; mitinmitomycin; mitosper; mitotane: mitoxantrone; mycophenolic acid; nafarelin; nagrestip; napavin; nedaplatin; nemorubicin; neridronic acid; nilutamide; nisamycin; oblimersen (GENASENSE); octreotide; okicenone; onapristone; ondansetron; ormaplatin; oxisuran; oxaloplatin; osaterone; oxaliplatin; oxaunomycin; palauamine; palbociclib (IBRANCE); panitumumab (VECTIBIX); panomifene; pegaspargase; picibanil; pirarubicin; piritrexim; prednisone; prednisolone, paclitaxel; nab-paclitaxel (ABRAXANE): prednimustine: procarbazine; puromycin; raltitrexed; ramosetron: rapamycin (RAPAMUNE); rhizoxin; ribociclib (KISQALI), rituximab; rogletimide; rohitukine; romurtide; roquinimex; romidepsin; safingol; saintopin; sargramostim; semustine; sizofiran; sobuzoxane; sorafenib (NEXAVAR); sunitinib; spiromustine; squalamine; suradista: suramin: swainsonine: spiroplatin; streptonigrin; streptozocin; sulofenur: tallimustine: tamoxifen; tauromustine; tazarotene; tellurapyrylium: temoporfin; temozolomide; teniposide; tetrachlorodecaoxide; tetrazomine; thrombopoietin; thymalfasin; thymotrinan; tirapazamine: toremifene; tretinoin; trimetrexate; triptorelin; tropisetron: talisomycin; taxotere; teroxirone; testolactone; thiamiprine: thiotepa; tirapazamine: toremifene; trastuzumab; trastuzumab emtansine; trestolone acetate; triciribine phosphate; trimetrexate; uracil mustard; vandetanib (CAPRELSA); variolin B; velaresol; veramine; verteporfin; vemurafenib; vinorelbine; vinxaltine; vitaxin; vinblastine: vincristine; vindesine; vinepidine; vinglvcinate; vinleurosine: vinorelbine; vinrosidine: vinzolidine; vorozole; wortmannin; zanoterone: zeniplatin; zilascorb; zinostatin stimalamer: zinostatin; and zorubicin. In some embodiments, the anti-cancer agent includes, for example, idelalisib (ZYDELIG), docetaxel, fluorouracil, gemcitabine (GEMZAR), cisplatin, cis-diamine, carboplatin, paclitaxel, nab-paclitaxel, trastuzumab (HERCEPTIN), temozolomide, tamoxifen, 4-hydroxytamoxifen, and doxorubicin.

Also included in the definition of anti-cancer agent are: (i) anti-estrogens and selective estrogen receptor modulators (SERMs), including, for example, tamoxifen, raloxifene, droloxifene, 4-hydroxytamoxifen, trioxifene, ketoxifene, LY117018, onapristone, and toremifine citrate; (ii) selective estrogen receptor modulators (SERDs) such as brilanestrant. GDC-0927, GDC-9545, AZ9496, AZ9833, GNE-274, and fulvestrant (FASLODEX): (iii) aromatase inhibitors such as, for example, 4(5)-imidazoles, aminoglutethimide, megestrol acetate, exemestane, formestanie, fadrozole, vorozole, letrozole, and anastrozole; (iv) anti-androgens such as apalutamide, abiraterone, enzalutamide, flutamide, nilutamide, bicalutamide, leuprolide, and goserelin. Further included in the definition of anti-cancer agents are: (v) MEK inhibitors such as cobimetinib; (vi) lipid kinase inhibitors, such as taselisib; (vii) antisense oligonucleotides such as oblimersen; (viii) ribozymes such as VEGF expression inhibitors such as angiozyme; (ix) vaccines such as gene therapy vaccines, for example, ALLOVECTIN, LEUVECTIN, and VAXID; (x) topoisomerase 1 inhibitors such as LURTOTECAN®; ABARELIX® rmRH; and (xi) anti-angiogenic agents such as bevacizumab. In some embodiments herein, the anti-cancer agents is a therapeutic antibody such as atezolizumab, nivolumab, daratumumab, pembrolizumab, alemtuzumab, bevacizumab; cetuximab; panitumumab, rituximab, pertuzumab, trastuzumab, trastuzumab emtansine, or tositumomab.

A “metabolite” is a product produced through metabolism in the body of a specified compound or salt thereof. Metabolites of a compound can be identified using routine techniques and their activities determined using tests such as those described herein. Such products can result for example from the oxidation, reduction, hydrolysis, amidation, deamidation, esterification, deesterification, enzymatic cleavage, and the like, of the administered compound. Accordingly, provided herein are metabolites of compounds or pharmaceutically acceptable salts thereof described herein, including compounds produced by a process comprising contacting a Formula (I) compound or a pharmaceutically acceptable salt thereof with a mammal for a period of time sufficient to yield a metabolic product thereof.

The term “package insert” is used to refer to instructions customarily included in commercial packages of therapeutic products, that contain information about the indications, usage, dosage, administration, contraindications and/or warnings concerning the use of such therapeutic products.

Any formula or structure given herein, including Formula (I) compounds, is also intended to represent hydrates, solvates, and polymorphs of such compounds, and mixtures thereof.

### Preparation of Formula (I) Compounds

Formula (I) compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958): Arzneimittel-Forschung, 40(12): 1328-31, (1990), each of which are expressly incorporated by reference. Starting materials are generally available from commercial sources such as Aldrich Chemicals (Milwaukee, WI) or are readily prepared using methods (e.g., prepared by methods generally described in Louis F. Fieser and Mary Fieser, Reagents for Organic Synthesis, v. 1-23. Wiley, N.Y. (1967-2006 ed.), or Beilsteins Handbuch der organischen Chemie, 4, Aufl, ed. Springer-Verlag, Berlin, including supplements (also available via the Beilstein online database). Formula (I) compounds can also be made following the procedures found in U.S. Pat. Nos. 8,476,434, 7,880,000, WO 2005/113494, U.S. Pat. No. 7,868,177, and WO 2007/100646.

Synthetic chemistry transformations and protecting group methodologies (protection and deprotection) useful in synthesizing Formula (I) compounds and necessary reagents and intermediates include, for example, those described in R. Larock, Comprehensive Organic Transformations, VCH Publishers (1989); T. W. Greene and P. G. M. Wuts. Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds of Formula (I) can be prepared singly or as compound libraries comprising at least 2, for example 5 to 1,000 compounds, or 10 to 100 compounds. Libraries of compounds of Formula (I) can be prepared by a combinatorial split and mix approach or by multiple parallel syntheses using, for example, either solution phase or solid phase chemistry. Thus according to a further aspect provided herein is a compound library comprising at least 2 compounds, or pharmaceutically acceptable salts thereof as described herein.

The Examples provide exemplary methods for preparing Formula (I) compounds. Those skilled in the art will appreciate that other synthetic routes can be used to synthesize the Formula (I) compounds. Although specific starting materials and reagents are depicted and discussed in the Examples, other starting materials and reagents can be easily substituted to provide a variety of derivatives and/or reaction conditions. In addition, many of the exemplary compounds prepared by the described methods can be further modified in light of this disclosure using conventional chemistry.

In preparing compounds of Formula (I), protection of remote functionality (e.g., primary or secondary amine) of intermediates can be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection can be readily determined. For a general description of protecting groups and their use, see T. W. Greene, Protective Groups in Organic Synthesis, John Wiley & Sons, New York, 1991.

In the methods of preparing Formula (I) compounds, it can be advantageous to separate reaction products from one another and/or from starting materials. The desired products of each step or series of steps is separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus: small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

Diastereomeric mixtures can be separated into their individual diastereomers on the basis of their physical chemical differences by methods such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds or pharmaceutically acceptable salts thereof described herein can be atropisomers (e.g., substituted biaryls). Enantiomers can also be separated by use of a chiral HPLC column.

A single stereoisomer, e.g., an enantiomer, substantially free of its stereoisomer can be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds or pharmaceutically acceptable salts thereof described herein can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, a-methyl-P-phenylethylamine (amphetamine), and the like with asymmetric compounds bearing acidic functionality, such as carboxylic acid and sulfonic acid. The diastereomeric salts can be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (-) menthyl chloroformate in the presence of base, or Mosher ester, a-methoxy-a-(trifluoromethyl)phenyl acetate (Jacob III. J. Org. Chem. (1982) 47:4165), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

Compounds of Formula (I) can be prepared by procedures in the Examples, the General Procedures, and generally by Schemes 1-4, where R groups are as described herein.

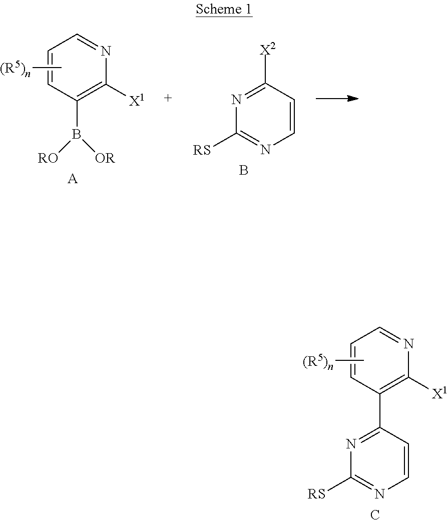

Scheme 1 shows coupling of a (2-halopyridin-3-yl)boronic acid or ester compound A (R=H, C₁-C₆ alkyl, pinacol; X¹=halogen) with a 4-chloro-2-(methylthio)pyrimidine compound B (R=C₁-C₆ alkyl, X²=halogen) under palladium catalysis to form 4-(2-halopyridin-3-yl)-2-(alkylthio)pyrimidine compound C.

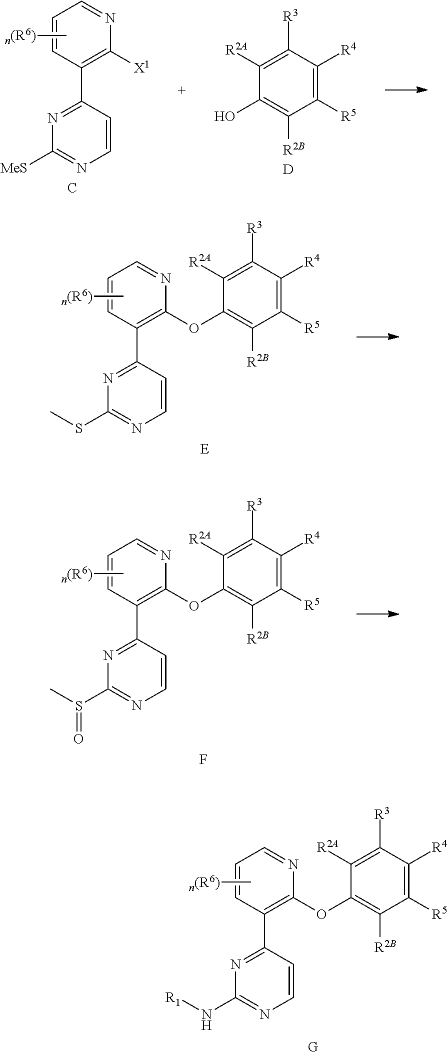

Scheme 2 shows coupling of a phenol compound D with a 4-(2-halopyridin-3-yl)-2-(alkylthio)pyrimidine compound C to form a 4-(2-(phenyloxy)pyridin-3-yl)pyrimidine-2-alkylthiol E compound. Oxidation of the sulfur atom forms 2-(alkylsulfinyl)-4-(2-(phenyloxy)pyridin-3-yl)pyrimidine compound F. The sulfoxide is displaced with a primary amine (R¹—NH₂) to form a Formula (I) or G compound, or an intermediate to be converted to a Formula (I) or G compound.

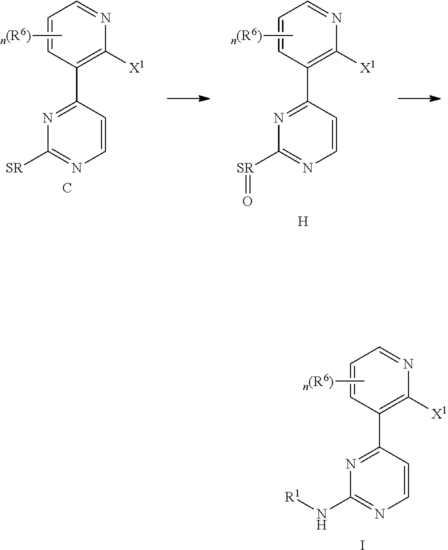

Scheme 3 shows an alternative route to Formula (I) compounds where a 4-(2-halopyridin-3-yl)-2-(alkylthio)pyrimidine compound C is oxidized to a 4-(2-halopyridin-3-yl)-2-(alkylsulfinyl)pyrimidine compound H. The sulfoxide is displaced with a primary amine (R¹—NH₂) to form a 4-(2-halopyridin-3-yl)-I-alkylpyrimidin-2-amine compound I. Coupling of compound I with a phenol compound D forms a Formula (I) compound, or an intermediate to be converted to a Formula (I) compound.

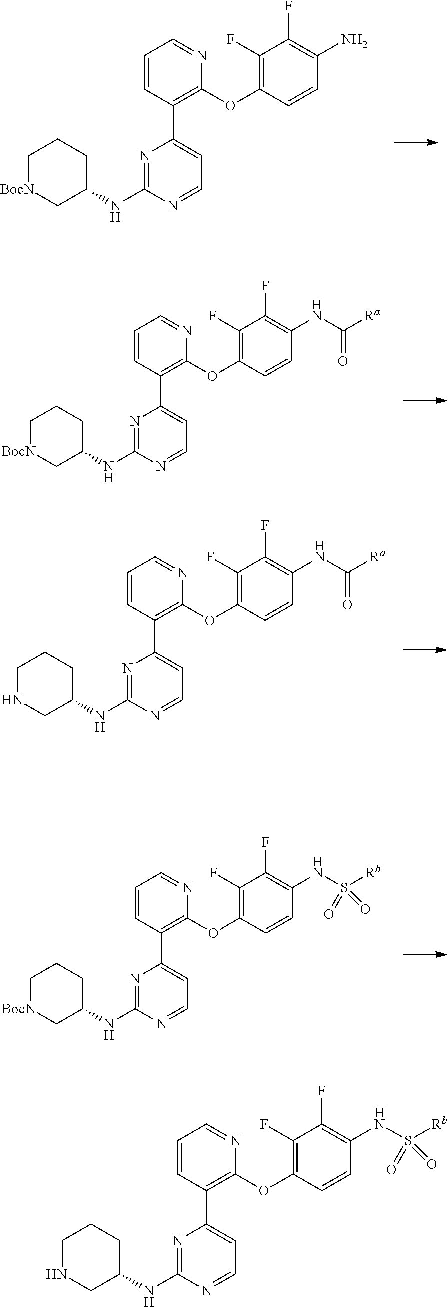

Scheme 4 shows the general preparation of exemplary compounds from aniline intermediate, tert-butyl (S)-3-((4-(2-(4-amino-2,3-difluorophenoxy)pyridin-3-yl)pyrimidin-2-yl)amino)piperidine-1l-carboxylate. The aniline intermediate is treated with a carboxylic acid (R^{a}COOH) and a coupling reagent, such as 1-[Bis(dimethylamino)methylene]-1H-1,2,3-triazolo|4,5-b]pyridinium 3-oxid hexafluorophosphate (HATU) as in General Procedure C, or alternatively with an acid chloride (R^{a}COCl) and an amine base such as diisopropylethylamine (DIPEA) or pyridine, to form Boc-protected, amide intermediates (top). The aniline intermediate is treated with a sulfonyl chloride (R^{b}SO₂Cl) and an amine base to form Boc-protected, sulfonamide intermediates (middle) as in General Procedure A. The Boc-protected intermediates are deprotected with acid, such as hydrochloric acid as in General Procedure B, to form exemplary compounds, such as those in Table 2 and the Examples.

The following General Procedures illustrate synthetic reactions and operations useful to prepare certain Example compounds (e.g., Tables 1 and 2). The reagents, solvents, amounts, equivalents, and conditions are illustrative and exemplary, and not meant to be limiting.

### Analytical Methods

LCMS (Liquid Chromatography Mass Spectrometry) methods to separate and characterize the exemplary compounds are performed on one or more of the following:

SHIMADZU LC-MS 2010EV coupled with SHIMADZU LC20AB using ESI as ionization source. The LC separation was using Column: MERCK, RP-18e 25-2 mm; Detector: PDA, ELSD; Wavelength: UV 220 nm; Column temperature: 50° C.; mobile Phase: 1.5 mL/4 LTFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 mL/min;

SHIMADZU LC-MS 201 OEV coupled with SHIMADZU LC20AB using ESI as ionization source. The LC separation was using Column: MERCK, RP-18e 25-2 mm; Detector: PDA, ELSD; Wavelength: UV 220 nm; Column temperature: 50° C.; mobile Phase: 1.5 mL/4 L TFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 mL/min;

Agilent 1200 Series coupled with 6110 Quadrupole mass spectrometer, using ESI as ionization source. The LC separation was using Column: Xtimate C18 2.1×30 mm, 3 pm; Wavelength: UV 220 nm; Column temperature: 50° C.; Detector: PDA&ELSD. mobile Phase: 1.5 mL/4 L TFA in water (solvent A) and 0.75 mL/4 LTFA in acetonitrile (solvent B), using the elution gradient 10%-80% (solvent B) over 0.9 minutes and holding at 80% for 0.6 minutes at a flow rate of 1.2 mL/min.

SHIMADZU 2020 HPLC coupled with SHIMADZU MSD mass spectrometer using ESI as ionization source. The LC separation was using an Shim-Pack XR-ODS-C18, 50×3.0 mm column with a 1 ml/minute flow rate. Solvent A is water with 0.05% TFA and solvent B is acetonitrile with 0.05% TFA. The gradient consisted with 5-100% solvent B over 2.2 minutes and hold 100% B for 1 minute. LC column temperature is 40° C. UV absorbance was collected from 190 nm to 400 nm and mass spec full scan was applied to all experiments.

SHIMADZU 2020 HPLC coupled with SHIMADZU MSD mass spectrometer using ESI as ionization source. The LC separation was using an Gemini-NX 3μ C18 110A, 50×3.0 mm column with a 1.2 ml/minute flow rate. Solvent A is water with 0.4% NH₄HCO3 and solvent B is acetonitrile. The gradient consisted with 10-50% solvent B over 4 minutes and hold 50% B for 1.2 minutes. LC column temperature is 40° C. UV absorbance was collected from 190 nm to 400 nm and mass spec full scan was applied to all experiments.

SHIMADZU UFLC-MS 2010EV coupled with SHIMADZU MSD mass spectrometer using ESI as ionization source. The LC separation was using a Shim-pack XR-ODS-C18, 50×3.0 mm column with a 1 ml/minute flow rate. Solvent A is water with 0.05% TFA and solvent B is acetonitrile with 0.05% TFA. The gradient consisted with 5-100% solvent B over 2.2 minutes and hold 100% B for 1 minute. LC column temperature is 40° C. UV absorbance was collected from 190 nm to 400 nm and mass spec full scan was applied to all experiments.

Waters Alliance 2695 HPLC with column heater coupled with Waters ZQ 2000 mass spectrometer using ESI as ionization source (ES+, 100-1200 amu). The LC separation was using an XBridge C18, 3.5 μm. 4.6×30 mm column at 25° C. with a 3.0 mL/minute flow rate. Solvent A is Milli-Q H2O+10 mM Ammonium Formate pH: 3.8, and solvent B is acetonitrile. The gradient consisted of isocratic 5% solvent B for 0.2 min, 5% to 100% B in 1.8 minutes; hold 100% B for 1 minute. LC column temperature is 25° C. UV absorbance was collected from 195-320 nm using a Waters PDA 996 UV detector and mass spec full scan was applied to all experiments.

Waters Alliance 2695 HPLC with column heater coupled with Waters ZQ 2000 mass spectrometer using ESI as ionization source (ES+. 100-1200 amu). The LC separation was using an XBridge C18, 3.5 μm, 4.6×30 mm column at 25° C. with a 3.0 mL/minute flow rate. Solvent A is Milli-Q H2O+10 mM Ammonium Bicarbonate pH: 10, and solvent B is acetonitrile. The gradient consisted of isocratic 5% solvent B for 0.2 min, 5% to 100% B in 1.8 minutes: hold 100% B for 1 minute. LC column temperature is 25° C. UV absorbance was collected from 195-320 nm using a Waters PDA 996 UV detector and mass spec full scan was applied to all experiments.

### EXAMPLES

Selected abbreviations: ACN: acetonitrile; DCM: dichloromethane; DMF: N,N-dimethylformamide; DMSO: dimethyl sulfoxide; EtOAc: ethyl acetate; PE: petroleum ether; THF: tetrahydrofuran; TLC: thin layer chromatography.

### General Procedure A—Sulfonamide Synthesis:

To a solution of the aniline (1.0 equiv) in pyridine (5 mL/mmol) and a solvent such as dichloromethane (DCM) at 0° C., or room temperature (rt) was added the corresponding sulfonyl chloride (1.2 equiv typically otherwise noted). After the addition was completed, the reaction solution was stirred at rt for about 16 h. Water (10 mL) was added. The mixture was extracted with DCM. The organic layer was dried over anhydrous sodium sulfate, filtered and concentrated in vacuo.

### General Procedure B—Boc Deprotection

To a mixture of the tert-butyl-carbamate (Boc) intermediate (100 mg, 0.16 mmol) in DCM (25 mL/mmol) or EtOAc was added hydrochloric acid (4 M in dioxane, 10 mL/mmol). The mixture was then stirred at rt for 1 h and concentrated in vacuo or the resulting HCl salt was isolated by filtration.

### General Procedure C—Amide Coupling Using Carboxylic Acids:

To a solution of the aniline intermediate (1.05 equiv otherwise indicated) and DIPEA (3.0 equiv otherwise indicated) in DCM (16 mL/mmol otherwise indicated) was added HATU (2.0 equiv otherwise indicated). The mixture was stirred at rt for 0.5 h. The acid (1.0 equiv otherwise indicated) was added and the resulting mixture was refluxed overnight. The mixture was diluted in DCM, washed with water and brine, dried on anhydrous sodium sulfate, and concentrated in vacuo. Alternatively, to a solution of the aniline in CH2Cl2 was added the acid chloride followed by addition of (iPr)₂NEt or Et₃N or pyridine and stirring at rt. Alternatively, to a solution of the aniline in DMF was added the carboxylic acid and HATU followed by addition of (iPr)₂NEt or EtsN and stirring at rt. Other coupling reagents can be used in General Procedure C. The crude product was isolated and purified using known methods or as described in the Examples.

### General Procedure D—Cbz Deprotection:

A mixture of benzyl-carbamate, palladium on carbon (Pd/C) and ammonium formate in iPrOH was heated at 60° C. When complete, the mixture was filtered through celite using MeOH/CH2Ch and concentrated in vacuo.

### Synthetic Methods

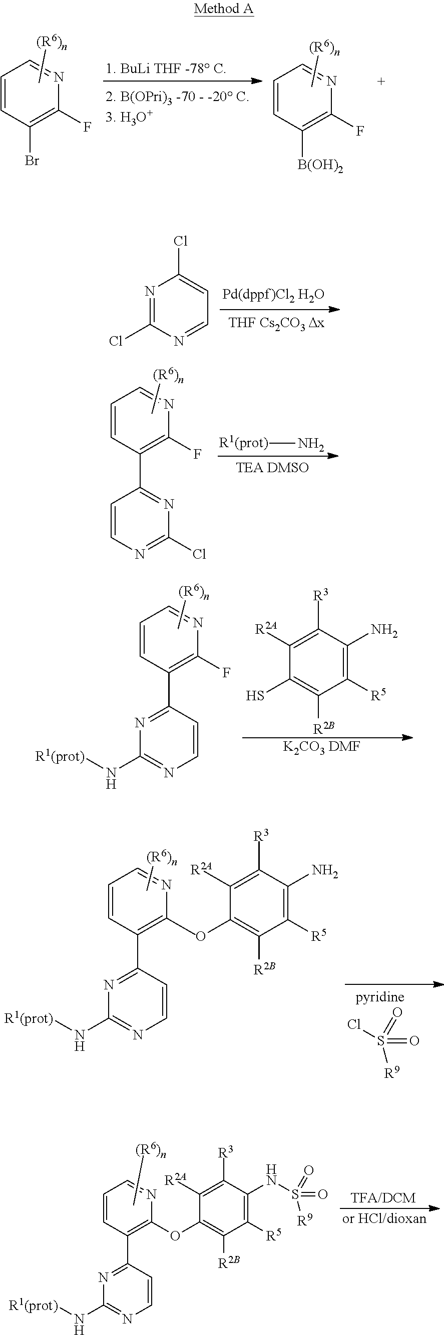

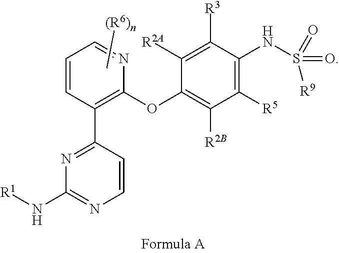

Compounds of general Formula A can be synthesized by routes familiar to one of skill in the art, such as that illustrated above. A Suzuki coupling between 2,4-dichlopyrimidine and a 2-fluoropyridine-3-boronic acid forms a 2-chloro-4-(2-fluoropyridin-3-yl)pyrimidine. Under mild basic conditions, 2-chloropyrimidine can be displaced with the appropriate amine, which may contain a suitably protected primary or secondary amine substituent, to give the corresponding 2-aminopyrimidine. A phenoxide derived from a suitable 4-aminophenol can selectively displace the 2-fluoro on the pyridine ring to form a 4-amino diaryl ether. The aniline amine group can be sulfonated with a suitable sulfonyl halide, such as the chloride, to give a 4-sulfonamidophenoxy ether product, which may require a final deprotection of a C2-side chain amine to give the final product of Formula A.

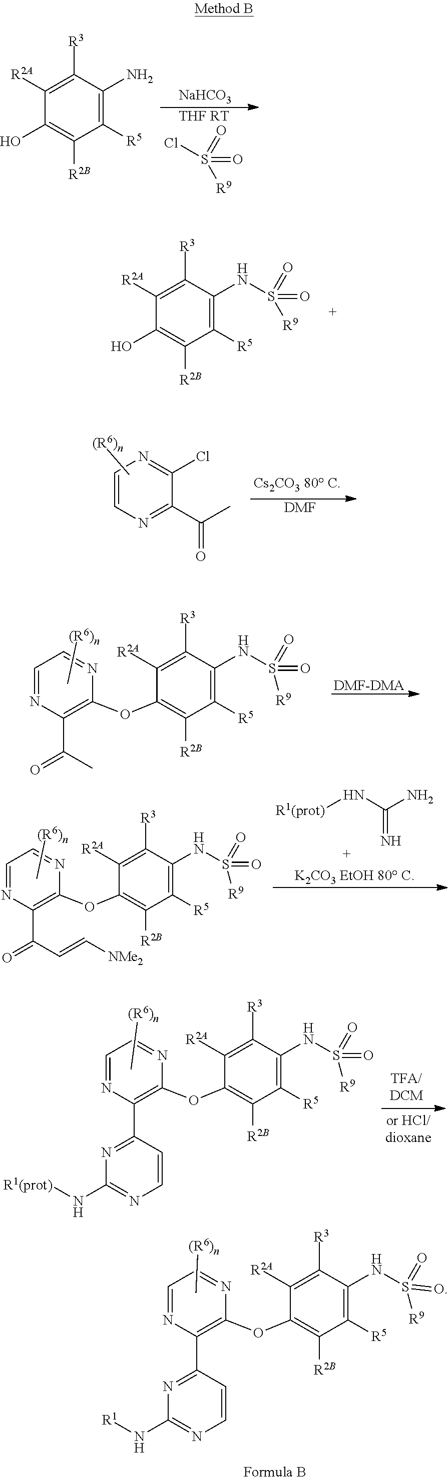

Compounds of general Formula B can be synthesized by routes familiar to one of skill in the art, such as that illustrated above. Reaction of an appropriate sulfonyl halide with a 4-aminophenol under very mild Schotten-Baumann conditions leads to selective formation of a sulfonamide ester rather than the sulfonate. Under much stronger basic conditions the aryloxide ion of the sulfonanilide displaces the chlorine in a suitable 2-acetyl-3-chloropyrazine to form a pyrazyl phenyl ether/. A suitably protected 2-aminopyrimidine is then annulated onto the pyrazine acetyl group in a 2 step process involving formation of a Mannich base with DMF dimethyl acetal followed by cyclization with a suitably protected guanidine derivative completing the synthesis if R¹ does not contain a protecting group. If the R¹ group is protected the synthesis is completed by removing the protecting group under suitable conditions.

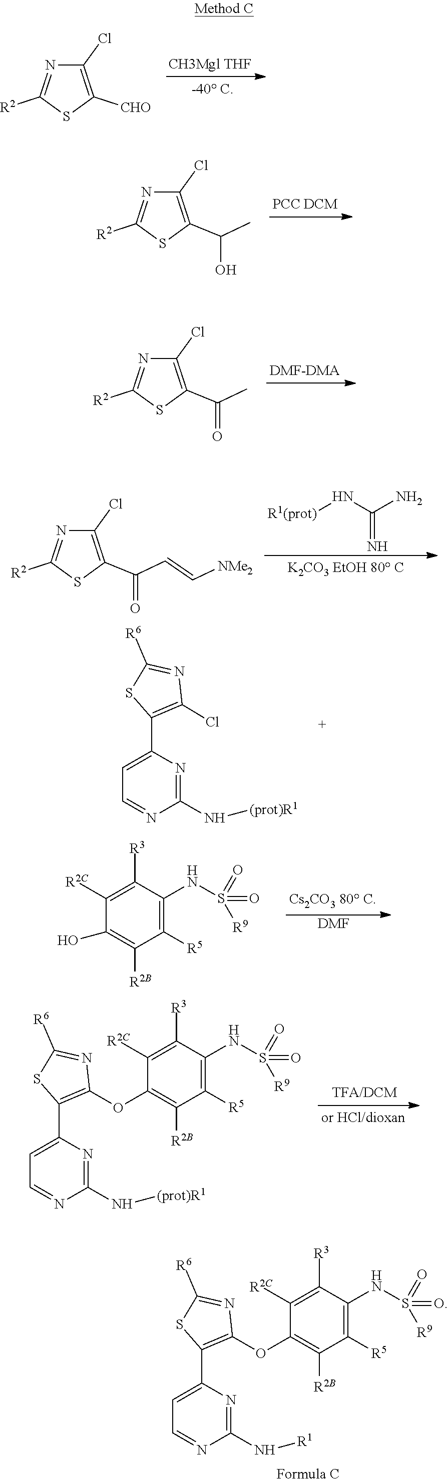

Compounds of general Formula C can be synthesized by routes familiar to one of skill in the art, such as that illustrated above. A suitably substituted 4-chlorothiazole-5-carbaldehyde is C1 homologated with methyl Grignard reagent at low temperature, followed by oxidation to the 2-acetylthiazole. A suitably protected 2-aminopyrimidine is then annulated onto the thiazole acetyl group in a 2 step process involving formation of a Mannich base with DMF dimethyl acetal followed by cyclization with a suitably protected guanidine derivative. The 4-chlorine on the thiazole is then displaced by a suitable 4-sulfonamidophenol under forcing basic conditions, to form an aryl-thiazol-4-yl ether, completing the synthesis if R¹ does not contain a protecting group. If the R¹ group is protected the synthesis is completed by removing the protecting group under suitable conditions.

The following compounds were made using Method A:

### Example 1. N-[4-[(1r,4r)[3-[2-[(4-Aminocyclohexyl)amino]pyrimidin-4-yl]pyrid-2-yl]oxy]-3-fluorophenyl]2-chlorobenzenesulfonamide (Compound Number 410)

### Step 1: O-t-Butyl N-((1r,4r)-4-((4-(2-fluoropyridin-3-yl)pyrimidin-2-yl)amino)cyclohexyl) carbamate

To a solution of 2-chloro-4-(2-fluoro-3-pyridyl)pyrimidine (600 mg, 2.81 mmol, 98.0%, 1 eq) in DMSO (2 mL) was added K₂CO₃ (1.16 g, 8.42 mmol, 3 eq) and 0-tert-butyl N-((1r,4r)-4-aminocyclohexyl)carbamate (541.06 mg, 2.52 mmol, 0.9 eq). The mixture was stirred at 120° C. for 1 h. The mixture was diluted with water (80 mL) and extracted with EtOAc (30 mL×3). The combined organic layers were washed with brine (50 mL), dried over Na₂SO₄, filtered and concentrated under reduced pressure to yield a residue which was purified by flash silica gel chromatography (PE/EtOAc=1/1 to 0/1, TLC: PE/EtOAc=1/1, R_{f}=0.45) to yield O-t-butyl N-((1r,4r)-4-((4-(2-fluoropyridin-3-yl)pyrimidin-2-yl)amino)cyclohexyl) carbamate (400 mg, 929.15 μmol, 33.1% yield, 90.0% purity) as a yellow solid.

¹H NMR (500 MHz, DMSO-d₆) δ ppm 8.49 (d, J=7.5 Hz, 1H), 8.43-8.30 (m, 2H), 7.55 (s, 1H), 7.24 (d, 0.1=8.0 Hz, 1H), 7.05-6.94 (m, 1H), 6.74 (d, J=8.0 Hz, 1H), 3.70 (brs, 1H), 3.25-3.11 (m, 1H), 2.02-1.88 (m, 2H), 1.80 (d, J=11.0 Hz, 2H), 1.38 (s, 9H), 1.35-1.23 (m, 4H); ES-LCMS m/z 388.2 [M+H]⁺.

### Step 2: O-t-Butyl N-[4-[(1r,4r)[4-[2-(4-amino-2-fluorophenoxy)pyrid-3-yl]pyrimidin-2-yl]amino]cyclohexyl]carbamate

To a mixture of O-t-butyl N-((1r,4r)-4-((4-(2-fluoropyridin-3-yl)pyrimidin-2-yl)amino)cyclohexyl) carbamate (111.11 mg, 258.10 umol, 90% purity, 1 eq) and 4-amino-2-fluoro-phenol (32.81 mg, 258.10 μmol, 1 eq) in DMSO (3 mL) was added Cs₂CO₃ (168.19 mg, 516.20 μmol, 2 eq). Then the mixture was stirred at 100° C. for 2 h. The reaction mixture was diluted with H₂O (20 mL) and extracted with EtOAc (20 mL×3). The combined organic layers were washed with brine (20 mL×3), dried over Na₂SO₄, filtered and concentrated under reduced pressure to yield a residue which was purified by prep-TLC (PE/EtOAc=1/1 to 0/1, TLC: PE/EtOAc=1:1, R_{f}=0.42) to yield O-t-butyl N-[4-[(1r,4r)[4-[2-(4-amino-2-fluorophenoxy)pyrid-3-yl]pyrimidin-2-yl]amino]cyclohexyl]carbamate (76 mg, 151.67 μmol, 58.8% yield, 98.7% purity) as a brown oil. ES-LCMS m/z 495.3 [M+H]+.

### Step 3: O-t-Butyl N-[4-[(1r,4r)[4-[2-[4-[(2-chlorophenyl)sulfonylamido]-2-fluoro-phenoxy]pyrid-3-yl]pyrimidin-2-yl]amino]cyclohexyl]carbamate

To a mixture of O-t-butyl N-[4-[(1r,4r)[4-[2-(4-amino-2-fluorophenoxy)pyrid-3-yl]pyrimidin-2-yl]amino]cyclohexyl]carbamate (75 mg, 147.10 μmol, 97.0%, 1 eq). 2-chlorobenzenesulfonyl chloride (31.05 mg, 147.10 μmol, 20.03 μL, 1 eq) in THF (2 mL) was added pyridine (58.18 mg, 735.50 μmol, 59.37 μL, 5 eq), degassed and purged with N₂ for 3 times, and then the mixture was stirred at 20° C. for 4 h. The reaction mixture was concentrated under reduced pressure. The residue was diluted with H₂O (20 mL) and extracted with EtOAc (40 mL×3). The combined organic layers were washed with brine (20 mL×3), dried over Na₂SO₄, filtered and concentrated under reduced pressure to yield a residue which was purified by prep-HPLC purification (column: Boston Green ODS 150*30 mm*5 μm; mobile phase: [water(FA)-ACN]; B %: 51%-81%, 14 min) to yield O-t-butyl N-[4-[(1r,4r)[4-[2-[4-[(2-chlorophenyl)sulfonylamido]-2-fluoro-phenoxy]pyrid-3-yl]pyrimidin-2-yl]amino]cyclohexyl]carbamate (50 mg, 59.78 μmol, 40.6% yield, 80.0% purity) as a yellow solid. ES-LCMS m/z 669.3 [M+H]⁺.

### Step 4: N-[4-[(1r,4r)[3-[2-[(4-Aminocyclohexyl)amino]pyrimidin-4-yl]pyrid-2-yl]oxy]-3-fluorophenyl]2-chlorobenzenesulfonamide (Compound Number 410)

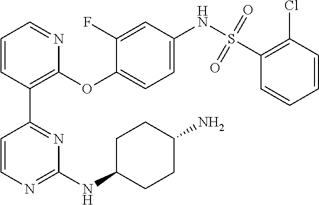 [SMILES: N[C@H]1CC[C@H](Nc2nccc(-c3cccnc3Oc3ccc(NS(=O)(=O)c4ccccc4Cl)cc3F)n2)CC1]

To a solution of O-t-butyl N-[4-[(1r,4r)[4-[2-[4-[(2-chlorophenyl)sulfonylamido]-2-fluoro-phenoxy]pyrid-3-yl]pyrimidin-2-yl]amino]cyclohexyl]carbamate (50 mg, 59.78 μmol, 80.0%, 1 eq) in DCM (3 mL) was added TFA (1.54 g, 13.51 mmol, 1 mL, 225.94 eq). The mixture was stirred at 25° C. for 1 h. The reaction mixture was neutralized to pH=7 with NH₃—H₂O and then concentrated to yield a residue which was purified by preparative HPLC (column: Boston Prime C18 150*30 mm*5 μm; mobile phase: [water(NH₃H₂O+NH₄HCO3)-ACN]; B %: 30%-60%, 10 min), followed by lyophilization to yield N-[4-[(1r,4r)[3-[2-[(4-aminocyclohexyl)amino]pyrimidin-4-yl]pyrid-2-yl]oxy]-3-fluorophenyl]2-chlorobenzenesulfonamide (14.5 mg, 25.48 μmol, 42.6% yield, 100.0% purity) as a white solid.

¹H NMR (500 MHz, DMSO-d₆) δ ppm 8.38-8.25 (m, 2H), 8.16 (dd, J=2.0, 5.0 Hz, 1H), 7.98 (dd, J=2.0, 7.5 Hz, 1H), 7.45 (dd, J=1.5, 7.9 Hz, 1H), 7.43-7.34 (m, 2H), 7.25-7.20 (m, 2H), 7.19 (d, J=5.0 Hz, 1H), 6.89 (t, J=9.0 Hz, 1H), 6.71 (dd. J=2.2, 14.0 Hz, 1H), 6.60 (dd, J=2.2, 8.7 Hz, 1H), 3.78-3.69 (m, 1H), 3.00-2.93 (m, 1H), 2.07-1.90 (m, 4H), 1.44-1.29 (m, 4H); ES-LCMS m/z 569.1 [M+H]⁺.

### Example 2. N-[4-[(1r,4r)[3-[2-[(4-Aminocyclohexyl)amino]pyrimidin-4-yl]-2-pyridyl]oxy]phenyl]2-chlorobenzenesulfonamide (Compound Number 411)

### Step 1: O-t-Butyl N-[4-[(1r,4r)[4-[2-(4-aminophenoxy)-3-pyridyl]pyrimidin-2-yl]amino]cyclohexyl]carbamate

To a solution of O-t-butyl N-[4-[(1r,4r)[4-(2-fluoropyrid-3-yl)pyrimidin-2-yl]amino]cyclohexyl]carbamate (150 mg, 348.43 μmol, 90.0%, 1 eq) in DMF (8 mL) was added Cs₂CO₃ (227.05 mg, 696.86 μmol, 2 eq) and 4-aminophenol (57.03 mg, 522.65 μmol, 81.48 μL, 1.5 eq). The mixture was stirred at 100° C. for 2 h. The mixture was diluted with water (20 mL) and extracted with EtOAc (15 mL×3). The combined organic layers were washed with brine (20 mL), dried over Na₂SO₄, filtered and concentrated under reduced pressure to yield a residue which was purified by preparative TLC (PE/EtOAc=1/1, TLC: PE/EtOAc=1/1, R_{f}=0.24) to yield O-t-butyl N-[4-[(1r,4r)[4-[2-(4-aminophenoxy)-3-pyridyl]pyrimidin-2-yl]amino]cyclohexyl]carbamate (100 mg, 197.24 μmol, 56.6% yield, 94.0% purity) as a black brown oil.

¹H NMR (400 MHz, DMSO-d₆) δ ppm 8.33 (d, J=5.0 Hz, 1H), 8.32 (slbrs, 1H), 8.15 (dd, J=2.0, 4.8 Hz, 1H), 7.21 (d, J=5.0 Hz, 1H), 7.21 (slbrs, 1H), 7.08 (1H, d, J=4.8 Hz), 6.80 (d, J=8.8 Hz, 2H), 6.75 (1H, d, J=4.8 Hz), 6.57 (d, J=8.8 Hz, 2H), 4.90 (brs, 2H), 3.70 (m, 1H), 3.20 (m, 1H), 2.00-1.78 (m, 4H), 1.38 (s, 9H), 1.34-1.23 (m, 4H); ES-LCMS m/z 477.4 [M+H]⁺.

### Step 2: O-t-Butyl N-[4-[(1r,4r)[4-[2-[4-[2-chlorophenylsulfonylamido]phenoxy]pyrid-3-yl]pyrimidin-2-yl]amino]cyclohexyl]carbamate

To a solution of O-t-butyl N-[4-[(1r,4r)[4-[2-(4-aminophenoxy)-3-pyridyl]pyrimidin-2-yl]amino]cyclohexyl]carbamate (100 mg, 197.24 μmol, 94.0%, 1 eq) in THF (2 mL) was added pyridine (78.01 mg, 986.22 μmol, 79.60 μL, 5 eq) and 2-chlorobenzenesulfonyl chloride (124.89 mg, 591.73 μmol, 80.58 μL, 3 eq). The mixture was stirred at 20° C. for 2 h. The mixture was diluted with water (20 mL) and extracted with EtOAc (15 mL×3). The combined organic layers were washed with brine (20 mL), dried over Na₂SO₄, filtered and concentrated under reduced pressure to yield a residue which was purified by preparative TLC (PE/EtOAc=1/1, TLC: PE/EtOAc=1/1, R_{f}=0.31) to yield 0-t-butyl N-[4-[(1r,4r)[4-[2-[4-[2-chlorophenylsulfonylamido]phenoxy]pyrid-3-yl]pyrimidin-2-yl]amino]cyclohexyl]carbamate (60 mg, 92.14 μmol, 46.7% yield, N/A purity) as a white oil. ES-LCMS m/z 651.2 [M+H]⁺.

### Step 3: N-[4-[(1r,4r)[3-[2-[(4-Aminocyclohexyl)amino]pyrimidin-4-yl]-2-pyridyl]oxy]phenyl]2-chlorobenzenesulfonamide (Compound Number 411)

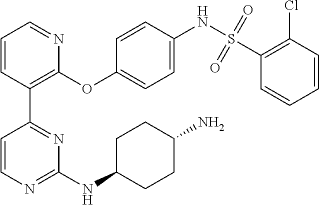 [SMILES: N[C@H]1CC[C@H](Nc2nccc(-c3cccnc3Oc3ccc(NS(=O)(=O)c4ccccc4Cl)cc3)n2)CC1]

To a solution of O-t-butyl N-[4-[(1r,4r)[4-[2-[4-[2-chlorophenylsulfonylamido]phenoxy]pyrid-3-yl]pyrimidin-2-yl]amino]cyclohexyl]carbamate (60 mg, 92.14 μmol, 1 eq) in DCM (3 mL) was added TFA (1.54 g, 13.51 mmol, 1 mL, 146.58 eq). The mixture was stirred at 25° C. for 1 h. The reaction mixture was neutralized to pH7 with NH₃—H₂O and concentrated to yield a residue which was purified by preparative HPLC (column: Boston Prime C18 150*30 mm*5 μm; mobile phase: [water (NH₃H₂O+NH₄HCO₃)-ACN]; B %: 30%-60%, 10 min), followed by lyophilization to yield N-[4-[(1r,4r)[3-[2-[(4-aminocyclohexyl)amino]pyrimidin-4-yl]-2-pyridyl]oxy]phenyl]2-chlorobenzenesulfonamide (49.2 mg, 88.13 μmol, 95.7% yield, 98.7% purity) as a white solid.

¹H NMR (500 MHz, DMSO-d₆) δ ppm 8.32 (d, J=5.0 Hz, 1H), 8.31 (slbrs, 1H), 8.15 (dd, J=4.7, 1.8 Hz, 1H), 7.98 (dd, J=1.5, 8.0 Hz, 1H), 7.51 (dd, J=1.5, 7.9 Hz, 1H), 7.46 (dt, J=1.5, 7.5 Hz, 1H), 7.40 (dt. J=1.5, 7.7 Hz, 1H), 7.22 (m, 1H), 7.16 (d, J=4.8 Hz, 1H), 7.14 (brs, 1H), 6.96-6.89 (d, J=8.7 Hz, 2H), 6.83 (d, J=8.7 Hz, 2H), 3.67 (brs, 1H), 2.82 (brs, 1H), 1.99-1.81 (m, 4H), 1.36-1.22 (m, 4H); ES-LCMS in z 551.1 [M+H]⁺.

### Example 3. N—((S)-4-((3-(2-(Piperidin-3-ylamino)pyrimidin-4-yl)pyridin-2-yl)oxy)phenyl) 2-chlorobenzenesulfonamide (Compound Number 412)

Step 1: O-t-Butyl 1N—[(S)-3-((4-(2-(4-aminophenoxy)pyridin-3-yl)pyrimidin-2-yl)amino)piperidin-1-yl]carboxylate

To a solution of O-t-butyl 1I-[(3S)-3-[[4-(2-fluoro-3-pyridyl)pyrimidin-2-yl]amino]piperidin-1-yl]carboxylate (100 mg, 255.47 μmol, 95.4% purity, 1 eq) in DMF (5 mL) was added Cs₂CO₃ (166.48 mg, 510.95 μmol, 2 eq) and 4-aminophenol (41.82 mg, 383.21 μmol, 59.74 μL, 1.5 eq). The mixture was stirred at 155° C. for 2 h. The mixture was diluted with water (20 mL) and extracted with EtOAc (15 mL×3). The combined organic layers were washed with brine (15 mL), dried over Na₂SO₄, filtered and concentrated under reduced pressure to yield a residue which was purified by preparative TLC (PE/EtOAc=3/1, TLC: PE/EtOAc=3/1, R_{f}=0.25) to yield O-t-butyl 1N—[(S)-3-((4-(2-(4-aminophenoxy)pyridin-3-yl)pyrimidin-2-yl)amino)piperidin-1-yl]carboxylate (50 mg, 107.02 μmol, 41.8% yield, 99.0% purity) as a black brown oil.

¹H NMR (500 MHz, DMSO-d₆) δ ppm 8.38 (d, J=5.5 Hz, 1H), 8.16 (dd, J=2.0, 4.7 Hz, 1H), 7.39-7.25 (m, 1H), 7.25-7.13 (m, 2H), 6.81 (d, J=8.5 Hz, 2H), 6.58 (d, J=8.5 Hz, 2H), 4.98 (s, 2H), 4.38 (brs, 1H), 3.79 (brs, 2H), 3.17-2.99 (m, 1H), 2.03-1.68 (m, 2H), 1.49-1.09 (m, 11H); ES-LCMS m/z 463.3 [M+H]⁺.

### Step 2: O-t-Butyl 1N—[(S)-3-((4-(2-(4-(2-chlorophenylsulfonamido)phenoxy)pyridin-3-yl)pyrimidin-2-yl)amino)piperidin-1-yl]carboxylate

A mixture of O-t-butyl 1N—[(S)-3-((4-(2-(4-aminophenoxy)pyridin-3-yl)pyrimidin-2-yl)amino)piperidin-1-yl]carboxylate (50 mg, 107.02 μmol, 99.0% purity, 1 eq), pyridine (67.72 mg, 856.14 μmol, 69.10 μL, 8 eq) in THF (1 mL) was degassed and purged with N₂ for 3 times, and then to the mixture was added 2-chlorobenzenesulfonyl chloride (45.18 mg, 214.03 μmol, 29.15 μL, 2 eq). The mixture was stirred at 25° C. for 1 h under N₂ atmosphere. The reaction mixture was quenched by the addition of water (10 ml), extracted with EtOAc (10 mL×3). The combined organic layers were washed with brine (10 mL), dried over Na₂SO₄, filtered and concentrated under reduced pressure to yield a residue which was purified by flash silica gel chromatography (PE/EtOAc=1/1, TLC: PE/EtOAc=1/1. R_{f}=0.30) to yield O-t-butyl 1N—[(S)-3-((4-(2-(4-(2-chlorophenylsulfonamido)phenoxy)pyridin-3-yl)pyrimidin-2-yl)amino)piperidin-1-yl]carboxylate (30 mg, 45.20 μmol, 42.2% yield, 96.0% purity) as a white oil.

¹H NMR (500 MHz, MeOD-d₄) δ ppm 8.61-8.38 (m, 1H), 8.28 (d, J=4.9 Hz, 1H), 8.11 (dd, J=1.8, 4.8 Hz, 1H), 8.04 (dd, J=1.2, 7.9 Hz, 1H), 7.59-7.52 (m, 2H), 7.42 (ddd, J=1.8, 6.6, 8.0 Hz, 1H), 7.28 (d, J=4.6 Hz, 1H), 7.24-7.17 (m, 3H), 6.97 (d, J=8.9 Hz, 2H), 4.00-3.90 (m, 1H), 3.85-3.25 (m, 4H), 3.13-2.71 (m, 1H), 2.07-1.93 (m, 1H), 1.83-1.20 (m, 12H); ES-LCMS m/z 637.1 [M+H]⁺.

### Step 3: N—((S)-4-((3-(2-(Piperidin-3-ylamino)pyrimidin-4-yl)pyridin-2-yl)oxy)phenyl) 2-chlorobenzenesulfonamide (Compound Number 412)

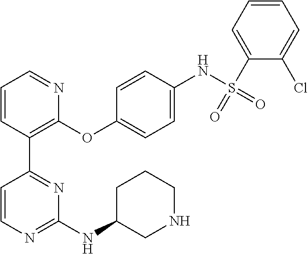 [SMILES: O=S(=O)(Nc1ccc(Oc2ncccc2-c2ccnc(N[C@H]3CCCNC3)n2)cc1)c1ccccc1Cl]

To a solution of O-t-butyl 1N—[(S)-3-((4-(2-(4-(2-chlorophenylsulfonamido)phenoxy)pyridin-3-yl)pyrimidin-2-yl)amino)piperidin-1-yl]carboxylate (30 mg, 45.20 μmol, 96.0% purity, 1 eq) in DCM (1.5 mL) was added TFA (770.00 mg, 6.75 mmol, 0.5 mL, 149.40 eq). The mixture was stirred at 25° C. for 30 min. The mixture was neutralized to pH=7 with NH₃—H₂O and concentrated. Then water (100 mL) was added. The mixture was extracted with EtOAc (100 mL×3). The combined organic layers were washed with brine (100 mL), dried over Na₂SO₄, filtered and concentrated. The residue was purified by prep-HPLC (column: Boston Prime C18 150*30 mm*5 m; mobile phase: [water(NH₃H₂O+NH₄HCO₃)-ACN]; B %: 30%-60%, 10 min) to yield N—((S)-4-((3-(2-(piperidin-3-ylamino)pyrimidin-4-yl)pyridin-2-yl)oxy)phenyl) 2-chlorobenzenesulfonamide (9.7 mg, 17.70 μmol, 39.1% yield, 98.0% purity) as a white solid.

¹H NMR (500 MHz, DMSO-d₆) δ ppm 8.36 (brs, 1H), 8.33 (d, J=5.2 Hz. 1H), 8.15 (dd, J=2.2, 5.1 Hz, 1H), 8.02 (dd, J=1.7, 7.9 Hz, 1H), 7.61 (dd, J=7.9, 1.4 Hz, 1H), 7.59 (dt, J_{D}=1.8 Hz, J_{T}=8.1 Hz, 1H), 7.49 (ddd, J=8.1, 7.3, 1.4 Hz, 1H), 7.25 (dd, J=7.5, 4.4 Hz, 1H), 7.19 (brs, 1H), 7.14 (d, J=7.8 Hz, 1H), 7.06 (d, J=8.5 Hz, 2H), 6.98 (d, J=8.5 Hz, 2H), 3.87 (brs. 1H), 3.16-3.08 (m, 1H), 2.89-2.83 (m, 1H), 2.48-2.41 (m, 1H), 1.94-1.86 (m, 1H), 1.69-1.63 (m, 1H), 1.51-1.41 (m, 2H); ES-LCMS m/z 537.2 [M+H]⁺.

### Example 4. N—((S)-4-((3-(2-(piperidin-3-ylamino)pyrimidin-4-yl)pyridin-2-yl)oxy)-3-fluorophenyl) 2-chlorobenzenesulfonamide (Compound Number 413)

### Step 1: O-t-Butyl N-[(3S)-3-[[4-(2-fluoropyrid-3-yl)pyrimidin-2-yl]amino]piperidin-1-yl]carboxylate

To a solution of 2-chloro-4-(2-fluoropyridin-3-yl)pyrimidine (500 mg, 2.39 mmol, 100.0% purity, 1 eq) and O-t-butyl-N-[(3S)-3-aminopiperidin-1-yl]carboxylate (573.29 mg, 2.86 mmol, 1.2 eq) in DMSO (6 mL) was added K₂CO₃ (494.52 mg, 3.58 mmol, 1.5 eq) which was stirred at 70° C. for 2 h. The mixture was concentrated and then water (150 mL) was added. The mixture was extracted with EtOAc (150 mL×3). The combined organic layers were washed with brine (450 mL), dried over Na₂SO₄, filtered and concentrated. The residue was purified by preparative HPLC (column: Boston Prime C18 150*30 mm*5 μm; mobile phase: [water(NH₃H₂O+NH₄HCO₃)-ACN]; B %: 49%-79%, 10 min) to yield O-t-butyl N-[(3S)-3-[[4-(2-fluoropyrid-3-yl)pyrimidin-2-yl]amino]piperidin-1-yl]carboxylate (160 mg. 411.33 μmol, 17.2% yield, 96.0% purity) as a white solid.

¹H NMR (500 MHz, CD₃OD-d₄) δ ppm 8.81-8.59 (m, 1H), 8.39 (d, J=5.0 Hz, 1H), 8.31 (d, J=4.6 Hz, 1H), 7.47 (s, 1H), 7.16 (d, J=2.6 Hz, 1H), 3.96 (d, J=6.6 Hz, 1H), 3.88-3.75 (m, 1H), 3.65-3.46 (m, 1H), 3.27-2.70 (m, 2H), 2.08-2.03 (m, 1H), 1.82 (d, J=0.9 Hz, 1H), 1.63-1.54 (m, 2H), 1.42-1.20 (m, 9H); ES-LCMS m/z 374.2 [M+H]⁺.

### Step 2: O-t-Butyl N-[(3S)-3-[[4-(2-(4-amino-2-fluorophenoxy)pyrid-3-yl)pyrimidin-2-yl]amino]piperidin-1-yl]carboxylate

To a solution of O-t-butyl N-[(3S)-3-[[4-(2-fluoropyrid-3-yl)pyrimidin-2-yl]amino]piperidin-1-yl]carboxylate (160 mg, 411.33 μmol, 1 eq) and 4-amino-2-fluoro-phenol (62.74 mg, 493.59 μmol, 1.2 eq) in DMF (4 mL) was added Cs₂CO₃ (201.03 mg, 616.99 μmol, 1.5 eq). The reaction was stirred at 155° C. for 2 h. The reaction mixture was concentrated and then water (100 mL) was added. The mixture was extracted with EtOAc (100 mL×3). The combined organic layers were washed with brine (300 mL), dried over Na₂SO₄, filtered and concentrated. The crude material was purified on silica gel column chromatography (PE/EtOAc=1/0 to 1/1, TLC: PE/EtOAc=1/1, R_{f}=0.2) to yield O-t-butyl N-[(3S)-3-[[4-(2-(4-amino-2-fluorophenoxy)pyrid-3-yl)pyrimidin-2-yl]amino]piperidin-1-yl]carboxylate (65 mg, 127.15 μmol, 30.9% yield, 94.0% purity) as a brown solid.

¹H NMR (500 MHz, DMSO-d₆) δ ppm 8.39 (d, J=5.2 Hz, 1H), 8.16 (dd, J=1.9, 4.8 Hz, 1H), 7.31-7.20 (m, 3H), 6.95 (t, J=8.8 Hz, 1H), 6.46 (dd, J=2.5, 12.9 Hz, 1H). 6.38 (dd, J=2.5, 8.6 Hz, 1H), 5.30-5.25 (m, 2H), 3.79 (brd, J=3.1 Hz, 1H), 3.73-3.48 (m, 1H), 3.31-3.31 (m, 1H), 3.20-2.81 (m, 2H), 1.97-1.91 (m, 1H), 1.80-1.72 (m, 1H), 1.67-1.46 (m, 2H), 1.36-1.15 (m, 9H); ES-LCMS m/z 481.2 [M+H]⁺.

### Step 3: O-t-Butyl N-[(3S)-3-[[4-(2-(4-(2-chlorophenylsulfonamido)-2-fluorophenoxy)pyrid-3-yl)pyrimidin-2-yl]amino]piperidin-1-yl]carboxylate

A mixture of O-t-butyl N-[(3S)-3-[[4-(2-(4-amino-2-fluorophenoxy)pyrid-3-yl)pyrimidin-2-yl]amino]piperidin-1-yl]carboxylate (60 mg, 117.37 μmol, 1 eq). 2-chlorobenzenesulfonyl chloride (29.73 mg, 140.84 μmol, 19.18 μL, 1.2 eq), pyridine (74.27 mg, 938.96 μmol, 75.79 μL, 8 eq), in THF (3 mL) was degassed and purged with N₂ for 3 times, and then the mixture was stirred at 25° C. for 2 h under N₂ atmosphere. The mixture was concentrated and then water (100 mL) was added. The mixture was extracted with EtOAc (100 mL×3). The combined organic layers were washed with brine (300 mL), dried over Na₂SO₄, filtered and concentrated. The crude material was purified on silica gel column chromatography (PE/EtOAc=1/0 to 1/1, TLC: PE/EtOAc=1/1, R_{f}=0.55) to yield O-t-butyl N-[(3S)-3-[[4-(2-(4-(2-chlorophenylsulfonamido)-2-fluorophenoxy)pyrid-3-yl)pyrimidin-2-yl]amino]piperidin-1-yl]carboxylate (65 mg, 98.22 μmol, 83.6% yield. 99.0% purity) as a yellow solid.

¹H NMR (500 MHz, DMSO-d₆) δ ppm 10.94 (s, 1H), 8.36 (d, J=5.2 Hz, 2H), 8.15-8.10 (m, 2H), 7.69-7.67 (m, 2H), 7.57 (ddd, J=2.7, 5.6, 8.1 Hz, 1H), 7.29-7.21 (m, 4H), 7.04 (dd, J=2.3, 12.1 Hz, 1H), 6.96 (dd. J=1.7, 8.7 Hz, 1H), 3.77 (brs, 1H), 3.67-3.45 (m, 1H), 3.33-3.32 (m, 1H), 3.20-2.74 (m, 2H), 1.94-1.89 (m, 1H), 1.78-1.70 (m, 1H), 1.64-1.45 (m, 2H), 1.25-1.15 (m, 9H); ES-LCMS m/z 655.3 [M+H]⁺.

### Step 4: N—((S)-4-((3-(2-(piperidin-3-ylamino)pyrimidin-4-yl)pyridin-2-yl)oxy)-3-fluorophenyl) 2-chlorobenzenesulfonamide (Compound Number 413)

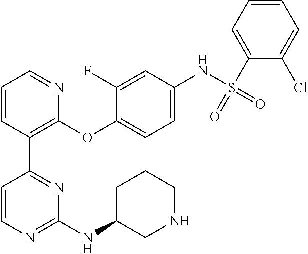 [SMILES: O=S(=O)(Nc1ccc(Oc2ncccc2-c2ccnc(N[C@H]3CCCNC3)n2)c(F)c1)c1ccccc1Cl]

To a solution of O-t-butyl N-[(3S)-3-[[4-(2-(4-(2-chlorophenylsulfonamido)-2-fluorophenoxy)pyrid-3-yl)pyrimidin-2-yl]amino]piperidin-1-yl]carboxylate (60 mg, 91.58 μmol, 1 eq) in DCM (3 mL) was added TFA (1.54 g, 13.51 mmol, 1 mL). The mixture was stirred at 25° C. for 2 h. The reaction mixture was filtered and concentrated under reduced pressure to give a residue. The residue was purified by preparative HPLC (column: Boston Prime C18 150*30 mm*5 μm; mobile phase: [water (NH₃H₂O+NH₄HCO₃)-ACN]; B %: 29%-59%, 10 mm), followed by lyophilization to yield N—((S)-4-((3-(2-(piperidin-3-ylamino)pyrimidin-4-yl)pyridin-2-yl)oxy)-3-fluorophenyl) 2-chlorobenzenesulfonamide (29.0 mg, 52.25 μmol, 57.0% yield, 100.0% purity) as a white solid.

¹H NMR (500 MHz, DMSO-d₆) δ ppm 8.39 (brs, 1H), 8.38 (d, J=5.0 Hz, 1H), 8.16 (dd, J=1.8, 4.9 Hz, 1H), 8.07 (dd, J=7.9, 1.5 Hz, 1H), 7.60-7.54 (m, 2H), 7.45 (dt, J_{D}=1.4 Hz, J_{T}=7.5 Hz, 1H), 7.37 (d, J=6.6 Hz, 1H), 7.27 (dd, J=4.9, 7.6 Hz, 2H), 7.11 (t, J=8.9 Hz, 1H), 6.90 (d, J=12.1 Hz, 1H), 6.81 (d, J=8.2 Hz, 1H), 4.14-4.02 (m, 1H), 3.28-3.26 (m, 1H), 3.14-3.06 (m, 1H), 2.76-2.67 (m, 2H), 1.97-1.93 (m, 1H), 1.86-1.80 (m, 1H), 1.64-1.54 (m, 2H); ES-L CMS m/z 555.1 [M+H]⁻.

The following compounds have been made by processes similar to those described immediately above using Method A:

### Example 5. N-[4-[(1r,4r)[3-[2-[(4-Aminocyclohexyl)amino]pyrimidin-4-yl]pyrid-2-yl]oxy]-6-chloro-3-fluorophenyl]2-chlorobenzenesulfonamide (Compound Number 414)

### Example 6. N-[4-[(1r,4r)[3-[2-[(4-Aminocyclohexyl)amino]pyrimidin-4-yl]pyrid-2-yl]oxy]-3-fluorophenyl]naphthalene-1-sulfonamide (Compound Number 415)

### Example 7. N-[4-[(1r,4r)[3-[2-[(4-Aminocyclohexyl)amino]pyrimidin-4-yl]pyrid-2-yl]oxy]-6-chloro-3-fluorophenyl]naphthalene-1-sulfonamide (Compound Number 416)

### Example 8. N-[4-[(1r,4r)[3-[2-[(4-Aminocyclohexyl)amino]pyrimidin-4-yl]pyrid-2-yl]oxy]-3-fluorophenyl]naphthalene-2-sulfonamide (Compound Number 417)

The following compounds were made using Method B:

### Example 9. N-[3-Fluoro-4-[[3-[2-[[(3S)-3-piperidinyl]amino]pyrimidin-4-yl]-2-pyrazinyl]oxy]phenyl]2-chlorobenzenesulfonamide (Compound Number 390)

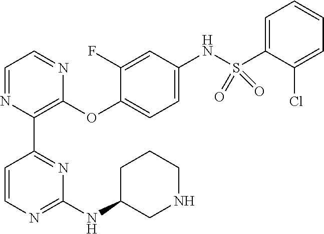 [SMILES: O=S(=O)(Nc1ccc(Oc2nccnc2-c2ccnc(N[C@H]3CCCNC3)n2)c(F)c1)c1ccccc1Cl]

### Step 1: (E)-1-(3-Chloropyrazin-2-yl)-3-(dimethylamino)prop-2-en-1-one

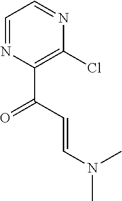

A solution of 1-(3-chloropyrazin-2-yl)ethanone (2 g, 12.77 mmol, 1 eq) in DMF-DMA (17.94 g, 150.55 mmol, 20.00 mL, 11.79 eq) was stirred at 80° C. for 12 h. The mixture was concentrated under reduced pressure to yield a residue which was purified by prep-HPLC purification (column: YMC Triart C18 250*50 mm*7 μm; mobile phase: [water (FA)-ACN]; gradient: 20%-30% B over 20 min), followed by lyophilization to yield (E)-1-(3-chloropyrazin-2-yl)-3-(dimethylamino)prop-2-en-1-one (2.2 g, 8.84 mmol, 69.2% yield, 85.0% purity) as a red solid. ¹H NMR (400 MHz, CD₃OD) δ ppm 8.56-8.54 (m, 1H), 8.47-8.46 (m, 1H), 7.98-7.10 (m, 1H), 5.59-5.39 (m, 1H), 3.29-3.04 (m, 6H).

### Step 2: tert-Butyl (3S)-3-[[4-(3-chloropyrazin-2-yl)pyrimidin-2-yl]amino]piperidine-1-carboxylate

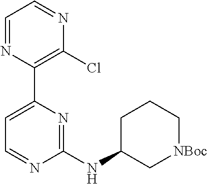 [SMILES: CC(C)(C)OC(=O)N1CCC[C@H](Nc2nccc(-c3nccnc3Cl)n2)C1]

To a solution of (E)-1-(3-chloropyrazin-2-yl)-3-(dimethylamino)prop-2-en-1-one (900 mg, 3.61 mmol, 1 eq) in EtOH (50 mL) was added K₂CO₃ (1.50 g, 10.84 mmol, 3 eq) and tert-butyl (3S)-3-guanidinopiperidine-1-carboxylate (2.06 g, 7.23 mmol, 2 eq). The mixture was stirred at 40° C. for 8 h. The mixture was concentrated under reduced pressure to yield a residue which was purified by column chromatography (SiO₂, Petroleum ether/Ethyl acetate=100:1 to 0:1) to yield tert-butyl (3S)-3-[[4-(3-chloropyrazin-2-yl)pyrimidin-2-yl]amino]piperidine-1-carboxylate (300 mg, 652.40 μmol, 18.1% yield, 85.0% purity) as a blue oil. ¹H NMR (400 MHz, DMSO-d₆) δ ppm 8.78 (s, 1H), 8.64 (d, J=2.4 Hz, 1H), 8.50 (d, J=4.9 Hz, 1H), 7.42 (d, J=7.4 Hz, 1H), 6.99 (d, J=5.0 Hz, 1H), 3.79 (d, J=15.5 Hz, 2H), 3.65-3.53 (m, 1H), 3.17 (d, J=5.1 Hz, 2H), 1.76-1.71 (m, 1H), 1.55 (s, 1H), 1.47-1.40 (m, 2H), 1.39-1.28 (m, 9H); ES-LCMS m/z 391.1 [M+H]⁻.

### Step 3: tert-Butyl (3S)-3-[[4-[3-(4-amino-2-fluoro-phenoxy)pyrazin-2-yl]pyrimidin-2-yl]amino]piperidine-1-carboxylate

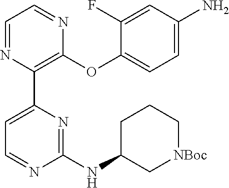 [SMILES: CC(C)(C)OC(=O)N1CCC[C@H](Nc2nccc(-c3nccnc3Oc3ccc(N)cc3F)n2)C1]

To a solution of tert-butyl (3S)-3-[[4-(3-chloropyrazin-2-yl)pyrimidin-2-yl]amino]piperidine-1-carboxylate (200 mg, 434.93 μmol, 1 eq) in DMF (4 mL) was added Cs₂CO₃ (425.13 mg, 1.30 mmol, 3 eq) and 4-amino-2-fluoro-phenol (221.15 mg, 1.74 mmol, 4 eq). The mixture was stirred at 150° C. for 2 h. The mixture was concentrated to yield a residue which was purified by prep-TLC (Petroleum ether:Ethyl acetate=0:1) to yield tert-butyl (3S)-3-[[4-[3-(4-amino-2-fluoro-phenoxy)pyrazin-2-yl]pyrimidin-2-yl]amino]piperidine-1-carboxylate (160 mg, 282.44 μmol, 64.9% yield, 85.0% purity) as a brown solid. ¹H NMR (400 MHz, DMSO-d₆) δ ppm 8.46 (d, J=2.5 Hz, 1H), 8.41-8.34 (m, 1H), 8.26 (d, J=2.5 Hz, 1H), 7.32 (dt, J=1.6, 3.5 Hz, TH), 7.11 (d, J=4.9 Hz, TH), 6.95 (d, J=1.5 Hz, TH), 6.47-6.43 (m, 1H), 6.37 (dd, J=2.2, 8.4 Hz, 1H), 5.35 (s, 2H), 3.76 (d, J=8.4 Hz, 3H), 3.22-3.13 (m, 1H), 3.04-2.94 (m, 1H), 1.94-1.89 (m, 1H), 1.73 (d, J=5.3 Hz, 1H), 1.56 (s, 2H), 1.40-1.32 (m, 9H); ES-LCMS m/z 482.1 [M+H]⁺.

### Step 4: tert-Butyl (3S)-3-[[4-[3-[4-[(2-chlorophenyl)sulfonylamino]-2-fluoro-phenoxy]pyrazin-2-yl]pyrimidin-2-yl]amino]piperidine-1-carboxylate

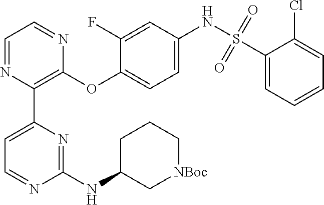 [SMILES: CC(C)(C)OC(=O)N1CCC[C@H](Nc2nccc(-c3nccnc3Oc3ccc(NS(=O)(=O)c4ccccc4Cl)cc3F)n2)C1]

To a solution of tert-butyl (3S)-3-[[4-[3-(4-amino-2-fluoro-phenoxy)pyrazin-2-yl]pyrimidin-2-yl]amino]piperidine-1-carboxylate (80 mg, 141.22 μmol, 1 eq) in DCM (1 mL) was added pyridine (111.70 mg, 1.41 mmol, 113.98 μL, 10 eq) and 2-chlorobenzenesulfonyl chloride (149.03 mg, 706.10 μmol, 96.27 μL, 5 eq). The mixture was stirred at 20° C. for 12 h. The reaction mixture was diluted with H₂O (50 mL) and extracted with EtOAc (50 mL×3). The combined organic layers were washed with brine (50 mL), dried over Na₂SO₄, filtered and concentrated under reduced pressure to yield a residue which was purified by preparative TLC (PE/EtOAc=0/1, TLC: PE/EtOAc=0/1, R_{f}=0.40) to yield tert-butyl (3S)-3-[[4-[3-[4-[(2-chlorophenyl)sulfonylamino]-2-fluoro-phenoxy]pyrazin-2-yl]pyrimidin-2-yl]amino]piperidine-1-carboxylate (50 mg, 70.11 μmol, 65.7% yield, 92.0% purity) as a yellow oil. ¹H NMR (400 MHz, DMSO-d₆) δ ppm 10.97 (s, 1H), 8.54-8.41 (m, 2H), 8.25 (d, J=2.5 Hz, 1H), 8.10 (d, J=8.3 Hz, 1H), 7.68 (d, J=4.0 Hz, 2H), 7.61-7.53 (m, 1H), 7.35-7.21 (m, 2H), 7.13-7.07 (m, 1H), 7.05 (d, J=2.3 Hz, 1H), 6.95 (d, J=9.3 Hz, 1H), 3.80-3.56 (m, 3H), 3.22-3.11 (m, 2H), 1.92-1.86 (m, 1H), 1.70-1.61 (m, 1H), 1.55-1.43 (m, 2H), 1.40-1.21 (m, 9H); ES-LCMS m/z 656.0 [M+H]⁺.

### Step 5: 2-Chloro-N-[3-fluoro-4-[3-[2-[[(3S)-3-piperidyl]amino]pyrimidin-4-yl]pyrazin-2-yl]oxy-phenyl]benzenesulfonamide

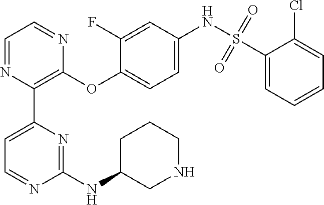 [SMILES: O=S(=O)(Nc1ccc(Oc2nccnc2-c2ccnc(N[C@H]3CCCNC3)n2)c(F)c1)c1ccccc1Cl]

To a stirred solution of tert-butyl (3S)-3-[[4-[3-[4-[(2-chlorophenyl)sulfonylamino]-2-fluoro-phenoxy]pyrazin-2-yl]pyrimidin-2-yl]amino]piperidine-1-carboxylate (50 mg, 70.11 μmol, 1 eq) in DCM (2 mL) was added TFA (2 mL) at 0° C. The reaction mixture was stirred at 25° C. for 0.5 h. The reaction mixture was neutralized to pH=7 with NH₃·H₂O at 0° C. and extracted with DCM (20 mL×3). The combined organic layers were washed with brine (30 mL), dried over Na₂SO₄, filtered and concentrated under reduced pressure to yield a residue which was purified by preparative HPLC (FA condition: column: Welch Xtimate C18 150*25 mm*5 μm; mobile phase: [water (FA)-ACN]; gradient: 11%-41% B over 10 min) to yield 2-chloro-N-[3-fluoro-4-[3-[2-[[(3S)-3-piperidyl]amino]pyrimidin-4-yl]pyrazin-2-yl]oxy-phenyl]benzenesulfonamide (13.9 mg, 23.45 μmol, 33.4% yield, 98.552% purity, 0.8FA) as a yellow solid. ¹H NMR (400 MHz, DMSO-d₆) δ ppm 8.48-8.45 (m, 2H), 8.28 (s, 1H), 8.25 (d, J=2.6 Hz, 1H), 8.03 (dd, J=1.4, 7.7 Hz, 1H), 7.57-7.40 (m, 4H), 7.13 (d, J=5.0 Hz, 1H), 7.06 (t, J=9.0 Hz, 1H), 6.84 (dd, J=2.4, 13.3 Hz, 1H), 6.74 (dd, J=1.8, 8.7 Hz, 1H), 4.02 (dd, J=2.4, 5.3 Hz, 2H), 3.27 (d, J=10.4 Hz, 1H), 3.08-2.98 (m, 1H), 2.78-2.66 (m, 2H), 1.99-1.90 (m, 1H), 1.83-1.66 (m, 1H), 1.63-1.44 (in, 2H); ES-LCMS m/z 556.2 [M+H]⁺.

The following compound was made by processes similar to those described immediately above using Method B:

### Example 10. N-[4-[(1r,4r)[3-[2-[(4-Aminocyclohexyl)amino]pyrimidin-4-yl]pyrazin-2-yl]oxy]-3-fluorophenyl]2-chlorobenzenesulfonamide (Compound 402)

The following compounds were made using Method C:

### Example 11. N-[3-Fluoro-4-[[5-[2-[[(3S)-3-piperidinyl]amino]pyrimidin-4-yl]-4-thiazolyl]oxy]phenyl]2-chlorobenzenesulfonamide (Compound Number 391)

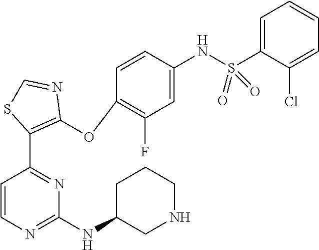 [SMILES: O=S(=O)(Nc1ccc(Oc2ncsc2-c2ccnc(N[C@H]3CCCNC3)n2)c(F)c1)c1ccccc1Cl]

### Step 1: 1-(4-Chlorothiazol-5-yl)ethanol

To a solution of 4-chlorothiazole-5-carbaldehyde (1 g, 6.76 mmol, 1.0 eq.) in THF (30 mL) was added methyl magnesium iodide (3.0 M in THF. 3.4 mL, 10.14 mmol, 1.5 eq.) at −40° C. and stirred for 30 min at same temperature. After reaction completion, saturated NH₄Cl solution (2 mL) was added to the reaction mixture and followed by extraction with EtOAc (3×20 ml). The combined organic layers were dried over sodium sulfate, concentrated and purified by column chromatography (60-120 silica gel; gradient elution with 10% EtOAc in hexane) to afford 1-(4-chlorothiazol-5-yl)ethanol (970 mg, 98%) as a pale yellow solid.

¹H-NMR (300 MHz, CDCl₃): δ 8.64 (s, 1H), 5.32-5.24 (m, 1H), 1.57 (d, J=2.7 Hz, 3H). MS (ESI pos. ion) m/z: 164.2.

### Step 2: 1-(4-Chlorothiazol-5-yl)ethanone

To a solution of 1-(4-chlorothiazol-5-yl)ethanol (970 mg, 5.95 mmol, 1.0 eq.) in DCM (30 mL) was added pyridinium chlorochromate (3.85 g, 17.85 mmol, 3.0 eq.) portion-wise at 25° C. over a period of 5 min and the reaction was stirred for 6 h at same temperature. After reaction completion, the mixture was filtered through celite pad and washed with DCM (15 ml). The filtrate was concentrated and purified by column chromatography (60-120 silica gel; gradient elution with 20% EtOAc in hexane) to afford 1-(4-chlorothiazol-5-yl)ethanone (800 mg, 83%) as a white solid.

¹H-NMR (300 MHz, CDCl₃): δ 8.84 (s, 1H), 2.72 (s, 3H). MS (ESI pos. ion) m/z: 162.1.

### Step 3: E,1-(4-chlorothiazol-5-yl)-3-(N,N-dimethylamino)prop-2-en-1-one

A solution of 1-(4-chlorothiazol-5-yl)ethanone (800 mg, 4.90 mmol, 1.0 eq.) in DMF dimethylacetal (8 mL) was stirred at 80° C. for 8 h. After reaction completion, the mixture was poured into ice water and extracted with EtOAc (3×20 mL). The combined organic layers were dried and concentrated to obtain E,1-(4-chlorothiazol-5-yl)-3-(N,N-dimethylamino)prop-2-en-1-one (880 mg, 82%) as a brown solid.

¹H-NMR (300 MHz, CDCl₃): δ 8.71 (s. 1H), 7.85 (d, J=7.2 Hz, 1H), 6.03 (d, J=7.2 Hz, 1H), 3.20 (s, 3H), 2.97 (s, 3H). MS (ESI pos. ion) m/z: 217.2.

### Step 4: 2-((S)-1,N-(t-Butoxycarbonyl)piperidin-3-ylamino)-4-(4-chlorothiazol-5-yl)pyrimidine

To a solution of N—((S)-1-(N-t-butoxycarbonyl)piperidin-3-yl) guanidine (1.28 g, 4.62 mmol, 2.0 eq.) in EtOH (10 mL) was added K₂CO₃ (1.91 g, 13.9 mmol, 6.0 eq.) followed by E,1-(4-chlorothiazol-5-yl)-3-(N,N-dimethylamino)prop-2-en-1-one (500 mg, 2.31 mmol, 1.0 eq.) at 25° C. and the mixture was stirred at 80° C. for 18 h. After reaction completion, the mixture was filtered, concentrated and purified by column chromatography (60-120 silica gel, gradient elution with 20% EtOAc in hexane) to afford 2-((S)-1,N-(t-butoxycarbonyl)piperidin-3-ylamino)-4-(4-chlorothiazol-5-yl)pyrimidine (350 mg, 38%) as a white solid.

¹H-NMR (500 MHz, CDCl₃): δ 8.75 (s, 1H), 8.39 (d, J=5 Hz, 1H), 7.49 (d, J=5 Hz, 1H), 5.21 (d, J=7 Hz, 1H), 3.99-3.96 (m, 1H), 3.71-3.66 (m, 1H), 3.47-3.42 (m, 2H), 1.98-1.94 (m, 1H), 1.71-1.66 (m, 2H), 1.61-1.57 (m, 2H), 1.40 (s, 9H). MS (ESI pos. ion) m/z: 396.4.

### Step 5: O-tert-Butyl N-)(3S)-3-[[4-[4-[4-[(2-chlorophenyl)sulfonylamino]-3-fluorophenoxy]thiazol-5-yl]pyrimidin-2-yl]amino]piperidin-1-yl) carboxylate

4-(2-Chlorophenylsulfonamido)-2-fluorophenol (100 mg, 0.30 mmol) in DMF (3 mL), 2-((S)-1,N-(t-butoxycarbonyl)piperidin-3-ylamino)-4-(4-chlorothiazol-5-yl)pyrimidine (131 mg, 0.33 mmol) and Cs₂CO₃ (395 mg, 0.99 mmol) were added to a 10 mL microwave tube and heated to 180° C. for 45 min. The reaction mixture was diluted with EtOAc (30 mL) and filtered. The solvent was evaporated in vacuo. The residue was purified by silica gel column chromatography (60-120 silica gel) using hexane/ethyl acetate as eluent (70:30) to afford O-tert-Butyl N-)(3S)-3-[[4-[4-[4-[(2-chlorophenyl)sulfonylamino]-2-fluorophenoxy]thiazol-5-yl]pyrimidin-2-yl]amino]piperidin-1-yl) carboxylate (60 mg, 55%) as a gummy material. MS (ESI pos. ion) m/z: 661.3.

### Step 6: N-[3-Fluoro-4-[[5-[2-[[(3S)-3-piperidinyl]amino]pyrimidin-4-yl]-4-thiazolyl]oxy]phenyl]2-chlorobenzenesulfonamide (Compound Number 391)

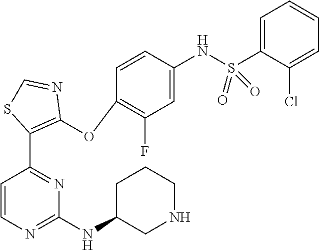 [SMILES: O=S(=O)(Nc1ccc(Oc2ncsc2-c2ccnc(N[C@H]3CCCNC3)n2)c(F)c1)c1ccccc1Cl]

To a stirred solution of O-tert-Butyl N-)(3S)-3-[[4-[4-[4-[(2-chlorophenyl)sulfonylamino]-2-fluorophenoxy]thiazol-5-yl]pyrimidin-2-yl]amino]piperidin-1-yl) carboxylate (60 mg, 0.09 mmol) in DCM (3 mL) was added TFA (0.3 mL) dropwise at 0° C. The mixture was stirred 2 h at room temperature, concentrated in vacuo and purified by prep-HPLC to afford N-[3-fluoro-4-[[5-[2-[[(3S)-3-piperidinyl]amino]pyrimidin-4-yl]-4-thiazolyl]oxy]phenyl]2-chlorobenzenesulfonamide TFA salt (15 mg, 30%) as a brown colored solid.

¹H NMR (300 MHz, DMSO-d₆): δ 10.33 (s, 1H), 9.08 (s, 1H), 8.68 (bs, 2H), 8.35 (d, J=5.4 Hz, 1H), 7.88-7.85 (m, 1H), 7.71-7.62 (m, 2H), 7.50-7.45 (m, 2H), 7.22 (t, J=8.7 Hz, 1H), 7.14-7.10 (m, 2H), 6.93-6.90 (m, 1H), 4.09 (br, 1H), 3.35 (d, J=9.9 Hz, 1H), 3.18 (d, J=14.1 Hz, 1H), 2.88-2.85 (m, 2H), 1.92-1.87 (m, 2H), 1.70-1.55 (m, 2H). MS (ESI pos. ion) m/z: 561.3.

The following compound was made by processes similar to those described immediately above using Method C:

### Example 11. N-[4-[(1r,4r)[5-[2-[(4-Aminocyclohexyl)amino]pyrimidin-4-yl]thiazol-4-yl]oxy]-3-fluorophenyl]2-chlorobenzenesulfonamide (Compound 403)

### Example 12. Biological Data

### Surface Plasmon Resonance (SPR) Method Description

Compound interaction was profiled using a Biacore 8K instrument (Cytiva). Biotinylated N-terminal avi-tagged Cdk2(2-298) or Cdk1(1-297) was immobilized on a series S sensor Chip SA at a capture level between 2500 RU and 3000 RU (Cdk2) or 7000 RU (Cdk1) in buffer (25 mM HEPES, 150 mM NaCl, 0.05% Tween20, pH 7.5). A compound dilution plate (8 concentrations, 3-fold dilution) in DMSO was prepared (100× assay concentration). From this plate, compound DMSO stock solution was transferred into buffer, yielding a final DMSO concentration of 1% in assay. The remaining buffer was supplemented with 1% DMSO. Compounds were injected in a series of increasing concentrations in multi-cycle kinetics (MCK, “M”) or single-cycle kinetics (SCK, “S”) mode at a flow rate of 30 μl/min for 120 sec and dissociation was profiled for 180 sec (MCK) or 1800 sec (SCK). Data were analysed using Biacore Insight Evaluation Software (Cytiva). Results are summarized in Table 3.

In Table 3 below. “A” indicates a K_{D} of 10 nM or less; “B” indicates a K_{D} of 11-100 nM; “C” indicates a K_{D} of 100-1000 nM; “D” indicates a K_{D} of 1000-5000 nM; “E” indicates a K_{D} of >5000 nM.

TABLE 3
Biological Data for Selected Compounds

| Compound Number | Activity rating* | M/S (CK) |
|---|---|---|
| 390 | B | S |
| 391 | A | S |
| 402 | C | S |
| 403 | A | S |
| 410 | A | S |
| 411 | B | S |
| 412 | B | S |
| 413 | A | S |
| 414 | A | S |
| 415 | B | S |
| 416 | B | S |
| 417 | C | S |

1. A method of inhibiting CDK2 activity in a subject in need thereof, comprising administering to the subject an effective amount of a compound of Formula (I): 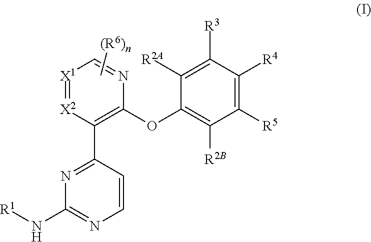
or a pharmaceutically acceptable salt thereof, wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1c}, or —(C₁₋₆ alkylene)-NR^{1a}R^{1b}; wherein the C₃₋₁₂ cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the C₁₋₆ alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}—, C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen, C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{e}, —S(O)₂R^{e}, —S(O)(═NH)R^{c}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, —N(R^{f})S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R¹¹ are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{e} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(R′)C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkenyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C6-10 aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(R^{f2})S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
X¹ and X² are both C, optionally substituted with R⁶;
or X¹ is C, optionally substituted with R⁶, and X² is N;
or X¹ and X² taken together are a sulfur atom;
or a pharmaceutical composition comprising: (a) a compound of Formula (I) or a pharmaceutically acceptable salt thereof, and (b) a pharmaceutically acceptable excipient.

2. A method treating, preventing, and/or reducing the risk or severity of a CDK2-related disease or disorder in a subject in need thereof, comprising administering to the subject an effective amount of a compound of Formula (I): 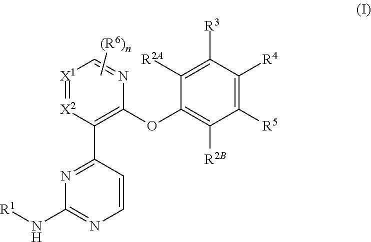
or a pharmaceutically acceptable salt thereof, wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1c}, or —(C₁₋₆ alkylene)-NR^{1a}R^{1b}; wherein the C₃₋₁₂ cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the C₁₋₆ alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R′)SO₂R⁹, —C(O)NR^{8A}R^{8B}, —C(O)R⁷, —C(O)OR^{7A}, —SO₂R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen, C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{e}, —S(O)(═NH)R^{e}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, —N(R^{f})S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{e} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(R^{f1})C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C6-10 aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(R^{f2})S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
X¹ and X² are both C, optionally substituted with R⁶;
or X¹ is C, optionally substituted with R⁶, and X2 is N;
or X¹ and X² taken together are a sulfur atom;
or a pharmaceutical composition comprising: (a) a compound of Formula (I) or a pharmaceutically acceptable salt thereof, and (b) a pharmaceutically acceptable excipient.

3. The method of claim 2, wherein the CDK2-related disease or disorder is cancer identified as having amplification or overexpression of cyclin E.

4. The method of claim 3, wherein cyclin E is CCNE1 or CCNE2.

5. The method of claim 2, wherein the CDK2-related disease or disorder is cancer identified as having amplification or overexpression of cyclin A.

6. The method of any one of claims 3-5, wherein the cancer is breast cancer, ovary cancer, cervix cancer, prostate cancer, testis cancer, genitourinary tract cancer, esophagus cancer, larynx cancer, glioblastoma, neuroblastoma, stomach cancer, skin cancer, keratoacanthoma, lung cancer, epidermoid carcinoma, large cell cancer, non-small cell lung cancer (NSCLC), small cell carcinoma, lung adenocarcinoma, bone cancer, colon cancer, adenoma, pancreatic cancer, adenocarcinoma, thyroid cancer, follicular carcinoma, undifferentiated carcinoma, papillary carcinoma, seminoma, melanoma, sarcoma, bladder carcinoma, liver carcinoma and biliary passages, kidney carcinoma, buccal cavity cancer, naso-pharyngeal cancer, pharynx cancer, lip cancer, tongue cancer, mouth cancer, small intestine cancer, colon-rectum cancer, large intestine cancer, rectum cancer, bronchial cancer, hepatocellular cancer, gastric cancer, endometrial cancer, melanoma, renal cancer, urinary bladder cancer, uterine corpus cancer, or uterine cervix cancer.

7. The method of claim 2, wherein the CDK2-related disease or disorder is an autoimmune disorder or disease identified as having amplification or overexpression of cyclin E.

8. The method of claim 7, wherein cyclin E is CCNE1 or CCNE2.

9. The method of claim 2, wherein the CDK2-related disease or disorder is an autoimmune disorder or disease identified as having amplification or overexpression of cyclin A.

10. The method of any one of claims 7-9, wherein the autoimmune disorder or disease is rheumatoid arthritis, Lupus, Crohn's Disease, Addison disease, celiac disease, dermatomyositis, Graves' disease, thyroiditis, multiple sclerosis, pernicious anemia, reactive arthritis, pemphigus vulgaris, diabetes mellitus type 1 (IDDM), systemic lupus erythematosus (SLE), Sjogren's syndrome, Churg-Strauss Syndrome, Hashimoto's thyroiditis, idiopathic thrombocytopenic purpura, or rheumatoid arthritis.

11. The method of claim 2, wherein the CDK2-related disease or disorder is an inflammatory disease or disorder identified as having amplification or overexpression of cyclin E.

12. The method of claim 11, wherein cyclin E is CCNE1 or CCNE2.

13. The method of claim 2, wherein the CDK2-related disease or disorder is an inflammatory disease or disorder identified as having amplification or overexpression of cyclin A.

14. The method of any one of claims 11-13, wherein the inflammatory disease or disorder is asthma, chronic peptic ulcers, psoriasis, inflammatory bowel disease, tuberculosis, rheumatoid arthritis, periodontitis, ulcerative colitis, hepatitis chronic prostatitis, glomerulonephritis, hypersensitivities, pelvic inflammatory disease, reperfusion injury, transplant rejection, or vasculitis.

15. The method of claim 2, wherein the CDK2-related disease or disorder is a neurodegenerative disease or disorder identified as having amplification or overexpression of cyclin E.

16. The method of claim 15, wherein cyclin E is CCNE1 or CCNE2.

17. The method of claim 2, wherein the CDK2-related disease or disorder is a neurodegenerative disease or disorder identified as having amplification or overexpression of cyclin A.

18. The method of any one of claims 15-17, wherein the neurodegenerative disease or disorder is Alzheimer's disease, AIDS-related dementia, Parkinson's disease, amyotrophic lateral sclerosis, retinitis pigmentosa, spinal muscular atrophy, cerebellar degeneration, Alexander's disease, Alper's disease, Ataxia telangiectasia, Batten disease (also known as Spielmeyer-Vogt-Sjogren-Batten disease), Bovine spongiform encephalopathy (BSF), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

19. The method of any one of claims 1-18, further comprising administering to the subject an additional therapeutic agent.

20. A method of killing a cell over-expressing or amplifying cyclin E, comprising contacting the cell with an effective amount of a compound of Formula (I): 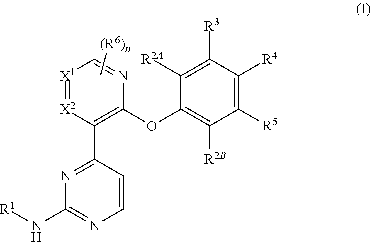
or a pharmaceutically acceptable salt thereof, wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1e}, or —(C₁₋₆ alkylene)-NR^{1a}R^{1b}; wherein the C3-12 cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the CL-6 alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}, —C(O)R⁷, —C(O)OR^{7A}, —SO2R⁹, —NR⁸S(O)(═NR^{8C})R⁹, or —SO₂NR^{8A}R^{8B}; wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen, C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁-s alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{e}, —S(O)(═NH)R^{e}, —S(O)₂NR^{c}R^{d}, —NR^{c}R^{d}, —N(R)C(O)R^{a}, —N(R^{f})C(O)OR^{b}, —N(R^{f})C(O)NR^{c}R^{d}, —N(R^{f})S(O)₂R^{e}, —N(R^{f})S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R¹¹ are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{o} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{e1}, —S(O)₂R^{e1}, —S(O)₂NR^{c1}R^{d1}, —NR^{c1}R^{d1}, —N(R^{f1})C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{e1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C6-10 aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(R^{f2})S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
X¹ and X² are both C, optionally substituted with R⁶;
or X¹ is C, optionally substituted with R⁶, and X2 is N;
or X¹ and X² taken together are a sulfur atom;
or a pharmaceutical composition comprising: (a) a compound of Formula (I) or a pharmaceutically acceptable salt thereof, and (b) a pharmaceutically acceptable excipient.

21. A method of killing a cell over-expressing or amplifying cyclin A, comprising contacting the cell with an effective amount of a compound of Formula (I): 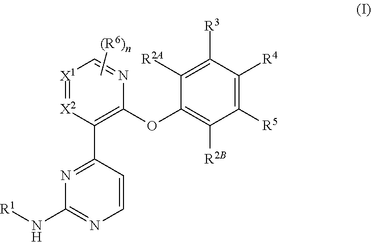
or a pharmaceutically acceptable salt thereof, wherein:
R¹ is C₃₋₁₂ cycloalkyl, 3- to 14-membered heterocyclyl, 5- to 14-membered heteroaryl, —(C₁₋₆ alkylene)-(C₃-C₁₂ cycloalkyl), or —(C₁₋₆ alkylene)-(3- to 14-membered heterocyclyl), —(C₁₋₆ alkylene)-OR^{1c}, or —(C₁₋₆ alkylene)-NR^{1g}R^{1h}; wherein the C3-12 cycloalkyl, 3- to 14-membered heterocyclyl, 5- to 14-membered heteroaryl, and C₁₋₆ alkylene of R¹ are independently optionally substituted with 1, 2, 3, 4 or 5 substituents independently selected from R¹⁰;
each R^{1a}, R^{1b} and R^{1c} is independently hydrogen, C₁₋₆ alkyl, C₃₋₈ cycloalkyl, C₆₋₁₀ aryl, 5- to 14-membered heteroaryl or 3- to 12-membered heterocyclyl; wherein the C₁₋₆ alkyl, C₃₋₈ cycloalkyl, C₆₋₁₀ aryl, 5- to 14-membered heteroaryl and 3- to 12-membered heterocyclyl of R^{1a} and R^{1b} are optionally substituted with 1, 2, 3, 4 or 5 substituents independently selected from R¹⁰;
R^{2A} and R^{2B} are independently hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R³ is hydrogen, halogen, cyano, C₁₋₆ alkyl, C₁₋₆ haloalkyl, C₃₋₆ cycloalkyl, —O(C₁₋₆ alkyl), or —O(C₁₋₆ haloalkyl);
R⁴ and R⁵ are independently hydrogen, halogen, cyano, nitro, C₁₋₆ alkyl, C₂₋₆ alkenyl, C₂₋₆ alkynyl, C₃₋₁₂ cycloalkyl, C₆₋₂₀ aryl, 3- to 14-membered heterocyclyl, 5- to 14-membered heteroaryl, —OR^{7A}, —NR^{8A}R^{8B}, —NR⁸C(O)R⁷, —NR⁸C(O)OR^{7A}, —NR⁸C(O)NR^{8A}R^{8B}, —NR⁸SO₂R⁹, —NR⁸SO₂NR^{8A}R^{8B}, —NR⁸S(O)(═NR^{8C})R⁹, —C(O)N(R⁸)SO₂R⁹, —C(O)NR^{8A}R^{8B}, —C(O)R⁷, —C(O)OR^{7A}, —SO2R⁹, —NR⁸S(O)(═NR^{8C})R⁹, or —SO₂NR^{8A}R^{8B}, wherein the C₁₋₆ alkyl, C₃₋₁₂ cycloalkyl, C₆₋₂₀ aryl, 3- to 14-membered heterocyclyl, and 5- to 14-membered heteroaryl of R⁴ and R⁵ are optionally substituted with 1, 2, 3, 4 or 5 substituents independently selected from R¹⁰;
n is 0, 1, 2, or 3;
each R⁶ is independently halogen, cyano, nitro, C₁₋₆ alkyl, C₁₋₆ haloalkyl, C₃₋₆ cycloalkyl, —O(C₁₋₆ alkyl), —O(C₁₋₆ haloalkyl), —SO₂(C₁₋₆ alkyl) or —SO₂(C₁₋₆ haloalkyl);
each R⁷ is independently hydrogen, NHR⁹, C₁₋₆ alkyl, C₂₋₆ alkenyl, C₃₋₈ cycloalkyl, 5- to 14-membered heteroaryl, or 3- to 12-membered heterocyclyl; wherein the C₁₋₆ alkyl, C₂₋₆ alkenyl, C₃₋₈ cycloalkyl, 5- to 14-membered heteroaryl and 3- to 12-membered heterocyclyl of R⁷ and R^{7A} are optionally substituted with 1, 2, 3, 4 or 5 substituents independently selected from R¹⁰;
each R^{7A} is independently hydrogen, C₁₋₆ alkyl, C₂₋₆ alkenyl, C₃₋₈ cycloalkyl, C₆₋₁₀ aryl, 5- to 14-membered heteroaryl, or 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl of R⁷ and R^{7A} are optionally substituted with 1, 2, 3, 4 or 5 substituents independently selected from R¹⁰;
each R⁸ is independently hydrogen or C₁-C₆ alkyl;
each R^{8A} is independently hydrogen or C₁-C₆ alkyl;
each R^{8B} is independently hydrogen, C₁₋₆ alkyl, C₂₋₆ alkenyl, C₃₋₈ cycloalkyl, C₆₋₁₀ aryl, or 3- to 12-membered heterocyclyl; wherein the C₁₋₆ alkyl, C₂₋₆ alkenyl, C₃₋₈ cycloalkyl, C₆₋₁₀ aryl, and 3- to 12-membered heterocyclyl of R^{8B} are optionally substituted with 1, 2, 3, 4 or 5 substituents independently selected from R¹⁰;
each R^{8C} is independently hydrogen or C₁-C₆ alkyl;
each R⁹ is independently C₁₋₆ alkyl, C₂₋₆ alkenyl, C₃₋₈ cycloalkyl, C₆₋₁₀ aryl, 5- to 14-membered heteroaryl and 3- to 12-membered heterocyclyl; wherein the C₁₋₆ alkyl, C₂₋₆ alkenyl, C₃₋₈ cycloalkyl, C₆₋₁₀ aryl, 5- to 14-membered heteroaryl and 3- to 12-membered heterocyclyl are optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{e}, —S(O)₂R^{e}, —S(O)(═NH)R^{e}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{e}, —N(R^{f})S(O)₂NR^{c}R^{d}, —P(O)R″R^{h} or —SiR′RiR^{k}; wherein the C₁₋₆ alkyl, C₂₋₆ alkenyl, C₂₋₆ alkynyl, C₃₋₈ cycloalkyl, Ce-i4 aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{e} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{o} are each optionally substituted with 1, 2, 3 or 4 substituents independently selected from R¹¹;
each R^{f} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl, or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each Rⁱ, R^{j} and R^{k} is independently C₁₋₆ alkyl;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{c1}, —S(O)₂R^{e1}, —S(O)₂NR^{c1}R^{d1}, —NR^{c1}R^{d1}, —N(R^{f1})C(O)R^{a1}, —N(Rⁿ)C(O)OR^{b1}, —N(R^{f1})C(O)NR^{c1}R^{d1}, —N(R^{f1})S(O)₂R^{e1}, —N(R^{f1})S(O)₂NR^{c1}R^{d1}, —P(O)R^{g1}R^{h1}, or —SiRⁱ¹R^{j1}R^{k1}; wherein the C₁₋₆ alkyl, C₂₋₆ alkenyl, C₂₋₆ alkynyl, C₃₋₆ cycloalkyl, C₆₋₁₄ aryl, 5- to 14-membered heteroaryl and 3- to 14-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{e1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R₆₋₁₀ are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each Rⁱ¹, R^{j1} and R^{k1} is independently C₁₋₆ alkyl;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{e2}, —S(O)₂NR^{c2}R^{d2}, —NR^{c2}R^{d2}, —N(R^{f2})C(O)R^{a2}, —N(R^{f2})C(O)OR^{b2}, —N(R^{f2})C(O)NR^{c2}R^{d2}, —N(R^{f2})S(O)₂R^{e2}, —N(R^{f2})S(O)₂NR^{c2}R^{d2}, or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³; each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{e2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{e2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
X¹ and X² are both C, optionally substituted with R⁶;
or X¹ is C, optionally substituted with R⁶, and X2 is N;
or X¹ and X² taken together are a sulfur atom;
or a pharmaceutical composition comprising: (a) a compound of Formula (I) or a pharmaceutically acceptable salt thereof, and (b) a pharmaceutically acceptable excipient.

22. The method of any one of claims 1-21, wherein the compound is of Formula (Ia): 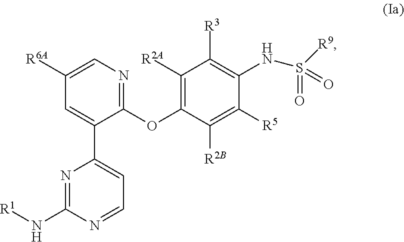
or a pharmaceutically acceptable salt thereof, wherein R^{6A} is hydrogen or R⁶.

23. The method of any one of claims 1-21, wherein the compound is of Formula (Ib): 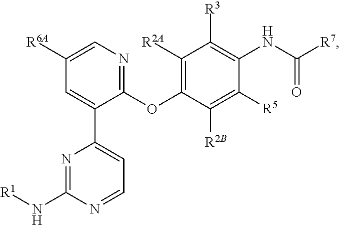
or a pharmaceutically acceptable salt thereof, wherein R^{6A} is hydrogen or R⁶.

24. The method of any one of claims 1-21, wherein the compound is of Formula (Ic): 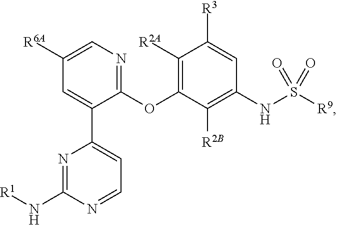
or a planimetrically acceptable salt thereof, wherein R^{6A} is hydrogen or R⁶.

25. The method of any one of claims 1-21, wherein the compound is of Formula (Id): 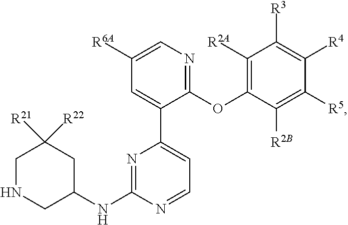
or a pharmaceutically acceptable salt thereof, wherein R^{6A} is hydrogen or R⁶; R²¹ and R²² are independently H, F, —CH₃ or —NH₂.

26. The method of any one of claims 1-21, wherein the compound is of Formula (Id-1), (Id-2), (Id-3), (Id-4), (Id-5), (Id-6) or (Id-7): 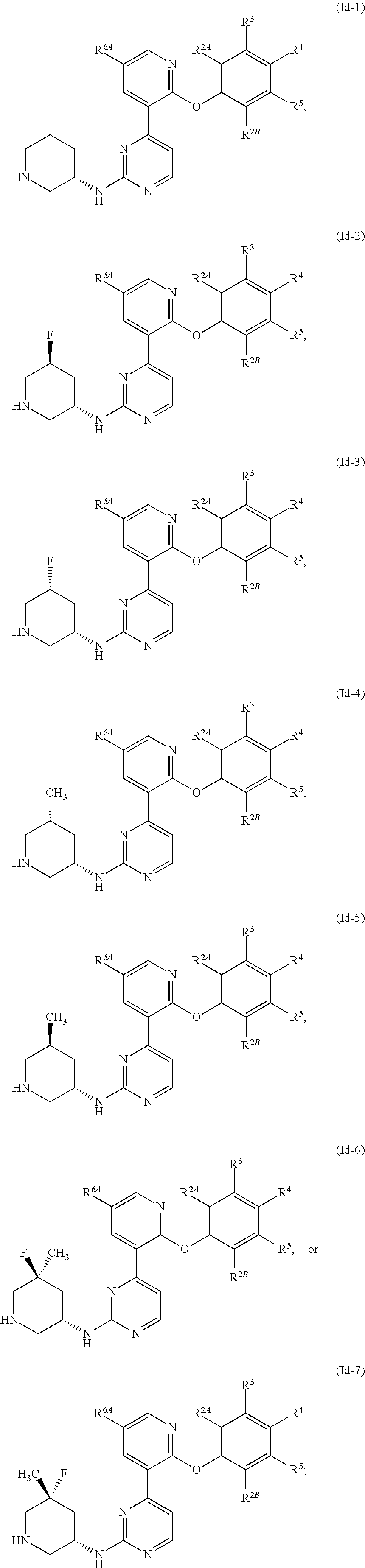
or a pharmaceutically acceptable salt thereof, wherein R^{6A} is hydrogen or R⁶.

27. The method of any one of claims 1-21, wherein the compound is of Formula (Ie): 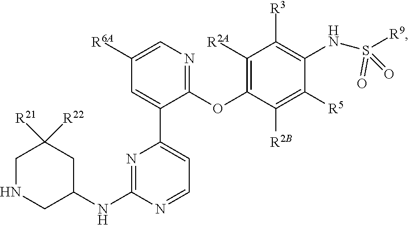
or a pharmaceutically acceptable salt thereof, wherein R^{6A} is hydrogen or R⁶; R²¹ and R²² are independently H, F, —CH₃ or —NH₂.

28. The method of any one of claims 1-21, wherein the compound is of Formula (Ie-1), (Ie-2), (Ie-3), (Ie-4), (Ie-5), (Ie-6) or (Ie-7): 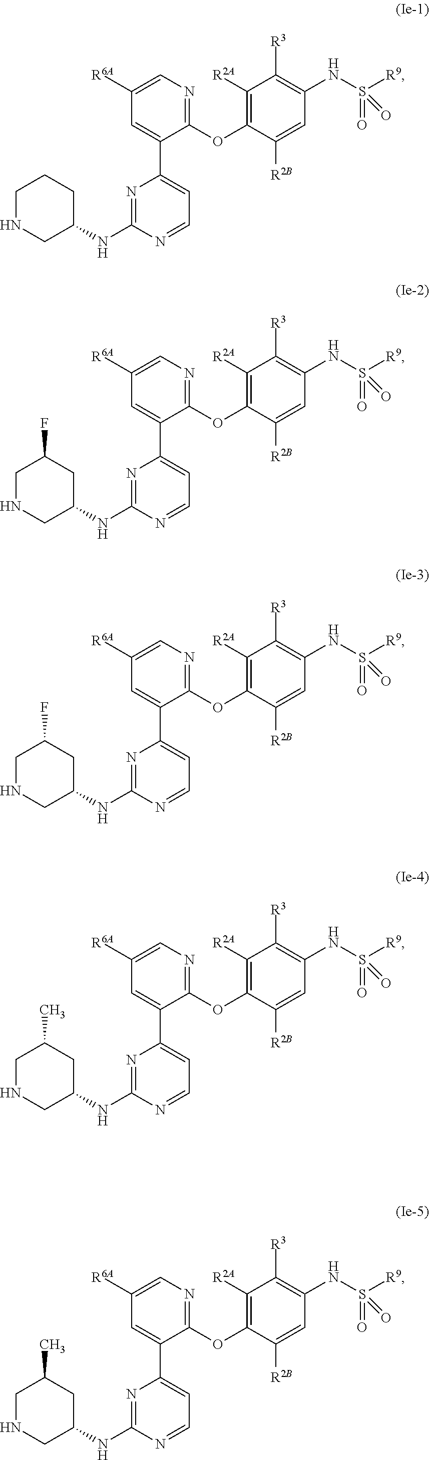 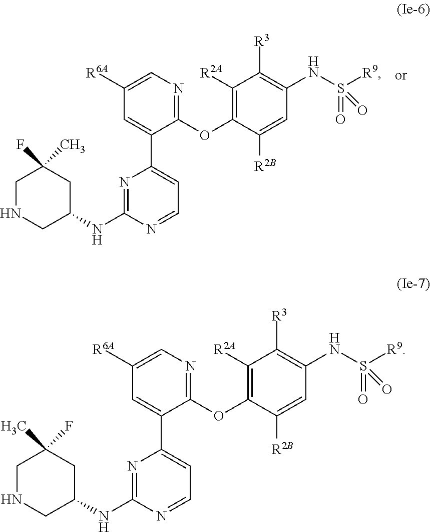
or a pharmaceutically acceptable salt thereof, wherein R^{6A} is hydrogen or R⁶.

29. The method of any one of claims 1-21, wherein the compound is selected from Compound Nos. 101-247 in Table 2, or a pharmaceutically acceptable salt thereof.

30. The method of any one of claims 1-21, wherein the compound is selected from Compound Nos. 101-238, 248-380 and 382-389 in Table 2, or a pharmaceutically acceptable salt thereof.

31. The method of any one of claims 1-21, wherein the compound is selected from Compound Nos. 248-380 and 382-389 in Table 2, or a pharmaceutically acceptable salt thereof.

32. The method of any one of claims 1-21, wherein the compound is: 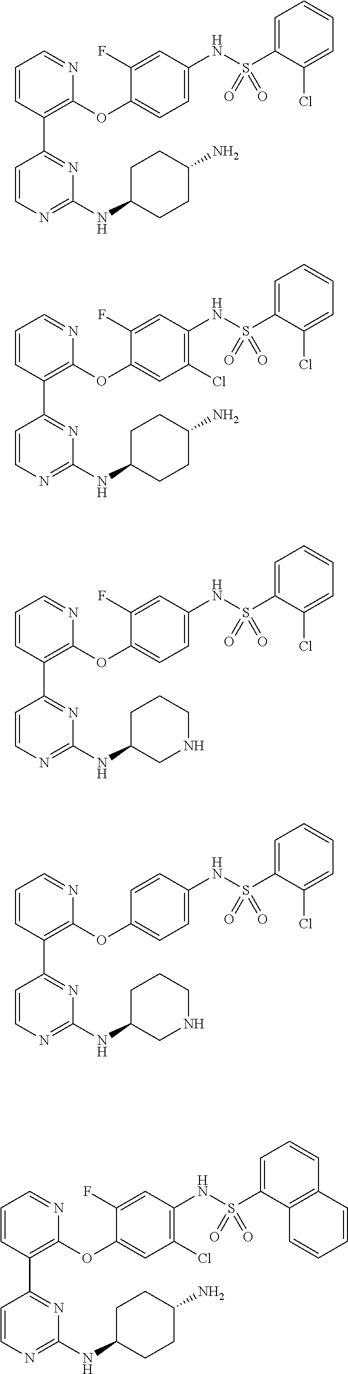 [SMILES: N[C@H]1CC[C@H](Nc2nccc(-c3cccnc3Oc3ccc(NS(=O)(=O)c4ccccc4Cl)cc3F)n2)CC1 ; N[C@H]1CC[C@H](Nc2nccc(-c3cccnc3Oc3cc(Cl)c(NS(=O)(=O)c4ccccc4Cl)cc3F)n2)CC1 ; O=S(=O)(Nc1ccc(Oc2ncccc2-c2ccnc(N[C@H]3CCCNC3)n2)c(F)c1)c1ccccc1Cl ; O=S(=O)(Nc1ccc(Oc2ncccc2-c2ccnc(N[C@H]3CCCNC3)n2)cc1)c1ccccc1Cl ; N[C@H]1CC[C@H](Nc2nccc(-c3cccnc3Oc3cc(Cl)c(NS(=O)(=O)c4cccc5ccccc45)cc3F)n2)CC1] 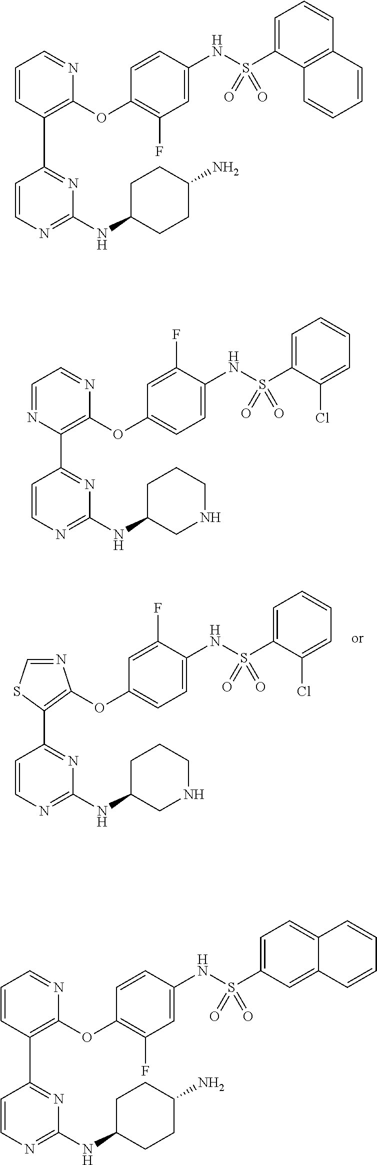 [SMILES: N[C@H]1CC[C@H](Nc2nccc(-c3cccnc3Oc3ccc(NS(=O)(=O)c4cccc5ccccc45)cc3F)n2)CC1 ; O=S(=O)(Nc1ccc(Oc2nccnc2-c2ccnc(N[C@H]3CCCNC3)n2)cc1F)c1ccccc1Cl ; O=S(=O)(Nc1ccc(Oc2ncsc2-c2ccnc(N[C@H]3CCCNC3)n2)cc1F)c1ccccc1Cl ; N[C@H]1CC[C@H](Nc2nccc(-c3cccnc3Oc3ccc(NS(=O)(=O)c4ccc5ccccc5c4)cc3F)n2)CC1]
or a pharmaceutically acceptable salt thereof.
